SEQUENCE LISTING (144 sequences)

SEQ ID NO: 1 - DNA, 2322 bp, unidentified
  source 1..2322: mol_type = genomic DNA; note = Schizosaccharomyces pombe
atgacagaaatagaaaacattggcgcattagaagttctcttctctcctgaatccatcgagcagagcctcaaacggtgtcaactcccctccactttattatacgatgaaaaaggtttacgactgtttgatgagattacgaatttaaaagaatactacctgtatgaaagtgagcttgatattctgaagaagttcagcgattccattgccaaccagttactgtctccagatcttcctaacacggttatagaattagggtgtggaaatatgcgcaaaacaaaacttcttttagatgcgtttgaaaagaagggctgtgatgtgcatttttacgcccttgaccttaatgaagccgagttgcaaaaaggactgcaggagcttcgtcaaactaccaattatcagcatgttaaggtgtctggtatttgcggttgctttgaaagattgctacaatgtttggacaggtttcgtagtgagcccaatagtcgaattagcatgttgtacttgggtgcttcgattggtaattttgataggaaatccgcagcatcatttttacgttcgtttgccagtcgtttgaatattcatgacaaccttttaatctccttcgatcatagaaacaaggctgagctagtccaactagcttacgatgatccttatcgtattactgaaaagtttgaaaagaatattttggctagtgtcaatgcggtttttggtgaaaaccttttcgacgaaaatgattgggaatataaaagtgtctacgatgaagatctcggtgttcatagggcctacttacaagccaaaaatgaagttactgttattaagggtccaatgttttttcaatttaaacctagtcatttaattttgatcgaagaaagttggaagaatagcgatcaagaatgtcgtcaaatcattgagaaaggtgattttaaattagtctctaagtatgaaagtacgattgcagattactcgacctatgttattaccaaacaatttcctgctatgcttcaactccctcttcagccttgtccttcgttagcagaatgggatgctctacgcaaagtatggctttttattacaaataaattgcttaacaaagataacatgtacaccgcatggattcctttgagacatcctccaattttttacatcggacatgtccctgtttttaatgatatttatctcacaaagattgtcaaaaacaaagcaactgctaacaaaaaacatttttgggaatggtttcaacgtggtatagatccggacattgaagatccctccaagtgccattggcattctgaagttcctgaaagctggccttctcctgaccaacttcgtgaatatgagaaagagtcttgggaatatcatattgtaaagttgtgcaaagcaatggatgaattgtctacttctgaaaagagaattctctggctttgttacgaacatgtagccatgcatgtggagacaactctttacatctacgtacagtcatttcaaaatgcaaaccagactgtatcaatttgcggatcacttcctgaaccagctgaaaaacttacgaaagctccgttatgggtgaatgtacctgaaacggaaattgcagttggtatgcccttgacaacacaatacacgagtgttggatcaaatttgcaatcatccgatcttagtgcccatgaaaatacagatgaacttttttattttgcgtgggataatgagaaaccaatgaggaagaaactggtttctagcttttctattgccaatcgtccaatttctaacggtgaatatttagattttatcaataaaaagtcaaaaacagaaagggtgtatccaaagcaatgggcggagattgatggaacgctttacatacgaaccatgtacggcttattaccccttgacgactacttgggttggcctgttatgacttcatacgacgatctaaacaattatgcgagctcccaaggatgcagactaccaactgaggatgaactgaactgtttttacgatcgggttctcgagagaactgatgagccttatgttagtaccgaaggaaaggcaactggttttcaacaattgcaccctttagccctaagtgataattcaagtaatcaaatattcacaggagcatgggaatggacaagtacagttctggagaagcacgaggattttgaacctgaagagctttatccagattatacacgagatttctttgatggaaagcataatgtcgttttgggtggtagctttgctacggctacgcgcatttcaaatagaagaagcttcaggaacttttaccaagctggctataaatatgcatggattggagctagactagtcaaaaactaa

SEQ ID NO: 2 - protein, 773 aa, unidentified
  source 1..773: note = Schizosaccharomyces pombe
MTEIENIGALEVLFSPESIEQSLKRCQLPSTLLYDEKGLRLFDEITNLKEYYLYESELDILKKFSDSIANQLLSPDLPNTVIELGCGNMRKTKLLLDAFEKKGCDVHFYALDLNEAELQKGLQELRQTTNYQHVKVSGICGCFERLLQCLDRFRSEPNSRISMLYLGASIGNFDRKSAASFLRSFASRLNIHDNLLISFDHRNKAELVQLAYDDPYRITEKFEKNILASVNAVFGENLFDENDWEYKSVYDEDLGVHRAYLQAKNEVTVIKGPMFFQFKPSHLILIEESWKNSDQECRQIIEKGDFKLVSKYESTIADYSTYVITKQFPAMLQLPLQPCPSLAEWDALRKVWLFITNKLLNKDNMYTAWIPLRHPPIFYIGHVPVFNDIYLTKIVKNKATANKKHFWEWFQRGIDPDIEDPSKCHWHSEVPESWPSPDQLREYEKESWEYHIVKLCKAMDELSTSEKRILWLCYEHVAMHVETTLYIYVQSFQNANQTVSICGSLPEPAEKLTKAPLWVNVPETEIAVGMPLTTQYTSVGSNLQSSDLSAHENTDELFYFAWDNEKPMRKKLVSSFSIANRPISNGEYLDFINKKSKTERVYPKQWAEIDGTLYIRTMYGLLPLDDYLGWPVMTSYDDLNNYASSQGCRLPTEDELNCFYDRVLERTDEPYVSTEGKATGFQQLHPLALSDNSSNQIFTGAWEWTSTVLEKHEDFEPEELYPDYTRDFFDGKHNVVLGGSFATATRISNRRSFRNFYQAGYKYAWIGARLVKN

SEQ ID NO: 3 - DNA, 1179 bp, unidentified
  source 1..1179: mol_type = genomic DNA; note = Schizosaccharomyces pombe
atggcagagaacaacgtttacggccacgagatgaagaagcacttcatgctcgacccagactacgttaacgtcaacaacggctcctgcggtaccgaatccctggctgtttacaacaagcacgttcagctcctgaaggaggcacagtccaagccagatttcatgtgtaacgcttacatgcctatgtacatggaggcaacccgcaacgaagtcgctaagcttatcggcgcagactcttccaacatcgttttctgcaactccgctactgacggcatctccaccgttctcctgaccttcccatgggagcagaacgatgagatcctcatgctgaacgtcgcatacccaacctgtacttacgctgcagacttcgctaagaaccagcacaaccttcgtctcgacgtgatcgatgttggtgttgaaatcgacgaggacctgttcctcaaggaggtcgaacagcgcttcctgcagtctaagcctcgtgcattcatctgcgatatcctttcctccatgccagttatcctcttcccatgggagaaggttgtcaagctgtgtaagaagtacaacatcgtttccatcatcgacggcgctcacgcaatcggccacatccctatgaacctcgctaacgttgacccagatttcctgttcaccaacgcacacaagtggcttaactctccagctgcatgcaccgtcctctacgtgtccgctaagaaccacaacctgatcgaggcactccctctgtcctacggttacggccttcgcgaaaaggagtccatcgctgttgacaccctcactaaccgtttcgttaactctttcaagcaggacctgccaaagttcatcgcagtcggcgaggctatcaagttccgcaagtccatcggtggcgaagagaagatccagcagtactgtcacgagatcgcactcaagggcgctgaaatcatctccaaggagctgggtacctccttcatcaagccaccttacccagttgcaatggttaacgtcgaggttccacttcgtaacatcccttctatcgaaacccagaaggttttctggccaaagtacaacaccttcctccgcttcatggagttcaagggcaagttctacactcgtctgtccggcgctgtctacctcgaggaatccgatttctactacatcgcaaaggtgatcaaggacttctgctccctgtaa

SEQ ID NO: 4 - protein, 392 aa, unidentified
  source 1..392: note = Schizosaccharomyces pombe
MAENNVYGHEMKKHFMLDPDYVNVNNGSCGTESLAVYNKHVQLLKEAQSKPDFMCNAYMPMYMEATRNEVAKLIGADSSNIVFCNSATDGISTVLLTFPWEQNDEILMLNVAYPTCTYAADFAKNQHNLRLDVIDVGVEIDEDLFLKEVEQRFLQSKPRAFICDILSSMPVILFPWEKVVKLCKKYNIVSIIDGAHAIGHIPMNLANVDPDFLFTNAHKWLNSPAACTVLYVSAKNHNLIEALPLSYGYGLREKESIAVDTLTNRFVNSFKQDLPKFIAVGEAIKFRKSIGGEEKIQQYCHEIALKGAEIISKELGTSFIKPPYPVAMVNVEVPLRNIPSIETQKVFWPKYNTFLRFMEFKGKFYTRLSGAVYLEESDFYYIAKVIKDFCSL

SEQ ID NO: 5 - DNA, 1002 bp, Chlorobium limicola
  source 1..1002: mol_type = genomic DNA
atggcttattccaagaccaacctgtcggaattgccacttgcagatatagataaccacttgactgaaataggatttgataccacaataagtgagataattaccggtttgactgctaatgcgaagtacatccagtcgaagtatttttacgataaaagaggctcagcacttttcgaaaaaatcaccagtctgtctgaatactatccatcgcgtacagaaaaggctattattagccaacttccaccagctcttatagaagaccttgctgacatagatattatcgagcttggttgtggtgaccattcaaagattagtttgttgattcgccgtataccagcagagtccgtaccgggtttaagatattttcctatcgatattagccaaacggcactgaagcagtcgatcgaagaccttcgtgatcttttccctgcgctgaaagttaaagggatacttgcagattatgtccaccaaatgcatttattccctgaagaacgtaaacggctgttttgctttttcggttctacaatcgggaaccttagccgtgaggagacgcttgatttcatgcagaacatgggcactactatgcatccgggtgacatgcttttggttggcatggacagagtaaagaatatagcattgctggaaaaggcgtacaatgacgaccaatttatcacagctatgtttaacaaaaatatactgcgggtgattaacggcttaataaaatccgattttaatcccgatgattttgaacaccgggctttctataatgctgacttcaacagaatcgagatgcacttggaagcaactgggaatatttctgtcaagtccgcgttcatgcctgaacttattcggatcaagaaaggcgaaacaatccatactgagaacagtcacaaattcgaaaaggccgacattctgttaatgggtcagcacgccgggttagccattaaaaatatttattcggacaagaacgaattattttccttggctcattacgaaaaaaaataa

SEQ ID NO: 6 - protein, 333 aa, Chlorobium limicola
MAYSKTNLSELPLADIDNHLTEIGFDTTISEIITGLTANAKYIQSKYFYDKRGSALFEKITSLSEYYPSRTEKAIISQLPPALIEDLADIDIIELGCGDHSKISLLIRRIPAESVPGLRYFPIDISQTALKQSIEDLRDLFPALKVKGILADYVHQMHLFPEERKRLFCFFGSTIGNLSREETLDFMQNMGTTMHPGDMLLVGMDRVKNIALLEKAYNDDQFITAMFNKNILRVINGLIKSDFNPDDFEHRAFYNADFNRIEMHLEATGNISVKSAFMPELIRIKKGETIHTENSHKFEKADILLMGQHAGLAIKNIYSDKNELFSLAHYEKK

SEQ ID NO: 7 - DNA, 1374 bp, Chlorobium limicola
  source 1..1374: mol_type = genomic DNA
atgcagaacaagaacttcagagcaccccaatcagaagcaattggcattctgtataagttaatcgaaactggatctaaacacaaaaacatgtatgaccacacggaaatcactaccgattcacttctggcgttattaggcagcgagaaggtcaaaataatcgatgtccgctcagcagatgcgtataacggctggcgtatgagaggtgaagtacgcggtgggcatataaaaggcgcaaaatccctgccggcgaaatggttaactgatccggagtggcttaacatagtgagattcaagcagatacggccggaggacgcaattgttctgtatggatacacacccgaggaatgcgaacagaccgcgacgcgcttcaaagaaaacggatataacaacgtgagtgtttttcatcgtttccatccggattggacaggtaacgacgcgttccccatggatcggcttgagcagtataatcgtctggtccctgcggaatgggtgaatggcctgatatcaggcgaagaaattcccgaatatgataatgacacatttatcgtctgccatgcgcactaccgtaaccgtgatgcctacctttctggtcacatccctggcgctactgatatggacactttggcacttgagtccccagaaacctggaatcggcgcacaccagaggagttaaaaaaggcgttagaagaacatgggataaccgcatctaccactgtggtcctgtacggaaagtttatgcaccctgataatgctgacgaatttccaggatctgcagctggtcacattggagctattcgtctggccttcataatgatgtatgcgggcgtcgaggatgtgcgtgtgttaaacgggggctaccaatcctggacagatgctggatttgcgatttcaaaggatgacgttccgaaaactacagtacctgagttcggtgctccgataccctcccggccggagtttgcggtcgatattgacgaggccaaagagatgctgcagtctgaggattccgatttggtgtgcgtccgctcgtatcctgagtacattggagaggtttcgggatataactatataaaaaagaaggggcgcatcccaggagctatctttgccgaatgcgggtccgatgcttatcacatggagaactaccgcaaccatgaccatactacgcgtgaataccacgagatagaggatatatgggcgaagagcggaatcatacccaaaaaacacttagccttctactgcgggacgggatggcgtggatctgaagcgtggtttaatgctttgctgatgggatggcctcgggtttcggtttacgacggcgggtggtttgagtggtcaaacgatccggagaatccttacgagacaggcgtgccaaaataa

SEQ ID NO: 8 - protein, 457 aa, Chlorobium limicola
MQNKNFRAPQSEAIGILYKLIETGSKHKNMYDHTEITTDSLLALLGSEKVKIIDVRSADAYNGWRMRGEVRGGHIKGAKSLPAKWLTDPEWLNIVRFKQIRPEDAIVLYGYTPEECEQTATRFKENGYNNVSVFHRFHPDWTGNDAFPMDRLEQYNRLVPAEWVNGLISGEEIPEYDNDTFIVCHAHYRNRDAYLSGHIPGATDMDTLALESPETWNRRTPEELKKALEEHGITASTTVVLYGKFMHPDNADEFPGSAAGHIGAIRLAFIMMYAGVEDVRVLNGGYQSWTDAGFAISKDDVPKTTVPEFGAPIPSRPEFAVDIDEAKEMLQSEDSDLVCVRSYPEYIGEVSGYNYIKKKGRIPGAIFAECGSDAYHMENYRNHDHTTREYHEIEDIWAKSGIIPKKHLAFYCGTGWRGSEAWFNALLMGWPRVSVYDGGWFEWSNDPENPYETGVPK

SEQ ID NO: 9 - DNA, 35 bp, synthetic construct
  misc_feature 1..35: note = Synthetic
  source 1..35: mol_type = other DNA
ttgacggctagctcagtcctaggtacagtgctagc

SEQ ID NO: 10 - DNA, 35 bp, synthetic construct
  misc_feature 1..35: note = Synthetic
  source 1..35: mol_type = other DNA
ttgacagctagctcagtcctaggtactgtgctagc

SEQ ID NO: 11 - DNA, 23 bp, synthetic construct
  misc_feature 1..23: note = Synthetic
  source 1..23: mol_type = other DNA
gtaacatttaataggaggaatta

SEQ ID NO: 12 - DNA, 35 bp, synthetic construct
  misc_feature 1..35: note = Synthetic
  source 1..35: mol_type = other DNA
tttacggctagctcagtcctaggtatagtgctagc

SEQ ID NO: 13 - DNA, 23 bp, synthetic construct
  misc_feature 1..23: note = Synthetic
  source 1..23: mol_type = other DNA
attaacgatactaaggagcgaat

SEQ ID NO: 14 - DNA, 21 bp, synthetic construct
  misc_feature 1..21: note = Synthetic
  source 1..21: mol_type = other DNA
agagaaagaggagaaatacta

SEQ ID NO: 15 - DNA, 2481 bp, unidentified
  source 1..2481: mol_type = genomic DNA; note = Microsporum canis
atgagcccgattgccaccagcaatgttgatattgtggatattcgtcgcaatagcctggaaagcagcctggttcaggatatctatcatggtctgcaggcaaaagaaaaaagcctgccgaccctgctgctgtatgataccaaaggtctgcgtctgtttgaagatattacctatctggatgaatactatctgaccaatgcagaaattgaagtgctgaccgccaatgcagccaaaattgcagcaattattccggaaaattgtcagctggtggaactgggtagcggcaatctgcgtaaaattgaaattctgctgaatgaactggaacgcaccaaaaaatcagtggaatattatgccctggatctgagcctggaagaactgcatcgcacctttgcagaactgccgagcaaaagctatcgctatgtgaaatgcggtggtctgtggggtacctatgatgatggcctggcctggctgaataagctggtgaatcgtaataagccgacctgggttatgagtctgggcagcagcatgggtaattttaatccgaccgaagccgcaggttttctgagtggttttgcacgtagtctgggcccggttgatagcatggttattgccctggacccttgtaaagcaagtgaaaaagtttttcgtgcatataatgacagtaagggcgttaccaaacagttttatctgaatggcctgagcaatgcaaataccattctgggttttgaagcctttaaactgggtgaatgggatgccattggtgaatttgatcagacccagggttgtcatcgtgcctattatgtgccgctgaccgataccgttattcgcgatattcatattaagaaaggcgaaaaaatcttcttcgaacaggcattcaaatttggcgcagatgaatgtgaaaaactgtggcgtgaagccggtctgcagccgacccgtaaatttggtgacgaatataatatctatatcctgagcagcgcaagtgccaccatgaatccgtatcagctgccgaccaaaggcccggaatatgtgaaaggcgttgtgccggccctgggcgattttgaagccgtgtggaaactgtgggataccgtgaccaccgccatggtgccgccgaatgatctgctgagccgtccgattaatctgcgtaatagcctgattttctatctgggtcatattccggcattcatggatcgccatctgacctgtgccaccagtggtattccgaccgaaccggccaattttcatagtatgtttgaacgcggtattgatccggatgttgataatccggatcattgtcatgatcatagtgcaattccggatgaatggccggcagttgaaaccctggttgaatatcagcagaaagtgcgtgtgcgcgccaaaagtctgtatagcgatgccaaaaccggcgatcgccgcattgcagaagccctgtggattagttttgaacatgaaaccatgcatctggaaacctttctgtatatgctgctgcagagtagcagcaccatgccgccgccgctggtgcctgaaccggattttaaacagctggcaagcaatagcgccgaaaaagcagttccgaatgaatggtttgatattccggaacagaccctggaaattggtctgaatgaaccggaaagcgatgaaattccgagtaatagctttggttgggataatgaaaaaccgcgccgcgttgtgcaggttccgggctttgtggccaaagcacgtccgattaccaatggcgaatatgcaaaatatctggaagaacgtggcattggtcaggtgccggcaagctgggttaaaaaacataccgaaaatgaaagcaccaatgatggcattgaaaccctgagcagcggtaatagtagccataatgttgttaccgaatatgccgtgcgtaccgtgtttggcccggttccgctggaatgggttctggattggccggtggcagcaagttatgaagaactgacccagtatgccaaatggatgaattgccgtattccgacctttgaagaagtgcgcagcctgtataaatatagcctgaccagcccgaccggtaccaatcatagcaccaatgaactgcgtccgagcaccaccgaactgaaaaaaccggaatattgtaccagtcatcagccggttcagaccccgatgaaaattcatggtccggtgtatgttgatctggatggttgtaatgtgggctttacccattggcatccgaccccggttacccagaaaggtaataagctgtgtggtcagggtgactttggcggtctgtgggaatggaccagcagtaccctgcaggcacatgaaggctttaaaccgatgagcctgtatccggcatataccgcagatttctttgatggcaaacataatattgtgctgggcggtagctgggccacccatccgcgtctggcaggccgtagtacctttgttaattggtatcagcgtaattatccgtatgtgtgggccggcgcccgtctggtgcgtgatgattaa

SEQ ID NO: 16 - protein, 826 aa, unidentified
  source 1..826: note = Microsporum canis
MSPIATSNVDIVDIRRNSLESSLVQDIYHGLQAKEKSLPTLLLYDTKGLRLFEDITYLDEYYLTNAEIEVLTANAAKIAAIIPENCQLVELGSGNLRKIEILLNELERTKKSVEYYALDLSLEELHRTFAELPSKSYRYVKCGGLWGTYDDGLAWLNKLVNRNKPTWVMSLGSSMGNFNPTEAAGFLSGFARSLGPVDSMVIALDPCKASEKVFRAYNDSKGVTKQFYLNGLSNANTILGFEAFKLGEWDAIGEFDQTQGCHRAYYVPLTDTVIRDIHIKKGEKIFFEQAFKFGADECEKLWREAGLQPTRKFGDEYNIYILSSASATMNPYQLPTKGPEYVKGVVPALGDFEAVWKLWDTVTTAMVPPNDLLSRPINLRNSLIFYLGHIPAFMDRHLTCATSGIPTEPANFHSMFERGIDPDVDNPDHCHDHSAIPDEWPAVETLVEYQQKVRVRAKSLYSDAKTGDRRIAEALWISFEHETMHLETFLYMLLQSSSTMPPPLVPEPDFKQLASNSAEKAVPNEWFDIPEQTLEIGLNEPESDEIPSNSFGWDNEKPRRVVQVPGFVAKARPITNGEYAKYLEERGIGQVPASWVKKHTENESTNDGIETLSSGNSSHNVVTEYAVRTVFGPVPLEWVLDWPVAASYEELTQYAKWMNCRIPTFEEVRSLYKYSLTSPTGTNHSTNELRPSTTELKKPEYCTSHQPVQTPMKIHGPVYVDLDGCNVGFTHWHPTPVTQKGNKLCGQGDFGGLWEWTSSTLQAHEGFKPMSLYPAYTADFFDGKHNIVLGGSWATHPRLAGRSTFVNWYQRNYPYVWAGARLVRDD

SEQ ID NO: 17 - DNA, 2571 bp, unidentified
  source 1..2571: mol_type = genomic DNA; note = Ajellomyces dermatitidis
atggcccctagtaaactgagtaatgttccgattatggatattcatgttaacgatctgaaggatagcctggtgaatgatatctatgccggtctgaaaccgagtcatggtggtgccaaaagtctgccgaccctgctgctgtatagtaccgaaggtctgcgtcagtttgaagatattacctatgttgatgaatactacctgaccaatgcagaaattgaagcactgaccacccatgccgcaaaaattgtgaatcagctgccggaaaatgcacagctgctggaactgggtagcggcaatctgcgcaaaattaagattctgctggatgaatttgagcgtaaacagaaagcagttgaatattttgccctggatctgagtcgcgaagaactgcatcgtacctttgcagaaattccgcagggcggttataaatatgtgcgttgtcgtggtctgcatggcacctatgatgatgcactgatttggctgacccgcccggaaaatcgccgtaatccgacctgtattctgagtatgggtagcagtattggcaattttacccgcccggaggccgcacagtttctgaatcgttttagcaaaatgctgggcccgagcgatagcatgctgattggtattgatagttgtcaggaccctgaacgcgtgtataaagcctataatgatagtcagggtgttacccgtgatttttatatgaccggcctgagccatgcaaatagtattctgggttttgaagcctttaaaaaagaagattggggtgttgcaggtcattatgatgtggttagtggtgcacatatggcctattatgtgccgaataaggatgttacctttgatggcgtggttctggaaaaaggtgaaaaaattttctttgagcaggcattcaaatacggcccgaaagattgcaaagatttgtttcagcatgcaggtctgaccccgattgcacagtttggtaataataccggcgaatattatgttcatctgctgagtagctgcgcactggaaatgccgacccgcgccgcacagtatgcagaaaatgtgattccgaatgtggccgattttgaaaatctgtggaaaacctgggatatggtgaccctgagcatgattccgcaggatgatctgctgagtaaaccgattcgcctgcgcaatgcactgattttctatctgggccatattccggcatttctggatattcatctgacccgtgcaaccgatggtaccccgaccgaaccgaaacattttcagagtatgtttgaacgcggcattgatccggatgttgataatccggaactgtgtcatgatcatagtgatattccgagtgaatggccgagcctgggcgaaattctggcatatcgcgatgccgttcgtagtcgcaccctggccctgctggaaaaacgcaccaaagatcgtcgcctggccgaagccctgtggattggttttgaacatgaagccatgcatctggaaacctttctgtatatgctgctgcagagcgataaagccctgccgccgccgggcgttctgcgtcctaattttgaaagtctggcacgccagagtgcactggaaagccgtgaaaatagctggtttaccattccggaacagcagattagcattggcctggatgatccgaatgaacatgttattccggaacatagctttagttgggataatgaaaaaccgaaacgtaatgttaacgtgagcgcctttgcagcacaggcccgtccgattaccaatggcgaatatgcaacctatctggaagaacatcatattaccaccattccggccacctggattgatctgaccccgaaaaccggcgatgtgaaacgtaatggttatgatagctgtaatggctatagtaccaatggtgttcgtcagagtagcaccagtgccaccaaaaaatttctggaaaaatttgccgttcgcaccgtgtttggtccggtgccgctgaaatgggcactgaattggccgctgatggcaagctataatgaactgcagggttatgccgaaagtgtgaattgccgtattccgacctttgaagaagtgcgcagtatctatcagtatagcgcatttctgaaaagcaaacagcgtaatggcgcaaccagtctgaccaatggccatagcaatgttccgaatggtaccgttaaaacctttgatcgcagcgtggaaccggccaaagatacccatggcaatccgcgtagtccggatcatcagccggtgcagtgcccgagcgcagatccgatgccggtttatattgatctggatgaaaattgcaacgtgggtctgaaacattggcatccgaccccggtgacccagaatggtgaccgcctgagtggccagggtgaactgggcggcgtttgggaatggaccagtaccccgctgcatgcccatgaaggctttaaagccatggatctgtatccgggctatagtgcagatttctttgatggcaaacataatattgtgctgggtggtagttgggccacccatccgcgtattgccggccgtaccacctttattaattggtatcagcgcaaatatatctacgtgtgggccggcgcacgcctggttcgcgatatttaa

SEQ ID NO: 18 - protein, 856 aa, unidentified
  source 1..856: note = Ajellomyces dermatitidis
MAPSKLSNVPIMDIHVNDLKDSLVNDIYAGLKPSHGGAKSLPTLLLYSTEGLRQFEDITYVDEYYLTNAEIEALTTHAAKIVNQLPENAQLLELGSGNLRKIKILLDEFERKQKAVEYFALDLSREELHRTFAEIPQGGYKYVRCRGLHGTYDDALIWLTRPENRRNPTCILSMGSSIGNFTRPEAAQFLNRFSKMLGPSDSMLIGIDSCQDPERVYKAYNDSQGVTRDFYMTGLSHANSILGFEAFKKEDWGVAGHYDVVSGAHMAYYVPNKDVTFDGVVLEKGEKIFFEQAFKYGPKDCKDLFQHAGLTPIAQFGNNTGEYYVHLLSSCALEMPTRAAQYAENVIPNVADFENLWKTWDMVTLSMIPQDDLLSKPIRLRNALIFYLGHIPAFLDIHLTRATDGTPTEPKHFQSMFERGIDPDVDNPELCHDHSDIPSEWPSLGEILAYRDAVRSRTLALLEKRTKDRRLAEALWIGFEHEAMHLETFLYMLLQSDKALPPPGVLRPNFESLARQSALESRENSWFTIPEQQISIGLDDPNEHVIPEHSFSWDNEKPKRNVNVSAFAAQARPITNGEYATYLEEHHITTIPATWIDLTPKTGDVKRNGYDSCNGYSTNGVRQSSTSATKKFLEKFAVRTVFGPVPLKWALNWPLMASYNELQGYAESVNCRIPTFEEVRSIYQYSAFLKSKQRNGATSLTNGHSNVPNGTVKTFDRSVEPAKDTHGNPRSPDHQPVQCPSADPMPVYIDLDENCNVGLKHWHPTPVTQNGDRLSGQGELGGVWEWTSTPLHAHEGFKAMDLYPGYSADFFDGKHNIVLGGSWATHPRIAGRTTFINWYQRKYIYVWAGARLVRDI

SEQ ID NO: 19 - DNA, 2508 bp, unidentified
  source 1..2508: mol_type = genomic DNA; note = Aspergillus niger
atgagcccgctgtgtccggttgttaaaggcgttgatattgtggatattcgtcagaatgatgtggaattttcactggtgaatgatattcagcgcggtattgatccgccggcaggtacctgccgcagcatgcctaccatgctgctgtatgatgcccagggcctgaaactgtttgaagatattacctatctggaagaatactatctgaccaatgcagaaattgatgtgctgcgtacccatgcaaaacgcattgttgaacgtattccggataatgcccagctgctggaactgggtagcggcaatctgcgcaaaattgaaattctgctgcaggaatttgaagccgcaagcaaaaaagttgattattatgcactggatctgagtctgagcgaactggaacgtacctttagcgaagtgagcctggatcagtatcagtatgttaaactgcatggtctgcatggtacctatgatgatgcactgacctggctggaaaatccggcaaatcgtaaagtgccgaccgttattatgagtatgggtagcagcattggcaattttgatcgtccggccgccgcaaaatttctgagccagtttgcacgcctgctgggcccgagcgatctgatggtgctgggtctggatagctgcaccgatagtgataaagtttataaagcctataacgacagtaagggcattacccgccagttttatgaaaatggcctgctgcatgcaaatgccgtgctgggctatgaagcctttaaactggatgaatgggatattgtgaccgaatatgataatgttgaaggccgccatcaggccttttatgcaccgaatcgtgatgttaccattaatggtgtgctgctgcagaaaggtgaaaaactgatttttgaagaagcattcaaatacgatccggaacagtgtgatcagctgtggcatgatgcaggtctgattgaagatgcagaatttggtaatgaaagtggtgactatctgattcatgttctgagcagcgccagtctgaattttagtacccgtccgagtcagtatgccgcacagagcattccgagttttgaagaatttcagagtctgtggaccgcatgggatattgttaccaaagccatggttccgcgcgaagaactgctgagtaaaccgattaagctgcgtaatgccctgattttctatctgggtcatattccgacctttctggatgttcatctgacccgtgccctgggcgaaaaaccgacccatccgaaaagctatcgcctgatttttgagcgtggtattgatcctgatgtggatgatccggaaaaatgccatagtcatagcgaaattccggatgaatggccggcactgggtgacattctggattatcaggtgcgtgttcgcagtcgcgtgcgtagtatttttcagaaacataatgttgccgaaaaccgtgttctgggcgaagcactgtggattggttttgaacatgaagcaatgcatctggaaacctttctgtatatgctgattcagagcgaacgcaccctgccgccgccggcagttcctagaccggattttcgtaaatttttccatgatgcccgtcaggaaagtcgcccgaatgaatggtttagtattccggaaaaaaccctgagcgttggcctgcatgatgatggtcatagcgttccgcgtgatagctttggctgggataatgaaaaaccgcagcgtaaaattaccgtgaaagcatttgaagcacaggcccgcccgattaccaatggtgaatatgcaaaatatctgcaggcaaatcagctgccgcagaaaccggaaagttgggttctgattaagccggaaacctatccgacctgtaatggcgtgagccaggatggtagttatgcaaccaatgagtttatggcccattttgccgttcgtaccgtttttggtagtgtgccgctggaactggcacaggattggccggttattgcaagttatgatgaactggccaaatatgcaaaatgggtggattgtcgcattccgaccttcgaagaagccaaaagcatctatgcacatgcagcacgtctgaaagaaaccagtcatggtctgaatggccatagcgaaaccaatggtgttaatggccatgaacatagcgaaacaaatccgctgcgtccgcgtaccccggatcatcagccggttcagcatccgagtcaggaaagtctgccggtgtttgtggaactggataattgcaatgtgggttttaaacattggcatccgaccccggttattcagaatggcgatcgcctggcaggccatggtgaactgggcggtgtttgggaatggaccagcaccgaactggccccgcatgaaggttttgaagccatgcagatatatccgggctataccagtgatttctttgatggtaaacataatatcatcctgggcggtagttgggcaacccatccgcgtattgccggccgcaccacctttgttaattggtatcagcgtaattatccgtatccgtgggccggtgcccgtctggttcgcgatgtgtaa

SEQ ID NO: 20 - protein, 835 aa, unidentified
  source 1..835: note = Aspergillus niger
MSPLCPVVKGVDIVDIRQNDVEFSLVNDIQRGIDPPAGTCRSMPTMLLYDAQGLKLFEDITYLEEYYLTNAEIDVLRTHAKRIVERIPDNAQLLELGSGNLRKIEILLQEFEAASKKVDYYALDLSLSELERTFSEVSLDQYQYVKLHGLHGTYDDALTWLENPANRKVPTVIMSMGSSIGNFDRPAAAKFLSQFARLLGPSDLMVLGLDSCTDSDKVYKAYNDSKGITRQFYENGLLHANAVLGYEAFKLDEWDIVTEYDNVEGRHQAFYAPNRDVTINGVLLQKGEKLIFEEAFKYDPEQCDQLWHDAGLIEDAEFGNESGDYLIHVLSSASLNFSTRPSQYAAQSIPSFEEFQSLWTAWDIVTKAMVPREELLSKPIKLRNALIFYLGHIPTFLDVHLTRALGEKPTHPKSYRLIFERGIDPDVDDPEKCHSHSEIPDEWPALGDILDYQVRVRSRVRSIFQKHNVAENRVLGEALWIGFEHEAMHLETFLYMLIQSERTLPPPAVPRPDFRKFFHDARQESRPNEWFSIPEKTLSVGLHDDGHSVPRDSFGWDNEKPQRKITVKAFEAQARPITNGEYAKYLQANQLPQKPESWVLIKPETYPTCNGVSQDGSYATNEFMAHFAVRTVFGSVPLELAQDWPVIASYDELAKYAKWVDCRIPTFEEAKSIYAHAARLKETSHGLNGHSETNGVNGHEHSETNPLRPRTPDHQPVQHPSQESLPVFVELDNCNVGFKHWHPTPVIQNGDRLAGHGELGGVWEWTSTELAPHEGFEAMQIYPGYTSDFFDGKHNIILGGSWATHPRIAGRTTFVNWYQRNYPYPWAGARLVRDV

SEQ ID NO: 21 - DNA, 2556 bp, unidentified
  source 1..2556: mol_type = genomic DNA; note = Coccidioides posadasii
atgggtctggcaatgggtggtgttaatattattgatattcgccgtaataacctgaataatagcctggccaaagatgtgacccgcggcctggaccctaaaaatggcacccagcgcagtctgccgaccctgctgctgtataataccgaaggcctgcgcctgtttgaagaaattacctatctggatgaatactatctgaccaatgccgaaattgaagttctgaccacccatgccgttagtattgttgaacgtgtgccggaaaatagtcagctggttgaactgggcagtggtaatctgcgcaaagttgaaattctgctgaatgaatttgagcgtaccaaaaaaccggttgaatatctggccctggatgtgagtctggaagaactgaatcgcacctttgccgaactgccgagtaaaagctatcagtatgttaaatgcagcggtctgctgggcacctatgatgatgcactgagctggctgaaacgtagtgaaaatcgtcgcaaaccgacctgggttatgagcatgggcagcagcattggcaattttacccgtagcgaagcagcccagtttctgggtggttttgcccgtaccctgggtgcagatgatgccctgctggtgggtctggatagctgtaaagatccgcagaaagtttttcgtgcctataatgatagtaagaatgttacccgtgaattttatctgaatggtctggcaaatgcaaatagcattctgggctttgaagcattcaaacgtgaagattgggatgtggccggtatctatgatgaagttgatggctgtcataaagcctattataccccgacccgtgatgttaccattgaagcatggagctttaccaaaggtgaacgcattttctttgaacaggcattcaaatatgcagaaaaagaatatcaggcactgtggcagcaggcaggcctgaccagtaccgcacgttttaccagtagcaccggtgaccataatattcatctgctgagcagtagcccgtatattctgccgacccagccggcagaatatgccgcaaccctgaccccgagtctgaaagaatttgaagcactgtggaaactgtgggataccgttaccaccggcatgctgccgcgcaatgaactgctgagcaaaccgattaatctgcgcaatgcactggtgttttatctgggccatattccgacctttctggatatgcatattacccgcgccattgatggtcagccgaccgaaccgaaaagctattggagcatttttgaacgtggcattgatccggatgttgatgatccgcgtaaatgccatgatcatagcgaaattccggatgcatggccgccggttgaagaaattctgcagtttcagaccaccgtgcgcaatcgtgcacgtagtctgctgcagaaaagccagcataccaccaatcgccgcattcatgaagccctgtggattggttttgaacatgaagcaatgcatctggaaacctttctgtatatgctgctgcagagcgataaagtgtgtccgccgccggaaattagcaccccggattttgaatatctggcgatgcgcagcgcccaggaaagtgttccgaatgaatggtttattgttccggaacagaccatttggattggtctggatgatccggaccctacccgcattccgtttggcagttttggttgggataatgaaaaaccgcagcgcaccgcaaaagttagtagctttgaagccaaaggccgtccgattaccaatggtgaatattgccgttatctggaagccaataagctggccaccgtgccggccagttggacccgtagcagtagtggctttagcgaaccgaatggtcatgccgccagccataccaatggtaccaatggtcgtgaaaccagcgaagcaagtgcctttagccagctgctgagtaaatatcatgttcgcaccgtgtttggtccggttccgctgcgctttgccctggattggccggttattgcaagctataatgaactggaacgctatgcaaattgggttaattgtcgcattccgaccttcgaagaagcacgcagcatctatcagtatagtgtgtttctgaaagatcaggaaattggtgtgcagagtagcctgattgatgcaagcagcaatgatatggaaggtccgccgcgcgatctgaatggttttgttgaacatcgtaatggtcgtccgcgtgccccggatcatcagccggttagccagccgccgagcacccagatgccggtgaatgtggatctggatggttataatgtgggttttaaacattggcatccgaccccggttacccagaatggtaataagctgagtggtcagggtggtatgggtggtgcctgggaatggaccagcagcaccctggaagcacatgaaggttttaaagcaatggatctgtatccggcctataccgccgatttctttgatggcaaacataatattgtgctgggcggcagctgggcaacccatccgcgtattgccggccgcaccacctttgtgaattggtatcagcgcaattatccgtatgtttgggcaggtgcacgcctggttcgcgatatttaa

SEQ ID NO: 22 - protein, 851 aa, unidentified
  source 1..851: note = Coccidioides posadasii
MGLAMGGVNIIDIRRNNLNNSLAKDVTRGLDPKNGTQRSLPTLLLYNTEGLRLFEEITYLDEYYLTNAEIEVLTTHAVSIVERVPENSQLVELGSGNLRKVEILLNEFERTKKPVEYLALDVSLEELNRTFAELPSKSYQYVKCSGLLGTYDDALSWLKRSENRRKPTWVMSMGSSIGNFTRSEAAQFLGGFARTLGADDALLVGLDSCKDPQKVFRAYNDSKNVTREFYLNGLANANSILGFEAFKREDWDVAGIYDEVDGCHKAYYTPTRDVTIEAWSFTKGERIFFEQAFKYAEKEYQALWQQAGLTSTARFTSSTGDHNIHLLSSSPYILPTQPAEYAATLTPSLKEFEALWKLWDTVTTGMLPRNELLSKPINLRNALVFYLGHIPTFLDMHITRAIDGQPTEPKSYWSIFERGIDPDVDDPRKCHDHSEIPDAWPPVEEILQFQTTVRNRARSLLQKSQHTTNRRIHEALWIGFEHEAMHLETFLYMLLQSDKVCPPPEISTPDFEYLAMRSAQESVPNEWFIVPEQTIWIGLDDPDPTRIPFGSFGWDNEKPQRTAKVSSFEAKGRPITNGEYCRYLEANKLATVPASWTRSSSGFSEPNGHAASHTNGTNGRETSEASAFSQLLSKYHVRTVFGPVPLRFALDWPVIASYNELERYANWVNCRIPTFEEARSIYQYSVFLKDQEIGVQSSLIDASSNDMEGPPRDLNGFVEHRNGRPRAPDHQPVSQPPSTQMPVNVDLDGYNVGFKHWHPTPVTQNGNKLSGQGGMGGAWEWTSSTLEAHEGFKAMDLYPAYTADFFDGKHNIVLGGSWATHPRIAGRTTFVNWYQRNYPYVWAGARLVRDI

SEQ ID NO: 23 - DNA, 2550 bp, unidentified
  source 1..2550: mol_type = genomic DNA; note = Cladophialophora immunda
atggcaacccataccccggccggtgtgccgattctggatattcgtagcgatcagagcgatcagagtctgctgcataccctgaaacagagtctgaatccgccgaaaggtcagccgcgtacctttccgaccctgctgctgtatgatgaaaaaggcctgaaactgtttgaagaaattacctatgtggatgaatactatctgaccaatgccgaaattgataccctgacccgtcatgccggcaaaattgttggccgcattccggatggcgcacgtctggtggaactgggtagtggcaatctgcgtaaagttaatattctgctgaaagcatttgaggaagccaataagaatgtggaatattatgccctggatctgagcctgagcgaactgaaacgtacctttgcacagctggatattaatgcatttcatcatgttaccttccgtgccctgcatggcacctatgatgatgccctgctgtggctgaaagaaagcgccaccgatgcccgcaccacctgcgtgatgaccatgggtagtagtctgggcaattttacccgtgaagaagccgcacagtttctggcaagctttaaaaaagttctggcagcaagcgattatgttatggttggcattgatgcatgtcagcagccggatcgcgtttttcgcgcctataatgatagcatgaatgtgaccgaacgcttttatcgtaatggcctgacccatgcaaataatattctgggttatgaagcatttcgtcaggatgaatggcagattgaaggcgtgtatgatgaaaatcagaataagcatcaggccagttatgtggcactgaaaaccattaataataaggatttcagcttcgaacagggcgaaaaagtgcatctggaagatgcattcaaatatagcgaagcccagcgtgatgcactgtggcatgccgcaggtctgattccgcagaccgcatataataataagaccaatgattactacatccacctgctgagcccgagtaccattaattttccgaccaaaccggccgaatttgccgccagcccggttccgagtctggatgattggcgtcagctgtgggccgcatgggataccgttaccaaaagtatggttccgaaagatgaactgctgaataagccgattaagctgcgtaatgatctgattttctatctgggccatattccgacctttgccgatattcattatatgaaagccaccaaagaaaaggccaccgatccggcctattatatgagtatttttgaacgcggcattgatccggatgttgataatccggaactgtgtcatgatcatagtgaaattccggatagctggccgccgctggaagatattctgaattatagccagcgtgtgcgcgaacgtattgttgaaggcattcatagcggtcgcgcctataccgatcgtcgtctgagtcgtggcctgtggctggcatatgaacatgaagccatgcatctggaaacctttctgtatatgctgctgcagagcgaacgcgtgctgccgccgcctggtgaaggtctgccggattttcgtaccctggccgccgaagcacgtgcaaatcgcaccagcaatcagtggcatcgtattccggccagcaaagttaaaattggtctggatgatccggaaaataattttggtccggatcgttattttggctgggataatgaacgcccgagccgcaccgtggcagtgaatgaatttgaagcccagagccgcccgattagtaatggcgaatatgcacgctttctggaagttacccataaagatagcctgccggccagctggaccgcaagtaaagtgggtgccgtgctgaatggcaccaatggcaccaacggcgccaatggccatgttcaggatgaactggatatggcaagtccgagctttgtggaagataaagccattcgcaccgtttatggcctgattccgctgaaatatgcactggattggccggttatggccagctatgatgaactggccgcatatgcagaatggagcaatggtcgcattccgaccctggaagaagcccgcagtctgtatcagtatgttgaaaatcaggatagtgttctgcagaaagtgaccagcaaactgattagcgccgttaatggtcatctgagcaatgatggcgttcaggaaaccccgccgagcacccatcagagcaatggcgttgtgaatggcagtgcagatggtccgagtctggacccgaatgaactgtttgtggaactgggcgatcgtaatgtggcctttcgtcattggcatccgaccccggttaccggtaatgcagatcgtctgcgtggccagagcgatctgggtggtctgtgggaatggaccagtaccccgctggccccgcatgaaggctttaaagccatggatctgtatccgggctataccgccgatttctttgatagtaaacataatgtgtgcctgggcggcagttgggcaaccgttccgcgtattgccctgaaaaagacttttgtgaattggtatcagcgcaattatccgtatgtttggtgcaccgcacgcctggtgcgcgatgttgttgtttaa

SEQ ID NO: 24 - protein, 849 aa, unidentified
  source 1..849: note = Cladophialophora immunda
MATHTPAGVPILDIRSDQSDQSLLHTLKQSLNPPKGQPRTFPTLLLYDEKGLKLFEEITYVDEYYLTNAEIDTLTRHAGKIVGRIPDGARLVELGSGNLRKVNILLKAFEEANKNVEYYALDLSLSELKRTFAQLDINAFHHVTFRALHGTYDDALLWLKESATDARTTCVMTMGSSLGNFTREEAAQFLASFKKVLAASDYVMVGIDACQQPDRVFRAYNDSMNVTERFYRNGLTHANNILGYEAFRQDEWQIEGVYDENQNKHQASYVALKTINNKDFSFEQGEKVHLEDAFKYSEAQRDALWHAAGLIPQTAYNNKTNDYYIHLLSPSTINFPTKPAEFAASPVPSLDDWRQLWAAWDTVTKSMVPKDELLNKPIKLRNDLIFYLGHIPTFADIHYMKATKEKATDPAYYMSIFERGIDPDVDNPELCHDHSEIPDSWPPLEDILNYSQRVRERIVEGIHSGRAYTDRRLSRGLWLAYEHEAMHLETFLYMLLQSERVLPPPGEGLPDFRTLAAEARANRTSNQWHRIPASKVKIGLDDPENNFGPDRYFGWDNERPSRTVAVNEFEAQSRPISNGEYARFLEVTHKDSLPASWTASKVGAVLNGTNGTNGANGHVQDELDMASPSFVEDKAIRTVYGLIPLKYALDWPVMASYDELAAYAEWSNGRIPTLEEARSLYQYVENQDSVLQKVTSKLISAVNGHLSNDGVQETPPSTHQSNGVVNGSADGPSLDPNELFVELGDRNVAFRHWHPTPVTGNADRLRGQSDLGGLWEWTSTPLAPHEGFKAMDLYPGYTADFFDSKHNVCLGGSWATVPRIALKKTFVNWYQRNYPYVWCTARLVRDVVV

SEQ ID NO: 25 - DNA, 2574 bp, unidentified
  source 1..2574: mol_type = genomic DNA; note = Glarea lozoyensis
atgaccattcatagcgcaaccaatggtaccgcaaaactgggtagccataccaaaccgatgagcagcagcaaaaccaataatattcagccgagtgcaaaagatgaactgaccagcggcgttgatattattgatattcgtcatgatgcagttgaaattaatctgaaagaagaaatcgacaagctgctgcatccggcagaaggcccgaaaaaactgccgaccctgctgctgtatgatgaacagtattatctgaccaatgcagaaattgatgtgctgaaacgtagtgcaggtagcattgcagatagcattccgagcggcagcatgctggtggaactgggcagtggcaatctgcgtaaagttagtattctgctgcgtgcactggaagccgccggcaaagaaattgattattatgccctggatctgagcctgagcgaactgaaacgcaccctggaacaggttccgaattttaaatatgttaagtgccatggcctgcatggtacctatgatgatggcctggattggctgaaagccgccgaacatggcagtcgcaccaaagttgttatgagcctgggcagcagcattggtaattttaaacgcagcgaagcagcaagctttctgcgcggttttagtgatgccctgggcccgagcaatatgatgctgattggtgtggatgcaaccagcgatccgagtaaagtgtatcatgcctataatgatcgtaaaggtaccacccatgagtttattctgaatggtctgaccaatgccaatggtattctgggtgaagatgcctttgatattaaggattggaaagttattggcgaatatgtgtttgatcatgaaggtggtcgtcatcaggcattttatgtgccgaaaaccgatgtgacctataaaaatattctgctgaaagcaggcgaacgtattcaggttgaagaaagtctgaaatatagcctggatgatgcaacccatctgtgggccgccagtggcctgcgtgaagtgagtcattggaccgccagtagcgatgcctataatattcatctgctgcagccgaaagaacgcatggaatttcataccgcaagtgcagtgtatgcagcaaccaccgtgccgagtgttgatgattggctgaatctgtggaaagtttgggatattgtgacccgtaaaatgattccggaacaggatctgctggaaaaaccgattaagctgcgtaatgcatgtattttctatctgggccatattccgacctttctggatattcagctgtgtaaagttagtggtgaaccgccgtgcgaaccgagtcattatccgaaaatttttgaacgcggtattgatccggatgttgataatccggaacattgtcatgcacatagcgatattccggatgaatggccgccgctggaagaaattctgcagtatcaggaacaggttcgcgcaaaagcactgaaactgaccgcagcaagcaaaattccgcgcgaaattggccgcgccctgtggattggtctggaacatgaaattatgcatctggaaaccctgctgtatatgctgctgcagagcgatcgcaccattccgccgaccagtcataccccgaattttaaggatgatgccaaagccgcagaaagcgcacgcgttgaaaatgaatggtttgaagttccgcgccagcagattaccattggcctggaagatccggaagataatagcggcggtgaccgccattttggctgggataatgaaaaaccgccgcgccaggttctggtgccgagctttctggccaaaggtcgtgccattaccaatgaagaatatgcccgctatctggaatataccaataagcatgaaatcccggcaagttgggccgatggtggtagcaccggcagtgataccaatggctttttcaatggcgcaaatggctatagtaatggccatagtaatggtggtgaaaagaaaagtagtaccaaagcatttctggaaggcaaaagcgttcgcaccgtgtatggtctggtgccgctgcagtatgcactggattggccggtgtttgcaagttatgatgaactggcaggttgtgcagcattcatgggcggccgtattccgaccgtggaagaagcccgcagcatctatagtcatgttgatggcctgaaactgaaagaagccgaacagcatctgggtaaaaccgtgccggccgttaatggtcatctggtgaatgatggcgttgaagaaagcccgccgagtcgtgccgtggttaatagtggtcgtagtcgcaatctgtttgcaaatctggatggtgcaaatgttggctttaaaaattggcatccggttgccgtgaccgccaatggcgataaactggcaggccaggccgaaatgggtggcgtttgggaatggaccagcagcccgctggttaaacatgaaggttttgaaccgatgccgctgtatccggcctataccagcgatttctttgatggtaaacataatatcgccctgggcggtagttgggcaacccatccgcgcattgccggtcgcaaaacctttgtgaattggtatcagcgtaattatccgtatgcctgggcaggcgcacgtctggttcgcgatatttaa

SEQ ID NO: 26 - protein, 857 aa, unidentified
  source 1..857: note = Glarea lozoyensis
MTIHSATNGTAKLGSHTKPMSSSKTNNIQPSAKDELTSGVDIIDIRHDAVEINLKEEIDKLLHPAEGPKKLPTLLLYDEQYYLTNAEIDVLKRSAGSIADSIPSGSMLVELGSGNLRKVSILLRALEAAGKEIDYYALDLSLSELKRTLEQVPNFKYVKCHGLHGTYDDGLDWLKAAEHGSRTKVVMSLGSSIGNFKRSEAASFLRGFSDALGPSNMMLIGVDATSDPSKVYHAYNDRKGTTHEFILNGLTNANGILGEDAFDIKDWKVIGEYVFDHEGGRHQAFYVPKTDVTYKNILLKAGERIQVEESLKYSLDDATHLWAASGLREVSHWTASSDAYNIHLLQPKERMEFHTASAVYAATTVPSVDDWLNLWKVWDIVTRKMIPEQDLLEKPIKLRNACIFYLGHIPTFLDIQLCKVSGEPPCEPSHYPKIFERGIDPDVDNPEHCHAHSDIPDEWPPLEEILQYQEQVRAKALKLTAASKIPREIGRALWIGLEHEIMHLETLLYMLLQSDRTIPPTSHTPNFKDDAKAAESARVENEWFEVPRQQITIGLEDPEDNSGGDRHFGWDNEKPPRQVLVPSFLAKGRAITNEEYARYLEYTNKHEIPASWADGGSTGSDTNGFFNGANGYSNGHSNGGEKKSSTKAFLEGKSVRTVYGLVPLQYALDWPVFASYDELAGCAAFMGGRIPTVEEARSIYSHVDGLKLKEAEQHLGKTVPAVNGHLVNDGVEESPPSRAVVNSGRSRNLFANLDGANVGFKNWHPVAVTANGDKLAGQAEMGGVWEWTSSPLVKHEGFEPMPLYPAYTSDFFDGKHNIALGGSWATHPRIAGRKTFVNWYQRNYPYAWAGARLVRDI

SEQ ID NO: 27 - DNA, 2562 bp, unidentified
  source 1..2562: mol_type = genomic DNA; note = Exophiala mesophila
atgacccgcaccattagccaggtgctgcatccggcctttaccatggccaccagccagcgtccggccgtgcgttttctggatattcgtggtgacaaaagcggccagagtctgctgagcatgctgaaagaaagtctggaccctccgaataagcagccgcgtagttttccgaccctgctgctgtatgatgaaaaaggtctgaaaattttcgaggaaattacctatctggatgaatattatctgaccaatgcagaaattgaagcactggaaacccatgcccgcgaaattgccacccagattccgcgcaatagtcgcattgtggaactgggtagtggtaatctgcgtaaaattaatattctgctggaagcctttgaagcagcaaaaaagaatgttgattactatgcactggatctgagctttccggaactgcagcgtacctttgccgaaattgataccagtcgttatcagcatgtgagctttaatgcactgcatggcacctatgatgatgccctgacctggctgagtaatagtagtggtgaccagagtacctgtgttatgaccatgggtagcagcctgggtaattttagccgtcaggatgcagcaggctttctgaccaaaattaagagcgttctgggtccggccgatctgattctggtgggtctggatgcctgccaggaccctcagcgtgtttttaaagcctataatgatagtcagctggtgaccgaacgtttttatcgcaatggtctggatcatgccaatagcctgctgggttatgaagtgtttcgtcaggaagattggagcgttgaaggccgttatgatgaacagctggatcgccatcatgcaacctatctggcccgcaaagatattattaccaaagattttagcttcaagcgtggcgaacgcctgccgtttgaagaaagctttaaatatagcgaagcacagagtgatcagctgtggcatgatagcggtctggtgcagcagatggcctttggcaataagagcgccgattattttattcatctgctgagcccggcagcaattaattttgccaccaaaccggccgaatatgcaaccaatccgattccgagtagcgatgattggcagcagctgtggaccgcatgggatgttgtgacccgtagcatgattccgaaagatgaactgctgaataagccgattaagctgcgtaatgatctgattttctatctgggccatattccgacctttgcagatattcattttaccaaagcaaccgatggcaaaccgaccgaaccggccagttattggagtatttttgaacgtggcattgatccggatgttgataatccggaactgtgtcatgatcatagtgaagttccggatagttggccggcactgaatgatattctgacctatgccaaacgtgtgcgcagtcgtattgcagatagcctggaaagcggccaggccgtgcaggatcgtcgcctgggtcgtggtctgtggctggcctatgaacatgaagccatgcatctggaaacctttctgtatatgctgctgcagagtgatcgcattctgccgccgccgggtaccgaacgcccggactttcgccagattgcagatgaagcacgtgcaaatcgcgtggccaataagtttcatcgcattccggcagcagaagtgaccctgggcctggatgatccggaaaatcatcatggtccggatcgttattttggctgggataatgaacgcccgagtcgcaccgttagcgttgcagcctttgaagcgcagagtcgtccgattagcaatggtgactatgcatattatctggaagttaccggcaatagcagcctgccggcaagctggattgcacgtcgcagtgttctgaatggtgtgaatggcgcagttagtaatggcgaagttggccgtgaagttgttagtagcgaatttctgggcgataaagcactgcgcaccgtttggggtccgctgccgctgaaacatgccctggattggccggttatggcaagctatgatgaactggccgcatatgcaaaatgggcaaatggtcgtattccgaccctggaagaagcacgtagtatctatcatcatgttgaaagccgcaaagataccctggaaaaagttccgagcaaactgattagtgcagttaatggccatctgagcaatgaaggcgtggaagaaaccccgccgagcaatcagagtagcggtgaagccgccaatggcagcctgccgccgaatccgaatgaactgtttgtggatctgaataattgcccggttggttttaaaacctggaccccgcagccgattacccatagcagtagcctgcgtggtcaggccgatgttggcggcctgtgggaatggaccagtaccccgctggccccgtatgatggctttaaagccatggatctgtatccgggctataccgcagatttctttgatggtaaacataatatctgcctgggtggcagctgggcaaccattccgcgtattgcaggtcgtaaaacctttgttaattggtatcagcgtaattatccgtatgtgtggtgcaccgcccgcctggttcgcgatattgcagaataa

SEQ ID NO: 28 - protein, 853 aa, unidentified
  source 1..853: note = Exophiala mesophila
MTRTISQVLHPAFTMATSQRPAVRFLDIRGDKSGQSLLSMLKESLDPPNKQPRSFPTLLLYDEKGLKIFEEITYLDEYYLTNAEIEALETHAREIATQIPRNSRIVELGSGNLRKINILLEAFEAAKKNVDYYALDLSFPELQRTFAEIDTSRYQHVSFNALHGTYDDALTWLSNSSGDQSTCVMTMGSSLGNFSRQDAAGFLTKIKSVLGPADLILVGLDACQDPQRVFKAYNDSQLVTERFYRNGLDHANSLLGYEVFRQEDWSVEGRYDEQLDRHHATYLARKDIITKDFSFKRGERLPFEESFKYSEAQSDQLWHDSGLVQQMAFGNKSADYFIHLLSPAAINFATKPAEYATNPIPSSDDWQQLWTAWDVVTRSMIPKDELLNKPIKLRNDLIFYLGHIPTFADIHFTKATDGKPTEPASYWSIFERGIDPDVDNPELCHDHSEVPDSWPALNDILTYAKRVRSRIADSLESGQAVQDRRLGRGLWLAYEHEAMHLETFLYMLLQSDRILPPPGTERPDFRQIADEARANRVANKFHRIPAAEVTLGLDDPENHHGPDRYFGWDNERPSRTVSVAAFEAQSRPISNGDYAYYLEVTGNSSLPASWIARRSVLNGVNGAVSNGEVGREVVSSEFLGDKALRTVWGPLPLKHALDWPVMASYDELAAYAKWANGRIPTLEEARSIYHHVESRKDTLEKVPSKLISAVNGHLSNEGVEETPPSNQSSGEAANGSLPPNPNELFVDLNNCPVGFKTWTPQPITHSSSLRGQADVGGLWEWTSTPLAPYDGFKAMDLYPGYTADFFDGKHNICLGGSWATIPRIAGRKTFVNWYQRNYPYVWCTARLVRDIAE

SEQ ID NO: 29 - DNA, 2583 bp, unidentified
  source 1..2583: mol_type = genomic DNA; note = Fusarium oxysporum
atgccgagtattaccgccagcagtgcaaccccgcagcgtgttaaaccgaccaccgcaaaaccgagcagtgccctgccgagtattattgatattcgtggtgaacatgttgaaattaatctgaaagatcagatcgcaagcatgtttaatccggatgaaggtccgcgcaaactgccgaccctgctgctgtataatgaaaaaggtctgcagatttttgaggatattacctatctggatgaatattatctgaccaattacgaaatcgaaatcctgaaaaaatccagcgccgaaattgcaagtcagattccggaaggtagtatggttattgaactgggcagtggcaatctgcgtaaagtgtgtctgctgctgcaggcctttgaagaactgaaaaaaccgattcagtattttgcactggatctgagtctgaaagaactggaacgcaccctggcccaggtgccggagtttaaatatgttagttgccatggtctgcatggcacctatgatgatggccgtgaatggctgaaacatccgagtctgaccagtcgcagtaaatgcattattcatctgggcagcagtattggcaattttacccgtgatggtgcagccgattttctgggcggttttgccgaagtgctgaccccgagtgatagtatgattgttgcagttgatagttgtagcaatccggcacaggtgtatcatgcctataatgatagtaaaggcgtgacccatcagtttgtgctgaatggtctgcagaatgccaatgaaattctgggcgaagaagcattcaataccgatgaatggcgcgttattggtgaatatgtttatgatgttgagggcggtcgtcatcaggcctttctgagtccgacccgcccgaccgatgccctgggtagtcgcgtgctgccgcatgaacgcattgaaattgaacagagcctgaaatatagtgaagcagaaaaagataagctgtggaaactggccggtctgaccgaaatgggtcgctggagccgcggcgatgaatatggtctgcattttctgcagaaaagcagcatgccgtttagtcgcattccgagcctgtatgccgccgaaccgctgccgaccgttcaggattggaaagccctgtggaaagcatgggatgttgtgaccaaagatatgatgccggatgaagaactgcaggaaaaaccgattaagctgcgcaatgcatgtattttctatctgggccatattccgacctttctggatattcagctgaccaaaaccaccggtaatgccccgaccgaaccggccacctattatagtatttttgaacgcggcattgatccggatgtggataatccggaacattgtcatacccatagcgaaattccggatgaatggccgctggtgcaggaaattatgatctatcaggatcgcgttcgtagtcgcctgcagaatctgtataaaaatggccaggataaaatcagtcgcgatattggtcgcgcaatttgggtgggctttgaacatgaactgatgcatattgaaaccctgctgtatatgatgctgcagagtgatcgcaccctgccgccgccgcataccgtgcaaccggattttgcaaaactggcccagcaggcccatgaagcccgcgtgcctaatcagtggtttgatgttccggaacagaccattaccctgggcatggatgatccggaagatggtaccgataatagtcgtcattttggctgggataatgaaaaaccggaacgtcagaccaaagttcatgcatttcgcgcacagggtcgcgcaatcaccaatgaagaatatgcccagtatctgtataatagcaaaattgaacacatcccggccagctggagtagcgttaccaatgcctataccaatggcgcaaccaatggcagccatgccaatggtaatagcaatggctatagcaatggcaatggtcatcatgcaagccaggttccggatagctttattcaggataaatttgttaagaccgtgtatggtctggtgccgctgaaatatgcactggattggccggtgtttgccagctatgatgaactggcaggctgtgcagcatggatgggcggtcgcattccgaccttcgaagaagccaaaagtatctatgcacatgtgaataagcagaaacgtgcagaagcagaacgcaccttaagcaaaaccgtgccggccgtgaatggtcatctggtgaatgatggtgtggaagaaaccccgccgagtaatagcagcgccctggtgaaagatagcagtagtgaactgtttttcgatctgaccggtgccaatgttggttttcgccattggcatccgatgccggtgaccagccgtggtaataagctggccggtcagagtgaaatgggcggcgtgtgggaatggaccagcagtagcctggaacgtcatgaaggctttgaaccgatgagtctgtatccgctgtataccaccgatttctttgatggtaaacataatgttgtgctgggcggcagctgggcaacccatccgcgtattgcaggccgcgcaagctttgtgaattggtatcagcgtaattatccgtatgcatgggttggtgcacgcctggtgcgcgatgtttaa

SEQ ID NO: 30 - protein, 860 aa, unidentified
  source 1..860: note = Fusarium oxysporum
MPSITASSATPQRVKPTTAKPSSALPSIIDIRGEHVEINLKDQIASMFNPDEGPRKLPTLLLYNEKGLQIFEDITYLDEYYLTNYEIEILKKSSAEIASQIPEGSMVIELGSGNLRKVCLLLQAFEELKKPIQYFALDLSLKELERTLAQVPEFKYVSCHGLHGTYDDGREWLKHPSLTSRSKCIIHLGSSIGNFTRDGAADFLGGFAEVLTPSDSMIVAVDSCSNPAQVYHAYNDSKGVTHQFVLNGLQNANEILGEEAFNTDEWRVIGEYVYDVEGGRHQAFLSPTRPTDALGSRVLPHERIEIEQSLKYSEAEKDKLWKLAGLTEMGRWSRGDEYGLHFLQKSSMPFSRIPSLYAAEPLPTVQDWKALWKAWDVVTKDMMPDEELQEKPIKLRNACIFYLGHIPTFLDIQLTKTTGNAPTEPATYYSIFERGIDPDVDNPEHCHTHSEIPDEWPLVQEIMIYQDRVRSRLQNLYKNGQDKISRDIGRAIWVGFEHELMHIETLLYMMLQSDRTLPPPHTVQPDFAKLAQQAHEARVPNQWFDVPEQTITLGMDDPEDGTDNSRHFGWDNEKPERQTKVHAFRAQGRAITNEEYAQYLYNSKIEHIPASWSSVTNAYTNGATNGSHANGNSNGYSNGNGHHASQVPDSFIQDKFVKTVYGLVPLKYALDWPVFASYDELAGCAAWMGGRIPTFEEAKSIYAHVNKQKRAEAERTLSKTVPAVNGHLVNDGVEETPPSNSSALVKDSSSELFFDLTGANVGFRHWHPMPVTSRGNKLAGQSEMGGVWEWTSSSLERHEGFEPMSLYPLYTTDFFDGKHNVVLGGSWATHPRIAGRASFVNWYQRNYPYAWVGARLVRDV

SEQ ID NO: 31 - DNA, 2565 bp, unidentified
  source 1..2565: mol_type = genomic DNA; note = Nectria haematococca
atgccgagcagcgtgaatgcaccgccggccgtttttcagggtgcacgtccggcagtgagcaaaccgagcccggcactgccggatattattgatattcgcggcgaacatgttgaaattaatctgaaagatcagatcatcagccagtttaatccggaagatggtccgcgtaaactgccgaccctgctgctgtataatgaaaaaggtctgcagatttttgaggatattacctatctggatgaatattatctgaccaattacgaaatcgaagtgctgaaacgcagtagtaccgaaattgcacgtcagattccggaaggcagcatggttattgaactgggtagcggtaatctgcgtaaagtgtgtctgctgctgcaggcctttgaagatttggccaaaccgattcagtattttgccctggatctgagccgcaaagaactggaacgtaccctggcccaggtgccggattttaaatatgtgagttgccatggcctgctgggcacctatgatgatggtcgtgaatggctgaaacatccgagtctgaccggtcgcagtaaatgcattctgcatctgggcagtagcattggtaattttagccgtgatgaagcagcagcctttctgggcggctttgccgatgttctgcgcccgagtgatagcatgattgttggcgtggatgcatgcaataatccggcaaaagtttataaaccgattatgaatcagccgcgtctgagtcgtaccaatcgcattcatcgctttattctgaatggtctgagtcatgccaatgaactgctgagcgaagaagcctttaaagttgaagaatggcgtgttattggcgaatatgtttatgatgatgaaggtggccgccatcaggcctttgttgccccgacccgcccgaccgatgtgctgggtagccgcgttatgccgcatgaacgcattgaaattgaacagagtctgaaatatagcgatgaagaaaccatgaccctgtgggcccagagcggtctgaccgaaatgggtcgttggagtcgcggcgatgaatatggtctgcatatgctgcagaaaagcgcaatgccgtttagtctgattccgagcctgtatgccgccgaaccgctgccggcagttcaggattgggaagcactgtggaaagcctgggatgttgtgacccagggtatgctgccgcatgaagaactgaatgaaaaaccgattaagctgcgcaatgcctgtattttctatctgggtcatattccgacctttctggatattcagctgaccaaaaccaccggtcaggccccgaccgatccggcttattattatagcatttttgaacgcggtatcgatccggatgtggataatccggaactgtgccatacccatagcgaaattccggatgaatggccgccggttggcgaaattattgaatatcagggtcgtgtgcgcagccgcgtgaaagcactgtatcgcgatggcgccagtaaaattccgcgccatattgcacgtgcaatttgggttggctttgaacatgaactgatgcatattgaaaccctgctgtatatgatgctgcagagtgataaaaccctgccgccgccgcataccgcccagcctaattttgaaaaaatggcaaaacaggcctatgaagcacgtgtgccgaatcagtggtttaatgttccggaacagaccattaccctgggtatggatgatccggaagattgcaccgataccagtggccattttggctgggataatgaaaaacctgcccgtcaggccaaagttcatacctttcaggcacagggccgcccgattaccaatgaagaatatgcacagtatctgtatagtaccaaaaccaatagcgttccggccagctggagctgggaccctagtaaaaccgtgaatggtagcgcaaatggtagctataccaatggccatagcaatgtgccggatagttttctggaaggcaaatatgttcgcaccgtttatggcctggtgccgctgaaacatgccctggattggccggtttttgcaagctatgatgaactgagtggttgcgcaagctggatgggcggccgtattccgaccttcgaagaagccaaaagcatctatgcatatgtgaataagcagaaacgcgccgatgcagaacgcattctgagtaaaaccgttccggcagtgaatggtcatctggttaatgatggtgtggaagaaaccccgccgagccagagtagtgaaagtgccaaagatagcccgagcaaactgtttctggatctggcagatgccaatgtgggctttcgtcattggcatccgatgccggttaccagtcagggtaatcgtctggcaggtcagagcgaaatgggtggcgtttgggaatggaccagtagcaatctgaaaccgcatgaaggctttgaaccgatgagtctgtatccgctgtataccaccgatttctttgatggcaaacataatattgtgctgggcggcagttgggccacccatccgcgtattgcaggccgcgcaagttttgttaattggtatcagcgtaattacccgtatgcatgggtgggtgcccgcctggttcgtgatatttaa

SEQ ID NO: 32 - protein, 854 aa, unidentified
  source 1..854: note = Nectria haematococca
MPSSVNAPPAVFQGARPAVSKPSPALPDIIDIRGEHVEINLKDQIISQFNPEDGPRKLPTLLLYNEKGLQIFEDITYLDEYYLTNYEIEVLKRSSTEIARQIPEGSMVIELGSGNLRKVCLLLQAFEDLAKPIQYFALDLSRKELERTLAQVPDFKYVSCHGLLGTYDDGREWLKHPSLTGRSKCILHLGSSIGNFSRDEAAAFLGGFADVLRPSDSMIVGVDACNNPAKVYKPIMNQPRLSRTNRIHRFILNGLSHANELLSEEAFKVEEWRVIGEYVYDDEGGRHQAFVAPTRPTDVLGSRVMPHERIEIEQSLKYSDEETMTLWAQSGLTEMGRWSRGDEYGLHMLQKSAMPFSLIPSLYAAEPLPAVQDWEALWKAWDVVTQGMLPHEELNEKPIKLRNACIFYLGHIPTFLDIQLTKTTGQAPTDPAYYYSIFERGIDPDVDNPELCHTHSEIPDEWPPVGEIIEYQGRVRSRVKALYRDGASKIPRHIARAIWVGFEHELMHIETLLYMMLQSDKTLPPPHTAQPNFEKMAKQAYEARVPNQWFNVPEQTITLGMDDPEDCTDTSGHFGWDNEKPARQAKVHTFQAQGRPITNEEYAQYLYSTKTNSVPASWSWDPSKTVNGSANGSYTNGHSNVPDSFLEGKYVRTVYGLVPLKHALDWPVFASYDELSGCASWMGGRIPTFEEAKSIYAYVNKQKRADAERILSKTVPAVNGHLVNDGVEETPPSQSSESAKDSPSKLFLDLADANVGFRHWHPMPVTSQGNRLAGQSEMGGVWEWTSSNLKPHEGFEPMSLYPLYTTDFFDGKHNIVLGGSWATHPRIAGRASFVNWYQRNYPYAWVGARLVRDI

SEQ ID NO: 33 - DNA, 2520 bp, unidentified
  source 1..2520: mol_type = genomic DNA; note = Penicilliopsis zonata
atgagcccgagtgtgtgcccggcaaataatgttgaaattgttgaaattcgccaggaaaattttgaattttcactggcagaagatatctataatggtattaagctgagcgaaaatggcacccgtagtctgccgaccatgctgctgtatgatgcaaaaggcctgaatctgtttgaagaaattacctatctggatgaatactatctgaccaataccgaaattgaagttctggaaacccatgcccagcgtattgttgaacgcattccggccaatgcacagctggtggaactgggcagcggtaatctgcgtaaaattgaaattctgctgaaagaatttgagcgtaccgaaaaacatgttcattattatgcactggatctgagcctgagcgaactgaaacgtacctttagtgaaattccggttgatcagtttgaatttgtgaaactgcatggcctgcatggtacctatgccgatgccctgacctggctgagtaatccgaaaaatcgcacccgtccgaccggcgtgattagtatgggtagtagcctgggtaattttagccgtccggatgccgccagctttctgcatggttttagtcgcctgctgggtccgagcgattttatggttctgggcctggatggctgtaaaaataccgataaagtttataaggcctacaatgatagtcgtggtgtgacccgtcagttttatgaaaatggtctggcacatgccaatgaagttctgggttatgaagcattcaaaccgagcgaatgggaaattgttacccgttataatgaagaaggtggtctgcatcagacctttgttcgtccgaaatgcgatgtgaccattaatggcattaagattagtaaaggtgagaatctgctgtatgaagaagcattcaaatatgatccggccgaacgcgaaagcctgtggcgtgatgccggtctgattcataatgttgcctttggcaataccagtgatgattatcatattcatatgctgagcccggcaaatctggatctgccgaccaatccgctggaatatgcagccagcccgattccgcgcatggaagaatttcagagcctgtggaccgcctgggatattgtgaccaaaggtatggttccgggcgccgaactgctgagcaaaccgattaatctgcgtaatgttctgctgttttatctgggtcatattccgacctttagtgatattcatgttacccgcgcactgcgtggtaaactgaccgaaccgcgtcattatcagctgatttttgaacgtggtattgatccggatgttgaagatccggaacagtgtcatgcccatagtgaaattcctgatcagtggccgccgctggttgaaattctggaatatcagtggaaagtgcgcagccgtattcagagcgttctgcagagtggcggcctgaaacataatcgcaccctgggcgaagccctgtggattggttttgaacatgaaattatgcatctggaaacctttctgtatatgctgctgcagagtgataaaaccctgccgccgccgggtgttgataccccggattttgaaaaaatttttcgtgaagcacgtaagcaggcaaccccgaatcagtggtttgttgttccggaacagaccctgctgattggtctggatgataaagatgatggtgttattccgccggttagctttggttgggataatgaaaaaccgcagcgtaccgccgccgtgagcgcatttgaagcacagggccgtgcaattaccaatggtgaatatgccgaatatctggaagccaatcatattgaacagattccggccagctgggttctggcaggttttaatggcgcctgccatgttagcaataagagtggcagcaatagtagcctgcgtccgaatggttttctgagtctgtataccgttcgtaccgtttttggtccggttagcctggaactggcccaggattggccggttgtggccagttatgatgaactggcaggttatgcagaatgggttaaatgccgcattccgacctatgaagaagtgcgtagtatctatcagtatagtgaacagctgaaacatgccaccaccccgccgaaaaccaatggccatggcaccagtaatgaaagcaatcgtattaagggtgaaaccaatggcaccaaaccgtatagcaaagatcatcatcagccggttcgcccgccggttagtagcaccagcccggtgtttctggatattgaaggttgcaatgtgggttttaaacattggcatccgaccccggttattcagaatggtaataagctggcaggtcagagcgaactgggcggtgtgtgggaatggaccagtaccccgctggttccgcatgatggttttaaaccgatggatgtttatccgggttataccgccgatttctttgatggcaaacataatattgtgcagggcggtagctgggcaacccatccgcgcattgcaggtcgtaccagttttgttaattggtatcagcataattacccgtatgcctgggccggcgcccgtctggttcgcgacttataa

SEQ ID NO: 34 - protein, 839 aa, unidentified
  source 1..839: note = Penicilliopsis zonata
MSPSVCPANNVEIVEIRQENFEFSLAEDIYNGIKLSENGTRSLPTMLLYDAKGLNLFEEITYLDEYYLTNTEIEVLETHAQRIVERIPANAQLVELGSGNLRKIEILLKEFERTEKHVHYYALDLSLSELKRTFSEIPVDQFEFVKLHGLHGTYADALTWLSNPKNRTRPTGVISMGSSLGNFSRPDAASFLHGFSRLLGPSDFMVLGLDGCKNTDKVYKAYNDSRGVTRQFYENGLAHANEVLGYEAFKPSEWEIVTRYNEEGGLHQTFVRPKCDVTINGIKISKGENLLYEEAFKYDPAERESLWRDAGLIHNVAFGNTSDDYHIHMLSPANLDLPTNPLEYAASPIPRMEEFQSLWTAWDIVTKGMVPGAELLSKPINLRNVLLFYLGHIPTFSDIHVTRALRGKLTEPRHYQLIFERGIDPDVEDPEQCHAHSEIPDQWPPLVEILEYQWKVRSRIQSVLQSGGLKHNRTLGEALWIGFEHEIMHLETFLYMLLQSDKTLPPPGVDTPDFEKIFREARKQATPNQWFVVPEQTLLIGLDDKDDGVIPPVSFGWDNEKPQRTAAVSAFEAQGRAITNGEYAEYLEANHIEQIPASWVLAGFNGACHVSNKSGSNSSLRPNGFLSLYTVRTVFGPVSLELAQDWPVVASYDELAGYAEWVKCRIPTYEEVRSIYQYSEQLKHATTPPKTNGHGTSNESNRIKGETNGTKPYSKDHHQPVRPPVSSTSPVFLDIEGCNVGFKHWHPTPVIQNGNKLAGQSELGGVWEWTSTPLVPHDGFKPMDVYPGYTADFFDGKHNIVQGGSWATHPRIAGRTSFVNWYQHNYPYAWAGARLVRDL

SEQ ID NO: 35 - DNA, 2478 bp, unidentified
  source 1..2478: mol_type = other DNA; note = Penicillium digitatum Pd1
atgagtccgaccctgctgcgcaatgttgataccgtggaaattgtgaatattcatcagtgtgatatggaattttccctggtggatgatgtttataaaaatctggaccctccggcaggtaaacagcgtacctttccgaccctgttactgtatgatgccaaaggtctgaaactgtttgaagaaattacctatctggatgaatactatctgaccaataccgaaattgaaattctgaaaaagcacgcaaaaaagattgttgcacatattccggaaaatgcccagctggttgaactgggcagtggcaatctgcgtaaaattgaaattttactgcgtgaatgcgaacgcagtgaaaagaaagtggattattatgccctggatctgagcctgggtgaactgcagcgtaccttcagcgaaattagtccggaaagttttattcatgttggctttcatggtctgcatggtacctatgatgatgccgtgggttggctgaaaagtccggaaaatcgtaaacgcccgaccctggttctgagcatgggcagcagtatgggcaattttagtccgccggatgcagccgattttctgggtggctttagtaaactgctgggtccgagtgattttctgctggtgggtctggatgcatgcaaaaatccggaaaaagtttttcgtgcctataatgatagcaaaggtattacccgcaaattttatgaaaacggcctgctgcatgcaaatcgtgtgctgggctttaaagcctttaaagcagatgaatgggaaattctgaccgattatgataatcgtgaaggtcgtcatcaggccttttatgttagcaaagttgatgtgattatcaacggcattaagattcgtaaaggtgaaaaactgatctttgaagaagcatggaaatatggtcgtaatgaacgcgatcagctgtggcgtaatgcaaatctgattagccaggtggaatttggcaatagcaccgatgattatcatctgcatctgctgagtccggccgccctggatcttagcatgaatccgagcaaatatgcagcacagccgattccgagtattgaaaattttcagagtctgtggaccgcatgggatctggcaacccgcaccatggtgccgcatgaagaactgctgagccagccgattaagctgcgtaatgccctgattttctattttggtcatattccgacctttctggatattcatctgacccgtgccctgcaggaagaaagtaccgaaccgagcaattataaaaccatttttgaacgtggtatcgatccggatgttgaagatccgcagcagtgtcatagtcatagcgaaattccggatgaatggccgccgctggatgaaattctggattatcaggatcgtgttcgtaatcgtgccctgagcattctgcagcagggctatgcaagccaggatcgcgccctgggtgaagcactgtggattggttatgaacatgaagcaatgcatctggaaacctttctgtatatgctgattcagagcgataaaaccctgccgccgaccggtgttgatcgtccggattttgaacagattaatcgccaggcaaaaattaataagaagccgaataagtggttccgcattccgcgtcagaccattgaaattggcctgaatgatagcaatgaagaagttgttccgaatcagagttttggttgggataatgaaaaaccgcagcgcaaagtgaccgtgcatgcatttgaagcccaggcccgtccgattaccaatggtgaatatgcaaaatatatccaggataaaggtatcaaaacctatccggcaagttgggtttttaaaccgagccaggataatccggtgagcaaaggcattagcagcagtgatgcccaggccggtagtagcagtagcccggccggtctgagcctgaaagatattaccgttcgtaccgtgtttggtccggttgcactggaagttgcccaggattggccgctggcagccagctatgatgaagtggccagttatgccaaatgcatgaaatgtcgtattccgaccttcgaagaaacccgtagtatctatcattatagtgatcagctgaaaggtgaccgtgtgaccaatgatcatcgtaatggtgtgaatggcctggcaaatgatagcaagccgaatagcaccgaccagaccgtttttcgcgatctgaccggttgcaatgttggttttaataattggcatccgattccggttaccagcaatggcgatcagctggccggtcagggtgaaatgggcggtgtgtgggaatggaccagtaccccgctgatgccgcatgatgattttaaagccatggatatctatccgggttataccagtgatttctttgatggtaaacataatatcgtgctgggtggcagttgggccaccctgccgcgtattgcaggccgtaccacctttgtgaattggtatcagcataattatcgttatgcatgggcaggtgcacgcctggttcgcgatatttaa

SEQ ID NO: 36 - protein, 825 aa, unidentified
  source 1..825: note = Penicillium digitatum Pd2
MSPTLLRNVDTVEIVNIHQCDMEFSLVDDVYKNLDPPAGKQRTFPTLLLYDAKGLKLFEEITYLDEYYLTNTEIEILKKHAKKIVAHIPENAQLVELGSGNLRKIEILLRECERSEKKVDYYALDLSLGELQRTFSEISPESFIHVGFHGLHGTYDDAVGWLKSPENRKRPTLVLSMGSSMGNFSPPDAADFLGGFSKLLGPSDFLLVGLDACKNPEKVFRAYNDSKGITRKFYENGLLHANRVLGFKAFKADEWEILTDYDNREGRHQAFYVSKVDVIINGIKIRKGEKLIFEEAWKYGRNERDQLWRNANLISQVEFGNSTDDYHLHLLSPAALDLSMNPSKYAAQPIPSIENFQSLWTAWDLATRTMVPHEELLSQPIKLRNALIFYFGHIPTFLDIHLTRALQEESTEPSNYKTIFERGIDPDVEDPQQCHSHSEIPDEWPPLDEILDYQDRVRNRALSILQQGYASQDRALGEALWIGYEHEAMHLETFLYMLIQSDKTLPPTGVDRPDFEQINRQAKINKKPNKWFRIPRQTIEIGLNDSNEEVVPNQSFGWDNEKPQRKVTVHAFEAQARPITNGEYAKYIQDKGIKTYPASWVFKPSQDNPVSKGISSSDAQAGSSSSPAGLSLKDITVRTVFGPVALEVAQDWPLAASYDEVASYAKCMKCRIPTFEETRSIYHYSDQLKGDRVTNDHRNGVNGLANDSKPNSTDQTVFRDLTGCNVGFNNWHPIPVTSNGDQLAGQGEMGGVWEWTSTPLMPHDDFKAMDIYPGYTSDFFDGKHNIVLGGSWATLPRIAGRTTFVNWYQHNYRYAWAGARLVRDI

SEQ ID NO: 37 - DNA, 2520 bp, unidentified
  source 1..2520: mol_type = genomic DNA; note = Pyrenophora tritici-repentis
atggcaaccaaaattatcgatatccgcgtggataccgccgaaagcgatattctggccgatattaagaaaggcctgcgtccggaaaatggcggtgaaaagaaactgccgaccctgctgctgtatgatcaggaaggtctgcgcctgtttgaaaaaattacctatcaggaagaatactacctgaccaatgcagaaattgaagtgctggaaacctatgcagatagtattgcagaacgtattagtagtccgagcattattgtggaactgggcagcggcaatctgcgcaaagttaatattctgctgcaggccctggatcgcctgggcaaagatgttgaatattatgcagtggatctgagtctgccggaactggaacgtacctttggtcagattccgattgaaggctataaacatgtgaaatgtttcggtctgcatggcacctatgatgatgccctgggttggctgaaaagcccggcaattgaagcaaaaccgaaaaccattctgtggctgggcagcagcctgggtaattttaaacgccatgaagttccgccgtttctggccggctttggcgaagtgctgcagaccggcgataccatgctgattggcattgatagttgtaaagatccggaacgcgtttttcatgcatataatgatcgtaatggtgttacccatcgttttattctgaatggcctgaaacatgcaaatgcactgatgggcgaaaatgcctttaatctggatgattgggaagttattggtgaatatgataaacaggcaggccgccatcatgcctttgtggcaccgcgcaaagatgttgtgattgatggcgtgccggtgaaaaaaggtgaacgcattcgcattgaagaaagctataaatatagcggtgaagaagcaaaagaactgtgggaaatggccaaactgaccgaaaatgtggtgtggccgaatgcaaaaggtgactatggcctgcattttgtgagtaaaccggccgtgtttttcccgaccaaaccggaagaatatgcagcaaaaccggtgccgagcctgaccgaatggcaggaactgtggaaagcatgggatgccgttagcaaacagatgattccggaagaagaactgctgagtaaaccgattaagctgcgcaatgaatgcattttctatctgggtcatattccgacctttctggatattcatattgcacgtgccaccggtaaaaaaccgagcgatccggcatatttttggaaaatttttgaacgtggtgtggaccctgatgtggaagatccgacccgctgccatgcacatagcgaagtgccggaagaatggccgccgctgaaaaccattttaatgtttcagcagagtgttcgcgataatgttgaagcactgtatagcagcggtgaagccgaaagcaatggtcgtgtgagccgcgccctgtggctggcttttgaacatgaagcaatgcatctggaaaccctgctgtatatgctgattcagagtgataaagttctgccgccgccgggtaccaccgttccggattttgcagcatttgccgcccgtagtaatagcctggcagtggaaaatgaatggtttaccattccggcaagcgatgttagcgttggtctggaagatccggaaggtgactatgatagccagcgttattttggttgggataatgaacgtccgcatcgtagtacccatattaagagctttcgtgccaaagcccgtccgattaccaatggcgaatatgcaacctatctgagcgaaaccggcaaaaccgttattccggcaagttggtgcgaacagccgtattataatgcaaaagccaccagtaccgcaaaacgcgatagtgttattaatggccatcagaatggtaccaatggtagcaccaccggcattaccgatggcaaatttgttcgcaccgtgtttggcaccgttccgctgaaactggccctgaattggccggttgtggcaagttatgatgaactggcaggctgtgcccagtggatgggtggtcgtattccgaccatggaagaagcccgcagcatctatagttatgtggaaagcatgaaagaagaatttgaaaaaagcctgggtaacaccattccggccgtgaatggccatctgattaatgaaggtgtgtttgaaaccccgccgagcaaaccgctgagtaatggtaatagtggtgcaggtccgagcctgaatccgcatgatctgtttattgatctggaaggcaccaatgtgggttttaaacattggcatccggttagcgtggcagaacgcggtaataagctgtgtggtcagagcgatctgggcggtgtgtgggaatggaccagtaccgttctggaaaaacatgatggttttgaaccgatggaactgtatccgggctataccgcagatttctttgatggcaaacataatattaccctgggtggtagctgggcaacccatccgcgtattgcaggccgtaaaacctttgttaattggtatcagcgcaattatccgtatatgtgggccggcgcacgtattgtgagcgatgtttaa

SEQ ID NO: 38 - protein, 839 aa, unidentified
  source 1..839: note = Pyrenophora tritici-repentis
MATKIIDIRVDTAESDILADIKKGLRPENGGEKKLPTLLLYDQEGLRLFEKITYQEEYYLTNAEIEVLETYADSIAERISSPSIIVELGSGNLRKVNILLQALDRLGKDVEYYAVDLSLPELERTFGQIPIEGYKHVKCFGLHGTYDDALGWLKSPAIEAKPKTILWLGSSLGNFKRHEVPPFLAGFGEVLQTGDTMLIGIDSCKDPERVFHAYNDRNGVTHRFILNGLKHANALMGENAFNLDDWEVIGEYDKQAGRHHAFVAPRKDVVIDGVPVKKGERIRIEESYKYSGEEAKELWEMAKLTENVVWPNAKGDYGLHFVSKPAVFFPTKPEEYAAKPVPSLTEWQELWKAWDAVSKQMIPEEELLSKPIKLRNECIFYLGHIPTFLDIHIARATGKKPSDPAYFWKIFERGVDPDVEDPTRCHAHSEVPEEWPPLKTILMFQQSVRDNVEALYSSGEAESNGRVSRALWLAFEHEAMHLETLLYMLIQSDKVLPPPGTTVPDFAAFAARSNSLAVENEWFTIPASDVSVGLEDPEGDYDSQRYFGWDNERPHRSTHIKSFRAKARPITNGEYATYLSETGKTVIPASWCEQPYYNAKATSTAKRDSVINGHQNGTNGSTTGITDGKFVRTVFGTVPLKLALNWPVVASYDELAGCAQWMGGRIPTMEEARSIYSYVESMKEEFEKSLGNTIPAVNGHLINEGVFETPPSKPLSNGNSGAGPSLNPHDLFIDLEGTNVGFKHWHPVSVAERGNKLCGQSDLGGVWEWTSTVLEKHDGFEPMELYPGYTADFFDGKHNITLGGSWATHPRIAGRKTFVNWYQRNYPYMWAGARIVSDV

SEQ ID NO: 39 - DNA, 2481 bp, unidentified
  source 1..2481: mol_type = genomic DNA; note = Talaromyces stipitatus
atgagcccggcactgctgagcaatggcagtgttaatattgttgatattcgtgataaggacgccaattttagcgccgcagcagcaattcaggatggtctggaccctccggccggcaaagcccgtagctttccgaccgtgctgctgtatgatgccgttggcctgcgtctgtttgaagaaattacctatctggatgaatactatctgaccaataccgaaattgaagtgctggaaaaacatgcacgtaccattgcagaacgtctgccggatcagagccagctggttgaactgggtagcggcaatctgcgcaaagttgaaattctgctgcgcgaatttgaaaatctgcagaaacgcgtggattattatgcactggatctgagcctggaagaactgcagcgtacctttgcacaggttagtccgcagagttatcattatgttcgttttcagggcctgcatggcacctatgatgatgccctggaatggctgaaaaatccgcagaatcgtaaacgcccgacctgtgttctgagcctgggtagtagcattggcaattttaatcgcaaagccgccgcagattttctgcgccagtatagtcagctgctgggcccgaccgatagcattattattggtctggatggctgtaaagataaagatcgtgtttatcgtgcatataatgatagtaaaggcattacccatcagttttatctgaatggtctgagccatgcaaatcaggtgctgggttataatagttttcgcccggatcagtgggatattgaatgtctgtatgatgaagcagatggttgtcatcgcgccttttatgtgccgacccaggatgtgaccattaatggtattagtctgcgtaaaggtgaaaaaattatctttgaagaggcctataagtacgatgcccaggaacgcgaagaactgtggcgtgatgccggtctgattaatgttgcagcactgggcaatagccatgataattatcatctgaatatgctgagcccggcaaaagtgagttttccgagtcgcccgagcgaatatgcaccgagtgccgtgccggcctgggaagaatggcgtagcctgtggaccagctgggatgtggttagcaaaaccatggtgccgcgtgatgaactgctgagcaaaccgattaagctgcgtaatgcactgattttctatctgggccatattccgacctttctggatattcatctgacccgcgccacccgcggcaaaccgacagatccgaaatattatccgcagatttttgaacgcggtattgatccggatgtggataatccggaacagtgccatgcacatagtgaaattccggatgaatggccggccctgggcgaaattctgcgctatcaggaacaggtgcgtagccgtgtgcagagcctgctgcgtaccgaagatgttagccagaatcgcctgctgggtgaagcactgtggattggctttgaacatgaagttatgcatctggaaacctttctgtatatgctgctgcagagtgatcgtattctgccgccgctgggtgttgatagcccggattttaaaggcattgcccgccaggccgaactggatgccaaaccgaatcagtggtttagtattccggaacagaccattaccattggtgtggatgatagtgatctgagcaaactgccggcccagagctttacctgggataatgaaaaaccgaaacgtagcgttcgtgtgcatgcatttgaagcacagggccgcgccgttaccaatcgtgaatatgcacattatctgaaagaaaatagcatccatcgcattccgaccagctgggttattcaggcagcaaatagctggaataatacctttaatggtctgcataccaatggcgttagcaatggtcatggtaatagcatggataattatgcagttcgtaccgtttttggcccggttagctttgcctgggcagccgattggccggttatggcaagctatgatgaactggcaggctatgcagaatggaaaaaatgtcgtctgccgacctttgaagaagtgcgcagtatctataaatatgccgcagccctgaaaggtcagccgaatggtgtggatgccgtgagtaatggtctggccattctgaaaaagaaagatccgaccgatgccgttattaatggcgcaaatccggaaagcatttttgtggatctggcagatgccaatgttggttttaaaaattggcatccggttccggttaccccgaatggtgacaaactggccggtcagggcgaaatgggtggtgtttgggaatggaccagcaccccgctgagtgcacatgatggctttgaagaaatgaaaatctatccgggctataccgccgatttctttgatggcaaacataatattgtgcagggtggcagttgggcaacccatccgcgcattgcaggccgcaccagttttgtgaattggtatcagcataattacccgtatgcatgggtgggcgcccgtctggttcgtgatctgtaa

SEQ ID NO: 40 - protein, 826 aa, unidentified
  source 1..826: note = Talaromyces stipitatus
MSPALLSNGSVNIVDIRDKDANFSAAAAIQDGLDPPAGKARSFPTVLLYDAVGLRLFEEITYLDEYYLTNTEIEVLEKHARTIAERLPDQSQLVELGSGNLRKVEILLREFENLQKRVDYYALDLSLEELQRTFAQVSPQSYHYVRFQGLHGTYDDALEWLKNPQNRKRPTCVLSLGSSIGNFNRKAAADFLRQYSQLLGPTDSIIIGLDGCKDKDRVYRAYNDSKGITHQFYLNGLSHANQVLGYNSFRPDQWDIECLYDEADGCHRAFYVPTQDVTINGISLRKGEKIIFEEAYKYDAQEREELWRDAGLINVAALGNSHDNYHLNMLSPAKVSFPSRPSEYAPSAVPAWEEWRSLWTSWDVVSKTMVPRDELLSKPIKLRNALIFYLGHIPTFLDIHLTRATRGKPTDPKYYPQIFERGIDPDVDNPEQCHAHSEIPDEWPALGEILRYQEQVRSRVQSLLRTEDVSQNRLLGEALWIGFEHEVMHLETFLYMLLQSDRILPPLGVDSPDFKGIARQAELDAKPNQWFSIPEQTITIGVDDSDLSKLPAQSFTWDNEKPKRSVRVHAFEAQGRAVTNREYAHYLKENSIHRIPTSWVIQAANSWNNTFNGLHTNGVSNGHGNSMDNYAVRTVFGPVSFAWAADWPVMASYDELAGYAEWKKCRLPTFEEVRSIYKYAAALKGQPNGVDAVSNGLAILKKKDPTDAVINGANPESIFVDLADANVGFKNWHPVPVTPNGDKLAGQGEMGGVWEWTSTPLSAHDGFEEMKIYPGYTADFFDGKHNIVQGGSWATHPRIAGRTSFVNWYQHNYPYAWVGARLVRDL

SEQ ID NO: 41 - DNA, 2547 bp, unidentified
  source 1..2547: mol_type = genomic DNA; note = Trichoderma virens
atgccggcagtgcgcgaaagcgttctgctggcacagcagcatattaccgatgatgccatggcactgaaaaccgtgaatgccaccgcacgcagtctggatattattgatattcagaatagccgtatcgatattaatctgaaagatgaaatcctgacccagatgaatccggaagaaggtccgcgcaccctgccgaccctgctgttatatgatgaacgtggcctgcagctgtttgaagatattacctatctggatgaatactatctgaccaattatgaaatcgaactgctgaaaaaatccgcagccgaaatggccagcaaaattccggaaggtgccattgtggttgaactgggcagtggtaatctgcgtaaagtttgcctgctgctgcaggcctttgaagatgcaaaaaagaaaattgattactacgcactggatctgagccagaccgaactggaacgtaccctggcagaagccccggcctttgaatatgttagctgccgtggtctgcgcggtacctatgatgatggctgcgaatggctgaaacaggaagccattctggcacgtccgaaatgcattctgcatctgggcagcagcattggtaattttaatcgcgatgaagcagcagattttctgcgcagttttgccgaaattctgcagccgaccgatctgatgattgtgggtgtggatagttgccagaatccggataaagtgtatcatgcatataatgatagcaagggcattacccatcagtttgttctgaatggcctgacccatgccaatgaaattctgggtaatgaagtttttaacgtggaagaatggaatgtgaccggtgaatatgtttatgatgttgatggcggccgtcatcaggcctttgttagcccgctggaagtggcaagtgttctgggccatattattaagccgcatgaacgcattaagattgaacagagcctgaaatatagtgatattggtgttgccaaactgtggaaaaccgcaggcctggaagaagtgacccgctggagccataatggtgaatatggcctgcatatgctgaaaaaagccaaaatgccgtttccgcgtctgccggaactgtatgccagcggtaccctgccgacatgggcagattgggaaagcctgtgggcagcatgggataccgtgacccgtaaaatgctgccggatgaagaactgaatgaaaaaccgattaagctgcgcaatgcatgtattttctatctgggtcatattccggcctttctggatattcagctgaaaaagactaccaaagcaggcggcaccgaaccgctgtattttcatagcatttttgaacgtggtatcgatccggatgtggataatccggaaaattgccatgatcatagcgaaattccggatgaatggccgccgctggaagatattctggcctatcaggatcgcgtgcgtgaacgtctgcagaaaatgtatagtaatccggatgaactggttggtgacgtgcgtcgcgcagtgtggattggctttgaacatgaagttctgcatctggaaaccctgctgtatatgctgctgcagagtgataaaaccctgccgccgccgcataccgttattccggattttccgaaaatggcccagaaagcctatgcacagcgcgttccgaatcagtggtttgaaattccggaacagaccattaccattggcatggatgatccggaagatgaacatgatagcaaacgccattttggttgggataatgaaaaacctgcacgtcaggaaaaagtgcatgcatttgaagccaaagcccgtccgattaccaatgaagaatatgcaaaatatctgtacagcagccatattgaagcactgccggccagttggagtattattccgccgaattatcatcataataccaatgcaaccaccccgggtaaaccgattctgagtgaactgccggaaagttttattcatgataaagcagttcgtaccgtttatggcctggtgccgctgcgctatgccctggattggccggtgtttgccagctatgatgaactggccggctgcgcagcatggatgggcggtcgtattccgaccatggaagaagcaaaaagtatctatgcctatgttgaaaaacagaaagatatcgccaaacagagcaaactgagcaataaggtgccggccgttaatggtcatctggttaatgatggcgttcaggaaaccccgccgagtgcaaccagcccgagcagtctgtttaccgatctgagtaccaccaataccggttttctgcattggcatccggtgccggttaccccgaaaggtggcagtctggccggccagggcgattttggtggtgtgtgggaatggaccagcaccattctgcgcccgcatgaaggctttcgtccgatgagtatctatccgggttataccgcagatttctttgatgaaaaacataatgtggtgctgggtggtagctgggccacccatccgcgcgttgccggtcgtaaaagctttattaattggtatcagcgcaattatctgtatgcatgggttggtgcccgtctggttcgcgatctgtaa

SEQ ID NO: 42 - protein, 848 aa, unidentified
  source 1..848: note = Trichoderma virens
MPAVRESVLLAQQHITDDAMALKTVNATARSLDIIDIQNSRIDINLKDEILTQMNPEEGPRTLPTLLLYDERGLQLFEDITYLDEYYLTNYEIELLKKSAAEMASKIPEGAIVVELGSGNLRKVCLLLQAFEDAKKKIDYYALDLSQTELERTLAEAPAFEYVSCRGLRGTYDDGCEWLKQEAILARPKCILHLGSSIGNFNRDEAADFLRSFAEILQPTDLMIVGVDSCQNPDKVYHAYNDSKGITHQFVLNGLTHANEILGNEVFNVEEWNVTGEYVYDVDGGRHQAFVSPLEVASVLGHIIKPHERIKIEQSLKYSDIGVAKLWKTAGLEEVTRWSHNGEYGLHMLKKAKMPFPRLPELYASGTLPTWADWESLWAAWDTVTRKMLPDEELNEKPIKLRNACIFYLGHIPAFLDIQLKKTTKAGGTEPLYFHSIFERGIDPDVDNPENCHDHSEIPDEWPPLEDILAYQDRVRERLQKMYSNPDELVGDVRRAVWIGFEHEVLHLETLLYMLLQSDKTLPPPHTVIPDFPKMAQKAYAQRVPNQWFEIPEQTITIGMDDPEDEHDSKRHFGWDNEKPARQEKVHAFEAKARPITNEEYAKYLYSSHIEALPASWSIIPPNYHHNTNATTPGKPILSELPESFIHDKAVRTVYGLVPLRYALDWPVFASYDELAGCAAWMGGRIPTMEEAKSIYAYVEKQKDIAKQSKLSNKVPAVNGHLVNDGVQETPPSATSPSSLFTDLSTTNTGFLHWHPVPVTPKGGSLAGQGDFGGVWEWTSTILRPHEGFRPMSIYPGYTADFFDEKHNVVLGGSWATHPRVAGRKSFINWYQRNYLYAWVGARLVRDL

SEQ ID NO: 43 - DNA, 2556 bp, unidentified
  source 1..2556: mol_type = genomic DNA; note = Uncinocarpus reesii
atggttctgccgatggcaggcgtggatattattgatattcgccgcagcaattttaatcatagcctggcaaaagaagttctggatggcctgcgcgccaaagatggcagccagcgtagtctgccgaccctgctgctgtatgataccgaaggtctgcgcctgtttgaagaaattacctatctggatgaatactatctgaccaatgcagaaattgaagttctgaccagtcatgccgcaggtattgtggaacgtgtgccggaaaatgcacagctggttgaactgggtagtggtaatctgcgtaaaattgaaattctgctgaaagaatttgagcgtgttcgcaaaagcgttgaatatctggccctggatgttagtctggaagaactgcatcgtacctttgccgaaattccgagtaaaagttataaatatgtgaagtgcggtggcctgctgggtacctatgatgatgccctggcatggctgaaacgcagtgaaaatcgccgtaaaccgacctgggttatgagcatgggtagtagcatgggtaattttacccgcaccgaagccgcccagtttctgggcggttttgccaaaaccctgggtccggatgatgcactgtttattggtctggatagttgcaaagatccgcagaaagtttttcgcgcctataatgatagcaaaaatgttacccgcgaattttatctgaatggcctggtgaatgcaaatagtattctgggttttgaagcctttcgtcgtatggattgggatgttgttggcgaatatgatgaagaaaatggttgccataaagcatattatagcccgctgaaagatgtgaccattcaggatctgagcattcagaaaggtgaaaaaattttctttgagcaggcattcaaatacagcaaacaggaatatgaagccctgtggcagcagagtggcctgaaaccgattgcccgctttagtaataccaccggcgatcatcatattcatctgctgagtagcagcccgtatattgttccgacccagccggccgaatatgccccgagtgcaaccccgagtctgaaagaatttgaagcactgtggaaactgtgggataccgtgaccaccgaaatgctgccgcgcaatgaactgctgagtaaaccgattaagctgcgcaatagtctgattttctatctgggtcatattccggcatttctggatattcagattgcaaaagccaccaccggtcagccgaccgaaccgaaaagctatcatagcacctttgaacgcggcattgatccggatgttgatgatccgaccaaatgtcatgatcatagcgaaattccggcagaatggccgccggttgaagaaattctgcgctatcagaccgcagttcgtaatcgcgcccgtctgctgctgcagaaaagccagagcgtgctggatcgtcgcattcatgaagccctgtggattggctttgaacatgaagcaatgcatctggaaacctttctgtatatgctgctgcagagtgataaagttctgccgccgccggaaattatgcagccggattttgaatatctggcaattcgcagcgcccaggaaagtgtgccgaatgaatggtttaccgttccggaacagaccattagcattggtctggacgatccgggtagtgcccagattccgacccagagttttagttgggataatgaacagccgcgtcgtagtgccaaagtgcatagctttgaagccaaaggccgcccgattaccaatggtgaatatgcaaaatatctggaagcaaatgaaccgcgtgccattccggccagctggaccaaaagtccgaaaagttttagcaaaagtaacggtctggtgaatggcaataccaatggtgcaaatggtcatggcattaatggcgccagcaccgcaccgcagtttctggaaaaatattgtgttcgcaccgtttttggtccggtgccgctgcgttttgcagcagattggccggttattgcaagttataatgaactggaaggttatgccaattgggcaaattgccgcattccgacctttgaagaagcccgtagcctgtatcagtatagcgcctttctgaaaagcagtgcagatagcagcgtgtgtgcagcagtgaatggcaacagcaataccgttaaaaaagcacatggcaatagcaatggttttgtgcatcagcagaatggtaaaccgcgtgccccggatcatcagccggttagtctggcaagcgcaagccaggttccggtttatattgatctggatggctataatgtgggttttaaacattggcatccgagtccggtgacccagaatggtaataagctgagcggtcagggcgatatgggtggtgtttgggaatggaccagcagcgccctgcagccgcatgaaggttttaaagccatggatctgtatccggcctataccgcagatttctttgatggtaaacataatatcgtgctgggcggcagttgggcaacccatccgcgcattgccggtcgcaccacctttgttaattggtatcagcgcaattatccgtttgcctgggcaggtgcccgtctggtgcgcgatgtgtaa

SEQ ID NO: 44 - protein, 851 aa, unidentified
  source 1..851: note = Uncinocarpus reesii
MVLPMAGVDIIDIRRSNFNHSLAKEVLDGLRAKDGSQRSLPTLLLYDTEGLRLFEEITYLDEYYLTNAEIEVLTSHAAGIVERVPENAQLVELGSGNLRKIEILLKEFERVRKSVEYLALDVSLEELHRTFAEIPSKSYKYVKCGGLLGTYDDALAWLKRSENRRKPTWVMSMGSSMGNFTRTEAAQFLGGFAKTLGPDDALFIGLDSCKDPQKVFRAYNDSKNVTREFYLNGLVNANSILGFEAFRRMDWDVVGEYDEENGCHKAYYSPLKDVTIQDLSIQKGEKIFFEQAFKYSKQEYEALWQQSGLKPIARFSNTTGDHHIHLLSSSPYIVPTQPAEYAPSATPSLKEFEALWKLWDTVTTEMLPRNELLSKPIKLRNSLIFYLGHIPAFLDIQIAKATTGQPTEPKSYHSTFERGIDPDVDDPTKCHDHSEIPAEWPPVEEILRYQTAVRNRARLLLQKSQSVLDRRIHEALWIGFEHEAMHLETFLYMLLQSDKVLPPPEIMQPDFEYLAIRSAQESVPNEWFTVPEQTISIGLDDPGSAQIPTQSFSWDNEQPRRSAKVHSFEAKGRPITNGEYAKYLEANEPRAIPASWTKSPKSFSKSNGLVNGNTNGANGHGINGASTAPQFLEKYCVRTVFGPVPLRFAADWPVIASYNELEGYANWANCRIPTFEEARSLYQYSAFLKSSADSSVCAAVNGNSNTVKKAHGNSNGFVHQQNGKPRAPDHQPVSLASASQVPVYIDLDGYNVGFKHWHPSPVTQNGNKLSGQGDMGGVWEWTSSALQPHEGFKAMDLYPAYTADFFDGKHNIVLGGSWATHPRIAGRTTFVNWYQRNYPFAWAGARLVRDV

SEQ ID NO: 45 - DNA, 2301 bp, unidentified
  source 1..2301: mol_type = genomic DNA; note = Schizosaccharomyces octosporus
atgatcagcaataacatcatcaacatcggcagtctggaagttctgtttagcccggaaattattgaacagtgtctgaaagtttgccagctgccgaccagcctgctgtatgatgaaaaaggtctgcagctgtttgataaaattaccggcaccgaagaatattatctgtttgattgcgaactgagcattctgcagcgcgatagcgatgcaattgcccaggaactgctgagtccggatctgccgaataccgtggtggaactgggttgtggcgcaatgcataaaaccaaacatctgctggatgcatttgaacgtaccggcaaagatgtgaatttttatgccctggatctgaatgaagatgaactgcgccgtggcctgagccagctggaacagcatgccagctataaacatgttaaagttgcaggtatttgcggctgctttgatatgtttctgaaaaatattgacaagttccgtggtagtagcaatggccagattagcattctgtatctgggtagcagcattggcaattttaatcgcgatagtgcaaccaaattcattaagagttttagcgatcgtctggcaattggcgataaatttctgctgagttttgatcatcgcaatagtgccgaactggtggaacgcgcctatgatgatagcacccgtgttaccgaaacctttgaaaagaatattctgaccagcgccaatcgcgtgtttggcgaagatttgtttaatgaaaatgactgggattacgttagtaaatatgaagaagatttgggtgtgcatcgtgcctatctgcgtgccaaaaaagatttgaccattgccaaaggcccgatggtttttaattttaaagccggtcatctgctgctgtgtgaagaaagttggaaaagtaatgataacgaatgtcgcgaaattattcataatggcaattttgttgtggacaatgttcataccaataccaccccgagttatagcgtgtatgttggtagtaaaagttttccgattctgccgcagattccgaaagaagccagcattagtctggaagaatggagccagacccgtgatatttggctgtttgtgaccaataagctgctgaatgatagtaatattttcaacgtgtggattcctctgcgccatccgtttattttctatatgggccatattccggtttttaatgatatctatctgagtcgtatcttcgaaaatccggcaaccgccagcaaacgtgaatattgggattggtttcagcgcggtattgatccggatgttgaaaatccggaacagtgccattggcatagtcagaccccgccgaaatggccgagcccgaatgaactgcgtagttatgaagttgcaagctggcagaatcatattctgaaactgctggatggcagccatgccctgagcccgagtcagaaacgtattctgtggctgtgttatgaacatgttgccatgcatattgaaaccaccctgtatatctatgtgcagagttttcagcatccgaaacagaataataccctgtgtggtctgccgccgagtaaaaatctgaaactgaaaaaagatccgagctggattaagtttccgaatgcacaggttctgcagggtctgccgattcgcagtgatcagaaaaccaaactgaatagcgaagaaccggatgaacaggaatttttcggctgggataatgaaaaaccgctgcgcatgaaacagccgagttttcagattgcaaatcgcccgattagcaatggtgaatatctggattatctggaaagtaaaccggccgatgataaacattatccgaaaagctggaaagtgattgatggtaaactgtatgtgaccaccatgtatggcctgctgccgctggaaagttatcatagctggccggttatggcaagctttgaagaactgaatgattatgcagcaagtaaaggttgtcgtctgccgaccgaagaagaactgaaccatttttatgatcatgtgctgcatcgcaaaagcgaaacctatgtgagtaccaaaggcatggcaaccggttttcagcagctgcatccggccaatctgaaagatgatggcacccatcagatttttaccggtgcctgggaatggagtagcaccgttctggataaacatgaaggctttgaaccggaagccctgtatccggattataccaaagatttctttgatggtaaacacaatgtggtgctgggcggcagttttgcaaccgttccgcgcattgcaaatcgtcgcagctttcgtaatttttatcagcgtcagtatcagtatgcctggattaccgcacgtctggccaaaagcatttaa

SEQ ID NO: 46 - protein, 766 aa, unidentified
  source 1..766: note = Schizosaccharomyces octosporus
MISNNIINIGSLEVLFSPEIIEQCLKVCQLPTSLLYDEKGLQLFDKITGTEEYYLFDCELSILQRDSDAIAQELLSPDLPNTVVELGCGAMHKTKHLLDAFERTGKDVNFYALDLNEDELRRGLSQLEQHASYKHVKVAGICGCFDMFLKNIDKFRGSSNGQISILYLGSSIGNFNRDSATKFIKSFSDRLAIGDKFLLSFDHRNSAELVERAYDDSTRVTETFEKNILTSANRVFGEDLFNENDWDYVSKYEEDLGVHRAYLRAKKDLTIAKGPMVFNFKAGHLLLCEESWKSNDNECREIIHNGNFVVDNVHTNTTPSYSVYVGSKSFPILPQIPKEASISLEEWSQTRDIWLFVTNKLLNDSNIFNVWIPLRHPFIFYMGHIPVFNDIYLSRIFENPATASKREYWDWFQRGIDPDVENPEQCHWHSQTPPKWPSPNELRSYEVASWQNHILKLLDGSHALSPSQKRILWLCYEHVAMHIETTLYIYVQSFQHPKQNNTLCGLPPSKNLKLKKDPSWIKFPNAQVLQGLPIRSDQKTKLNSEEPDEQEFFGWDNEKPLRMKQPSFQIANRPISNGEYLDYLESKPADDKHYPKSWKVIDGKLYVTTMYGLLPLESYHSWPVMASFEELNDYAASKGCRLPTEEELNHFYDHVLHRKSETYVSTKGMATGFQQLHPANLKDDGTHQIFTGAWEWSSTVLDKHEGFEPEALYPDYTKDFFDGKHNVVLGGSFATVPRIANRRSFRNFYQRQYQYAWITARLAKSI

SEQ ID NO: 47 - DNA, 2277 bp, unidentified
  source 1..2277: mol_type = genomic DNA; note = Schizosaccharomyces japonicus
atgatggccgaaagtattattgatatcggcgccaccgcagatatttttagtgcacagagcgtgagcgcaaatctgaaacagagccgtctgagtagcagcctgctgtatgatgaaaccggtctgcagctgtttggccagattacccaggaagatgaatattatccgtttcgtctggaaatgcagctgctgcagaaacatgcagattggattgcagaacatgtgcgtagtaaaaccagtaccaccattattctggaactgggttgtggtagcatgcgtaaaaccaaagtgctgctggatgcctttgaaaatacctgcagtccggttcattattatgcactggatctgaatcgcaaagaactgcagaatagcctgaataccctggaagcaagtaccagctatcgcaatgtgaaaattagtggtatttgtggttgttttaagcatgcactgagttatctgccgattctgcgcagtagtccgaatagtaaatttgtgctgacctatctgggtagcagcattggcaattttagccgtgaagaaagtgcaacctttctgcaggcattttctagtaaactgaaaccggatgatcagattattgtgagttttgatcatcgtcatgaaaaagaaaccattattagcgcctataatgataaacatcacattaccgaaaagttcgaactgaatattctgaatcatgtgaatcatatcttcggtgcacgcctgtttcatctggatgattggcgctatcagggcgaatatgatgaacataccggtgtgcataaagcatttctgattagtaaacgcccggtgaccattccggaactgcagctgagttttccgcagaatcataaactgctgtgcgaagaaagctggaaaagcagcagtgaagaagccaataagattctgcataatggcggtttctttaccgaagcagaactgaaaagtaatcatggttttagcctgtttatcgcaagcgtgccgacctttgatgtgagccgtaatccggaaaccccgtgcccgaccctggaagaatggacccagattcgcctggcatggctgtatctggtttttaaactgtatccgcgcgatctgtattttaccgaactgattccggttcgtcatccgtttattttctatattggtcatgttccggcctttaatgatatctatctggcccgtctgaccgatggcaaaccgaccctgggccgcaaagattattgggattggtttcagcgtggcattgatccggatctggataataccaaaaaatgccattggcatagccagccgccggaaaaatggccgagtgttgaagaagtgaatgaatatgaacgcaatgtttggagtcgcctggttagcatctataaacagggtgaaatgagcgccaatatgcagcgcgcaatgtggatgatctatgaacataccgccatgcatctggaaaccagctattatattctgctgcagagcgattatcatattattagtccgaataacttcccgccgccgattgcaccggccctgcagacagatccgacctgggttcgcgtgccggaaagctttattaccatgggtattccgaccaccgcagatggtaaagaaaccttttattatggctgggataatgaaaaaccggaacgtcaggttagcgtgcgctgttttgaaattgccaatcgcccgattagtaatggcgaatatctgagctttctgaaagaaaccacccagagcaaagaagaatttgaagcagcaattccgaaaacctggctgctgaaagatgaaatgctgtttgcaaaaagtatgtatggtccgctgccgattgaacatgtgctgggctggccggttgccaccagctatgatgaactgaaacagtatgccaatgcaaaaggctgtcgtctgccgaccgattatgaactgcgcgcattttatgatcatgtgctgaaaccgaatgaagaaacctatgttgataccgccggttatgccaccgcctttcagcagtggtatccgaaaagtctgcaggatgaagaaaaaccgcagatatataccggcctgtgggaatggaccagtaccgtgctgaaagaagatttggattttaccccggaagaactgtatccggattatacccgcgatttctttgatggtaaacataatgttgtgatgggcggtagctttaccaccgttgcacgcattgcaaatcgtcgtagttttcgtaatttttatcagcgtaaatacccgtatgcctggattggcgcacgtctggtgaaagtgaccaataccctgagctaa

SEQ ID NO: 48 - protein, 758 aa, unidentified
  source 1..758: note = Schizosaccharomyces japonicus
MMAESIIDIGATADIFSAQSVSANLKQSRLSSSLLYDETGLQLFGQITQEDEYYPFRLEMQLLQKHADWIAEHVRSKTSTTIILELGCGSMRKTKVLLDAFENTCSPVHYYALDLNRKELQNSLNTLEASTSYRNVKISGICGCFKHALSYLPILRSSPNSKFVLTYLGSSIGNFSREESATFLQAFSSKLKPDDQIIVSFDHRHEKETIISAYNDKHHITEKFELNILNHVNHIFGARLFHLDDWRYQGEYDEHTGVHKAFLISKRPVTIPELQLSFPQNHKLLCEESWKSSSEEANKILHNGGFFTEAELKSNHGFSLFIASVPTFDVSRNPETPCPTLEEWTQIRLAWLYLVFKLYPRDLYFTELIPVRHPFIFYIGHVPAFNDIYLARLTDGKPTLGRKDYWDWFQRGIDPDLDNTKKCHWHSQPPEKWPSVEEVNEYERNVWSRLVSIYKQGEMSANMQRAMWMIYEHTAMHLETSYYILLQSDYHIISPNNFPPPIAPALQTDPTWVRVPESFITMGIPTTADGKETFYYGWDNEKPERQVSVRCFEIANRPISNGEYLSFLKETTQSKEEFEAAIPKTWLLKDEMLFAKSMYGPLPIEHVLGWPVATSYDELKQYANAKGCRLPTDYELRAFYDHVLKPNEETYVDTAGYATAFQQWYPKSLQDEEKPQIYTGLWEWTSTVLKEDLDFTPEELYPDYTRDFFDGKHNVVMGGSFTTVARIANRRSFRNFYQRKYPYAWIGARLVKVTNTLS

SEQ ID NO: 49 - DNA, 2520 bp, unidentified
  source 1..2520: mol_type = genomic DNA; note = Bipolaris sorokiniana
atggccgcaaatattattgatatccgcgttgataaagccgaaagcgatattctggccgatattaagaaaggtctgcgcccggttgccgatgcagaaaaaaccctgccgaccctgctgctgtatgatcaggaaggcctgcgcctgtttgaacagattacctatcaggaagaatattatctgaccaatgccgaaattgaagttctggaaacctatgccgataaaattgcccagcgcattagtccgggcagcattgttgttgaactgggcagcggtaatctgcgcaaagttaatattctgctgcaggccgtggatcgcctgggcaaagatattgaatattatgccgtggatctgagcctgccggaactggaacgcacctttaaacagattccgattgaaggttatagtcatgtgaaatgtttcggtctgcatggtacctatgatgatgccctggaatggctgaaaagcccggcagttgaagccaaaccgaaaaccattctgtggctgggtagcagcctgggcaattttaaacgccatgaagtgccgccgtttctggcaggctttggtaaagttctgcagaccggtgacaccatgctgattggtattgatagctgcaaagatccgaaacgtgtgtttcatgcatataatgatcgtgatggtgtgacccatcgctttattctgaatggtctgaaacatgcaaatgccctgatgggtgaaaatgcattcaatctggatgattgggaagttattggcgaatatgataccaaagcaggtcgccatcatgcatttgttgccccgcgcaaagatgttgtggtggatggtgttccgatgaaacagggtgaacgcattcgcattgaagaaagttataaatatagccgcgaagaagcaaaaaagctgtgggaactggccaaactggccgaaaatgcagtgtgggccaatagtaaaggtgactatggtctgcatatggtgagcaaaccgagctttttctttccgaccaccccggaagaatatgcagaaaaaccggttccgagcctgaccgaatggcaggaactgtggaaagcctgggatgcagttagtaaacagatgattccgaatagtgaactgctggccaaaccgattaagctgcgcaatgaatgcattttctatctgggtcatattccgacctttctggatattcatattgcacgtgccaccgatggcaaaccgaccgaaccggcctatttttggaaaatttttgaacgcggcgtggaccctgatgttgatgatccgaccctgtgccatgcccatagcgaagtgccggaagaatggccgccgctgggtaccattctgcagtatcagcagaccattcgtaaaaatctggaagcactgtatgatagcggcgaagcagaaaagaattgtcgcattagtcgtggtctgtggattgcatttgaacatgaagcaatgcatctggaaaccctgctgtatatgctgattcagagcgataaagtgctgccgccgccgggcattaagcagccggattttgccgcatttgcagcacagagtgaagttatggccgtggaaaatgaatggtttaccattccggaaagtgatattgatattggcctgaatgatccggaaaaagattttggtagtaaacgctatttcggctgggataatgaacgcccgtgtcgtagtgttcatgttaaaagctttcgtgcaaaagcacgtccgattaccaatggtgaatatgcaacctatctgctgcagaccggcaaaaaagaaattccggccagttggtgtgataaagcatatagtaatggtcatgataccaataccaccaaacgtgatagcgttgtgaatggtcagagcaatggtaatggtgaaagtagccagggcattattgaaggcaaatttgtgcgtaccgtttatggtaccattccgctgaaactggcaatgggctggccggttgtggcaagctatgatgaactggtgggttgtgcacagtggatgggcggccgtattccgaccatggaagaagcacgcagtatctatgcctatgttgatagtattaagccggaatttgaacagagcctgggtaataccattccggccgtgaatggtcatctgctgaatgaaggcgtttttgaaaccccgccgagccatcatctgagcaatggcaatagtggtgcagtgaccggcctgaaaccgcgtgatctgtttattgatctggaaggcaccaatgtgggttttaaacattggcatccggtgagtgtggcagaaaaaggtgacaaactgtgtggtcagagtgatctgggcggcgtttgggaatggaccagcaccgtgctggaaaaacatgatggctttgaaccgatggaactgtatccgggctataccgccgatttctttgatggcaaacataatattaccctgggcggtagctgggcaacccatccgcgcattgcaggccgtaaaacctttgtgaattggtatcagcgtaattatccgtatgtttgggccggtgcacgcattgttaccgatctgtaa

SEQ ID NO: 50 - protein, 839 aa, unidentified
  source 1..839: note = Bipolaris sorokiniana
MAANIIDIRVDKAESDILADIKKGLRPVADAEKTLPTLLLYDQEGLRLFEQITYQEEYYLTNAEIEVLETYADKIAQRISPGSIVVELGSGNLRKVNILLQAVDRLGKDIEYYAVDLSLPELERTFKQIPIEGYSHVKCFGLHGTYDDALEWLKSPAVEAKPKTILWLGSSLGNFKRHEVPPFLAGFGKVLQTGDTMLIGIDSCKDPKRVFHAYNDRDGVTHRFILNGLKHANALMGENAFNLDDWEVIGEYDTKAGRHHAFVAPRKDVVVDGVPMKQGERIRIEESYKYSREEAKKLWELAKLAENAVWANSKGDYGLHMVSKPSFFFPTTPEEYAEKPVPSLTEWQELWKAWDAVSKQMIPNSELLAKPIKLRNECIFYLGHIPTFLDIHIARATDGKPTEPAYFWKIFERGVDPDVDDPTLCHAHSEVPEEWPPLGTILQYQQTIRKNLEALYDSGEAEKNCRISRGLWIAFEHEAMHLETLLYMLIQSDKVLPPPGIKQPDFAAFAAQSEVMAVENEWFTIPESDIDIGLNDPEKDFGSKRYFGWDNERPCRSVHVKSFRAKARPITNGEYATYLLQTGKKEIPASWCDKAYSNGHDTNTTKRDSVVNGQSNGNGESSQGIIEGKFVRTVYGTIPLKLAMGWPVVASYDELVGCAQWMGGRIPTMEEARSIYAYVDSIKPEFEQSLGNTIPAVNGHLLNEGVFETPPSHHLSNGNSGAVTGLKPRDLFIDLEGTNVGFKHWHPVSVAEKGDKLCGQSDLGGVWEWTSTVLEKHDGFEPMELYPGYTADFFDGKHNITLGGSWATHPRIAGRKTFVNWYQRNYPYVWAGARIVTDL

SEQ ID NO: 51 - DNA, 2520 bp, unidentified
  source 1..2520: mol_type = genomic DNA; note = Exserohilum turcica
atggccacccagattattgatattcgtgttgataccgccgaaagtgatattctggcacatattaagaaaggcctgcgcccggaagcaggcggcgaaaaaaccctgccgaccctgctgctgtatgatcaggaaggtctgcgtctgtttgaacagattacctatgaagaagaatactatctgaccaatgcagaaattgaagttctggaaaaatacgcccatgaaattgcacagcgtgttccgagcggcagcattgttgttgaactgggcagcggcaatctgcgcaaagttaatattctgctgcaggccatggatcgcctggccaaagatgttgaatattatgcagttgatctgagcctgccggaactgcagcgtacctttagtcagattccgattgaaggctatagccatgttaaatgctttggcctgcatggcacctatgatgatgccctggaatggctgaaaagtccggccgttgaagccaaaccgaaaaccattctgtggctgggtagtagcctgggcaattttaaacgccatgaagttccgccgtttctggcaggctttggtcgcgttctgcagaccggtgacaccatgctgattggtattgatagttgcaaagatccggaacgtgtgtttcatgcatataatgatcgcaatggtgtgacccataaattcattctgaatggcctgaaacatgccaatgccctgatgggcgatcgtacctttaatatggatgattgggaaattatcggcgaatatgatgtgaaagcaggccgtcatcatgcctttgtggcaccgcgtaaagatgtggttgtggatggcgttaccgtgaaacagggcgaacgtattcgcattgaagaaagctataaatatagccataaggaagcaaaaaagctgtgggaactggcacgtctgagtgaaagtgcagtttgggcaaatagcatgggcgattatggtctgcatctggttagtaaaccggcatttttctttccgaccaatccggaagaatatgccgcaagtccggttccgagcctggcagaatggcaggaactgtggaaaagttgggatgccgtgagcaaacagatgattccggaagccgaactgctgagcaaaccgattaagctgcgcaatgaatgcattttctatctgggtcatattccgacctttctggatattcatattgcacgtgcaaccgatggccagccgaccgaaccggcatatttttggaaaatttttgaacgtggcgtggaccctgatgtggataatccgacccagtgccatgcccatagtgaagtgccggaagaatggccggcagtgaaaaccattttagaatatcagcagaccattcgtaaaaataccgaagcactgtatcagagcggtgaagccgaaaataatgtgcgtgttagtcgtggcctgtggattgcctttgaacatgaagcaatgcatctggaaaccctgctgtatatgctgattcagagcgataaaattctgccgccgccgggtaccaaagttccggattttgccgcctatgcagcacatagtgatgttctggcagttgaaaatcagtggtttaccattccggaaagcgatattgatattggcctggatgatccggaaaatgattttaaaaccaaacgttatttcggctgggataatgaacgtccgcgtcgtagtgcccatgtgaaagcctttcgtagtaaagcccgcccgattaccaatggtgaatatgccacctatctgtttcagaccggtaaaaaagaaattccggcaagctggagcgaaaccagttatagtaatgccaatggcaccaccatgaccaaacgcgatagcgtgattaatggccatgcaaatggcgatagtgatccgagttatagtatgattgaaggtaaatttgtgcgtaccgtttatggtaccgtgccgctgcgttttgccatgggctggccggtggtggcaagctatgaagaactggccggctgcgcacgctggatgggtggtcgtattccgaccatggaagaagcacgcagcatctatgcctatgtggatcgcatgaaaccggaatttgaaaaaagcctgggtaataccattccggccgttaatggtcatctgattaatgaaggcgtttttgaaaccccgccgagtccgcatctgagtaatggcaatagcggtgccgccaccagtctgaatccgcatgatctgtttattgatctggaaggcgccaatatgggctttaaacattggcatccggttagcgttgcagataaaggcaataagctgtgtggtcagtatgatctgggtggcgtttgggaatggaccagtaccgttctggaaaagcatgaaggttttgaaccgatggaactgtatccgggttataccgccgatttctttgatggtaaacataatgttaccctgggtggcagctgggccacccatccgcgtctggcaggccgtaaaacctttattaattggtatcagcgcaattatccgtatgtttgggccagcgcccgtattgttgcagatttgtaa

SEQ ID NO: 52 - protein, 839 aa, unidentified
  source 1..839: note = Exserohilum turcica
MATQIIDIRVDTAESDILAHIKKGLRPEAGGEKTLPTLLLYDQEGLRLFEQITYEEEYYLTNAEIEVLEKYAHEIAQRVPSGSIVVELGSGNLRKVNILLQAMDRLAKDVEYYAVDLSLPELQRTFSQIPIEGYSHVKCFGLHGTYDDALEWLKSPAVEAKPKTILWLGSSLGNFKRHEVPPFLAGFGRVLQTGDTMLIGIDSCKDPERVFHAYNDRNGVTHKFILNGLKHANALMGDRTFNMDDWEIIGEYDVKAGRHHAFVAPRKDVVVDGVTVKQGERIRIEESYKYSHKEAKKLWELARLSESAVWANSMGDYGLHLVSKPAFFFPTNPEEYAASPVPSLAEWQELWKSWDAVSKQMIPEAELLSKPIKLRNECIFYLGHIPTFLDIHIARATDGQPTEPAYFWKIFERGVDPDVDNPTQCHAHSEVPEEWPAVKTILEYQQTIRKNTEALYQSGEAENNVRVSRGLWIAFEHEAMHLETLLYMLIQSDKILPPPGTKVPDFAAYAAHSDVLAVENQWFTIPESDIDIGLDDPENDFKTKRYFGWDNERPRRSAHVKAFRSKARPITNGEYATYLFQTGKKEIPASWSETSYSNANGTTMTKRDSVINGHANGDSDPSYSMIEGKFVRTVYGTVPLRFAMGWPVVASYEELAGCARWMGGRIPTMEEARSIYAYVDRMKPEFEKSLGNTIPAVNGHLINEGVFETPPSPHLSNGNSGAATSLNPHDLFIDLEGANMGFKHWHPVSVADKGNKLCGQYDLGGVWEWTSTVLEKHEGFEPMELYPGYTADFFDGKHNVTLGGSWATHPRLAGRKTFINWYQRNYPYVWASARIVADL

SEQ ID NO: 53 - DNA, 2562 bp, unidentified
  source 1..2562: mol_type = genomic DNA; note = Paraphaeosphaeria sporulosa
atggatgtgacccgtgccctgagtggcgcacgtctgcgccctctgcgttttctgaaagaaaccagctttgaaaaaatgagtgccaaaaccagcaccgaaattattgatattcgcccgggcccgaccgaatttgatattctgcaggatattaaggatggcctgcgtccggaacatggcggcgaaaaaaccctgccgaccatgctgctgtatgatgaagcaggcctgcgtctgtttgaaaaaattacctatgttaaggactactacctgaccgatagcgaaattgaagtgctggatacctatgcagatcagattgccgaacgcattaaggccggtagcgttctggtggaactgggtagtggtaatctgcgtaaagtgaatattctgctgcaggcaattgaacgtctgggtaaagatgttgaatattatgcagtggatctgagcctgccggaactggaacgcacctttgccgaaattccgaccaattatcagcatgtgaaactgaaaggtctgtatggtacctatgatcatgccctggaatggctgaaaagcccgaaagtgagcgcaaaaccgaaaaccattctgtggctgggcagtagcctgggtaattttacccgtgcagatgtgccgccgtttctgaccggttttcgtgaagcactgcagccgggtgacaccatgctgattggtattgatagctgcaaagaaccggaacgcgtttttcgcgcctataatgataccgatggtgttacccgtgattttaccctgaatggtctgaaaaatgcaaatcgcattatgggcaccgaagcctttaaaccgcatgaatgggaacattgtggtgaatttgtggaaaaagatggttatcatcgcgcatttgttagcccgctgaaagatgttattattgatggcgttcatatcaaaaagggtgaacgtattcgcattgaagaaagctggaaattttctaaagaggaaattgaacacctgtggagcgaagccggcctgattccgaataccacctttagtaccgcccgtggtgactatggtctgcattttgtgagcaaaccggcatttttctttccgaccaaaccggaacgttatgcagcaaaaccggtgccgagtattgccgaatggcgcgaactgtggaccgcctgggatgatattgcccagaaaatggtgccgaccgaagcactgctgagcaaaccgattaagctgcgtaatgcagtgattttctatctgggccatattccgacctttctggatattcatctgacccgcgcaaccgatgaaaaaccgaccgaaccggccgcatataccaaaatttttgaacgtggcattgatccggatgttgataatccggaacagtgtcatgatcatagtgaaattccggaaacctggccgccgcgcgatgaagttctggcctttcaggatgcagttcgtaaacgtaccaaagcactgtatgatagtaatgcagcccatgaaaatccgcgtgtgagccgtgccctgtggctggcttttgaacatgaagcaatgcatctggaaaccctgctgtatatgctgattcagagtgaacgtattctgccgccgccgggcagcgtgatgccgagctttgatgccctggcacgcaaaagcaaaaaagcacgcgtggaaaatcagtggtttaccattccggaagccgaaattcgtgatggtctggatgatccggaagatggcagcgccgcaaaacgctattttggctgggataatgaaaaaccgacacgcagcctgcgcgtgaaaagttttaaagcaaaagcacgcccgattaccaatggcgaatatgccgattatctggttcagaccggcaaatgtgccgtgccggcaagttggtgcgatggcctggaccctgcagcaaaagtggtggttaatggtgtgaatcgccgtaatggtaccaatagcattaatagcagtattgataaggttatgcagggtaaatatgtgcgcaccgtttttggtaccgttccgctgcattatgccctggattggccggttgtggcaagctatgatgaactggcaagttgtgcccagtggatgggcggtcgcattccgaccctggaagaagcacgcagtatctataattatgtggaacatggcaaagcccaggaatttgaaaaaacccatggcaataagattccggcagtgaatggtcatctgattaataatggtgttagtgaaagcccgccgagtcagcatctgagcaatggtagtagcggcaccggcagtgatccgaaaccgggcgatctgtttattgatctggaaggcaccaataccgcctttcagcattggcatccgattagcgtggcagaaaaaggtgacaaactgtgtggtcaggccgatctgggcggtgcatgggaatggaccagtaccgtgctggaaaaacatgatggttttagcgcaatgccgctgtatccgggttataccgcagatttctttgatggcaaacataatattatgctgggcggtagctgggccacccatagccgtattgccggtcgtaaaacctttgtgaattggtatcagcgtaattatccgtaa

SEQ ID NO: 54 - protein, 853 aa, unidentified
  source 1..853: note = Paraphaeosphaeria sporulosa
MDVTRALSGARLRPLRFLKETSFEKMSAKTSTEIIDIRPGPTEFDILQDIKDGLRPEHGGEKTLPTMLLYDEAGLRLFEKITYVKDYYLTDSEIEVLDTYADQIAERIKAGSVLVELGSGNLRKVNILLQAIERLGKDVEYYAVDLSLPELERTFAEIPTNYQHVKLKGLYGTYDHALEWLKSPKVSAKPKTILWLGSSLGNFTRADVPPFLTGFREALQPGDTMLIGIDSCKEPERVFRAYNDTDGVTRDFTLNGLKNANRIMGTEAFKPHEWEHCGEFVEKDGYHRAFVSPLKDVIIDGVHIKKGERIRIEESWKFSKEEIEHLWSEAGLIPNTTFSTARGDYGLHFVSKPAFFFPTKPERYAAKPVPSIAEWRELWTAWDDIAQKMVPTEALLSKPIKLRNAVIFYLGHIPTFLDIHLTRATDEKPTEPAAYTKIFERGIDPDVDNPEQCHDHSEIPETWPPRDEVLAFQDAVRKRTKALYDSNAAHENPRVSRALWLAFEHEAMHLETLLYMLIQSERILPPPGSVMPSFDALARKSKKARVENQWFTIPEAEIRDGLDDPEDGSAAKRYFGWDNEKPTRSLRVKSFKAKARPITNGEYADYLVQTGKCAVPASWCDGLDPAAKVVVNGVNRRNGTNSINSSIDKVMQGKYVRTVFGTVPLHYALDWPVVASYDELASCAQWMGGRIPTLEEARSIYNYVEHGKAQEFEKTHGNKIPAVNGHLINNGVSESPPSQHLSNGSSGTGSDPKPGDLFIDLEGTNTAFQHWHPISVAEKGDKLCGQADLGGAWEWTSTVLEKHDGFSAMPLYPGYTADFFDGKHNIMLGGSWATHSRIAGRKTFVNWYQRNYP

SEQ ID NO: 55 - DNA, 2493 bp, unidentified
  source 1..2493: mol_type = genomic DNA; note = Marssonina brunnea
atggcaccgaaaattgatattatcgatattcgtcataacgcagtggaaatgagtctgaaagatgaaattgttaagagcctgaaaccgcaggaaggcccgaaacgcctgccgaccctgctgctgtatgatgaacgcggcctgcagctgtttgaagaaattacctatctggaagaatactatctgaccaatgcagaaattgatgtgctgcagcgcagcgcatgcaatattgcagaagcaattccgccgggtagtatggtggtggaactgggtagtggtaatctgcgtaaagttagtattctgctgcaggcactggatcaggccggcaaagatattgattattatgccctggatctgagtctgaaagaactgtatcgtaccctggaacaggttccggcctttaaacatgtgacctgccatggtctgcatggcacctatgatgatggtctggattggctgaaaattccggaaaatattacccgcccgaaatgcgtgatgagcctgggtagtagcattggcaattttagtcgtgccggtggcgcagaatttctgaaaggttttgccgaagtgatgcaggatagcgatctgatgctggttggtctggatgccaccgaagatccggcaaaagtgtatcatgcatataatgatcgtgaaggtaaaacccataaattcattctgaatggtctgaccaatgccaatggtatctataatgaagaaatctttgagccgaatgattggaaagtgattggcgaatatgtgtttgatgcagaaggcggccgtcatcaggccttttgcagcccggttcatgatgtgagtgttaaaggtgttcagattaaggcaggtgaacgcgtgcagattgaagaaagcctgaaatatagtccggaaggtagcgcccagctgtggaaagccagtggcctgattgaagttgatcgtatgagtgcaagcagtgatagctatagcctgcatctgctgaaacgtaatatggcctttaaaaccgatccgagtctgtatgccgccagtaccgtgccgacccgcaaagattggaaaggtctgtggaccgtgtgggatctgattacccagaatatgattccgaaaaccgaactgaatgaaaaaccgattaagctgcgcaatgcctgtattttctatctgggtcatattccgacctttaccgatattcagctggaaaaagtgaccaaacagccgcgttgcgaaccgggctattttaaagaaatttttgaacgtggcatcgatccggatgtggataatccggaacgctgtcatgatcatagcgaagttccggaagaatggccgccgctgcaggatattctgggttatcaggatcaggtgcgtgccaaaattgaaaaaattaccgcaagcgaaagtattccgcgtgatgttggccgtgccctgtggattggttttgaacatgaaattatgcatctggaaaccctgctgtatatgctgctgcagagtgataaaaccctgccgccgaccaaattcaaaccgaattttgaagaactggcagccgccgatgaagcagcccgcgttggcaatgaatggtttgaaattccggaacagcgtattaccattggtctggatgatccggaagataatagtggcggtgaccgccattttggctgggattgcgaaaaaccgccgcgcagcgttgtggttccggccttcaaagcacaggcccgcgccattaccaatgaagattatgcccgttatctggaacagacccatgccagtaaaattccggcaagctggaccgaaagcgtgaccaatggtcataccaatggcgtgagcaatgtgtatagcaatggtaatagcaatggtcatgcaattaccagcaccccgctgaccaaagaatatctggatggtaaatttgtgcgcaccgtgtatggcctggttccgctgatgtttgcactgcattggccggtttttgcaagctatgatgaactggcaggctgtgcaaaatggatgggtggtcgcattccgaccctggaagaagcacgtagcatctatagtcatgtggatggcctgcgtctgaaagaagccgaacagcatctggttaaaaccgttccggccgttaatggtcatctggttaatgaaggcgttgaagaaagcccgccgagtcgcggcgcatggcctggtgaaggcagcgaactgtttaccgatctggccaatgccaatgttggctttaaacattggcatccgattggcgtgaccagcaatggcgataaactggcaggtcaggcagaaatgggtggcgtgtgggaatggaccagtagcgaactgctgcgccatgatggctttgaaccgatgaaactgtatccggcctataccgcagatttctttgatggcaaacataatattgtgctgggtggcagctgggcaacccatccgcgcattgcaggccgcaaaacctttattaattggtatcagcgtaactatccgtatgcctgggccggcgcacgcctggttcgcgacgtttaa

SEQ ID NO: 56 - protein, 830 aa, unidentified
  source 1..830: note = Marssonina brunnea
MAPKIDIIDIRHNAVEMSLKDEIVKSLKPQEGPKRLPTLLLYDERGLQLFEEITYLEEYYLTNAEIDVLQRSACNIAEAIPPGSMVVELGSGNLRKVSILLQALDQAGKDIDYYALDLSLKELYRTLEQVPAFKHVTCHGLHGTYDDGLDWLKIPENITRPKCVMSLGSSIGNFSRAGGAEFLKGFAEVMQDSDLMLVGLDATEDPAKVYHAYNDREGKTHKFILNGLTNANGIYNEEIFEPNDWKVIGEYVFDAEGGRHQAFCSPVHDVSVKGVQIKAGERVQIEESLKYSPEGSAQLWKASGLIEVDRMSASSDSYSLHLLKRNMAFKTDPSLYAASTVPTRKDWKGLWTVWDLITQNMIPKTELNEKPIKLRNACIFYLGHIPTFTDIQLEKVTKQPRCEPGYFKEIFERGIDPDVDNPERCHDHSEVPEEWPPLQDILGYQDQVRAKIEKITASESIPRDVGRALWIGFEHEIMHLETLLYMLLQSDKTLPPTKFKPNFEELAAADEAARVGNEWFEIPEQRITIGLDDPEDNSGGDRHFGWDCEKPPRSVVVPAFKAQARAITNEDYARYLEQTHASKIPASWTESVTNGHTNGVSNVYSNGNSNGHAITSTPLTKEYLDGKFVRTVYGLVPLMFALHWPVFASYDELAGCAKWMGGRIPTLEEARSIYSHVDGLRLKEAEQHLVKTVPAVNGHLVNEGVEESPPSRGAWPGEGSELFTDLANANVGFKHWHPIGVTSNGDKLAGQAEMGGVWEWTSSELLRHDGFEPMKLYPAYTADFFDGKHNIVLGGSWATHPRIAGRKTFINWYQRNYPYAWAGARLVRDV

SEQ ID NO: 57 - DNA, 2748 bp, unidentified
  source 1..2748: mol_type = genomic DNA; note = Pseudogymnoascus destructans
atgaccaatagtaccaccccgccgccggatgttgttgatctggataaatttcatacccatgatgatccgcgtcatacccgcctgaccaccccgagcaaagccaccctgccgccggctaccccgcctagccctgcacaaagcaccctggattttattgatattattgatatccgccgtgatgccctgggcagtagtctggatctgggtcgcgatattatggcccagctggcaccggcccgcggtcctaaaaagatgccgaccctgctgctgtatgatgaaaaaggcctgcagacctttgaagaaattacctatctggaagaatactatctgaccaatgcagaaattgaagtgctggaacgcaatgcagaagaaatggcccgcaatattcaggcagaaagcatggttattgaactgggtagcggtaatctgcgtaaagtgagcattctgctgaatgccctggaaaaagccgaaaaaagtattcattattacgcactggatctgagcaaacgcgaactggaacgtaccctgagcagtgttccgcgttttgaacatgtggtttgccatggtctgctgggcacctatgatgatggcctggaatggattcgtagtggttgcaatgcaagctggccgaaatgcattatgagtctgggtagtagtattggtaattttaatcgcggcgatgccgcagaatttctgaaaggttttgcagatatgctgcgtccgagtgatagcatgattattggcctggatgcctgcaatgatccggccaaagtttatcatgcctataatgatagcctgggcattacccataaattcattctgaatggtctggataatgcaaatagcattctgggcgaaaatgtgtttgataccaatgattgggaagtgattggtgaatatgtttgtgataaagatggtggtcgccatcgcgccttttatgccccgaaacgtgatattaccattcgtggtgtttttattgaacagggtgaacgcgttcaggttgaacagagcctgaaatatagccaggccgaaagcgaaggcatgtgggcagcagccggcctgaaagaagtgggcaaatggggtgcaaccaaagaacagtataatattcatatgctgaccaaacgtgcaaaaccgtttcagctgcatccgagtcagtatgccctgaccccgaccccgaccctggaagattggcgtggcctgtggagcacctggaataccgtggcccgtggtatgattccgaataatgaactgctggcaaaaccgattaagctgcgtaatgcctgcattttctatctgggccatattccgacctttctggatctgcagctgagcaaagccacaggtgtgccgctgtgtgaaccgagtcattatccgcagatttttgaacgtggtattgatccggatgtggataatccggataattgccatgcacatagtgaaattccggatcagtggccgccggtggaagaaattctggaatatcaggcacaggtgcgccgtaaagtggaaggtctgtatgccagcggtgtgccggaagcaagtcgtaaagtgggtcgtagtctgtggattggcctggaacatgaaattatgcatctggaaaccctgctgtatatgctgctgcagagcgataattgtatgccgccgccgcgcaccgtgaaaccggattttgaagaacaggcccgtcgtgatgcagaacgtgaagtggaaaatcagtggtttaccattccggaacaggatattgccctgggcctggatgatccggaagataatagcggtgacggtcattttggctgggataatgaaaaaccggttcgtaaagcccatgttcgtagctttcaggcaaaaggtcgtccgattaccaatgaagaatatgcaatctatctggatgcaaccgataatgaaaatctgccggcaagttggacccgtcagcatgccaatggcgatctgagcgcacataccccgaatggcaataccaatggttataccaatggcaatggtcataccaatggtaatggtctgaccaatggcaacggccataccgatggtagcggccataccaatggcaatggctatctgagtaatggctataccaatggtctgacaaaactgcatccgtcatatattagtaatatcctggtgcgtaccgtgtatggtccggttagcctggcccatgcactgcattggccggttagtgcctgttatgatgaactgcgtcgctgcgcaaaatggatgggtggccgcattccgaccgttgaagaagcccgtagcatctatagctatgttgatgaacgccgtctgaaagaagttcgcaatgcccgccgtgttccggccgtgaatgcacatctggttaataatggcgtggaagaaagtccgccgctgcgtgatccggccggtagtcctgccaatccgcatagtgccctgtttaccgatctggaaggtgccaatgtgggctttaaacattggcatccggtggccgttaccgccgatggtgacaaactggccggtcagggcgaaatgggcggtgtttgggaatggaccagcagtgtgctggaacgtcatgaaggctttcgcgaaatggaactgtatccggcctatagtgaagatttctttgatggcaaacataatgtggtgctgggtggcagctgggcaacccatccgcgcattgccggccgcaaaagttttattaattggtatcagcgtaactacccgtatgtgtgggcaggcgcccgcctggtgcgcgacatttaa

SEQ ID NO: 58 - protein, 915 aa, unidentified
  source 1..915: note = Pseudogymnoascus destructans
MTNSTTPPPDVVDLDKFHTHDDPRHTRLTTPSKATLPPATPPSPAQSTLDFIDIIDIRRDALGSSLDLGRDIMAQLAPARGPKKMPTLLLYDEKGLQTFEEITYLEEYYLTNAEIEVLERNAEEMARNIQAESMVIELGSGNLRKVSILLNALEKAEKSIHYYALDLSKRELERTLSSVPRFEHVVCHGLLGTYDDGLEWIRSGCNASWPKCIMSLGSSIGNFNRGDAAEFLKGFADMLRPSDSMIIGLDACNDPAKVYHAYNDSLGITHKFILNGLDNANSILGENVFDTNDWEVIGEYVCDKDGGRHRAFYAPKRDITIRGVFIEQGERVQVEQSLKYSQAESEGMWAAAGLKEVGKWGATKEQYNIHMLTKRAKPFQLHPSQYALTPTPTLEDWRGLWSTWNTVARGMIPNNELLAKPIKLRNACIFYLGHIPTFLDLQLSKATGVPLCEPSHYPQIFERGIDPDVDNPDNCHAHSEIPDQWPPVEEILEYQAQVRRKVEGLYASGVPEASRKVGRSLWIGLEHEIMHLETLLYMLLQSDNCMPPPRTVKPDFEEQARRDAEREVENQWFTIPEQDIALGLDDPEDNSGDGHFGWDNEKPVRKAHVRSFQAKGRPITNEEYAIYLDATDNENLPASWTRQHANGDLSAHTPNGNTNGYTNGNGHTNGNGLTNGNGHTDGSGHTNGNGYLSNGYTNGLTKLHPSYISNILVRTVYGPVSLAHALHWPVSACYDELRRCAKWMGGRIPTVEEARSIYSYVDERRLKEVRNARRVPAVNAHLVNNGVEESPPLRDPAGSPANPHSALFTDLEGANVGFKHWHPVAVTADGDKLAGQGEMGGVWEWTSSVLERHEGFREMELYPAYSEDFFDGKHNVVLGGSWATHPRIAGRKSFINWYQRNYPYVWAGARLVRDI

SEQ ID NO: 59 - DNA, 2595 bp, unidentified
  source 1..2595: mol_type = genomic DNA; note = Pestalotiopsis fici
atgccgagcgccaccgaaatgttttatgaaagtcaggcaattccgaccgcctttgaactgagtaaaggtctgagcaaaaatccgaaaagcagtcgccgtctggatattattgatattcgccaggccgcagtggaactgaatctgaaagaagaaattcatcagctgctgcgtccgcaggaaggtccgcgcaaactgccgaccctgctgctgtatgatgaacgtggtctgcagctgtttgaacagattacctatctggaagaatattatctgaccaatagcgaaattcaggtgctgcgtagtagtgcacaggcaattgccaaagcaattccgagtggtagcatggtggttgaactgggtagcggtaatctgcgtaaagttcagattctgctgcaggccctggaagatgccggtaaagatattgattattatgccctggatctggataaacgtgaactggaacgtaccctggcacaggtgccggcctttcgttttgttacctgccatggcctgcatggtacctatgatgatggccgcgtgtggctgaaaaatagtagcgttagcgcacgcccgaaatgtgtgatgagcctgggtagcagtattggtaattttcatcgcagtgatgcagccgcatttctgcgcagttttagtgatgtgctgcagccgagcgatacctttctgctgggtctggatagttgtaccaatccgagcaaagtgtatcatgcatataatgatcgtcatggcgttacccatcagtttattctgaatggtctgcgtcatgccaatgaagtgctggaagatgaagtttttaatctggatgaatggcaggttattggtgaatatgtgtatgatgtggaaggcggccgtcatcaggcattttatagcccgagccgcgatgtgaccattctgggcgaaaatattaaggcccaggaacgtattcaggttgaacagagcctgaaatatagtgaagatggtatgaaaaagctgtggagcgaagccggtgttgttgaaaccgatcgctggatgaccgataataatgaatatggtctgcatctgctgaccaaaccgaccaccatgccgtttagcctggaccctcgccagtatgcaggtagtgtgctgccgaccttagatgattggaaagccctgtggagcacctgggataccgttacccagcgtatgctgccggatgaagaactgctggaaaaaccgattaagctgcgtaatgcatgtattttctatctgggtcatattccgacctttctggatattcagctgaccaaaaccaccaaacagccgccgaccgaaccggttagctatagcgccatttttgaacgcggtattgatccggatgtggataatccggaacattgccatagtcatagcgaaattccggatgaatggccgaaacaggcagatattctgcgctatcagaataatgtgcgcgtgcgtctgaccggtctgtatagccatggcccggaaagcattccgcgtgatgttgcccgtgcaatttgggtgggttatgaacatgaactgatgcatatggaaaccctgctgtatatgatgctgcagagtgataaaaccctgccgccgccgcatattccgcgtccggattttaaaggtctggcacgtaaagcatatgccgaacgtaccgcaaatgaatggtttaccattccggaacagcatattctggttggtctgaatgatccggaagatgatgaacgttttaaaggtcattttggttgggataatgaaaaaccggcccgcaaaattaatgtgaaaagttttcaggcaaagggtcgcccgattaccaatgaagaatatgcatattatatgtacgagacaaaggtgaccaaaattccggccagttgggcagaagccccgcagcatcatgtgaatggtagcaatggcaccagtcatgaacataccaatggtcaggccaatggtcatgcaaatggccatgtgaatggccatgttaatggtagccatgataccggtagcaccctgctgccgagcagctttctggatgataaaaccgtgcgcaccatctatggcctggttccgctggaatatgccctggactggccggtttttgcaagttatgatgaactggccggttgtgcagcatggatgggcggtcgcattccgaccttcgaagaagcacgtagtgtttatgcatatgtggattatctgaaaaagaaggaagccgaacgtaaactgggtaaaaccgttccggccgtgaatggccacctggtgaatgatggcgtggaagaaaccccgccgagtcgtggttttggtatgcaggtgaatggcgaagcaagtgaaaatgatgatagctttgtggatctggaaggtagcaatgttggttttaatcattggcatccgatgccgattaccagccgcggcaatcgtctggccggccagagtgaaatgggtggtgtgtgggaatggaccagtagtcatctgacccgtcatgaaggctttgaaccgatggcactgtatccggcatataccagtgatttctttgatggcaaacataatgttgttctgggcggtagctgggcaacccatccgcgtattgccggtcgcaaaagcctgtaa

SEQ ID NO: 60 - protein, 864 aa, unidentified
  source 1..864: note = Pestalotiopsis fici
MPSATEMFYESQAIPTAFELSKGLSKNPKSSRRLDIIDIRQAAVELNLKEEIHQLLRPQEGPRKLPTLLLYDERGLQLFEQITYLEEYYLTNSEIQVLRSSAQAIAKAIPSGSMVVELGSGNLRKVQILLQALEDAGKDIDYYALDLDKRELERTLAQVPAFRFVTCHGLHGTYDDGRVWLKNSSVSARPKCVMSLGSSIGNFHRSDAAAFLRSFSDVLQPSDTFLLGLDSCTNPSKVYHAYNDRHGVTHQFILNGLRHANEVLEDEVFNLDEWQVIGEYVYDVEGGRHQAFYSPSRDVTILGENIKAQERIQVEQSLKYSEDGMKKLWSEAGVVETDRWMTDNNEYGLHLLTKPTTMPFSLDPRQYAGSVLPTLDDWKALWSTWDTVTQRMLPDEELLEKPIKLRNACIFYLGHIPTFLDIQLTKTTKQPPTEPVSYSAIFERGIDPDVDNPEHCHSHSEIPDEWPKQADILRYQNNVRVRLTGLYSHGPESIPRDVARAIWVGYEHELMHMETLLYMMLQSDKTLPPPHIPRPDFKGLARKAYAERTANEWFTIPEQHILVGLNDPEDDERFKGHFGWDNEKPARKINVKSFQAKGRPITNEEYAYYMYETKVTKIPASWAEAPQHHVNGSNGTSHEHTNGQANGHANGHVNGHVNGSHDTGSTLLPSSFLDDKTVRTIYGLVPLEYALDWPVFASYDELAGCAAWMGGRIPTFEEARSVYAYVDYLKKKEAERKLGKTVPAVNGHLVNDGVEETPPSRGFGMQVNGEASENDDSFVDLEGSNVGFNHWHPMPITSRGNRLAGQSEMGGVWEWTSSHLTRHEGFEPMALYPAYTSDFFDGKHNVVLGGSWATHPRIAGRKSL

SEQ ID NO: 61 - DNA, 2496 bp, unidentified
  source 1..2496: mol_type = genomic DNA; note = Aureobasidium subglaciale
atggataccagtaccgcaccgaaaattattgatattcgtcaggatggcggtggtctgaccccgctggtgccggaaattcgtgaaggtctgaatgcccgtgaaggtcaggaaaagaaactgccgaccctgctgctgtatagtgaagatggtctgaaactgtttgaaaaaattacctatctggaggaatactatccgaccggtcaggaaattcaggttctggaagcatatgcagatcgcattgccgatcgtattgccctggaaagtaatagcatgctggtggaactgggtagtggcaatctgcgcaaagtgcgcattctgctggatgccctggatcgcaaaggtaaagatgttagttattatgccctggatgttagcgaagttgaactggaacgcaccctggccgaagttccgcagggcacctttaaacatgttcagtgccatggtctgctgggtacctatgatgaaggtctggattggctgaaaaaaccggaaaatgcacatcgcagtaaaaccgttctgagcctgggtagcagtattggtaattttagtcgtgatgaagccgcaaaatttctgagccagtttagcgaaaccctggaccctaatgataccctgctgttaggtattgatgcatgtaccgatgcagataaagtttatcatgcatataacgatcgcgaaggtctgacccatgagtttattctgtgtggcctgaaacaggccaatcgcctgctgggttatgatgcctttgataccaaaatgtgggaagttattggccgttataataaggaaaccgatcgtcatgaagcctttgttagcccgaaaaaagatgttaccattgaaggtgcactgattcgtgccggtgaacaggttcgcattgaagaaagttataaatataacagcgtgcagagtgaacgcctgtggagtgatgccggcctgaccgaaggtgccaaatggaccaataccgatggcgattatgcactgcatctgctgaataagccgaaagttcagtatccgctggtggccgaaaaatatgccgcccagccggtgccgagcctggaagaatgggatcagctgtgggcagcatgggatgcagtgaccctggaaatgattccggaagaagatttgctggaaaaaccgattaagctgcgcaatgcctgtattttctatctgggccatattccgacctttatggatattcatctgacccgcgcaacccatggcaaaccgaccgaaccgagcagctataccagtatttttgaacgtggtattgatccggatgttgatgatccggaacagtgtcatgcccatagcgaaattccggatagctggccgccggccaccgaaattctggattttcagagcaaagttcgcattcgtgtgaaaaaactgtatgcaaccggccaggccgttaaagatcgtgccgttagtcgtgcaatgtggctgagttatgaacatgaaattatgcatctggaaaccctgctgtatatgctgattcagagtgaaaaaaccatggccccgccgaccaccgttatgccggattttgaagcactggcagttcaggcacgccagcgcgcagtggaaaatcagtggtttgatattccggcacagaaagttgaaattggcattgaagatccggatgataatagtggtccggaacatttctttggctgggataatgaaaaaccgaaacgcaccgttcatgttccggcctttaaagcaaaaggtcgtccgattaccaatggtgaatttgccaaatatctggaagaaaatcatctggataccctgccggccagctggcataccctgagccatacccgtggcaccgaaaccaatggtcataccagcggtattagtagcagctttctgcagggtaaagccgttcgtaccgtttatggtccgattagtctgaaactggcactggattggccggtttgcgccagttatgatgaactgcgcggctgtgcccgttggatgggcggtcgtattccgagcatggaagaagcccgcagcatctatcgccatgttgatgaagccaaaaccctgcaggcccatgaaagcctgggtgccaatattccggcagttaatgcccatctggtgaatgatggcgtggaagaaagtccgccgagcaaagccctgagcagcagcagcaccggtccgaatccgaatgatctgtttgttgatctgctgggtgcaaatgttggttttcgtcattggcatccgatgccggtgagccagcatggtaataagctggccggtcagagtgaaatgggcggtgtttgggaatggaccagtagcgtgctggataaacaggaaggctttgaagccatgccgctgtatccgggttataccgcagatttctttgatggcaaacataatattgtgctgggtggcagttgggcaacccatccgcgtattgccggccgtaaaagttttgttaattggtatcagcgcaattatccgtttgtttgggccggcgcccgcattgtgaaagatgcctaa

SEQ ID NO: 62 - protein, 831 aa, unidentified
  source 1..831: note = Aureobasidium subglaciale
MDTSTAPKIIDIRQDGGGLTPLVPEIREGLNAREGQEKKLPTLLLYSEDGLKLFEKITYLEEYYPTGQEIQVLEAYADRIADRIALESNSMLVELGSGNLRKVRILLDALDRKGKDVSYYALDVSEVELERTLAEVPQGTFKHVQCHGLLGTYDEGLDWLKKPENAHRSKTVLSLGSSIGNFSRDEAAKFLSQFSETLDPNDTLLLGIDACTDADKVYHAYNDREGLTHEFILCGLKQANRLLGYDAFDTKMWEVIGRYNKETDRHEAFVSPKKDVTIEGALIRAGEQVRIEESYKYNSVQSERLWSDAGLTEGAKWTNTDGDYALHLLNKPKVQYPLVAEKYAAQPVPSLEEWDQLWAAWDAVTLEMIPEEDLLEKPIKLRNACIFYLGHIPTFMDIHLTRATHGKPTEPSSYTSIFERGIDPDVDDPEQCHAHSEIPDSWPPATEILDFQSKVRIRVKKLYATGQAVKDRAVSRAMWLSYEHEIMHLETLLYMLIQSEKTMAPPTTVMPDFEALAVQARQRAVENQWFDIPAQKVEIGIEDPDDNSGPEHFFGWDNEKPKRTVHVPAFKAKGRPITNGEFAKYLEENHLDTLPASWHTLSHTRGTETNGHTSGISSSFLQGKAVRTVYGPISLKLALDWPVCASYDELRGCARWMGGRIPSMEEARSIYRHVDEAKTLQAHESLGANIPAVNAHLVNDGVEESPPSKALSSSSTGPNPNDLFVDLLGANVGFRHWHPMPVSQHGNKLAGQSEMGGVWEWTSSVLDKQEGFEAMPLYPGYTADFFDGKHNIVLGGSWATHPRIAGRKSFVNWYQRNYPFVWAGARIVKDA

SEQ ID NO: 63 - DNA, 2598 bp, unidentified
  source 1..2598: mol_type = genomic DNA; note = Verruconis gallopava
atgatcggtgacagtgcaggtctgggctttgcagattttagtagtagtaatgttcacaccaaagccgccaaaccgcatagtccgctgagtagcattattgaaattcgtcaggatcgtgaagaactggatctgctgattgatattaagagtggtctgcgtgcaagcggcccgggccgtaaaaccctgccgaccctgctgctgtatgatgaaccgggtctgaaactgtttgaaaaaattacctttctggacgaatactatctgaccaatgccgaaattgaagtgctgcataaatgggcaggcaatattgcagatcgtattagtccgaatagcattgtgctggaactgggtagcggcaatctgcgtaaaattaagattctgctggatgcatttgaagcagcaaaaaagccggtggaatattatgcactggatgtgagccgtgtggaactggaacgcaccctggcagccattccggttggcgcattcaaacatgtgaaatgttttggtctgcatggcacctatgatgatggcctgcagtggctgaaaagcgaacagattgcaaaacgtagtaaagcaattctgagtatgggtagcagcattggtaattttgcccgccatgaagcagtgcgctttctgcgtagttttagcgatgtgctgcagaccagtgatgttctgctgattggcattgatgcatgtaaagatccggataaagtgtttcgcgcatataatgatagccagggtgttacccatgaatttgtgctgaatggcctgcagcatgcaaatcagctgctgggccatgatgcctttgatgtggaaaaatggcgtgtgattggtgaatatgatgaagttaatggtaaacatcacgcatttgttagtccggttcaggatatgaatattgatggtattctgattaaggcaggtgaacgcattcgtattgaagaaagttttaaatacgacctgatcgatcgtagccgtctgtgggaaggtgcaggtctgattgaaggtgcaagttggaccaatgccgaggccaattatggtctgcatatggcatataaaccgaaagttcagtttagtagcaaaccggaagaatatgccgccagcgcagtgccgaccctggccgagtggaaagatttgtggaccatttgggatctggttacccagcagatgattccgaaagaagaactgctggcaaaaccgattaagctgcgtaatgcatgcattttctatctgggccatattccgacctttctggccattcatctggaaaaagccagctgtgaaggcgtggccggcctgcgtgattatcagcgtatttttgaacgcggcattgatccggatgttgataatccggaactgtgtcatgatcatagtgaaattccggatgaatggccgccggaagttgaaattctggcctttcagagtaaagtgcgtgatcaggttaaacagatatatgatagtcgtatgcatgaaagcagtcatgccattcgtaaagcactgtggattagttttgaacatgaagttatgcatatcgaaaccctgctgtatatgctgattcagagtgaaaaaaccctgcctccgccgggcgttattcatccggattttgaagcactggcctgtgaagccaaagcaaaaaccgttccgaataagtggtttaccgtgccgagtcgtaccgttaccctgggcctgaatgatgatgataaagataccaccaccaatcgttattttggttgggataatgaaaagccgcagcgtcgcgttaaagttaaaagctttagcgcccaggcacgcccgattaccaatggcgaatatgtggaatatctgaaagcaattggcagtgaaaaactgccggcaagctggagtagtaccaaaagtaccccgaatggtcattttaatggccatatgaatggcgatagcaatggtgtggcaaatgccgcagtgaatggtgaaaattttgttgatggtaaagaggtgaaaaccgtttttggtagcattccgctgaaactggcactggattggccggttatggcaagctatgatgaactgctgggttttgcccattgggcaggtggccgcattccgaccatggaagaagtgaaaagcatctatgaatatgccgaagaactgaaagttaaagattttgtgaacgcactgggcgaaaccattccggcagttaatggccatctgattaatgatggcgtggaagaaaccccgccgcatcgccatagtgcaaatggcgaaccgagtgccagcgttggtccgagtccgcatcgcctgtttgttgatctggaagatgcaaatgttggctttaaacattggcatccgctgccggttaccgcacatggtgacaatctggcaggccagggtgaactgggcggcgtttgggaatggaccagtaccgttctggaaaaacatgaaggctttgaaccgatgcagctgtatccgggctataccgccgatttctttgataaaaaacataatatcgtgctgggtggcagctgggcaacccatccgcgtattgccggccgtcgtagctttgttaattggtatcagcgtaattatccgtttgtgtgggccggtgcacgcctggttcgcgatctgtaa

SEQ ID NO: 64 - protein, 865 aa, unidentified
  source 1..865: note = Verruconis gallopava
MIGDSAGLGFADFSSSNVHTKAAKPHSPLSSIIEIRQDREELDLLIDIKSGLRASGPGRKTLPTLLLYDEPGLKLFEKITFLDEYYLTNAEIEVLHKWAGNIADRISPNSIVLELGSGNLRKIKILLDAFEAAKKPVEYYALDVSRVELERTLAAIPVGAFKHVKCFGLHGTYDDGLQWLKSEQIAKRSKAILSMGSSIGNFARHEAVRFLRSFSDVLQTSDVLLIGIDACKDPDKVFRAYNDSQGVTHEFVLNGLQHANQLLGHDAFDVEKWRVIGEYDEVNGKHHAFVSPVQDMNIDGILIKAGERIRIEESFKYDLIDRSRLWEGAGLIEGASWTNAEANYGLHMAYKPKVQFSSKPEEYAASAVPTLAEWKDLWTIWDLVTQQMIPKEELLAKPIKLRNACIFYLGHIPTFLAIHLEKASCEGVAGLRDYQRIFERGIDPDVDNPELCHDHSEIPDEWPPEVEILAFQSKVRDQVKQIYDSRMHESSHAIRKALWISFEHEVMHIETLLYMLIQSEKTLPPPGVIHPDFEALACEAKAKTVPNKWFTVPSRTVTLGLNDDDKDTTTNRYFGWDNEKPQRRVKVKSFSAQARPITNGEYVEYLKAIGSEKLPASWSSTKSTPNGHFNGHMNGDSNGVANAAVNGENFVDGKEVKTVFGSIPLKLALDWPVMASYDELLGFAHWAGGRIPTMEEVKSIYEYAEELKVKDFVNALGETIPAVNGHLINDGVEETPPHRHSANGEPSASVGPSPHRLFVDLEDANVGFKHWHPLPVTAHGDNLAGQGELGGVWEWTSTVLEKHEGFEPMQLYPGYTADFFDKKHNIVLGGSWATHPRIAGRRSFVNWYQRNYPFVWAGARLVRDL

SEQ ID NO: 65 - DNA, 1422 bp, unidentified
  source 1..1422: mol_type = genomic DNA; note = Neurospora crassa
atggtggccaccaccgttgaactgccgctgcagcagaaagccgatgccgcacagaccgttaccggtccgctgccgtttggtaatagtctgctgaaagaatttgtgctggaccctgcatatcgtaatctgaatcatggtagttttggcaccattccgagtgccattcagcagaaactgcgtagttatcagaccgccgccgaagcccgcccgtgtccttttctgcgttatcagaccccggttctgctggatgaaagtcgtgcagcagtggcaaatctgctgaaagttccggttgaaaccgttgtgtttgtggcaaatgccaccatgggcgtgaataccgttctgcgtaatattgtttggagtgcagatggcaaagatgaaattctgtattttgataccatctacggcgcatgtggcaaaaccattgattatgttattgaagataagcgtggtattgtgagtagtcgctgcattccgctgatctatccggcagaagatgatgatgttgttgcagcatttcgcgatgccattaagaaaagccgcgaagaaggtaaacgcccgcgtctggcagttattgatgttgttagtagtatgccgggcgtgcgttttccgtttgaagatattgttaaaatctgcaaagaggaagaaatcattagctgtgtggatggtgcacagggcattggcatggtggatctgaaaattaccgaaaccgatccggattttctgattagtaattgccataaatggctgtttaccccgcgcggttgcgcagtgttttatgttccggttcgcaatcagcatctgattcgtagtaccctgccgaccagtcatggttttgtgccgcaggtgggcaatcgctttaatccgctggttccggccggcaataagagcgcatttgtgagcaattttgaatttgtgggtaccgtggataatagtccgtttttctgtgtgaaagatgccattaagtggcgcgaagaagtgctgggtggtgaagaacgcattatggaatatatgaccaaactggcccgtgaaggtggccagaaagtggccgaaattctgggcacccgcgttctggaaaatagcaccggtaccctgattcgctgtgccatggtgaatattgcactgccgtttgttgttggcgaagatccgaaagccccggttaaactgaccgaaaaagaagaaaaagatgttgaaggcctgtatgaaattccgcatgaagaagcaaatatggcattcaaatggatgtataatgtgctgcaggatgagtttaatacctttgtgccgatgacctttcatcgtcgtcgcttttgggcacgcctgagtgcccaggtttatctggaaatgagcgattttgaatgggccggcaaaaccctgaaagaactgtgcgaacgcgttgccaaaggtgaatataaagaaagtgcataa

SEQ ID NO: 66 - protein, 473 aa, unidentified
  source 1..473: note = Neurospora crassa
MVATTVELPLQQKADAAQTVTGPLPFGNSLLKEFVLDPAYRNLNHGSFGTIPSAIQQKLRSYQTAAEARPCPFLRYQTPVLLDESRAAVANLLKVPVETVVFVANATMGVNTVLRNIVWSADGKDEILYFDTIYGACGKTIDYVIEDKRGIVSSRCIPLIYPAEDDDVVAAFRDAIKKSREEGKRPRLAVIDVVSSMPGVRFPFEDIVKICKEEEIISCVDGAQGIGMVDLKITETDPDFLISNCHKWLFTPRGCAVFYVPVRNQHLIRSTLPTSHGFVPQVGNRFNPLVPAGNKSAFVSNFEFVGTVDNSPFFCVKDAIKWREEVLGGEERIMEYMTKLAREGGQKVAEILGTRVLENSTGTLIRCAMVNIALPFVVGEDPKAPVKLTEKEEKDVEGLYEIPHEEANMAFKWMYNVLQDEFNTFVPMTFHRRRFWARLSAQVYLEMSDFEWAGKTLKELCERVAKGEYKESA

SEQ ID NO: 67 - DNA, 1416 bp, unidentified
  source 1..1416: mol_type = other DNA; note = Thielavia terrestris NRRL 8126
atgggcctggcaccgctggaactgccggtgcgtcagaaagcagatgtggatgccacccagaatggtccggtgaaatttggtcatgaactgcgcgaacagcattttctgtttgatccgagctatcgtaatctgaatcatggcagctttggcaccattccgcgcgcaattcaggcaaaactgcgtagctatcaggatcaggccgaagcagccccggatgtgtttattcgttatgattatccgaaactgctggatcagagccgtgccgcaattgcaaaactgctgcgcgtgccgaccgataccgttgtttttgtgccgaatgcaaccaccggcgttaataccgtgctgcgtaatctggattggaatgccgatggcaaagatgaaattctgtattttgataccatctacggcggctgtgcccgcaccattgattatgttgttgaagatcgtcagggtcgcgttagtcatcgttgcattccgctgagttatccgtgcgaagatgatgcagttgtggcagcctttgaaagcgcagttgaagccagccgtcgcgatggtaaacgtccgcgtctgtgcctgtttgatgttgtgagcagtctgccgggcgttcgttttccgtttgaagcaattgccgccgcatgtcgtgccgccggcctgttaagcctggttgatggcgcacagggcgttggtatggtggatctggatctggcagccgttgatccggatttctttgttagcaattgccataaatggctgcatgttccgcgtggctgtgcagttttctatgtgccggaacgcaatcagccgctgatgcgtagtccgctggtgaccagtcatcgctttgttccgcgtgcaggtgcaacccagccgctgtttaatccgctgccgccgaccgataaaaccgaatttgttagtaatttcgagttcgtgggcaccgtggataatgcaccgtatctgtgtgttcgcgatagtctgcgctggcgcgaagaagtgctgggcggtgaagcccgtattctggccgcactgaccgcccaggcccgcgagggtggtagacgtgcagcagcaattctgggtaccgaagtgctggataatgcaagccagagcctgacccgttgcagcatggttaatgtggccctgccgctggccgttcagccggatggtgaaggcgaagcaccgccggccgcaggtggttttccggctctgccgaaagaagatgttagtgcagtgaccaattggatgctggaaaccctgatggatgagtttaaaacctttattgcactgtttgtgtacaaagatcgttggtgggcccgcctgagtgcacaggtgtatctggaactggatgattttgaatgggccggccagaccctgaaaaccgtgtgcgaacgtgcaggccgcggtgaatataaacaggatcgtccgtaa

SEQ ID NO: 68 - protein, 471 aa, unidentified
  source 1..471: note = Thielavia terrestris NRRL 8126
MGLAPLELPVRQKADVDATQNGPVKFGHELREQHFLFDPSYRNLNHGSFGTIPRAIQAKLRSYQDQAEAAPDVFIRYDYPKLLDQSRAAIAKLLRVPTDTVVFVPNATTGVNTVLRNLDWNADGKDEILYFDTIYGGCARTIDYVVEDRQGRVSHRCIPLSYPCEDDAVVAAFESAVEASRRDGKRPRLCLFDVVSSLPGVRFPFEAIAAACRAAGLLSLVDGAQGVGMVDLDLAAVDPDFFVSNCHKWLHVPRGCAVFYVPERNQPLMRSPLVTSHRFVPRAGATQPLFNPLPPTDKTEFVSNFEFVGTVDNAPYLCVRDSLRWREEVLGGEARILAALTAQAREGGRRAAAILGTEVLDNASQSLTRCSMVNVALPLAVQPDGEGEAPPAAGGFPALPKEDVSAVTNWMLETLMDEFKTFIALFVYKDRWWARLSAQVYLELDDFEWAGQTLKTVCERAGRGEYKQDRP

SEQ ID NO: 69 - DNA, 1374 bp, unidentified
  source 1..1374: mol_type = genomic DNA; note = Colletotrichum higginsianum
atgggcgaactggtgaaagaaaccagccagctgcagctgagcagcattccgtttggtaaaccgatgctgaaagaatttctgattgatccggcatatcataatatgaatcatggcagttttggcaccattccgcgccatattcagaccattctgcgcagttatcaggataaagcagaagcccgcccggacccttttattcgttgggaatatcatacctatctgaaagaaagccgtcaggccgttgcagatttgattaatgcaccggtggattgtaccgtttttgttccgaatgccaccgtgggcattaataccgttctgcgtaatctgatttgggccccggatggtctggatgaaattctgtattttagcaccgtgtatggtggctgtgcaaaaaccattgattatattgtggatacccgtctgggcctggtgagcagccgcagtattccgctgacctatccgctggaagatgatgaagtggtggcactgtttcgcgatgcagtggcacagagtcatgccgaaggtaaacgcccgaaaatttgtctgtttgatgtggttagttgtctgccgggtattcgctttccgtttgaagcaattaccgccgcatgtcgtgaactgggcattctgagtctggtggatggcgcacagggcgtgggtatggtgccgctggatattgcagcactggaccctgatttctttattagtaattgtcataagtggaccttcaccccgcgtgcaagtgccgtgttttatgtgagcgaacgcaatcatcatctggttccgagcaccattccgaccagtcatggctatgttccgcgtaccggtgttcagcgtcataatccgctgccgccgagtggtgaaccgccgtttgtgacccgttttggctttgtggcaacctttgataatagtccgaatctgtgcgtgaaacatagtattgaatggcgcaaaagtattggcggcgaagataaaattatggaatatctgtgggcactggccaaaaatggcggtaaaaaagcagcagcaattctgggtacctttattctggataataagagtgaaaccctgacccgctgcgcaatggttaatgttgcactgccgattgttatgggcgccgatgccgaaaccctgagtgttggcccggatggcaccattaccgttccggaaaaagaagccagtgttattgttaattggatgctgagtgccctggttaatgaatatctgacctttgtggccctgttttggcatcagggccgctggtatagtcgtattagtgcccagatatatctggatgaaaccgattttgaatgggttggcaataccattaaggaactgtgtcagcgcgttgccaaacaggaatataaagtgaaagcataa

SEQ ID NO: 70 - protein, 457 aa, unidentified
  source 1..457: note = Colletotrichum higginsianum
MGELVKETSQLQLSSIPFGKPMLKEFLIDPAYHNMNHGSFGTIPRHIQTILRSYQDKAEARPDPFIRWEYHTYLKESRQAVADLINAPVDCTVFVPNATVGINTVLRNLIWAPDGLDEILYFSTVYGGCAKTIDYIVDTRLGLVSSRSIPLTYPLEDDEVVALFRDAVAQSHAEGKRPKICLFDVVSCLPGIRFPFEAITAACRELGILSLVDGAQGVGMVPLDIAALDPDFFISNCHKWTFTPRASAVFYVSERNHHLVPSTIPTSHGYVPRTGVQRHNPLPPSGEPPFVTRFGFVATFDNSPNLCVKHSIEWRKSIGGEDKIMEYLWALAKNGGKKAAAILGTFILDNKSETLTRCAMVNVALPIVMGADAETLSVGPDGTITVPEKEASVIVNWMLSALVNEYLTFVALFWHQGRWYSRISAQIYLDETDFEWVGNTIKELCQRVAKQEYKVKA

SEQ ID NO: 71 - DNA, 1359 bp, unidentified
  source 1..1359: mol_type = other DNA; note = Fusarium oxysporum f. sp. lycopersici 4287
atgggcagcattggtcagggtagtagtcagctgccggtgcgtggcaaaaccaataccagtgtgtttggcagcgccattaagaaagagtttatgtttgatccggaatggcgcaatctgaatcatggcagctttggcacctatccgcaggcagttcgtaccaaatttcgcgaatatcaggatgccagtgaagcccgcccggacccttttattcgctatgaatatccgaaaattctggatgaaaaccgtgcagcagtggccaaactgctgaatgccccggttgatagcgtggtttttgttagtaatgcaaccaccggcgttaataccgtgtatcgcaatatgaaatggaatgaagatggcaaagatgttattattagctttagcaccatctatgaagcctgtggtaaagtggcagattattatgttgattattacaacgagaaggtgacccatcgtgaaattgaactgccgtatccgctggatgatgatgaaattattaagaaattcgaggacgccgttaaaaagattgaaagtgaaggcaaacgcgtgcgtatttgcacctttgatgttgtgagtagccgtccgggcgttgtttttccgtgggaagaaatggttaaaacctgtcgccgtctgaatgttctgagcatggtggatggtgcccagggtgtgggtatggtgaaactggatctgagtgccgccgatccggatttctttgtgagcaattgccataaatggctgcatgtgccgcgtggttgcgccgttttctatgttccgcagcgtaatcaggcactgattcagaccaccctggcaaccagccatggctatgttccgaaactggcaaatcgtattaccccgctgccgccgagtagtaaaagtccgtttgttattaatttcgagttcgtgggtaccctggataatagtccgtatctgtgcgtgaaagatgcaattaagtggcgtgaagaagcactgggtggcgaagatgccattctggaatatatttgggatctgaataagaaaggcagtgaactggttgcagaaaaactgggtaccacctatatggaaaatagcaccggcaccatgcgtaattgcggtatggcaaatattgccctgccggtgtggaccgttgaaggcaaagaaggcgaagtggttattagcgcagaagaaacccagaccgcctttcagtggattctgaataccctgattggcgattataaaacctttgtggcactgtttctgcatggcggtcgcttttggattcgtaccagcgcacaggtgtatctggaaattgaagattatgaatggctgggcggtgttctgaaagaagtttgtgaacgtgttggtaaaaaagaatatctgaaataa

SEQ ID NO: 72 - protein, 452 aa, unidentified
  source 1..452: note = Fusarium oxysporum f. sp. lycopersici 4287
MGSIGQGSSQLPVRGKTNTSVFGSAIKKEFMFDPEWRNLNHGSFGTYPQAVRTKFREYQDASEARPDPFIRYEYPKILDENRAAVAKLLNAPVDSVVFVSNATTGVNTVYRNMKWNEDGKDVIISFSTIYEACGKVADYYVDYYNEKVTHREIELPYPLDDDEIIKKFEDAVKKIESEGKRVRICTFDVVSSRPGVVFPWEEMVKTCRRLNVLSMVDGAQGVGMVKLDLSAADPDFFVSNCHKWLHVPRGCAVFYVPQRNQALIQTTLATSHGYVPKLANRITPLPPSSKSPFVINFEFVGTLDNSPYLCVKDAIKWREEALGGEDAILEYIWDLNKKGSELVAEKLGTTYMENSTGTMRNCGMANIALPVWTVEGKEGEVVISAEETQTAFQWILNTLIGDYKTFVALFLHGGRFWIRTSAQVYLEIEDYEWLGGVLKEVCERVGKKEYLK

SEQ ID NO: 73 - DNA, 1287 bp, unidentified
  source 1..1287: mol_type = genomic DNA; note = Phialocephala scopiformis
atgaccattaagccgccgtttggtcatccgattcgtaatacccattttagctttagcccgacctatgttccgctgaatcatggtagttttggcacctttccgctgagtgttacccagcatcagaatcagctgcagacccaggccctggaacgcccggatacctttattgtgtttgatctgccggtgctgattgatgaaagccgcgcagcaattgcaccgctgctgggtgttgatgtggatgaagttgtgtttgtgccgaatgccaccaccggcgttaatgttgttctgcgtaatctgcgttgggaagaaggtgacgtggttgtttgttttagtaccatctatggcgcatgcgaaaaaagcctggttagtgttggtgaagttctgccggttcagatggaagtggtggaactgcagtatccggttgaagatgaagaaattctgggtcgtctggaagaacgtgttggtaaagtgcgtcaggaaggcaaacgcattcgtctggcaatgtttgataccgtgctgacctttccgggcgcacgtatgccgtgggaacgcctggtggccaaatgcaaagaactggaagtgctgagcctgattgatggtgcacatggtattggccatattgatctgcgtgaactgggcaaagtggccccggatttctttgtgagtaattgccataaatggctgtataccccgcgtggttgcgccgtttttcatgtgccgtttaaaaatcagcatctgattcgtaccagcctgccgaccagtcatggttatcagcatccgaataagccgccggaaaaaattgatggcaaaaccccgtttgtgcatctgtttgaatttgttgcaaccattgattatagcccgtatgcatgcgtgccggcagccctgagctttcgtcagaaaatttgtggtggcgaagaagaaattcgcaaatattgttttaacctggcacgtaccggcggcgccgcagtggcaaaaattctgggcacccatgtgatggataccaaaagtggtaccatgagccagtgttgttttgcaaatgttgcactgccgctggcatttggcgaaggcaaaaaatttggcaccgatgaagccccgcgcattcagaaatggctgaatggcaccgcagtgcgtgaatttgatacctatctgcagattgccctgcatggtggtattatgtgggttcgcctgagtgcccagatatatctggaaggtaaagattttgaatgggtgggttatcgtctgaaagaactgtgcgttcgtattgaaggtggcgaagtggatcgctaa

SEQ ID NO: 74 - protein, 428 aa, unidentified
  source 1..428: note = Phialocephala scopiformis
MTIKPPFGHPIRNTHFSFSPTYVPLNHGSFGTFPLSVTQHQNQLQTQALERPDTFIVFDLPVLIDESRAAIAPLLGVDVDEVVFVPNATTGVNVVLRNLRWEEGDVVVCFSTIYGACEKSLVSVGEVLPVQMEVVELQYPVEDEEILGRLEERVGKVRQEGKRIRLAMFDTVLTFPGARMPWERLVAKCKELEVLSLIDGAHGIGHIDLRELGKVAPDFFVSNCHKWLYTPRGCAVFHVPFKNQHLIRTSLPTSHGYQHPNKPPEKIDGKTPFVHLFEFVATIDYSPYACVPAALSFRQKICGGEEEIRKYCFNLARTGGAAVAKILGTHVMDTKSGTMSQCCFANVALPLAFGEGKKFGTDEAPRIQKWLNGTAVREFDTYLQIALHGGIMWVRLSAQIYLEGKDFEWVGYRLKELCVRIEGGEVDR

SEQ ID NO: 75 - DNA, 1326 bp, unidentified
  source 1..1326: mol_type = genomic DNA; note = Nectria haematococca
atgggtagcgtgacccaggaactgccgctgcgcggtaaaccgagtgcaagtgtttttggcgccgcaatgaaagatgaatttctgtttgatccggaatggcgcaatctgaatcatggtagctttggcacctatccgaaagcaattaaggccaaatttcgcgatgaagcacgtccggatgtttttattcgttatgaatatccgaagctgctggatgaaagtcgtgttgccgttgccaaaattctgaatgcaccggaagatggcgtggtgtttgtgagcaatgccaccgtgggcgttaataccgtttttcgtaatatggcatggaataaggatggcaaagatgtgattattagctttagtaccatctatgaagcctgtggtaaagttgccgattatctggccgattattatgaaggcaatgttacccatcgtgaaattgaaattacctatccgattgatgatgatgtgattctgaaacgctttgaagataccgtgaaaaagattgaagaagaaggtaaacgcgcacgtatttgtacctttgatgttgttagtagtcgcccgggcgtggtgttcccgtgggaagaaatgattaagacctgtcgccgcctgaatgtgctgagcatggttgatggcgcccagggtattggcatggtgaaactggatctgagtgcagccgatccggatttctttgttagcaattgccataaatggctgcatgtgccgcgtggttgcgccgttttctatgttccgcagcgtaatcaggcactgctgccgaccaccctggcaaccagtcatggctatgttccgaaactggcaaatcgcattagtccgctgccgccgagtagcaaaccgcgctttgtgaccaattttgaatttgttggcaccctggataatagtccgtatctgtgcgttaaagatgcaattaagtggcgccaggatgttctgggtggtgaagatgcagttctgaaatatctgtgggatctgaataagaaaggtaccgatattgtggcaaaagcactgaataccccggttatggaaaatagtaccggtaccctgcgtaattgtggcatgggcaatgttgccctgccgctgtgggcaggtgaaggtgaaggcaccgtggtgccggcagatgaaacccagaaagcatttcagtggatgctgaccaccctgattgatgattataaaacctttctgagtctgtttatccatggcggccgtttttgggcacgcattagcgcccaggtttatctgggcattgaagattatgaatgggccggtaaagttctgaaagaactgtgtgaacgtgtggcaaaaaaggaatatctgtaa

SEQ ID NO: 76 - protein, 441 aa, unidentified
  source 1..441: note = Nectria haematococca
MGSVTQELPLRGKPSASVFGAAMKDEFLFDPEWRNLNHGSFGTYPKAIKAKFRDEARPDVFIRYEYPKLLDESRVAVAKILNAPEDGVVFVSNATVGVNTVFRNMAWNKDGKDVIISFSTIYEACGKVADYLADYYEGNVTHREIEITYPIDDDVILKRFEDTVKKIEEEGKRARICTFDVVSSRPGVVFPWEEMIKTCRRLNVLSMVDGAQGIGMVKLDLSAADPDFFVSNCHKWLHVPRGCAVFYVPQRNQALLPTTLATSHGYVPKLANRISPLPPSSKPRFVTNFEFVGTLDNSPYLCVKDAIKWRQDVLGGEDAVLKYLWDLNKKGTDIVAKALNTPVMENSTGTLRNCGMGNVALPLWAGEGEGTVVPADETQKAFQWMLTTLIDDYKTFLSLFIHGGRFWARISAQVYLGIEDYEWAGKVLKELCERVAKKEYL

SEQ ID NO: 77 - DNA, 1455 bp, unidentified
  source 1..1455: mol_type = genomic DNA; note = Hyaloscypha bicolor
atgggcgaagtgctgaatattaagctggaagaagttagtctgaataatgaacgtaccccgtttggtaaagaaatgctgaaacattttctgtttgacccggattataaaaatctgaatcagggcagttttggtagctttccgcgtgtggtgcgcgaaaaacagcagcagtatcagcgcgcatgcgaactgcgtccggacccttttattcgttatgaacatccgaaactgctggatgaaagccgtgcagcactggccaaagtgctgaatgccccgctgagcaccgttgtttgtgttccgaatgcaaccaccggcgtgaataccattattcgtaatattgtgtggaacgcagatcgcaaagatgaagtgctgtattttagcaccgcatattgcagctgcagcaataccattgattataatagtgaagtgcacccgaatctggtgggcagtcgtgaaattagtctgacctatccgattgaagatgaagatttgctgaaaatgttcaaagatgcaattaaggccagccgtgcaagcggcaaacgtccgcgtctggccatgtttgataccgtgagtagcctgccgggcgttcgtatgccgtttgaaagtctgaccgccatttgcaaacaggaaggcatttttagcctgattgatggtgcacatggtattggtctgctgccgctggatctgagcgccctggaccctgatttctttaccagcaatacccataaatggtttttcgtgccgcgcggctgtgcagtgctgtatgttccggaacgcaatcagcatctgattcgtagcagcctgccgaccagcgatggctttgtgagtaaaaccggcatggccagtcgcaatccgctgccgccgagtagtaaaagtgaatttgttaataccttcgagttcgtgggtaccctggataatgccaattatctggttattccggaagcaattgaatttcgtgaaaaagtttgtggcggcgaaaaagcaattatggaatattgtgttcacctggccaaagatggcggcaaagccgccgccaaaattctgggtaccagcattatggataatagcaccgaaaccctgaccaaaggttgtggtatggtgaatattctgctgccgttagaaattagcccgaccaaagtgcatggcaaaaattgtattgatccgcgtaatcgtaccgtggcaaccgaatggatgcaggaaaccctgattgcagattttaaaacctttattccgatctatctgttccaggaaaaatggtgggcccgtctgagtgcacagatatatctggaactggttgattttgaatgggcaggtaaagcactgaaagcaatttgcgaacgtgccggcaaaggcgaatttctgaaagccgaaaagaaagtgaaaaagatgggtgaagttgttggtggcggtggtgaacatggcccgcgtacccgtccggaactggaatgcaaactgtaa

SEQ ID NO: 78 - protein, 484 aa, unidentified
  source 1..484: note = Hyaloscypha bicolor
MGEVLNIKLEEVSLNNERTPFGKEMLKHFLFDPDYKNLNQGSFGSFPRVVREKQQQYQRACELRPDPFIRYEHPKLLDESRAALAKVLNAPLSTVVCVPNATTGVNTIIRNIVWNADRKDEVLYFSTAYCSCSNTIDYNSEVHPNLVGSREISLTYPIEDEDLLKMFKDAIKASRASGKRPRLAMFDTVSSLPGVRMPFESLTAICKQEGIFSLIDGAHGIGLLPLDLSALDPDFFTSNTHKWFFVPRGCAVLYVPERNQHLIRSSLPTSDGFVSKTGMASRNPLPPSSKSEFVNTFEFVGTLDNANYLVIPEAIEFREKVCGGEKAIMEYCVHLAKDGGKAAAKILGTSIMDNSTETLTKGCGMVNILLPLEISPTKVHGKNCIDPRNRTVATEWMQETLIADFKTFIPIYLFQEKWWARLSAQIYLELVDFEWAGKALKAICERAGKGEFLKAEKKVKKMGEVVGGGGEHGPRTRPELECKL

SEQ ID NO: 79 - DNA, 1332 bp, unidentified
  source 1..1332: mol_type = genomic DNA; note = Aspergillus candidus
atgggcgaagccaatgttgttctgggcagtggtccgaccccgtttggtaaagaaatgaaaaaacatttcagcttcgcaccgggctatcataatctgaatcatggcagttatggcacctgcccgaccgcaattcagcgtgaagccaatcgcctgcgcgatgaatgcgaagcccgtccgtgcccgtttattaagtatcgttttccggaactgctggatgaaagccgcgcagcagtggcccagtttctgggtgttccgcgtagtaccgttgtgtttgttaccaatgcaaccaccggcgttaataccgtttttcgcaatatgatttggaataccgatggcaaagatgaaattattgaatttgacgttgtgtacggtgcctgtggtaaaaccgccgattatatttgcgaaaccagtcgtgatctggtgcgtacccgccagattcagctgacctatccggttgaagatgatgattttgttgcagcctttcgtgaagccattgatgcaagtcgtcgcgatggccgtcgtccgcgtattgcaatttttgataccattagcagcaatccgggtattcgcctgccgtttgaagccctgaccgccgtttgtcgtagcgaaggtgttctgagcctgattgatgccgcacatggtattggccagattgatctgaatctgccgagcctggaccctgattttctggtgagtaattgccataaatggctgtttaccccgcgtggttgtgccgttttctatgtgccggaacgcaatcaggcaatgatgcgcagcaccattccgaccagccatggttttcgtccgcgtctggcacagaatgaagaaaagaaagtgagcattgcaccgcatacccatagcgaatttgaactgaattttgaacataccggtacctatgataatattgcatttctgaccgtgccggccgccattaagtggcgtcagaatgtgtgtggcggcgaagaaaaaattcgcggttattgtaccaatctggcccgcgaaggcggcaaaattgttgccgcagcactgggcaccagtgttctggataatccgacccatacctataccgattgctttatggtgaatattctgctgccggttccgccgaaagaaaatgaatgtatgaattggcgtggtcgtccggttaatattagtgaatggatgcagcgcaccatgattgaagaatggcagacctatatgccggtgttttggtttaaaggcgcctggtggtttcgcattagcgcacaggtttatctggaactgagcgattttgaatgggccggtagcgccatgaaagaagtgtgtcagaaagtgaataagctgctgggttaa

SEQ ID NO: 80 - protein, 443 aa, unidentified
  source 1..443: note = Aspergillus candidus
MGEANVVLGSGPTPFGKEMKKHFSFAPGYHNLNHGSYGTCPTAIQREANRLRDECEARPCPFIKYRFPELLDESRAAVAQFLGVPRSTVVFVTNATTGVNTVFRNMIWNTDGKDEIIEFDVVYGACGKTADYICETSRDLVRTRQIQLTYPVEDDDFVAAFREAIDASRRDGRRPRIAIFDTISSNPGIRLPFEALTAVCRSEGVLSLIDAAHGIGQIDLNLPSLDPDFLVSNCHKWLFTPRGCAVFYVPERNQAMMRSTIPTSHGFRPRLAQNEEKKVSIAPHTHSEFELNFEHTGTYDNIAFLTVPAAIKWRQNVCGGEEKIRGYCTNLAREGGKIVAAALGTSVLDNPTHTYTDCFMVNILLPVPPKENECMNWRGRPVNISEWMQRTMIEEWQTYMPVFWFKGAWWFRISAQVYLELSDFEWAGSAMKEVCQKVNKLLG

SEQ ID NO: 81 - DNA, 1350 bp, unidentified
  source 1..1350: mol_type = genomic DNA; note = Pyricularia oryzae
atggcaaccccgctgcgtcattatggcaccgaaaaaccgtatagccatccggaaccggttaccaaacgccagtttggtaaaaatgtgctgcaggattttctgattgatccgaaatttcgcaatatgaatcatggtagtttcggcgttattccgcgtccggttcatgccgcacgccgttattatcaggataaaagcgaagaacgtccggatgtgtggattcgctataattggagccagctgctggaaggcagccgcgccgctgtggcacctctgttaggtgtggataaagataccattgcatttgtgccgaatgccaccgttggtgttaataccgttctgcgcaatctggtgtggaatgatgataaaaaagatgaaatcctgtacttcaacaccatctatgcagcatgcggcaaaaccgtgcagtatatgattgaaattagtcgcggccatgttagcggtcgtagcgtgccgctggaatatccgctgaccgatgatgaactggtggccctgtttaaaaaaggtattcaggattgtcgtgcagcaggtaaacgtccgcgcgccgccgtgattgataccgttagcagtattccggcagttcgcctgccgtttgaagccctggtgcaggtttgtcatgatgaaggtattctgagtattgttgatggtgcccagggtgtgggtatgattgatctgaaacatctgggcacccaggttaaaccggatttctttattaccaattgtcataaatggctgtacaccccgcgcggttgcgccgttctgcatgtgccgaaacataatcaggccctgatgcgtagtaccctgccgaccagctggggttgggttccgagtggcgaaggtgacccggattttattgataattttgcctttgcaagcaccctggataatagtaattatatggccgttcagcatgcagttcagtggattcaggaagcactgggcggtgaagatgccgttattgaatatatgatgagtctgaataagaagggcggcaatatggtggcagaaatgctgggcaccaaagttctggataatgcagaaggcaccctgaccaattgcgccatgagtaatgttctgctgccgctgggtattaagggccgcgaaagtagtgcaaaagttctggtggatgaagaagatgccgcccgtctgggcgattggtgccagaaaaccctggccagtgattataatacctggctgccggttaccctgattaagggtcagtggtggacccgcattagtgcccaggcctatctggatgaaagtgattatgaagcagttggcaaaatttttctggaactggttgaacgtattggcaaaggcgatcataaaaaataa

SEQ ID NO: 82 - protein, 449 aa, unidentified
  source 1..449: note = Pyricularia oryzae
MATPLRHYGTEKPYSHPEPVTKRQFGKNVLQDFLIDPKFRNMNHGSFGVIPRPVHAARRYYQDKSEERPDVWIRYNWSQLLEGSRAAVAPLLGVDKDTIAFVPNATVGVNTVLRNLVWNDDKKDEILYFNTIYAACGKTVQYMIEISRGHVSGRSVPLEYPLTDDELVALFKKGIQDCRAAGKRPRAAVIDTVSSIPAVRLPFEALVQVCHDEGILSIVDGAQGVGMIDLKHLGTQVKPDFFITNCHKWLYTPRGCAVLHVPKHNQALMRSTLPTSWGWVPSGEGDPDFIDNFAFASTLDNSNYMAVQHAVQWIQEALGGEDAVIEYMMSLNKKGGNMVAEMLGTKVLDNAEGTLTNCAMSNVLLPLGIKGRESSAKVLVDEEDAARLGDWCQKTLASDYNTWLPVTLIKGQWWTRISAQAYLDESDYEAVGKIFLELVERIGKGDHKK

SEQ ID NO: 83 - DNA, 1413 bp, unidentified
  source 1..1413: mol_type = genomic DNA; note = Glarea lozoyensis
atgccggctccgctgaatatgccgattcatctgaaagccggcgaagttagtagcgatgttagtaataagtataagcgtgttccgtttggtaaagaaatgctgaaacagtttagttttgacccggaatatcgtaatgtgaatcatggtagctatggtagctttccgaaaccgattagcgaactgcgtcgccattatctggatgaatgtgaaaaaagcccggacccttttattcgctatgattttggtagtattctggatgaaaaccgtgccgcagtggccaaactggtggatgccccgctgcataccgttgtttttgtgccgaatgcaaccaccggcattaatgttgttctgcgcaatctgcagtggaatgaaaatggcaaagatgaaattctgtactttaacaccatctatggtgcatgtggtaaaaccgttagttataccagtgaatatagtcgtggtctggtgcagggccgtgaaattaccctggattatccgattagcgatgaagcactgattgaacagtttagcagtaccattcaggccagcattgatgcaggcaaaaatccgcgcattgcaatttttgataccattagtagtctgccgggcgttcgcatgccgtttgaagccctgaccgcagtgtgcgccagtagcggtgtgctgagcctgattgatggcgcccatggcattggtcatattccgctgagtctgagcaccctgaatccggatttctttgtgagcaatctgcataaatggctgtttgtgccgcgtggttgcgcactgttttatgttccgctgcgcaatcagcatctgattcgtaccagcctgccgaccagtcattattttgaaccgaaacagctgagcctgggcgccccgaatccgtttgccccgaccaccaaaagtggctttgtgatgcagtttgaaagtaatggcaccattgataatagtccgtatctgaccgtggccgaagcaattcgttggcgccgcgaagcatgtggcggcgaagaagcaattcatgattattgtctggatctgagccgtaaaggcgcaaccctgattgcaagtattctgaatacccatattctggataatccgcagcataccctgaccaattgtcatctgagcaatattctgctgccggttagcaccaaaccgtatcaggattttcatgttattccggaagaacatgcacatctggtgggtgaatggattcataccaccatgattaaggatcataaaacctttgtggccattttctattttcaggaaaaatggtggggccgcctgagtgcacagatatatctggaaattgaagattatgagtgggcaggtaatgttctgaaaggcctgtgcgaacgcgtgggccgcctggaatttctgggcgaagaagttccgcgcggcgatgcagcaccgtaa

SEQ ID NO: 84 - protein, 470 aa, unidentified
  source 1..470: note = Glarea lozoyensis
MPAPLNMPIHLKAGEVSSDVSNKYKRVPFGKEMLKQFSFDPEYRNVNHGSYGSFPKPISELRRHYLDECEKSPDPFIRYDFGSILDENRAAVAKLVDAPLHTVVFVPNATTGINVVLRNLQWNENGKDEILYFNTIYGACGKTVSYTSEYSRGLVQGREITLDYPISDEALIEQFSSTIQASIDAGKNPRIAIFDTISSLPGVRMPFEALTAVCASSGVLSLIDGAHGIGHIPLSLSTLNPDFFVSNLHKWLFVPRGCALFYVPLRNQHLIRTSLPTSHYFEPKQLSLGAPNPFAPTTKSGFVMQFESNGTIDNSPYLTVAEAIRWRREACGGEEAIHDYCLDLSRKGATLIASILNTHILDNPQHTLTNCHLSNILLPVSTKPYQDFHVIPEEHAHLVGEWIHTTMIKDHKTFVAIFYFQEKWWGRLSAQIYLEIEDYEWAGNVLKGLCERVGRLEFLGEEVPRGDAAP

SEQ ID NO: 85 - DNA, 1446 bp, unidentified
  source 1..1446: mol_type = genomic DNA; note = Exserohilum turcica
atgaccagtaatagcaaatacggtgttccggatattaagaccaaagatggtattgaatttggtaaagaactgcaggaaaaagaatttctgtttgataaaggttacatcggtctgaatcatggtagttttggtacctatccgcgcccggtgcgtgatcgtctgcgtgcatttcaggatgccagcgaagcccagccggataaattcattctgtatgattatccgcgttatctggatgaagcccgtgaagccatggcaaaactgctgaataccccgagtagcaccctggtttttgttccgaatgcaaccaccggtgtgaatattgttctgcgtaatctggtttttaccccggaagatcatattctgatttttagtaatatctacggcgcctgcgaacgtaccgttagttatattaccgaaaccaccccggcacagagtgttaaagttgaatatgccctgccgtttgaagatgattggctggttgaacagtttgaaagcaaagttcgtgatgttgaagcaaaaggcggtaaagtgaaaattgcaatttttgataccgtggtgagcatgccgggcattcgtctgccgtttgagcgcctgaccgcaaaaagcaaagaactgggcattctgagttgcattgatggtgcccatggcgttggccatgtggaaattgatctgggtaccctggaccctgatttctttgtgagtaattgtcataaatggctgcatgtgccgcgtggtaccgccatttttcatgttgcccatcgtgcccagcatctgattcgtagcaccctgccgaccagtcatggttttaccccgaaaaatggtaaatttgtgagcccgtttagcaaaccggtgtatcataatcgtagtcagcagaccggtgccgaacagaataccagcgaacagcagaccgccggtaccgccgcaagtagcgaaaaaccggaatttgtggcaaattttgaatttgttggcaccattgatagtagcccgtatctgtgcgttccgaccgcactgaaatggcgtgaaagcctgggcggcgaagcagtgattcgcagttattgtaccaccctggcccaggcagccggccagcatgtggctagtgttctgggtacccatgtgctggaaaatcgcacccgcaccctgggccagtgctgtctgagtaatgttctgctgccgattagcctggaaaaagttcatgccaccgcacgcctggccggtattgatccggatgatgccggtctgaaagttcgcgattggatgaaaaaactgagcagcgaacagtataatacctttattatggtttactggtacgccggcaaatggtggacccgcctgagtggtcaggtttatctggatatgcgtgattttgaatgggccgcacataccctgaaagaaatgtgcgcccgtgtggaaagcggtgaatgggcaggtgtgaaaggtcgcctgtaa

SEQ ID NO: 86 - protein, 481 aa, unidentified
  source 1..481: note = Exserohilum turcica
MTSNSKYGVPDIKTKDGIEFGKELQEKEFLFDKGYIGLNHGSFGTYPRPVRDRLRAFQDASEAQPDKFILYDYPRYLDEAREAMAKLLNTPSSTLVFVPNATTGVNIVLRNLVFTPEDHILIFSNIYGACERTVSYITETTPAQSVKVEYALPFEDDWLVEQFESKVRDVEAKGGKVKIAIFDTVVSMPGIRLPFERLTAKSKELGILSCIDGAHGVGHVEIDLGTLDPDFFVSNCHKWLHVPRGTAIFHVAHRAQHLIRSTLPTSHGFTPKNGKFVSPFSKPVYHNRSQQTGAEQNTSEQQTAGTAASSEKPEFVANFEFVGTIDSSPYLCVPTALKWRESLGGEAVIRSYCTTLAQAAGQHVASVLGTHVLENRTRTLGQCCLSNVLLPISLEKVHATARLAGIDPDDAGLKVRDWMKKLSSEQYNTFIMVYWYAGKWWTRLSGQVYLDMRDFEWAAHTLKEMCARVESGEWAGVKGRL

SEQ ID NO: 87 - DNA, 1179 bp, unidentified
  source 1..1179: mol_type = genomic DNA; note = Schizosaccharomyces cryophilus
atgagcgattgtatgccgtttggccatgcactgaaaccgtattatatgctggataaaaattacgtgagcgtgaataatggtagttatggcgtggtgtgtgccagtgcatttcagcgtcatctgcagctgctggaagaaagcgaaaaaacccaggatctgcagatgaaatatcgtctgccgaaactggcaaataataccctgctgcagattgccgaactgctggataccaccagtagcaatctggcattttgctttagtgcaacccaggccattagtagtattctgctgacctttccgtggagtgcaaatgataaaattctgagtctgaatgttgcctatccgacctgccagtttgccctggattttgttcgcaatcgttatgatgtgcaggttgataccctggaagtggagtttatctatgatccgagcgaatttctgagccgcgttgaaagctatctggttaaaaataagccgcgcgtttttatttttgattttattaccagcatgccggttacccagctgccgtgtaaagaactgattcagctgtgcaaaaaatatggtgtgattagtgtggttgatgcagcacatggcattggtttttgcccgctgagcctgagtagtctggaccctgattttctgtataccaatgcacataaatggctgaatgccccgagcggtaccaccattctgtatgttagcaaaaaatatcacaacttcatcgaagcactgccgattagctatggctatcatattcgtaaacagaatagtccgccggccgatagtctgggtattcgttttctgaatgcaagctttatggatctgccgaaattcattgccattgatgccgccattgcatttcgtaaaagtattggcggcgaacataaaattcagagttataatcatgacatcgccgtgcgtggcagtaaaattattgccgaaagcctgggtaccagctattttgcactggccagcccgattgcaatggttaatgttgaagttccgctgcgctgcattccgagtgccgattttctggaagaattttggcagagcaaaaatacctttctgcgctttgtggaatatcagggtcgttattatacccgcgtgggtggtgccccgtttctggaagagagtgattttgtgtatgtggccgatgtgctgaaagaactgtgccagaaataa

SEQ ID NO: 88 - protein, 392 aa, unidentified
  source 1..392: note = Schizosaccharomyces cryophilus
MSDCMPFGHALKPYYMLDKNYVSVNNGSYGVVCASAFQRHLQLLEESEKTQDLQMKYRLPKLANNTLLQIAELLDTTSSNLAFCFSATQAISSILLTFPWSANDKILSLNVAYPTCQFALDFVRNRYDVQVDTLEVEFIYDPSEFLSRVESYLVKNKPRVFIFDFITSMPVTQLPCKELIQLCKKYGVISVVDAAHGIGFCPLSLSSLDPDFLYTNAHKWLNAPSGTTILYVSKKYHNFIEALPISYGYHIRKQNSPPADSLGIRFLNASFMDLPKFIAIDAAIAFRKSIGGEHKIQSYNHDIAVRGSKIIAESLGTSYFALASPIAMVNVEVPLRCIPSADFLEEFWQSKNTFLRFVEYQGRYYTRVGGAPFLEESDFVYVADVLKELCQK

SEQ ID NO: 89 - DNA, 1290 bp, unidentified
  source 1..1290: mol_type = genomic DNA; note = Talaromyces stipitatus
atgagcaccagtaatccgacctttggtgcaccgctgctgccgtattttccgtttcagagcgattatctgaatattaatcacggtagctttggtggttatccgattaaggtgcgtgatgccctgcgtgaatatcagcgccagaccgatgccaaaccggatgattttattcgttatcgcctgccgggtctgattgataaaagccgcgccgcagttgcagaactgattaatgccgatgtgggcaatgttgtgctgattccgaatgccaccaccggtgttaataccgtgctgcgtaatctggtgtataatccgggtgacaaaattgtgtatctgggcaccacctatggcgcatgtgaaaaagccgtgatgcatattgtggatacctgtattccggccggtgccgttgaagcaattaaggttgaagttgaatatccggttaccagcaaagaaattctgcgccgctttgaagatgccattagtcagaaaggtgtgcgtattgccctgtttgataccgttagtagtctgccggccctgcgtctgccgtatgaaaatatgattagcctgtgtaaaaagtaccatgtgctgagcctgattgatggtgcccatgcagttggcgccattgaactggatatgcagcgcctggaccctgatttctttattagcaatctgcataaatggctgtataccccgcgtagctgcgcagtttttcatgtggcagcccgtagccagcatctgattaagaccagcctgccgaccagccatggctatcgtccggaagaacgcccgggtcgtctgcgcgtgagcaatccgctgccgaccagtagtaaaaccggttttgtggaactgtttggttatgtgggcaccatggattataccccgtatctgtgcattccggaagccattaagtttcgtaaagaagtgtgtggcggcgaacagaaactgctgcagtatattaccaccctggccaaacagggcggcaatctggttgcaaatattctgggcaccgaactgctgggtgacgaagatcagcgccgcagcccgatggttatggtgcgcctgccgctgaaattcactgccgatgaactgcagcagggtaaacagcatctgctgctggaagaaattgaacgtaccattagcgaaaaatatcgcacctttgttccgctgatctatcatggcggtcatgcctatggtcgtctgagtggccaggtgtatctgaccctggaagattttgaaaaagccggccagattctggccaaagcctgtaaagaatttgaacagaaaagcaaactgtaa

SEQ ID NO: 90 - protein, 429 aa, unidentified
  source 1..429: note = Talaromyces stipitatus
MSTSNPTFGAPLLPYFPFQSDYLNINHGSFGGYPIKVRDALREYQRQTDAKPDDFIRYRLPGLIDKSRAAVAELINADVGNVVLIPNATTGVNTVLRNLVYNPGDKIVYLGTTYGACEKAVMHIVDTCIPAGAVEAIKVEVEYPVTSKEILRRFEDAISQKGVRIALFDTVSSLPALRLPYENMISLCKKYHVLSLIDGAHAVGAIELDMQRLDPDFFISNLHKWLYTPRSCAVFHVAARSQHLIKTSLPTSHGYRPEERPGRLRVSNPLPTSSKTGFVELFGYVGTMDYTPYLCIPEAIKFRKEVCGGEQKLLQYITTLAKQGGNLVANILGTELLGDEDQRRSPMVMVRLPLKFTADELQQGKQHLLLEEIERTISEKYRTFVPLIYHGGHAYGRLSGQVYLTLEDFEKAGQILAKACKEFEQKSKL

SEQ ID NO: 91 - DNA, 1449 bp, unidentified
  source 1..1449: mol_type = genomic DNA; note = Arthrobotrys oligospora
atggccgctagtaatccgccgaaaaccccgacctttggccatagcctgcgtcgccagtttctgtttccggaaaattataccaatctgaatcatggcagctttggtgcaattccggccccggttctgacccatcgtcagaaactgcatattctgagcgaacagcatccggataattttatgcgctatcatagtattagcctgctggatgaaagccgcgccgccgttgccaaagtgctgaatgcaccgagcgaagaagttgtgtttgttaccaatgcaaccaccggtgtgaatattgttctgcgcaatctggtttatgaagaaggtgacgtgattctgcattttggtaccatctatggtgcatgcggccgtaccgttcagtatattgccgataccaccccggcaacctgtattagcattccgctggcatatccggttagcgatgcaagtattctgagtagctttaataccaccgttcaggaaattaaggccgcaggtaaaaaaccgaaactggttatttttgataccgtgagcagcatgccgggcatgcgctttccgtgggaaaaaatgattgtggccgcaaaagaagccggcgttctgagcctgattgatggcgcccatggtgttggtaatattaagattgatctgggtgccaatcagccggatttctttgtgagcaattgccataaatggctgtataccccgcgcccggcagcagttctgtttgttccgattcgtaatcagccgctgattaccaccagcgtgccgaccagtcattattatattccgaaaagtgcagcccagtattggagcccgctgagtccgggtaccaaaagcaattttattctgcagtttgagtttaatggcaccattgatgcaaccccgtatctgtgcgtgccggcagccctgaaatttcgccaggaaattggcggtgaagatgccattattaattattgtaacaccctggcattcgaaggtggcgaagcagttgcaaaaattctgggtaccgaaattatggccccggaccctgcagccgttgatggtggtcgctgcccgatggtgaatattcgtctgccgctgctgagtgtgccgaaaaccgaagttgaaccggtgtataatacctttaccaaagaagttggtatccgcgaaaatacctttgtgcaggtttatgttcataatgcccgttggtgggtgcgtattagtgcccaggtgtatctggaaatgaaagattttgtttggatcgccggcgtgctgaaaaaagaatgcgaaaaaattaacgagcgtattaagagtctggccaccattgcagcagcaaccggcgaaaaagcagatgttgcaaatggtgcagatgttcatgttgaagaagttcgcagcgccaaaaaagtggtgagcggcatgggtgacctgaaagttagcgaagccgaaggcgaaaccgtgaccgttaaaggctaa

SEQ ID NO: 92 - protein, 482 aa, unidentified
  source 1..482: note = Arthrobotrys oligospora
MAASNPPKTPTFGHSLRRQFLFPENYTNLNHGSFGAIPAPVLTHRQKLHILSEQHPDNFMRYHSISLLDESRAAVAKVLNAPSEEVVFVTNATTGVNIVLRNLVYEEGDVILHFGTIYGACGRTVQYIADTTPATCISIPLAYPVSDASILSSFNTTVQEIKAAGKKPKLVIFDTVSSMPGMRFPWEKMIVAAKEAGVLSLIDGAHGVGNIKIDLGANQPDFFVSNCHKWLYTPRPAAVLFVPIRNQPLITTSVPTSHYYIPKSAAQYWSPLSPGTKSNFILQFEFNGTIDATPYLCVPAALKFRQEIGGEDAIINYCNTLAFEGGEAVAKILGTEIMAPDPAAVDGGRCPMVNIRLPLLSVPKTEVEPVYNTFTKEVGIRENTFVQVYVHNARWWVRISAQVYLEMKDFVWIAGVLKKECEKINERIKSLATIAAATGEKADVANGADVHVEEVRSAKKVVSGMGDLKVSEAEGETVTVKG

SEQ ID NO: 93 - DNA, 1299 bp, unidentified
  source 1..1299: mol_type = genomic DNA; note = Rasamsonia emersonii
atgagtaccccgtttggccgtccgatgcgcgaacattttctgtttgaagaaggtaatatcaacatcaatcacggcagttttggcacctatccgaaaccggttctggatgcactgcgtagttatcagctgcagggcgaagccaatccggatcgctttctgcgctatgaagtgagcgaactgattgatcgtagtcgcgaacagctggcaaaactgctgcatgttgttgatgttgatgaactggtgctggttcagaatgcaaccaccggcgttaataccgtgctgcgtaatctgacctatgccccgggcgataaaattctgtatctgagcaccgcatatggtgcatgtgaaaaaaccgttgattatctgaccgaaaccaccccggccgaagcagtgcgtgttgaagttgcatatccgattagcgatgatgaactggttgcacgtgtggaaaaagtgctgaaagaaaatgcaccggttaaagttgcaatgtttgataccgtgagtagtctgccgggcgtgcgtattccgtttgaacgtctggttgccgtttgccgtgccgcaggtgttctgagcctgattgatggtgcccatggtgttggctgcattccgctggatctgggcaaactggatgccgatttctttgtgagtaattgtcataaatggctgtatgtgccgcgtggttgcgcagtgctgcatgtgccgaaacgtaatcaggatctgattcgtagcagtatgccgaccagtcatggttatcagccgcgtgaacgtccgggcaaaaagaaaattagtaatccgctgccgccgagcaccaaaagcggttttgttcgcatgtttgagtttattggtagcatggattatgccccgtatctgtgcgtgccggccgccttaaaatttcgtcaggaagtttgcggtggtgaagaagcaattatgagctattgtacccaggttgcacgcgatggtagccgtcgtgtggccgaaattctgggtaccgaagttatgcgtcatgatcagccgtgcccggttgttaatgtgcgcctgccgattgatccgccggccggtgacgttaccgccgctgcagcacaggcacgtattggtgccgtgaatgcctttgttgaaaaaatgatgctgagcgaatataaaacctttgttccggcctttttccataatggccgtttttgggttcgtctgagcggccagatatatctgaccgtggatgattttgaagaagttggccgtcagctgcgcgatatttgtagccgtgtgggtcgtaccagtcatctgaccgaactggaagataaataa

SEQ ID NO: 94 - protein, 432 aa, unidentified
  source 1..432: note = Rasamsonia emersonii
MSTPFGRPMREHFLFEEGNININHGSFGTYPKPVLDALRSYQLQGEANPDRFLRYEVSELIDRSREQLAKLLHVVDVDELVLVQNATTGVNTVLRNLTYAPGDKILYLSTAYGACEKTVDYLTETTPAEAVRVEVAYPISDDELVARVEKVLKENAPVKVAMFDTVSSLPGVRIPFERLVAVCRAAGVLSLIDGAHGVGCIPLDLGKLDADFFVSNCHKWLYVPRGCAVLHVPKRNQDLIRSSMPTSHGYQPRERPGKKKISNPLPPSTKSGFVRMFEFIGSMDYAPYLCVPAALKFRQEVCGGEEAIMSYCTQVARDGSRRVAEILGTEVMRHDQPCPVVNVRLPIDPPAGDVTAAAAQARIGAVNAFVEKMMLSEYKTFVPAFFHNGRFWVRLSGQIYLTVDDFEEVGRQLRDICSRVGRTSHLTELEDK

SEQ ID NO: 95 - DNA, 1257 bp, Escherichia coli
  source 1..1257: mol_type = genomic DNA
atgagtggactcaaacaagaactggggctggcccagggcattggcctgctatcgacgtcattattaggcactggcgtgtttgccgttcctgcgttagctgcgctggtagcgggcaataacagcctgtgggcgtggcccgttttgattatcttagtgttcccgattgcgattgtgtttgcgattctgggtcgccactatcccagcgcaggcggcgtcgcgcacttcgtcggtatggcgtttggttcgcggcttgagcgagtcaccggctggctgtttttatcggtcattcccgtgggtttgcctgccgcactacaaattgccgccgggttcggccaggcgatgtttggctggcatagctggcaactgttgttggcagaactcggtacgctggcgctggtgtggtatatcggtactcgcggtgccagttccagtgctaatctacaaaccgttattgccggacttatcgtcgcgctgattgtcgctatctggtgggcgggcgatatcaaacctgcgaatatcccctttccggcacctggtaatatcgaacttaccgggttatttgctgcgttatcagtgatgttctggtgttttgtcggtctggaggcatttgcccatctcgcctcggaatttaaaaatccagagcgtgattttcctcgtgctttgatgattggtctgctgctggcaggattagtctactggggctgtacggtagtcgtcttacacttcgacgcctatggtgaaaaaatggcggcggcagcatcgcttccaaaaattgtagtgcagttgttcggtgtaggagcgttatggattgcctgcgtgattggctatctggcctgctttgccagtctcaacatttatatacagagcttcgcccgcctggtctggtcgcaggcgcaacataatcctgaccactacctggcacgcctctcttctcgccatatcccgaataatgccctcaatgcggtgctcggctgctgtgtggtgagcactttggtgattcatgctttagagatcaatctggacgctcttattatttatgccaatggcatctttattatgatttatctgttatgcatgctggcaggctgtaaattattgcaaggacgttatcgactactggcggtggttggcgggctgttatgcgttctgttactggcaatggtcggctggaaaagtctctatgcgctgatcatgctggcggggttatggctgttgctgccaaaacgaaaaacgccggaaaatggcataaccacataa

SEQ ID NO: 96 - protein, 418 aa, Escherichia coli
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSWQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEKMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLLLPKRKTPENGITT

SEQ ID NO: 97 - DNA, 1414 bp, Escherichia coli
  source 1..1414: mol_type = genomic DNA
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatctttggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttactggcaaacttaaaagcgatgtacagcatcgcgaagaaatacgatattccggtggtaatggactccgcgcgctttgctgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctggaaggcggcgcgatggagcgtctggcggtaggtctgtatgacggcatgaatctcgactggctggcttatcgtatcgcgcaggtacagtatctggtcgatggtctggaagagattggcgttgtctgccagcaggcgggcggtcacgcggcattcgttgatgccggtaaactgttgccgcatatcccggcagaccagttcccggcacaggcgctggcctgcgagctgtataaagtcgccggtatccgtcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaaggattaacctttacgtacgaaccgaaagtattgcgtcacttcaccgcaaaacttaaagaagtttaa

SEQ ID NO: 98 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 99 - DNA, 1365 bp, Escherichia coli
  source 1..1365: mol_type = genomic DNA
gtgattagtctattcgacatgtttaaggtggggattggtccctcatcttcccataccgtagggcctatgaaggcaggtaaacagttcgtcgatgatctggtcgaaaaaggcttactggatagcgttactcgcgttgccgtggacgtttatggttcactgtcgctgacgggtaaaggccaccacaccgatatcgccattattatgggtcttgcaggtaacgaacctgccaccgtggatatcgacagtattcccggttttattcgcgacgtagaagagcgcgaacgtctgctgctggcacagggacggcatgaagtggatttcccgcgcgacaacgggatgcgttttcataacggcaacctgccgctgcatgaaaacggtatgcaaatccacgcctataacggcgatgaagtcgtctacagcaaaacttattattccatcggcggcggttttatcgtcgatgaagaacactttggtcaggatgctgccaacgaagtaagcgtgccgtatccgttcaaatctgccaccgaactgctcgcgtactgtaatgaaaccggctattcgctgtctggtctcgctatgcagaacgaactggcgctgcacagcaagaaagagatcgacgagtatttcgcgcatgtctggcaaaccatgcaggcatgtatcgatcgcgggatgaacaccgaaggtgtactgccaggcccgctgcgcgtgccacgtcgtgcgtctgccctgcgccggatgctggtttccagcgataaactgtctaacgatccgatgaatgtcattgactgggtaaacatgtttgcgctggcagttaacgaagaaaacgccgccggtggtcgtgtggtaactgcgccaaccaacggtgcctgcggtatcgttccggcagtgctggcttactatgaccactttattgaatcggtcagcccggacatctatacccgttactttatggcagcgggcgcgattggtgcattgtataaaatgaacgcctctatttccggtgcggaagttggttgccagggcgaagtgggtgttgcctgttcaatggctgctgcgggtcttgcagaactgctgggcggtagcccggaacaggtttgcgtggcggcggaaattggcatggaacacaaccttggtttaacctgcgacccggttgcagggcaggttcaggtgccgtgcattgagcgtaatgccattgcctctgtgaaggcgattaacgccgcgcggatggctctgcgccgcaccagtgcaccgcgcgtctcgctggataaggtcatcgaaacgatgtacgaaaccggtaaggacatgaacgccaaataccgcgaaacctcacgcggtggtctggcaatcaaagtccagtgtgactaa

SEQ ID NO: 100 - protein, 454 aa, Escherichia coli
MISLFDMFKVGIGPSSSHTVGPMKAGKQFVDDLVEKGLLDSVTRVAVDVYGSLSLTGKGHHTDIAIIMGLAGNEPATVDIDSIPGFIRDVEERERLLLAQGRHEVDFPRDNGMRFHNGNLPLHENGMQIHAYNGDEVVYSKTYYSIGGGFIVDEEHFGQDAANEVSVPYPFKSATELLAYCNETGYSLSGLAMQNELALHSKKEIDEYFAHVWQTMQACIDRGMNTEGVLPGPLRVPRRASALRRMLVSSDKLSNDPMNVIDWVNMFALAVNEENAAGGRVVTAPTNGACGIVPAVLAYYDHFIESVSPDIYTRYFMAAGAIGALYKMNASISGAEVGCQGEVGVACSMAAAGLAELLGGSPEQVCVAAEIGMEHNLGLTCDPVAGQVQVPCIERNAIASVKAINAARMALRRTSAPRVSLDKVIETMYETGKDMNAKYRETSRGGLAIKVQCD

SEQ ID NO: 101 - DNA, 1233 bp, Escherichia coli
  source 1..1233: mol_type = genomic DNA
atggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaaccaggttgatctggatgcggcggcaaagcgcgggatcccggtatttaacgcaccgttctcaaatacgcgctctgttgcggagctggtgattggcgaactgctgctgctattgcgcggcgtgccggaagccaatgctaaagcgcaccgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaataaactgccgctgggcaacgccactcaggtacagcatctttctgacctgctgaatatgagcgatgtggtgagtctgcatgtaccagagaatccgtccaccaaaaatatgatgggcgcgaaagaaatttcactaatgaagcccggctcgctgctgattaatgcttcgcgcggtactgtggtggatattccggcgctgtgtgatgcgctggcgagcaaacatctggcgggggcggcaatcgacgtattcccgacggaaccggcgaccaatagcgatccatttacctctccgctgtgtgaattcgacaacgtccttctgacgccacacattggcggttcgactcaggaagcgcaggagaatatcggcctggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctgaacaaaatcttcgccgagcagggcgtcaacatcgccgcgcaatatctgcaaacttccgcccagatgggttatgtggttattgatattgaagccgacgaagacgttgccgaaaaagcgctgcaggcaatgaaagctattccgggtaccattcgcgcccgtctgctgtactaa

SEQ ID NO: 102 - protein, 410 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSDNGSTLSAVNFPEVSLPLHGGRRLMHIHENRPGVLTALNKIFAEQGVNIAAQYLQTSAQMGYVVIDIEADEDVAEKALQAMKAIPGTIRARLLY

SEQ ID NO: 103 - DNA, 969 bp, Escherichia coli
  source 1..969: mol_type = genomic DNA
atgcctaacattacctggtgcgacctgcctgaagatgtctctttatggccgggtctgcctctttcattaagtggtgatgaagtgatgccactggattaccacgcaggtcgtagcggctggctgctgtatggtcgtgggctggataaacaacgtctgacccaataccagagcaaactgggtgcggcgatggtgattgttgccgcctggtgcgtggaagattatcaggtgattcgtctggcaggttcactcaccgcacgggctacacgcctggcccacgaagcgcagctggatgtcgccccgctggggaaaatcccgcacctgcgcacgccgggtttgctggtgatggatatggactccaccgccatccagattgaatgtattgatgaaattgccaaactggccggaacgggcgagatggtggcggaagtaaccgaacgggcgatgcgcggcgaactcgattttaccgccagcctgcgcagccgtgtggcgacgctgaaaggcgctgacgccaatattctgcaacaggtgcgtgaaaatctgccgctgatgccaggcttaacgcaactggtgctcaagctggaaacgctgggctggaaagtggcgattgcctccggcggctttactttctttgctgaatacctgcgcgacaagctgcgcctgaccgccgtggtagccaatgaactggagatcatggacggtaaatttaccggcaatgtgatcggcgacatcgtagacgcgcagtacaaagcgaaaactctgactcgcctcgcgcaggagtatgaaatcccgctggcgcagaccgtggcgattggcgatggagccaatgacctgccgatgatcaaagcggcagggctggggattgcctaccatgccaagccaaaagtgaatgaaaaggcggaagtcaccatccgtcacgctgacctgatgggggtattctgcatcctctcaggcagcctgaatcagaagtaa

SEQ ID NO: 104 - protein, 322 aa, Escherichia coli [sequence omitted]

SEQ ID NO: 105 - DNA, 1088 bp, Escherichia coli
  source 1..1088: mol_type = genomic DNA
atggctcaaatcttcaattttagttctggtccggcaatgctaccggcagaggtgcttaaacaggctcaacaggaactgcgcgactggaacggtcttggtacgtcggtgatggaagtgagtcaccgtggcaaagagttcattcaggttgcagaggaagccgagaaggattttcgcgatcttcttaatgtcccctccaactacaaggtattattctgccatggcggtggtcgcggtcagtttgctgcggtaccgctgaatattctcggtgataaaaccaccgcagattatgttgatgccggttactgggcggcaagtgccattaaagaagcgaaaaaatactgcacgcctaatgtctttgacgccaaagtgactgttgatggtctgcgcgcggttaagccaatgcgtgaatggcaactctctgataatgctgcttatatgcattattgcccgaatgaaaccatcgatggtatcgccatcgacgaaacgccagacttcggcgcagatgtggtggtcgccgctgacttctcttcaaccattctttcccgtccgattgacgtcagccgttatggtgtaatttacgctggcgcgcagaaaaatatcggcccggctggcctgacaatcgtcatcgttcgtgaagatttgctgggcaaagcgaatatcgcgtgtccgtcgattctggattattccatcctcaacgatacggctccatgtttaacacgccgccgacatttgcctggtatctatctggtctggtctttaaatggctgaaagcgaacggcggtgtagctgaaatggataaaatcaatcagcaaaaagcagaactgctatatggggtgattgataacagcgatttctaccgcaatgacgtggcgaaagctaaccgttcgcggatgaacgtgccgttccagttggcggacagtgcgcttgacaaattgttccttgaagagtcttttgctgctggccttcatgcactgaaaggtcaccgtgtggtcggcggaatgcgcgcttctatttataacgccatgccgctggaaggcgttaaagcgctgacagacttcatggttgagttcgaacgccgtcacggttaa

SEQ ID NO: 106 - protein, 362 aa, Escherichia coli
MAQIFNFSSGPAMLPAEVLKQAQQELRDWNGLGTSVMEVSHRGKEFIQVAEEAEKDFRDLLNVPSNYKVLFCHGGGRGQFAAVPLNILGDKTTADYVDAGYWAASAIKEAKKYCTPNVFDAKVTVDGLRAVKPMREWQLSDNAAYMHYCPNETIDGIAIDETPDFGADVVVAADFSSTILSRPIDVSRYGVIYAGAQKNIGPAGLTIVIVREDLLGKANIACPSILDYSILNDNGSMFNTPPTFAWYLSGLVFKWLKANGGVAEMDKINQQKAELLYGVIDNSDFYRNDVAKANRSRMNVPFQLADSALDKLFLEESFAAGLHALKGHRVVGGMRASIYNAMPLEGVKALTDFMVEFERRHG

SEQ ID NO: 107 - DNA, 912 bp, Escherichia coli
  source 1..912: mol_type = genomic DNA
gtgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcggggaaattaaaccgggtgatgtcttaatcgaagccaccagtggtaacaccggcattgcgctggcaatgattgccgcgctgaaaggctatcgcatgaaattgctgatgcccgacaacatgagccaggaacgccgtgcggcgatgcgtgcttatggtgcggaactgattcttgtcaccaaagagcagggcatggaaggtgcgcgcgatctggcgctggagatggcgaatcgtggcgaaggaaagctgctcgatcagttcaataatcccgataacccttatgcgcattacaccaccactgggccggaaatctggcagcaaaccggcgggcgcatcactcattttgtctccagcatggggacgaccggcactatcaccggcgtctcacgctttatgcgcgaacaatccaaaccggtgaccattgtcggcctgcaaccggaagagggcagcagcattcccggcattcgccgctggcctacggaatatctgccggggattttcaacgcttctctggtggatgaggtgctggatattcatcagcgcgatgcggaaaacaccatgcgcgaactggcggtgcgggaaggaatattctgtggcgtcagctccggcggcgcggttgccggagcactgcgggtggcaaaagctaaccctgacgcggtggtggtggcgatcatctgcgatcgtggcgatcgctacctttctaccggggtgtttggggaagagcattttagccagggggcggggatttaa

SEQ ID NO: 108 - protein, 303 aa, Escherichia coli
MSTLEQTIGNTPLVKLQRMGPDNGSEVWLKLEGNNPAGSVKDRAALSMIVEAEKRGEIKPGDVLIEATSGNTGIALAMIAALKGYRMKLLMPDNMSQERRAAMRAYGAELILVTKEQGMEGARDLALEMANRGEGKLLDQFNNPDNPYAHYTTTGPEIWQQTGGRITHFVSSMGTTGTITGVSRFMREQSKPVTIVGLQPEEGSSIPGIRRWPTEYLPGIFNASLVDEVLDIHQRDAENTMRELAVREGIFCGVSSGGAVAGALRVAKANPDAVVVAIICDRGDRYLSTGVFGEEHFSQGAGI

SEQ ID NO: 109 - DNA, 246 bp, Escherichia coli
  source 1..246: mol_type = genomic DNA
atgcgcattactatttacactcgtaacgattgcgttcagtgccacgccaccaaacgggcgatggaaaaccggggctttgattttgaaatgattaatgtcgatcgcgttcctgaagcggcagaagcgttgcgtgctcagggctttcgtcagttgccggtagtgattgctggcgatcttagctggtctggtttccgtccggacatgattaaccgtctgcatccagcgccacacgcggccagtgcatga

SEQ ID NO: 110 - protein, 136 aa, Escherichia coli
MSQLVYFSSSSENTQRFIERLGLPAVRIPLNERERIQVDEPYILIVPSYGGGGTAGAVPRQVIRFLNDEHNRALLRGVIASGNRNFGEAYGRAGDVIARKCGVPWLYRFELMGTQSDIENVRKGVTEFWQRQPQNA

SEQ ID NO: 111 - DNA, 822 bp, Escherichia coli
  source 1..822: mol_type = genomic DNA
atgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgatgattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaa

SEQ ID NO: 112 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVMIGAGAKILGNIEVGRGAKIGAGSVVLQPVPPHTTAAGVPARIVGKPDSDKPSMDMDQHFNGINHTFEYGDGI

SEQ ID NO: 113 - DNA, 900 bp, Escherichia coli
  source 1..900: mol_type = genomic DNA
atgtcgcgaaaagatggggtgttggcgctactggtagtggtcgtatgggggctaaattttgtggtcatcaaagtggggcttcataacatgccaccgctgatgctggccggtttgcgctttatgctggtcgcttttccggctatcttttttgtcgcacgaccgaaagtaccactgaatttgctgctggggtatggattaaccatcagttttgcgcagtttgcttttcttttttgtgccattaacttcggtatgcctgctggactggcttcgctggtgttacaggcacaggcgttttttactatcatgcttggcgcgtttactttcggggagcgactgcatggcaaacaattggcggggatcgccttagcgatttttggcgtactggtgttaatcgaagatagtctgaacggtcagcatgtggcgatgctcggctttatgttgaccctggcggcagcatttagttgggcgtgtggcaacatcttcaataaaaagatcatgtcgcactcaacgcgtccggcggtgatgtcgctggtaatctggagcgctttaatcccaatcattcccttctttgttgcctcgctgattctcgatggttccgcaaccatgattcacagtctggttactatcgatatgaccaccatcttgtctctgatgtatctggcgtttgtggcgacaattgttggttatgggatctgggggacgttactgggacgctatgaaacctggcgggttgcaccgttatcgttactggtgcccgtagtaggactggcaagtgcggcactattgttggatgaacgcttaacgggtctgcaatttttaggtgcggtgctcattatgaccgggctgtatatcaatgtatttggcttgcggtggcgtaaagcggtaaaggtgggaagttaa

SEQ ID NO: 114 - protein, 299 aa, Escherichia coli
MSRKDGVLALLVVVVWGLNFVVIKVGLHNMPPLMLAGLRFMLVAFPAIFFVARPKVPLNLLLGYGLTISFAQFAFLFCAINFGMPAGLASLVLQAQAFFTIMLGAFTFGERLHGKQLAGIALAIFGVLVLIEDSLNGQHVAMLGFMLTLAAAFSWACGNIFNKKIMSHSTRPAVMSLVIWSALIPIIPFFVASLILDGSATMIHSLVTIDMTTILSLMYLAFVATIVGYGIWGTLLGRYETWRVAPLSLLVPVVGLASAALLLDERLTGLQFLGAVLIMTGLYINVFGLRWRKAVKVGS

SEQ ID NO: 115 - DNA, 1311 bp, Escherichia coli
  source 1..1311: mol_type = genomic DNA
atgtttgattcgactttaaatccgttatggcagcgttacatcctcgccgttcaggaggaagtaaaaccggcgctgggatgtactgaaccgatttcactggcgctggcggcggcggttgctgcggcagaactggaaggtccggttgaacgtgtagaagcctgggtttcgccaaatctgatgaagaacggtctgggcgtcaccgttcccggcacgggaatggtggggctgccgattgcggcggcgctgggggcgttaggtggaaatgccaacgccgggctggaagtgctgaaagacgcaacagcgcaggcaattgccgatgccaaagcactgctggcggcggggaaagtctccgttaagatccaggaaccttgcgatgaaatcctcttctcacgcgccaaagtctggaacggtgagaagtgggcgtgtgtcaccatcgtcggcgggcataccaacattgtgcatatcgagacgcacgatggtgtggtgtttacccagcaggcgtgtgtggcagagggcgagcaagagtctccgctgacggtgctttccagaacgacgctggctgagatcctgaagttcgtcaatgaagtcccgtttgcggcgatccgctttattctcgattccgcgaagctaaattgtgcgttatcgcaggaaggtttgagcggtaagtgggggctgcatattggcgcgacgctggaaaaacagtgcgagcgcggtttgctggcgaaagatctctcttcatccattgtgattcgtaccagcgcggcatccgatgcgcgtatgggcggcgctacgcttccggctatgagtaactccggctcgggtaaccaggggattaccgcaacaatgcctgtggtggtggtagcagaacacttcggagcggatgatgaacggctggcgcgtgcgctgatgctttcgcatttgagcgcaatttacatccataaccagttaccgcgtttgtctgcgctgtgtgccgcaacgaccgcagcaatgggggccgccgccgggatggcatggctggtggatgggcgttatgaaaccatctcgatggcgatcagcagtatgatcggcgatgtcagcggcatgatttgcgatggtgcgtcgaacagctgcgcgatgaaggtttcgaccagtgcttcggctgcgtggaaagcggtgttaatggcgctggatgataccgccgtgaccggcaatgaagggattgtggcgcatgatgttgagcagtcgattgccaacctgtgtgcgttagcaagccattcgatgcagcaaacggatcggcagattatcgagattatggcgagcaaggccagataa

SEQ ID NO: 116 - protein, 436 aa, Escherichia coli
MFDSTLNPLWQRYILAVQEEVKPALGCTEPISLALAAAVAAAELEGPVERVEAWVSPNLMKNGLGVTVPGTGMVGLPIAAALGALGGNANAGLEVLKDATAQAIADAKALLAAGKVSVKIQEPCDEILFSRAKVWNGEKWACVTIVGGHTNIVHIETHDGVVFTQQACVAEGEQESPLTVLSRTTLAEILKFVNEVPFAAIRFILDSAKLNCALSQEGLSGKWGLHIGATLEKQCERGLLAKDLSSSIVIRTSAASDARMGGATLPAMSNSGSGNQGITATMPVVVVAEHFGADDERLARALMLSHLSAIYIHNQLPRLSALCAATTAAMGAAAGMAWLVDGRYETISMAISSMIGDVSGMICDGASNSCAMKVSTSASAAWKAVLMALDDTAVTGNEGIVAHDVEQSIANLCALASHSMQQTDRQIIEIMASKAR

SEQ ID NO: 117 - DNA, 975 bp, Escherichia coli
  source 1..975: mol_type = genomic DNA
atgaaattacaacaacttcgctatattgttgaggtggtcaatcataacctgaatgtctcatcaacagcggaaggactttacacatcacaacccgggatcagtaaacaagtcagaatgctggaagacgagctaggcattcaaattttttcccgaagcggcaagcacctgacgcaggtaacgccagcagggcaagaaataattcgtatcgctcgcgaagtcctgtcgaaagtcgatgccataaaatcggttgccggagagcacacctggccggataaaggttcactgtatatcgccaccacgcatacccaggcacgctacgcattaccaaacgtcatcaaaggctttattgagcgttatcctcgcgtttctttgcatatgcaccagggctcgccgacacaaattgctgatgccgtctctaaaggcaatgctgatttcgctatcgccacagaagcgctgcatctgtatgaagatttagtgatgttaccgtgctaccactggaatcgggctattgtagtcactccggatcacccgctggcaggcaaaaaagccattaccattgaagaactggcgcaatatccgttggtgacatataccttcggctttaccggacgttcagaactggatactgcctttaatcgcgcagggttaacgccgcgtatcgttttcacggcaacggatgctgacgtcattaaaacttacgtccggttagggctgggggtaggggtcattgccagcatggcggtggatccggtcgccgatcccgaccttgtgcgtgttgatgctcacgatatcttcagccacagtacaaccaaaattggttttcgccgtagtactttcttgcgcagttatatgtatgatttcattcagcgttttgcaccgcatttaacgcgtgatgtcgttgatgcggctgtcgcattgcgctctaatgaagaaattgaggtcatgtttaaagatataaaactgccggaaaaataa

SEQ ID NO: 118 - protein, 324 aa, Escherichia coli
MKLQQLRYIVEVVNHNLNVSSTAEGLYTSQPGISKQVRMLEDELGIQIFSRSGKHLTQVTPAGQEIIRIAREVLSKVDAIKSVAGEHTWPDKGSLYIATTHTQARYALPNVIKGFIERYPRVSLHMHQGSPTQIADAVSKGNADFAIATEALHLYEDLVMLPCYHWNRAIVVTPDHPLAGKKAITIEELAQYPLVTYTFGFTGRSELDTAFNRAGLTPRIVFTATDADVIKTYVRLGLGVGVIASMAVDPVADPDLVRVDAHDIFSHSTTKIGFRRSTFLRSYMYDFIQRFAPHLTRDVVDAAVALRSNEEIEVMFKDIKLPEK

SEQ ID NO: 119 - DNA, 972 bp, Escherichia coli
  source 1..972: mol_type = genomic DNA
atgagtaagatttttgaagataactcgctgactatcggtcacacgccgctggttcgcctgaatcgcatcggtaacggacgcattctggcgaaggtggaatctcgtaaccccagcttcagcgttaagtgccgtatcggtgccaacatgatttgggatgccgaaaagcgcggcgtgctgaaaccaggcgttgaactggttgaaccgaccagcggtaataccgggattgcactggcctatgtagctgccgctcgcggttacaaactcaccctgaccatgccagaaaccatgagtattgaacgccgcaagctgctgaaagcgttaggtgcaaacctggtgctgacggaaggtgctaaaggcatgaaaggcgcaatccaaaaagcagaagaaattgtcgccagcaatccagagaaatacctgctgctgcaacaattcagcaatccggcaaaccctgaaattcacgaaaagaccaccggtccggagatatgggaagataccgacggtcaggttgatgtatttattgctggcgttgggactggcggtacgctgactggcgtcagccgctacattaaaggcaccaaaggcaagaccgatcttatctctgtcgccgttgagccaaccgattctccagttatcgcccaggcgctggcaggtgaagagattaaacctggcccgcataaaattcagggtattggcgctggttttatcccggctaacctcgatctcaagctggtcgataaagtcattggcatcaccaatgaagaagcgatttctaccgcgcgtcgtctgatggaagaagaaggtattcttgcaggtatctcttctggagcagctgttgccgcggcgttgaaactacaagaagatgaaagctttaccaacaagaatattgtggttattctaccatcatcgggtgagcgttatttaagcaccgcattgtttgccgatctcttcactgagaaagaattgcaacagtaa

SEQ ID NO: 120 - protein, 323 aa, Escherichia coli
MSKIFEDNSLTIGHTPLVRLNRIGNGRILAKVESRNPSFSVKCRIGANMIWDAEKRGVLKPGVELVEPTSGNTGIALAYVAAARGYKLTLTMPETMSIERRKLLKALGANLVLTEGAKGMKGAIQKAEEIVASNPEKYLLLQQFSNPANPEIHEKTTGPEIWEDTDGQVDVFIAGVGTGGTLTGVSRYIKGTKGKTDLISVAVEPTDSPVIAQALAGEEIKPGPHKIQGIGAGFIPANLDLKLVDKVIGITNEEAISTARRLMEEEGILAGISSGAAVAAALKLQEDESFTNKNIVVILPSSGERYLSTALFADLFTEKELQQ

SEQ ID NO: 121 - DNA, 1098 bp, Escherichia coli
  source 1..1098: mol_type = genomic DNA
atgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 122 - protein, 365 aa, Escherichia coli
MSIEIANIKKSFGRTQVLNDISLDIPSGQMVALLGPSGSGKTTLLRIIAGLEHQTSGHIRFHGTDVSRLHARDRKVGFVFQHYALFRHMTVFDNIAFGLTVLPRRERPNAAAIKAKVTKLLEMVQLAHLADRYPAQLSGGQKQRVALARALAVEPQILLLDEPFGALDAQVRKELRRWLRQLHEELKFTSVFVTHDQEEATEVADRVVVMSQGNIEQADAPDQVWREPATRFVLEFMGEVNRLQGTIRGGQFHVGAHRWPLGYTPAYQGPVDLFLRPWEVDISRRTSLDSPLPVQVLEASPKGHYTQLVVQPLGWYNEPLTVVMHGDDAPQRGERLFVGLQHARLYNGDERIETRDEELALAQSA

SEQ ID NO: 123 - DNA, 1017 bp, Escherichia coli
  source 1..1017: mol_type = genomic DNA
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactga

SEQ ID NO: 124 - protein, 338 aa, Escherichia coli
MAVNLLKKNSLALVASLLLAGHVQATELLNSSYDVSRELFAALNPPFEQQWAKDNGGDKLTIKQSHAGSSKQALAILQGLKADVVTYNQVTDVQILHDKGKLIPADWQSRLPNNSSPFYSTMGFLVRKGNPKNIHDWNDLVRSDVKLIFPNPKTSGNARYTYLAAWGAADKADGGDKGKTEQFMTQFLKNVEVFDTGGRGATTTFAERGLGDVLISFESEVNNIRKQYEAQGFEVVIPKTNILAEFPVAWVDKNVQANGTEKAAKAYLNWLYSPQAQTIITDYYYRVNNPEVMDKLKDKFPQTELFRVEDKFGSWPEVMKTHFTSGGELDKLLAAGRN

SEQ ID NO: 125 - DNA, 852 bp, Escherichia coli
  source 1..852: mol_type = genomic DNA
atgacggaatcgttggtcggcgaacgccgcgcgccgcagttccgcgcgcgcctttccggccccgcgggccccccttccgttcgggtcggtatggcagtggtgtggctttcggtgatcgtgctgttgccgctggccgccatcgtctggcaggccgcgggcggtggttggcgggccttctggctggcggtctcgtcgcatgccgcgatggagtcgttccgggtaacgctgacgatttcgaccgcagtcacggtcatcaacctggtgttcggcttgctgatcgcctgggtgctggtgcgtgacgacttcgctggcaagcggatcgtcgatgcgattatcgatctgccgtttgcgttgcccaccatcgtcgccagcctggtgatgttggcactgtacgggaacaacagcccggtggggcttcattttcaacacaccgcgaccggtgttggggtggcgttggcgttcgtcacattgccgttcgtggtgcgcgccgtgcagccggtgctgctggaaatcgatcgcgagaccgaggaggcggcggcgtcgctgggcgctaatggtgccaaaatcttcacttcggtggtgttgccgtcgctgacgccggcattgttatccggtgcgggcctggcgttttcgcgcgctatcggcgagttcggttcggtggttctgatcggcggggccgtgccgggcaagaccgaggtgtcctcgcaatggattcgcaccctgatcgagaacgacgaccgcaccggagcggccgcgatatcggttgtattgctctcgatttcgttcattgtgctgctcatcctacgtgtcgtcggcgcgcgtgcggccaaacgtgaggagatggccgcatga

SEQ ID NO: 126 - protein, 283 aa, Escherichia coli
MTESLVGERRAPQFRARLSGPAGPPSVRVGMAVVWLSVIVLLPLAAIVWQAAGGGWRAFWLAVSSHAAMESFRVTLTISTAVTVINLVFGLLIAWVLVRDDFAGKRIVDAIIDLPFALPTIVASLVMLALYGNNSPVGLHFQHTATGVGVALAFVTLPFVVRAVQPVLLEIDRETEEAAASLGANGAKIFTSVVLPSLTPALLSGAGLAFSRAIGEFGSVVLIGGAVPGKTEVSSQWIRTLIENDDRTGAAAISVVLLSISFIVLLILRVVGARAAKREEMAA

SEQ ID NO: 127 - DNA, 876 bp, Escherichia coli
  source 1..876: mol_type = genomic DNA
atggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattga

SEQ ID NO: 128 - protein, 291 aa, Escherichia coli
MAEVTQLKRYDARPINWGKWFLIGIGMLVSAFILLVPMIYIFVQAFSKGLMPVLQNLADPDMLHAIWLTVMIALIAVPVNLVFGILLAWLVTRFNFPGRQLLLTLLDIPFAVSPVVAGLVYLLFYGSNGPLGGWLDEHNLQIMFSWPGMVLVTIFVTCPFVVRELVPVMLSQGSQEDEAAILLGASGWQMFRRVTLPNIRWALLYGVVLTNARAIGEFGAVSVVSGSIRGETLSLPLQIELLEQDYNTVGSFTAAALLTLMAIITLFLKSMLQWRLENQEKRAQQEEHHEH

SEQ ID NO: 129 - DNA, 1287 bp, Mycobacterium smegmatis
  source 1..1287: mol_type = genomic DNA
atgatcgcacgcgagacactggccgacgagctggccctggcccgcgaacgcacgttgcggctcgtggagttcgacgacgcggaactgcatcgccagtacaacccgctgatgagcccgctcgtgtgggacctcgcgcacatcgggcagcaggaagaactgtggctgctgcgcgacggcaaccccgaccgccccggcatgctcgcacccgaggtggaccggctttacgacgcgttcgagcactcacgcgccagccgggtcaacctcccgttgctgccgccttcggatgcgcgcgcctactgcgcgacggtgcgggccaaggcgctcgacaccctcgacacgctgcccgaggacgatccgggcttccggttcgcgctggtgatcagccacgagaaccagcacgacgagaccatgctgcaggcactcaacctgcgcgagggcccacccctgctcgacaccggaattcccctgcccgcgggcaggccaggcgtggcaggcacgtcggtgctggtgccgggcggcccgttcgtgctcggggtcgacgcgctgaccgaaccgcactcactggacaacgaacggcccgcccacgtcgtggacatcccgtcgttccggatcggccgcgtgccggtcaccaacgccgaatggcgcgagttcatcgacgacggtggctacgaccaaccgcgctggtggtcgccacgcggctgggcgcaccgccaggaggcgggcctggtggccccgcagttctggaaccccgacggcacccgcacccggttcgggcacatcgaggagatcccgggtgacgaacccgtgcagcacgtgacgttcttcgaagccgaggcctacgcggcgtgggccggtgctcggttgcccaccgagatcgaatgggagaaggcctgcgcgtgggatccggtcgccggtgctcggcgccggttcccctggggctcagcacaacccagcgcggcgctggccaacctcggcggtgacgcacgccgcccggcgccggtcggggcctacccggcgggggcgtcggcctatggcgccgagcagatgctgggcgacgtgtgggagtggacctcctcgccgctgcggccgtggcccggtttcacgccgatgatctacgagcgctacagcacgccgttcttcgagggcaccacatccggtgactaccgcgtgctgcgcggcgggtcatgggccgttgcaccgggaatcctgcggcccagcttccgcaactgggaccacccgatccggcggcagatattctcgggtgtccgcctggcctgggacgtctga

SEQ ID NO: 130 - protein, 428 aa, Mycobacterium smegmatis
MIARETLADELALARERTLRLVEFDDAELHRQYNPLMSPLVWDLAHIGQQEELWLLRDGNPDRPGMLAPEVDRLYDAFEHSRASRVNLPLLPPSDARAYCATVRAKALDTLDTLPEDDPGFRFALVISHENQHDETMLQALNLREGPPLLDTGIPLPAGRPGVAGTSVLVPGGPFVLGVDALTEPHSLDNERPAHVVDIPSFRIGRVPVTNAEWREFIDDGGYDQPRWWSPRGWAHRQEAGLVAPQFWNPDGTRTRFGHIEEIPGDEPVQHVTFFEAEAYAAWAGARLPTEIEWEKACAWDPVAGARRRFPWGSAQPSAALANLGGDARRPAPVGAYPAGASAYGAEQMLGDVWEWTSSPLRPWPGFTPMIYERYSTPFFEGTTSGDYRVLRGGSWAVAPGILRPSFRNWDHPIRRQIFSGVRLAWDV

SEQ ID NO: 131 - DNA, 684 bp, Mycobacterium smegmatis
  source 1..684: mol_type = genomic DNA
atgtgccggcatgtggcgtggctgggcgcgccgcggtcgttggccgacctggtgctcgacccgccgcagggactgctggtgcagtcctacgcaccgcgacgacagaagcacggtctgatgaacgccgacggttggggcgcagggtttttcgacgacgagggagtggcccgccgctggcgcagcgacaaaccgctgtggggtgatgcgtcgttcgcgtcggtggcacccgcactacgcagtcgttgcgtgctggccgcggtgcgctcggccaccatcggcatgcccatcgaaccgtcggcgtcggcgccgttcagcgacgggcagtggctgctgtcgcacaacggcctggtcgaccgcggggtgctcccgttgaccggtgccgccgagtccacggtggacagcgcgatcgtcgcggcgctcatcttctcccgtggcctcgacgcgctcggcgccaccatcgccgaggtcggcgaactcgacccgaacgcgcggttgaacatcctggccgccaacggttcccggctgctcgccaccacctggggggacacgctgtcggtcctgcaccgccccgacggcgtcgtcctcgcgagcgaaccctacgacgacgatcccggctggtcggacatcccggaccggcacctcgtcgacgtccgcgacgcccacgtcgtcgtgacacccctgtga

SEQ ID NO: 132 - protein, 227 aa, Mycobacterium smegmatis
MCRHVAWLGAPRSLADLVLDPPQGLLVQSYAPRRQKHGLMNADGWGAGFFDDEGVARRWRSDKPLWGDASFASVAPALRSRCVLAAVRSATIGMPIEPSASAPFSDGQWLLSHNGLVDRGVLPLTGAAESTVDSAIVAALIFSRGLDALGATIAEVGELDPNARLNILAANGSRLLATTWGDTLSVLHRPDGVVLASEPYDDDPGWSDIPDRHLVDVRDAHVVVTPL

SEQ ID NO: 133 - DNA, 966 bp, Mycobacterium smegmatis
  source 1..966: mol_type = genomic DNA
atgacgctctcactggccaactacctggcagccgactcggccgccgaagcactgcgccgtgacgtccgcgcgggcctcaccgcggcaccgaagagtctgccgcccaagtggttctacgacgccgtcggcagtgatctgttcgaccagatcacccggctccccgagtattaccccacccgcaccgaggcgcagatcctgcggacccggtcggcggagatcatcgcggccgcgggtgccgacaccctggtggaactgggcagtggtacgtcggagaaaacccgcatgctgctcgacgccatgcgcgacgccgagttgctgcgccgcttcatcccgttcgacgtcgacgcgggcgtgctgcgctcggccggggcggcaatcggcgcggagtaccccggtatcgagatcgacgcggtatgtggcgatttcgaggaacatctgggcaagatcccgcatgtcggacggcggctcgtggtgttcctggggtcgaccatcggcaacctgacacccgcgccccgcgcggagttcctcagtactctcgcggacacgctgcagccgggcgacagcctgctgctgggcaccgatctggtgaaggacaccggccggttggtgcgcgcgtacgacgacgcggccggcgtcaccgcggcgttcaaccgcaacgtgctggccgtggtgaaccgcgaactgtccgccgatttcgacctcgacgcgttcgagcatgtcgcgaagtggaactccgacgaggaacgcatcgagatgtggttgcgtgcccgcaccgcacagcatgtccgcgtcgcggcactggacctggaggtcgacttcgccgcgggtgaggagatgctcaccgaggtgtcctgcaagttccgtcccgagaacgtcgtcgccgagctggcggaagccggtctgcggcagacgcattggtggaccgatccggccggggatttcgggttgtcgctggcggtgcggtga

SEQ ID NO: 134 - protein, 321 aa, Mycobacterium smegmatis
MTLSLANYLAADSAAEALRRDVRAGLTAAPKSLPPKWFYDAVGSDLFDQITRLPEYYPTRTEAQILRTRSAEIIAAAGADTLVELGSGTSEKTRMLLDAMRDAELLRRFIPFDVDAGVLRSAGAAIGAEYPGIEIDAVCGDFEEHLGKIPHVGRRLVVFLGSTIGNLTPAPRAEFLSTLADTLQPGDSLLLGTDLVKDTGRLVRAYDDAAGVTAAFNRNVLAVVNRELSADFDLDAFEHVAKWNSDEERIEMWLRARTAQHVRVAALDLEVDFAAGEEMLTEVSCKFRPENVVAELAEAGLRQTHWWTDPAGDFGLSLAVR

SEQ ID NO: 135 - DNA, 1113 bp, Mycobacterium smegmatis
  source 1..1113: mol_type = genomic DNA
atgctcgcgcagcagtggcgtgacgcccgtcccaaggttgccgggttgcacctggacagcggggcatgttcgcggcagagcttcgcggtgatcgacgcgaccaccgcacacgcacgccacgaggccgaggtgggtggttatgtggcggccgaggctgcgacgccggcgctcgacgccgggcgggccgcggtcgcgtcgctcatcggttttgcggcgtcggacgtggtgtacaccagcggatccaaccacgccatcgacctgttgctgtcgagctggccggggaagcgcacgctggcctgcctgcccggcgagtacgggccgaatctgtctgccatggcggccaacggtttccaggtgcgtgcgctaccggtcgacgacgacgggcgggtgctggtcgacgaggcgtcgcacgaactgtcggcccatcccgtcgcgctcgtacacctcaccgcattggcaagccatcgcgggatcgcgcaacccgcggcagaactcgtcgaggcctgccacaatgcggggatccccgtggtgatcgacgccgcgcaggcgctggggcatctggactgcaatgtcggggccgacgcggtgtactcatcgtcgcgcaagtggctcgccggcccgcgtggtgtcggggtgctcgcggtgcggcccgaactcgccgagcgtctgcaaccgcggatccccccgtccgactggccaattccgatgagcgtcttggagaagctcgaactaggtgagcacaacgcggcggcgcgtgtgggattctccgtcgcggttggtgagcatctcgcagcagggcccacggcggtgcgcgaacgactcgccgaggtggggcgtctctctcggcaggtgctggcagaggtcgacgggtggcgcgtcgtcgaacccgtcgaccaacccaccgcgatcaccacccttgagtccaccgatggtgccgatcccgcgtcggtgcgctcgtggctgatcgcggagcgtggcatcgtgaccaccgcgtgtgaactcgcgcgggcaccgttcgagatgcgcacgccggtgctgcgaatctcgccgcacgtcgacgtgacggtcgacgaactggagcagttcgccgcagcgttgcgtgaggcgccctga

SEQ ID NO: 136 - protein, 370 aa, Mycobacterium smegmatis
MLAQQWRDARPKVAGLHLDSGACSRQSFAVIDATTAHARHEAEVGGYVAAEAATPALDAGRAAVASLIGFAASDVVYTSGSNHAIDLLLSSWPGKRTLACLPGEYGPNLSAMAANGFQVRALPVDDDGRVLVDEASHELSAHPVALVHLTALASHRGIAQPAAELVEACHNAGIPVVIDAAQALGHLDCNVGADAVYSSSRKWLAGPRGVGVLAVRPELAERLQPRIPPSDWPIPMSVLEKLELGEHNAAARVGFSVAVGEHLAAGPTAVRERLAEVGRLSRQVLAEVDGWRVVEPVDQPTAITTLESTDGADPASVRSWLIAERGIVTTACELARAPFEMRTPVLRISPHVDVTVDELEQFAAALREAP

SEQ ID NO: 137 - DNA, 2631 bp, unidentified
  source 1..2631: mol_type = genomic DNA; note = Neurospora crassa
atgccgagtgccgaatccatgaccccaagcagtgccctcggacagctcaaagcaactggacaacatgtgctatccaagcttcagcagcagacatcaaacgccgatatcatcgacatccgccgcgttgctgtagagatcaacctcaagaccgagataacctccatgttccgacctaaagatggccctagacagctacccaccttgcttctctacaacgagagaggcctgcagctgttcgagcgtatcacataccttgaagagtactatcttaccaatgacgagatcaaaatcctcaccaaacatgcgaccgaaatggctagcttcatcccgtcaggtgccatgatcattgagctcggaagcggaaatctgcgcaaagtaaaccttctattggaagccctagacaacgccggcaaggcaattgactattatgcccttgacctgtctcgggaggagctggagcgcactctcgctcaggtaccatcctacaagcacgtcaagtgccacggtcttctgggtacatatgacgatggacgtgactggctcaaggccccagagaacatcaataaacagaaatgcatcttgcacctcgggtcaagcattggcaactttaaccgcagtgacgccgctacctttctcaagggctttacggacgtccttggacccaatgacaagatgctcattggggttgacgcttgcaatgacccggcgagggtataccacgcttacaacgacaaggttggtattactcacgagttcatcttgaatggtcttcgcaacgccaatgaaattatcggagagacggccttcatcgagggcgattggagagtcattggcgaatatgtgtatgacgaagagggcggcagacaccaggccttttacgcccccactcgcgacaccatggttatgggggagttgattaggtcacacgacaggatccagatcgaacagagcctaaagtactcgaaagaggagtcagagaggctctggagcacggcgggattggaacaagtctcggaatggacgtacggcaacgaatatggactccatctgcttgccaagtcaaggatgtctttcagtctcatcccttcggtgtacgctcgcagcgcactcccaactctggacgactgggaggccctttgggcgacatgggatgtcgtcacacgtcagatgcttccccaggaagagcttctggagaagcccatcaagctccgaaacgcctgcatcttttacctcggtcacatcccgaccttcctcgacatccagctcacaaagaccaccaagcaggctccgtcagagcccgctcacttttgcaagatcttcgagcgaggcattgatcctgatgtcgacaacccggagctgtgtcatgcgcactcggagattcctgatgaatggccgccggtggaagaaatcctgacctaccaggagacggtacggtcccggttacgcggcctctatgcgcatggcatcgcgaatattccgcggaatgtgggtcgggccatttgggttgggtttgagcacgagcttatgcatatcgagacgctgttgtacatgatgctacagagcgacaagacgctgatcccaacccatattccacggcccgactttgacaagctcgcgaggaaggcagagtccgagagggttcccaatcagtggtttaagattccggcacaggagatcaccatcggtttggatgatcctgaggatggatctgatatcaacaagcattatggctgggacaacgagaagcctccaaggcgcgttcaagttgctgcctttcaggctcaagggaggccgatcaccaacgaagagtacgcgcaatatctgcttgaaaagaacatcgacaagctccctgcctcttgggcccgcctggacaacgagaacattagcaatggaacaacaaacagcgtgagcggtcaccacagcaacagaacctccaagcagcagctcccttcatctttcctcgagaagacagcagtccgcacagtctacggtctcgtgcctctcaagcacgctctcgactggcccgtgtttgcctcttacgacgaacttgccggttgcgcagcttacatgggcggccgtattcccaccttcgaagagacccggagcatttacgcttacgccgatgctctcaagaagaagaaggaagctgagagacaattgggaaggacggttccggctgttaatgcccacctaaccaacaacggcgtggaaatcactcccccatcctctccctcttccgagacccccgccgagtcttcctccccctccgacagcaacaccaccctcatcaccaccgaagacctcttctctgacctagacggtgccaatgtcggttttcacaactggcaccctatgcccatcacctccaaaggcaacacccttgtcgggcaaggcgagctcggcggcgtgtgggaatggacttcatcggtcctccgcaagtgggaggggttcgagccgatggagctgtaccccggctatacggcggattttttcgatgagaagcacaacattgtgctgggagggagctgggctacgcatccgaggattgcggggaggaagagctttgtgaattggtaccagaggaattatccttatgcttgggtgggggcgagagttgttagggatttgtga

SEQ ID NO: 138 - protein, 876 aa, unidentified
  source 1..876: note = Neurospora crassa
MPSAESMTPSSALGQLKATGQHVLSKLQQQTSNADIIDIRRVAVEINLKTEITSMFRPKDGPRQLPTLLLYNERGLQLFERITYLEEYYLTNDEIKILTKHATEMASFIPSGAMIIELGSGNLRKVNLLLEALDNAGKAIDYYALDLSREELERTLAQVPSYKHVKCHGLLGTYDDGRDWLKAPENINKQKCILHLGSSIGNFNRSDAATFLKGFTDVLGPNDKMLIGVDACNDPARVYHAYNDKVGITHEFILNGLRNANEIIGETAFIEGDWRVIGEYVYDEEGGRHQAFYAPTRDTMVMGELIRSHDRIQIEQSLKYSKEESERLWSTAGLEQVSEWTYGNEYGLHLLAKSRMSFSLIPSVYARSALPTLDDWEALWATWDVVTRQMLPQEELLEKPIKLRNACIFYLGHIPTFLDIQLTKTTKQAPSEPAHFCKIFERGIDPDVDNPELCHAHSEIPDEWPPVEEILTYQETVRSRLRGLYAHGIANIPRNVGRAIWVGFEHELMHIETLLYMMLQSDKTLIPTHIPRPDFDKLARKAESERVPNQWFKIPAQEITIGLDDPEDGSDINKHYGWDNEKPPRRVQVAAFQAQGRPITNEEYAQYLLEKNIDKLPASWARLDNENISNGTTNSVSGHHSNRTSKQQLPSSFLEKTAVRTVYGLVPLKHALDWPVFASYDELAGCAAYMGGRIPTFEETRSIYAYADALKKKKEAERQLGRTVPAVNAHLTNNGVEITPPSSPSSETPAESSSPSDSNTTLITTEDLFSDLDGANVGFHNWHPMPITSKGNTLVGQGELGGVWEWTSSVLRKWEGFEPMELYPGYTADFFDEKHNIVLGGSWATHPRIAGRKSFVNWYQRNYPYAWVGARVVRDL

SEQ ID NO: 139 - DNA, 962 bp, Methanosalsum zhilinae
  source 1..962: mol_type = genomic DNA
atgatcatacagaattttatgcctgagattggagaacgttcagttcaagaaagacttttaacttgtttaaggtctgagccaaagacattaccatctgtgttcttctatgaccagaaaggttcggaactgttcgaacaaataacaaaacttgaagaatattatttacctgacattgagatcccacttttaagatcgactgctaaaaaagttaattctgaactgaagaactgtaaccttgtagaactcgggagtggtgactgttctaagatttcagtgttccttgatgcagttccaaaagacattcgtgaaaccatcatttattatccaatagatgtttccaaggatgctatggaaaaatctggtcatattctacagaacagattccctgagataggtattcatggaattaatgccgatttccttgaaagtatggatttaatacctggagataggaacaggttcttctgttttttcgggagtacaataggtaatcttactcgtgctaagactactgaattcatgaaacggcttggacaagtcatgaatgaaaatgataggcttcttctcggagttgatatggtgaaagatatcaatgtacttgagagagcatataatgatagtctgggtattactgcggagtttaataagaatattttaaaggtcgcaaacaatcatataggaactgactttgatccagatgattttgaacatgttgcttttttcaacaaagaattttcacgaatcgagatgcacttgaaagcaaaaagggatctagtagttaaaagtgatttgtttaaggaacgtattatcttcaagaaaggggataccattcatacagaaaattcacataagtatactgtacagcacatctatgatatggcagatactgctggtctttttgtatctgatatttattctgatgataaaaaatggttctcacttgttgaaatggtgaaagaatg

SEQ ID NO: 140 - protein, 320 aa, Methanosalsum zhilinae
MIIQNFMPEIGERSVQERLLTCLRSEPKTLPSVFFYDQKGSELFEQITKLEEYYLPDIEIPLLRSTAKKVNSELKNCNLVELGSGDCSKISVFLDAVPKDIRETIIYYPIDVSKDAMEKSGHILQNRFPEIGIHGINADFLESMDLIPGDRNRFFCFFGSTIGNLTRAKTTEFMKRLGQVMNENDRLLLGVDMVKDINVLERAYNDSLGITAEFNKNILKVANNHIGTDFDPDDFEHVAFFNKEFSRIEMHLKAKRDLVVKSDLFKERIIFKKGDTIHTENSHKYTVQHIYDMADTAGLFVSDIYSDDKKWFSLVEMVKE

SEQ ID NO: 141 - DNA, 1263 bp, Methanosalsum zhilinae
  source 1..1263: mol_type = genomic DNA
atgaaatccatttcaactgatgaattattagaaaacttgcatagatacaaagtcattgatattagatctgtagatgcttataatggatggaaggagaatggggaaaacagaggtgggcatataagaagtgcaaaatcactaccttacaagtgggtaaactacattgactggatcgagatcgttaggagcaaggatattctcccacaagacaaacttgtaatttacggttatgactcagagaaagcagaagaggttgccagaatgtttgaaaaggctggttatactgacctgaacatatacccttcttttttcgagtgggtagaaaggaatctgccaatggaccgacttgagaggtaccgacacttagtatctcctgattggctgaaccaattgataactaccgacaatgcacctgaatatgataatgataagtatgtcatatgccattgccattacagaaatccagtggattatgaaaaaggtcatattccaggctcgatcccacttgataccaattcactcgaatccgaggatacatggaaccgtcgttcaccagaagaactaaaagatgcacttgaaaatgcaggtatttccagtgaaacaacagttattgtatatggaaggttctcctacccaaagaacgatgacccatttccaggcagtagcgcgggtcaccttggtgcaatgcgatgtgcattcataatgctttatgctggagtcaaggatgtaaggatccttaatggtggactccagtcctggcttgatgcaggttataatgtcacaacagaacctgctaaaataagtaaagtatcttttggtgccaatattcctttaaaccctaaaattgctgttgatcttgaggaagcaaaggagatactttcagaccctggcaaaaaactggtaagtgtcaggagttggagagaatatattggtgaagtaagtggttataactatattgagaaaaaaggtcgtatcccgggatctgtgttcggggattgcggaactgatgcttatcacatggagaactacaggaacctggaccacactatgcgagaataccatgaaattgaagataaatggaaagaattaggtataactcccgaaaaacgcaatgccttctattgtggtactggatggagaggaagtgaagcattccttaacgcttggctcatgggctgggacaatgcagcggtctttgacggtggatggtttgagtggagtaataatgatcttccttttgaaacaggtgtgccagaaaaatga

SEQ ID NO: 142 - protein, 420 aa, Methanosalsum zhilinae
MKSISTDELLENLHRYKVIDIRSVDAYNGWKENGENRGGHIRSAKSLPYKWVNYIDWIEIVRSKDILPQDKLVIYGYDSEKAEEVARMFEKAGYTDLNIYPSFFEWVERNLPMDRLERYRHLVSPDWLNQLITTDNAPEYDNDKYVICHCHYRNPVDYEKGHIPGSIPLDTNSLESEDTWNRRSPEELKDALENAGISSETTVIVYGRFSYPKNDDPFPGSSAGHLGAMRCAFIMLYAGVKDVRILNGGLQSWLDAGYNVTTEPAKISKVSFGANIPLNPKIAVDLEEAKEILSDPGKKLVSVRSWREYIGEVSGYNYIEKKGRIPGSVFGDCGTDAYHMENYRNLDHTMREYHEIEDKWKELGITPEKRNAFYCGTGWRGSEAFLNAWLMGWDNAAVFDGGWFEWSNNDLPFETGVPEK

SEQ ID NO: 143 - DNA, 318 bp, Escherichia coli
  source 1..318: mol_type = genomic DNA
atggctgaatggagcggcgaatatatcagcccatacgctgagcacggcaagaagagtgaacaagtcaaaaagattacggtttccattcctcttaaggtgttaaaaatcctcaccgatgaacgcacgcgtcgtcaggtgaacaacctgcgtcacgctaccaacagcgagctgctgtgcgaagcgtttctgcatgcctttaccgggcaacctttgccggatgatgccgatctgcgtaaagagcgcagcgacgaaatcccggaagcggcaaaagagatcatgcgtgagatggggattaacccggagacgtgggaatactaa

SEQ ID NO: 144 - protein, 105 aa, Escherichia coli
MAEWSGEYISPYAEHGKKSEQVKKITVSIPLKVLKILTDERTRRQVNNLRHATNSELLCEAFLHAFTGQPLPDDADLRKERSDEIPEAAKEIMREMGINPETWEY

(19) United States
(12) Patent Application Publication
(10) Pub. No.: US 2024/0309411 A1
(43) Pub. Date: Sep. 19, 2024

(54) MICROBIAL ERGOTHIONEINE BIOSYNTHESIS

(71) Applicant: Conagen Inc., Bedford, MA (US)

(72) Inventors: Jixiang Han, Maryland Heights, MO (US); Sonya Clarkson, Renton, WA (US); David Nunn, Carlsbad, CA (US)

(73) Assignee: Conagen Inc., Bedford, MA (US)

(21) Appl. No.: 18/449,008

(22) Filed: Aug. 14, 2023

Related U.S. Application Data
(63) Continuation of application No. PCT/US2022/016404, filed on Feb. 15, 2022.
(60) Provisional application No. 63/200,115, filed on Feb. 15, 2021.

(51) Int. Cl. C12P 17/10; C12N 9/02; C12N 9/88

(52) U.S. Cl. CPC C12P 17/10; C12N 9/0004; C12N 9/88

(57) ABSTRACT

The present invention relates to engineered microbial host cells comprising exogenous genes coding for proteins responsible for converting histidine and cysteine into ergothioneine in greater efficiency than the wild-type cells. Also provided in this invention are methods for producing ergothioneine using the engineered microbial host cells of the present invention.

### FIELD OF INVENTION

The present invention relates to a method of producing ergothioneine using engineered microbial host cells. This invention provides methods for constructing engineered microbial host cells useful in ergothioneine production. The invention also relates to recombinant nucleic acid constructs including vectors and recombinant host cells comprising the recombinant nucleic acid constructs useful in ergothioneine production.

### BACKGROUND

Ergothioneine is a trimethylated and sulphurized histidine derivative that can be found in many unicellular and multicellular organisms. Its biosynthesis, however, occurs only in certain bacteria belonging to mycobacteria, methylobacteria, cyanobacteria and fungi such as Neurospora crassa. Other bacteria such as Bacillus, Corynebacterium, Escherichia, Lactobacillus, Pseudomonas, Streptococcus, and Vibrio and other fungi belonging to the groups Ascomycetes and Deuteromycetes cannot synthesize ergothioneine. Animals also do not have the capacity to synthesize ergothioneine and they depend on dietary sources. The higher plants acquire ergothioneine from their environment.

Ergothioneine exists predominantly in its thione form with high redox potential (−60 mV) at physiological pH. Thus, unlike other thiol antioxidants such as glutathione, ergothioneine is characterized by its slow degradation and resistance to disulfide formation under physiological conditions. Ergothioneine is preferentially accumulated in certain cells and tissues such as liver, kidney, central nervous system, bone marrow and blood cells, which are often predisposed to high levels of oxidative stress and inflammation. Several lines of evidence in vitro and in vivo show that ergothioneine acts as an antioxidant, cation chelator, bioenergetics factor, and immune regulator. Thus, ergothioneine may play a role in mitigating inflammatory, cardiovascular disease, cognitive impairment, depression, dementia and other epiphenomena of aging. There is a growing interest in genetically engineering microbial host cells to produce ergothioneine in commercial quantities for pharmaceutical and nutraceutical applications in humans.

Mushrooms are traditionally considered as a source for ergothioneine production. However, their slow growth, low content of ergothioneine and time-consuming purification procedures lead to a high manufacturing cost. Therefore, alternative and sustainable sources of ergothioneine are necessary. One such reliable and practical method is a fermentation process using ergothioneine-producing microbes such as mycobacteria and cyanobacteria. But their ergothioneine productivities are very low (1.18 mg/g of dry mass after 4 weeks of cultivation of Mycobacterium avium and 0.8 mg/g of dry mass of Oscillatoria sp.). Thus, genetic and metabolic engineering involving microorganisms traditionally used in industrial fermentation is necessary for commercial scale production of ergothioneine. So far, several such efforts have been made, but the titer for ergothioneine production in those systems are still low. We have developed a method for ergothioneine production by using a combination of bioinformatics and synthetic biology. We have constructed an ergothioneine biosynthetic pathway within E. coli K12 strain. With this E. coli strain genetically engineered to produce ergothioneine, we have been able to produce ergothioneine in a way suitable for industrial scale production.

### BRIEF SUMMARY OF THE INVENTION

The present invention provides, among other things, a method for producing ergothioneine using genetically engineered microorganisms. The genetically engineered microorganisms according to the present invention has the ability to produce ergothioneine by using the amino acids produced internally within the genetically engineered microorganisms or by using the amino acids added to the growth medium. In a preferred embodiment, the present invention provides genetically engineered microorganisms with the ability to produce ergothioneine without the need for exogenously supplied amino acid.

In one embodiment, the present invention provides methods for introducing ergothioneine biosynthetic pathway into an industrially useful microorganism which does not have any genes coding for proteins functional in ergothioneine biosynthetic pathway. The industrially useful microorganism suitable for the present invention includes a number of bacterial and fungal species. The list of bacterial species suitable for the present invention includes, but not limited to, Escherichia, Salmonella, Bacillus, Acinetobacter, Streptomyces, Corynebacterium, Methylosinus; Methylomonas, Rhodococcus, Pseudomonas, Rhodobacter, Synechocystis, Arthrobotlys, Brevibacteria, Microbacterium, Arthrobacte, Citrobacter, Klebsiella, Pantoea, and Clostridium. The list of fungal species suitable for the present invention includes, but not limited to, Saccharomyces, Zygosaccharomyces, Kluyveromyces, Candida, Hansenula, Debaryomyces, Mucor, Pichia, Torulopsis, and Aspergillus.

The genes suitable for building an ergothioneine pathway with an industrially useful microorganism can be derived from bacterial and fungal species reported to have the natural ability to produce ergothioneine. In one aspect of the present invention, the bacterial genes coding for proteins reported to be involved in the ergothioneine biosynthesis in bacterial cells including, but not limited, EgtB, EgtC, EgtD, and EgtE are introduced into a microorganism which, to begin with, does not have any genes coding for ergothioneine biosynthesis. In another aspect of the present invention, the fungal genes coding for proteins reported to be involved in the ergothioneine biosynthesis including, but not limited, Egt1. Egt2 and variants thereof are introduced into a microorganism which, to begin with, does not have any genes coding for ergothioneine biosynthesis. In yet another aspect of the present invention, the anaerobic bacterial genes coding for proteins reported to be involved in the ergothioneine biosynthesis including, but not limited, EnaA, EnaB and variants thereof are introduced into a microorganism which, to begin with, does not have any genes coding for ergothioneine biosynthesis. In a preferred aspect, the present invention introduces two different fungal genes, namely egt1 and egt2 coding for proteins Egt1 and Egt2 proteins respectively involved in ergothioneine biosynthesis, into a microorganism which, to begin with, does not have any genes coding for ergothioneine biosynthesis. The fungal genes coding for ergothioneine biosynthesis are obtained from different species and the selection of individual enzyme is based on higher enzymatic activity for that particular enzyme as well as the combined activity of both enzymes.

In an embodiment, the present invention provides a screening method for selecting fungal genes coding Egt1 and Egt2 proteins for building an ergothioneine biosynthetic pathway in an industrially useful microorganism which, to begin with, does not have any genes coding for ergothioneine biosynthesis. In one aspect of this embodiment, the nucleotide sequence of a fungal gene coding for Egt1protein involved in ergothioneine pathway is used to conduct a blast search in the nucleotide database to identify homologous genes and a pool of genes coding for Egt1 protein is identified. In the same way, the nucleotide sequence of a fungal gene coding for Egt2 protein involved in ergothioneine pathway is used to conduct a blast search in the nucleotide database to identify homologous genes and a pool of genes coding for Egt2 protein is identified. The members of the gene pools coding for Egt1 or Egt2 proteins are used in a number of different combinations to transform an industrially useful microorganism and the transformants are assayed for the relative ergothioneine production to identify the highly efficient Egt1 and Egt2 proteins. In a preferred aspect of the present invention, the screening for the efficient Egt1 and Egt2 proteins is conducted in two steps. In the first step of the screening, the nucleotide sequence of the fungal gene coding for the Egt1 protein used in the initial screening step is cloned into a plasmid vector along with one of the nucleotide sequence coding for Egt2 protein selected from the pool of genes for Egt2 protein and the resulting plasmid is used to transform an industrially useful microorganism. The transformants are assayed for the level of ergothioneine production. The transformants having higher ergothioneine production are selected as having the nucleotide sequence coding for Egt2 protein with high level of enzyme activity and are grouped under Tier 1 nucleotide sequence coding for Egt2 protein. In the same way the nucleotide sequence of the fungal gene coding for the Egt2 protein used in the initial screening step is cloned into a plasmid vector along with one of the nucleotide sequence coding for Egt1 protein selected from the pool of genes for Egt1 protein and the resulting plasmid is used to transform an industrially useful microorganism. The transformants are assayed for the level of ergothioneine production. The transformants having higher ergothioneine production are selected as having the nucleotide sequence coding for Egt1 protein with high level of enzyme activity and are grouped under Tier 1 nucleotide sequence coding for Egt1 protein.

In the second level of screening, a set of nucleotide sequences coding for Egt1 protein with high level of activity selected from Tier 1 are combined with a set of nucleotide sequences coding for Egt2 protein with high level of activity to come out with a defined number of permutations. For example, when four nucleotide sequences coding for Egt1 protein are combined with four nucleotide sequences coding for Egt2 protein in a permutation complex, sixteen different Egt1-Egt2 pairings are possible. The nucleotide sequence coding for Egt1 protein and the nucleotide sequence coding for Egt2 protein in each of the pair is cloned into a plasmid expression vector and used to transform an industrially useful microbial cell. The resulting transformants are screened for ergothioneine production. The transformant showing the highest ergothioneine production is considered to have the Egt1 and Egt2 protein with highest level of enzyme activity in combination.

In one aspect of this embodiment, once a best performing ergothioneine strain is identified through plasmid transformation, the corresponding nucleotide sequences coding for Egt1 and Egt2 proteins are integrated into the host chromosomal DNA to achieve stable integration and to avoid using antibiotics in the growth medium to maintain the self-replicating plasmid. In one aspect of this embodiment, the nucleotide sequences coding for Egt1 and Egt2 proteins are under the control of a constitutively active promoter. In another aspect of this embodiment, the nucleotide sequences coding for Egt1 and Egt2 proteins are under the control of an inducible promoter.

Once a stably transformed industrially useful microorganism with an exogenous ergothioneine pathway is obtained, further improvement in ergothioneine production is achieved through other genetic manipulations aimed at increasing the pool size of substrates used in ergothioneine production. Since ergothioneine is a thiol derived from histidine, to further improve the ergothioneine production, it is necessary to increase the pool size of the co-substrate molecules such as methionine and cysteine.

In one of aspect of this embodiment, the industrial microbial strain engineered to have the exogenous pathway for ergothioneine biosynthesis is subjected to further genetic engineering to increase the uptake of methionine from the culture medium. In one aspect of the present invention, the industrial microbial strain engineered to have the exogenous ergothioneine pathway is further transformed with a nucleotide sequence coding for the transporter YjeH to increase the pool size of methionine which is necessary to supply S-adenosylmethionine required for the conversion of L-histidine to trimethyl histidine hercynine within the microbial cells.

Cysteine is yet another co-substrate in the biosynthesis of ergothioneine from L-histidine within the microbial cells. In another embodiment of the present invention, the industrial microbial strain engineered to have the exogenous pathway for ergothioneine biosynthesis is subjected to further genetic engineering to increase the pool size of the cysteine within the microbial cell.

Since cysteine is derived from serine, in one aspect of the present invention, serine pool within the host microbial cell is increased by means of enhancing the activity of D-3-phosphoglycerate dehydrogenase (SerA) and phosphoserine phosphatase (SerB and SerC) responsible for the conversion of 3-p-glycerate into L-serine. In one aspect of this embodiment, the activity of these enzymes is improved by means of expressing these genes using a constitutive promoter. In another aspect of this embodiment, the degradation of serine within the microbial cell is reduced by means of mutating the gene sdaA coding for the L-serine hydratase 1 wherein the mutation is deletion, frameshift or point mutation decreasing or eliminating L-serine hydratase 1.

L-serine is converted into L-cysteine in a two-step enzyme reaction. In the first step of this reaction, the seine acetylytransferase enzyme (CysE) converts L-serine into o-acetyl serine which in turn is converted into L-cysteine by the enzyme cysteine synthase B (CysM). In one aspect of this embodiment, the activity of the CysE and CysM enzymes are increased by means of expressing these enzymes using a constitutive promoter. In another aspect of this embodiment, the degradation of L-cysteine to pyruvate, ammonium and hydrogen sulfide within the microbial cell is reduced by means of mutating the tnaA gene coding for L-cysteine desulfhydrase and yhaM gene coding for L-cysteine desulfidase, wherein the mutation is deletion, frameshift or point mutation, decreasing or eliminating the function of these enzymes.

In another aspect of this embodiment, the activity of L-cysteine exporter is upregulated using the constitutive promoter to drive the expression of the corresponding gene ydeD. In yet another embodiment of the present embodiment, a constitutive promoter is used to upregulate the expression of cysB gene coding for the transcriptional regulator CysB protein, a positive regulator of gene expression for the cysteine regulon, a system of 10 or more loci involved in the biosynthesis of L-cysteine from inorganic sulfate.

In yet another aspect of the present invention, the ergothioneine producing strain having exogenous egt1 and egt2 gene is expected to have a disruption in the metJ gene coding for a transcriptional repressor controlling the methionine biosynthesis. With the disruption of metJ gene, the methionine pool size within the ergothioneine producing microbial strain is expected to increase with a consequent increase in the production of ergothioneine.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 illustrates the ergothioneine biosynthetic pathway. In the bacterial system, a set of five genes (egtABCDE) are involved in the biosynthesis of ergothioneine from L-histidine. In the fungal system, only two enzymes namely Egt1 and Egt2 enzymes are involved in the biosynthesis of ergothioneine from L-histidine. Similarly, in the anoxygenic bacterium Chlorobium lumicola, only two enzymes namely EanA and EanB are involved in the biosynthesis of ergothioneine from L-histidine.

FIG. 2 shows the map of a plasmid carrying the genes coding for Egt1 and Egt2 enzymes from Schizosaccharomyces pompe.

FIG. 3 shows a plasmid map carrying the genes coding for Egt1 enzyme from Ajellomyces dermatitidis and Egt2 enzyme from Talaromyces stipitatus.

FIG. 4 shows the sequence alignment of Egt1 amino acid sequences from 25 different species.

FIG. 5 shows the sequence alignment of Egt2 amino acid sequences from 15 different species.

FIG. 6 shows ergothioneine production from Escherichia coli cells transformed with plasmid vectors carrying various genes involved in the ergothioneine biosynthesis. The strains S1, S2 and S3 are transformants carrying the eanA and eanB genes from Chlorobium lumicola coding for EanA and EanB proteins, respectively. The strains S4, S5 and S6 are transformants carrying the eanA and eanB3 genes from Chlorobium lumicola coding for EanA and EanB3 proteins respectively. EanB3 is a variant of EanB protein. The strains S7, S8 and S9 are transformants carrying the plasmid carrying the genes coding for Egt1 and Egt2 proteins from Schizosaccharomyces pompe shown in FIG. 2. The strains S10, S11 and S12 are transformants carrying three bacterial genes coding for EgtB, EgtC and EgtE proteins reported to be functional in the ergothioneine biosynthesis. The strains S14, S15 and S16 are transformants carrying four bacterial genes coding for EgtB, EgtC, EgtD and EgtE reported to be present in the ergothioneine biosynthesis.

FIG. 7 shows ergothioneine production in two different strains of Escherichia coli, namely, JM109 and MG1655, transformed with three different plasmid constructs. C13 is an E. coli strain transformed with a Tier 2 plasmid construct carrying the genes coding for Egt1 protein from Ajellomyces dermatitidis (SEQ ID No: 18) and Egt2 proteins from Talaromyces stipitatus (SEQ ID NO: 90). C14 is an E. coli strain transformed with a Tier 2 plasmid construct carrying the genes coding for Egt1 protein from Aspergillus niger (SEQ ID No: 20) and Egt2 proteins from Talaromyces stipitatus (SEQ ID No: 90). Ck+ is an E. coli strain transformed with a plasmid carrying the genes coding for Egt1 and Egt2 proteins from Schizosaccharomyces pompe.

FIG. 8 shows ergothioneine production by a C13 E. coli strain (Tier 2 construct as in Table 3) in a 3 L fermenter. WCW: wet cell weight. Shown in the graph on the right side are the titer for ergothioneine production in three different fermenter runs.

FIG. 9 shows ergothioneine production by C13 E. coli strain (Tier 2 construct as in Table 3) in a 5,000 L fermenter

FIG. 10 shows the map of a plasmid carrying the yjeH gene.

FIG. 11 shows ergothioneine production in E. coli strains ET1 and ET2 transformed with the plasmid carrying yjeH gene. Vec0 is an empty plasmid vector without yjeH gene. Ergothioneine production was performed both with tube culture and flask culture. ET1 is an E. coli MG1655 strain transformed with a Tier 2 plasmid construct carrying the genes coding for Egt1 protein from Ajellomyces dermatitidis and Egt2 protein from Talaromyces stipitatus. ET2 is an E. coli JM109 strain transformed with a Tier 2 plasmid construct carrying the genes coding for Egt1 protein from Ajellomyces dermatitidis and Egt2 protein from Talaromyces stipitatus.

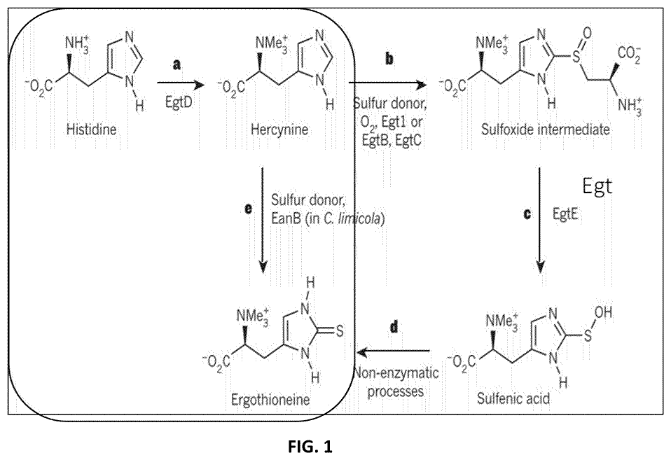

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Although ergothioneine was discovered one hundred years ago, only during the last decade there has been significant progress in gaining insight into the ergothioneine biosynthetic pathways in a few selected microbial organisms. However, the microbial organisms with native ergothioneine biosynthetic pathway are not suitable for commercial applications as the ergothioneine production using these organisms are not scalable. As such there is a growing need in the art for constructing recombinant microorganisms for ergothioneine production by means of introducing the known ergothioneine pathway into those microorganisms which are already in industrial use but are devoid of any ergothioneine biosynthetic pathway.

In mycobacteria, a gene cluster (egtABCDE) is responsible for five enzymatic steps that convert histidine to ergothioneine. Briefly, L-histidine is first methylated into hercynine by an S-adenosylmethionine (SAM)-dependent methyltransferase (EgtD), followed by adding γ-glutamylcysteine to form hercynyl γ-glutamylcysteine sulfoxide intermediate by a formylglycine-generating enzyme-like protein (EgtB). The γ-glutamylcysteine is formed from cysteine and glutamate by a γ-glutamyl cysteine synthetase (EgtA). Glutamate is released from the intermediate by a glutamine amidotransferase (EgtC) to generate hercynlcysteine sulfoxide that is converted into ergothioneine by a pyridoxal 5-phosphate-dependent β-lyase (EgtE). Genes homologous to Mycobacterium egtABCDE are also found in Methylobacterium aquaticum strain 22A and other Methylobacterium species, although not clustered in the chromosome or even located on the plasmid. Similarly, homologs of egtABCDE five-gene cluster also exists in the genome database of Streptomyces coelicolor, whereas only orthologs of egtB, egtC and egtD are found in cyanobacterial species. The crystal structures of EgtB, EgtC and EgtD have been recently determined. U.S. Pat. No. 10,544,437 has descried in detail the process of using exogenous genes egtB, egtC, egtD and egtE to transform Escherichia coli, Saccharomyces cerevisiae, or Pichia pastoris for the purpose of producing ergothioneine. The disclosure in the U.S. Pat. No. 10,544,437 is incorporated herein by reference.

Since the first fungal ergothioneine biosynthetic gene, egt1 was identified in N. crassa, several genes from filamentous fungi and other fungal species have been characterized. In N. crassa and Schizosaccharomyces pombe, two genes, egt1 and egt2, are responsible for the biosynthesis of Ergothioneine. Egt1 contains multiple domains functionally homologous to EgtD and EgtB of M. smegmatis and Egt2 is a homolog of EgtE from M. smegmatis. Egt1 is responsible for both trimethylation of histidine to hercynine and sulfoxidation of the hercynine to hercynylcysteine sulfoxide. Egt2 catalyzes the final step in ergothioneine biosynthesis that converts hercynylcysteine sulfoxide to 2-sulfenohercynine, which is reduced to ergothioneine non-enzymatically. Both N. crassa and S. pombe directly use cysteine rather than γ-glutamylcysteine to produce ergothioneine. These two fungal species seem to lack γ-glutamyl cysteine synthetase and glutamine amidotransferase genes as found in mycobacteria. In S. pombe, knockout of egt1 results in a loss of ergothioneine biosynthesis. However, when egt2 is knocked out, small amounts of ergothioneine is still produced, indicating an unrelated pyridoxal 5-phosphate-binding enzyme may exist. This is supported by a blast search that shows homologs of Egt2 are not only found in bacteria such as in cyanobacteria and proteobacteria but also in fungi such as in Saccharomyces cerevisiae, Leishmania donovani, and Dictyostelium discoideum. These candidates may represent unidentified enzymes that do not have homology with EgtE, but have homology to enzymes with a C—S lyase activity in other organisms. Taken together, as homologs of EgtB and EgtD not only occur in a number of diverse bacterial phyla including Actinobacterial, Proteobacterial, and Cyanobacterial species but also in fungi including N. crassa and S. pombe. The EgtB and EgtD genes appear to be a gene signature common to ergothioneine biosynthesis in microbes.

The present invention provides, among other things, a method for producing ergothioneine using genetically engineered microorganisms. The genetically engineered microorganisms according to the present invention has the ability to produce ergothioneine by using the amino acids produced internally within the genetically engineered microorganisms or by using the amino acids added to the growth medium. In a preferred embodiment, the present invention provided genetically engineered microorganisms with the ability to produce ergothioneine without the need for exogenously supplied amino acid.

In one embodiment, the present invention provides methods for introducing ergothioneine biosynthetic pathway into an industrially useful microorganism which does have any genes coding for proteins functional in ergothioneine biosynthetic pathway. The industrially useful microorganism suitable for the present invention includes a number of bacterial and fungal species. In a preferred embodiment of the present invention, the list of bacterial species includes, but not limited to, Escherichia, Salmonella, Bacillus, Acinetobacter, Streptomyces, Corynebacterium, Methylosinus, Methylomons, Rhodococcus, Pseudomonas, Rhodobacter, Synechocystis, Arthrobotlys, Brevibacteria, Microbacterium, Arthrobacter, Citrobacter, Klebsiella, Pantoea, and Clostridium. In another preferred embodiment of the present invention, the list of fungal species includes, but not limited to, Saccharomyces, Zygosaccharomyces, Kluyveromyces, Candida, Hansenula, Debaryomyces, Mucor, Pichia, Torulopsis, and Aspergillus.

The genes suitable for building an ergothioneine pathway with an industrially useful microorganism can be derived from bacterial and fungal species reported to have the natural ability to produce ergothioneine. In one aspect of the present invention, the bacterial genes coding for proteins reported to be involved in the ergothioneine biosynthesis including, but not limited, EgtA, EgtB, EgtC, EgtD, and EgtD are introduced into a microorganism which, to begin with, does not have any genes coding for ergothioneine biosynthesis. In another aspect of the present invention, the fungal genes coding for proteins reported to be involved in the ergothioneine biosynthesis including, but not limited, Egt1 and Egt2 are introduced into a microorganism which, to begin with, does not have any genes coding for ergothioneine biosynthesis. In yet another aspect of the present invention, the anaerobic bacterial genes coding for proteins reported to be involved in the ergothioneine biosynthesis including, but not limited. EnaA and EnaB are introduced into a microorganism which, to begin with, does not have any genes coding for ergothioneine biosynthesis. In a preferred aspect, the present invention introduces fungal gene coding for proteins involved in ergothioneine derived from different species into a microorganism which, to begin with, does not have any genes coding for ergothioneine biosynthesis and the selection of fungal genes from different species is based on a selection-criteria for higher enzymatic activity.

In an embodiment, the present invention the provides a screening method for selecting fungal genes coding Egt1 and Egt2 proteins for building an ergothioneine pathway in an industrially useful microorganism which, to begin with, does not have any genes coding for ergothioneine biosynthesis. In one aspect of this embodiment, the nucleotide sequence of a fungal gene coding for Egt1protein involved in ergothioneine pathway is used to conduct a blast search in the nucleotide data based to identify homologous genes and a pool of genes coding for Egt1 protein is identified. In the same way, the nucleotide sequence of a fungal gene coding for Egt2protein involved in ergothioneine pathway is used to conduct a blast search in the nucleotide data based to identify homologous genes and a pool of genes coding for Egt2 protein is identified. The members of the gene pools coding for Egt1 or Egt2 proteins are used in a number of different combinations to transform an industrially useful microorganism and the transformants are assayed for the relative ergothioneine production to identify the highly efficient Egt1 and Egt2 proteins. In a preferred aspect of the present invention, the screening for the efficient Egt1 and Egt2 proteins is conducted in two steps. In the first step of the screening, the nucleotide sequence of the fungal gene coding for the Egt1 protein used in the initial screening step is cloned into a plasmid vector along with one of the nucleotide sequence coding for Egt2 protein in the pool of genes for Egt2 protein and the resulting plasmid is used to transform an industrially useful microorganism. The transformants are assayed for the level of ergothioneine production. The transformants having higher ergothioneine production are selected as having the nucleotide sequence coding for Egt2 protein with high level of enzyme activity and grouped under Tier 1 for nucleotide sequence coding for Egt2 protein. In the same way the nucleotide sequence of the fungal gene coding for the Egt2 protein used in the initial screening step is cloned into a plasmid vector along with one of the nucleotide sequence coding for Egt1 protein in the pool of genes for Egt1 protein and the resulting plasmid is used to transform an industrially useful microorganism. The transformants are assayed for the level of ergothioneine production. The transformants having higher ergothioneine production are selected as having the nucleotide sequence coding for Egt1 protein with high level of enzyme activity and grouped under Tier 1 for nucleotide sequence coding for Egt1 protein.

In the second level of screening a set of nucleotide sequences coding for Egt1 protein with high level of activity are combined with a set of nucleotide sequences coding for Egt1 protein with high level of activity to come out with a defined number of permutations. For example, when four nucleotide sequences coding for Egt1 protein are combined with four nucleotide sequences coding for Egt2 protein in a permutation complex, sixteen different Egt1-Egt2 pairing are possible. The nucleotide sequence coding for Egt1 protein and the nucleotide sequence coding for Egt2 protein in each of the pair is cloned into a plasmid expression and used to transform an industrially useful microbial cell. The resulting transformants are screened for ergothioneine production. The transformant showing the highest ergothioneine production is considered to have the Egt1 and Egt2 protein with highest level of enzyme activity in combination.

In one aspect of this embodiment, once a best performing ergothioneine is identified through plasmid transformation, the corresponding nucleotide sequences coding for Egt1 and Egt2 proteins are integrated into the host chromosomal DNA to achieve stable integration and to avoid using antibiotics in the growth medium to maintain the self-replicating plasmid. In one aspect of this embodiment, the nucleotide sequences coding for Egt1 and Egt2 proteins are under the control of a constitutively active promoter. In another aspect of this embodiment, the nucleotide sequences coding for Egt1 and Egt2 proteins are under the control of an inducible promoter.

Once a stably transformed industrially useful microorganism with an exogenous ergothioneine pathway is obtained, further improvement in ergothioneine production is achieved through other genetic manipulations aimed at increasing the pool size of substrates used in ergothioneine production. Since ergothioneine is a thiol derived from histidine, to further improve the ergothioneine production, it is necessary to increase the pool size of the co-substrate molecules such as methionine and cysteine.

In one of aspect of this embodiment, the industrial microbial strain engineered to have the exogenous pathway for ergothioneine biosynthesis is subjected to further genetic engineering to increase the uptake of methionine from the culture medium. In one aspect of the present invention, the industrial microbial strain engineered to have the exogenous ergothioneine pathway is further transformed with a nucleotide sequence coding for the transporter YjeH to increase the pool size of methionine which is necessary to supply S-adenosylmethionine required for the conversion of L-histidine to trimethyl histidine hercynine within the microbial cells.

Cysteine is yet another co-substrate in the biosynthesis of ergothioneine from L-histidine within the microbial cells. In another embodiment of the present invention, the industrial microbial strain engineered to have the exogenous pathway for ergothioneine biosynthesis is subjected to further genetic engineering to increase the pool size of the cysteine within the microbial cell.

Since cysteine is derived from serine, in one aspect of the present invention, serine pool is increased by means of increasing the activity of D-3-phosphoglycerate dehydrogenase (SerA) and phosphoserine phosphatase (SerB and SerC) responsible for the conversion of 3-p-glycerate into L-serine. In one aspect of this embodiment, the activity of these enzymes is improved by means of expressing these genes using a constitutive promoter. In another aspect of this embodiment, the degradation of serine within the microbial cell is reduced by means of mutating the gene sdaA coding for the L-serine hydratase 1 wherein the mutation is deletion, frameshift or point mutation decreasing or eliminating L-serine hydratase 1.

L-serine is converted into L-cysteine in a two-step enzyme reaction. In the first step of this reaction, the seine acetylytransferase enzyme (Cys E) converts L-serine into o-acetyl serine which in turn is converted into L-cysteine by the enzyme cysteine synthase B (CysM). In one aspect of this embodiment, the activity of the CysE and CysM enzymes coded by cysE and cysM genes are increased by means of expressing these enzymes using a constitutive promoter. In another aspect of the present invention, the activity of NrdH enzyme encoded by nrdH gene is increased by means of expressing this enzyme using a constitutive promoter. In yet another aspect of this embodiment, the degradation of L-cysteine to pyruvate, ammonium and hydrogen sulfide within the microbial cell is reduced by means of mutating the tnaA gene coding for L-cysteine desulfhydrase and yhaM gene coding for L-cysteine desulfidase, wherein the mutation is deletion, frameshift or point mutation, decreasing or eliminating the function of these enzymes.

In another aspect of this embodiment, the activity of L-cysteine exporter is upregulated using the constitutive promoter to drive the expression of the corresponding gene ydeD.

In yet another embodiment of the present embodiment, a native promote is used to upregulate the expression of cysB gene coding for the transcriptional regulator CysB protein, a positive regulator of gene expression for the cysteine regulon, a system of 10 or more loci involved in the biosynthesis of L-cysteine from inorganic sulfate.

In yet another aspect of the present invention, the ergothioneine producing strain having exogenous egt1 and egt2 gene is expected to have a disruption in the metJ gene coding for a transcriptional repressor controlling the methionine biosynthesis. With the disruption of metJ gene, the methionine pool size within the ergothioneine producing microbial strain is expected to increase with a consequent increase in the production of ergothioneine.

A transcriptional repressor protein (MetJ) involved in methionine metabolism is encoded by metJ gene and the disruption of this gene is effective in further increasing the production of ergothioneine. Accordingly, in one aspect of the present invention, in the microbial cells expressing heterologous ergothioneins biosynthetic genes, the metJ gene is disrupted so that there is no expression of MetJ protein.

### Definitions

A host cell according to the present invention is any cell that is suitable for the expression of any exogenous protein functional in the ergothioneine biosynthetic pathway. Such a host cell expressing heterologous protein functional in the ergothioneine biosynthetic pathway results from the transformation of the host cell with a recombinant plasmid comprising at least one polynucleotide sequence coding for a protein functional in the ergothioneine biosynthetic pathway and such a host cell is referred as a engineered microbial host cell in the present invention. The list of host cells suitable for the present invention includes, but is not limited to, bacterial cells, and fungal cells including yeast cells. Bacterial cells suitable for the present invention include, without limitation, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., Pantoea spp, and Vibrio natriegens. Yeast cells suitable for the present invention include, without limitation, engineered Saccharomyces spp., Schizosaccharomyces, Hansenula, Candida, Kluyveromyces, Yarrowia, Candida boidinii, and Pichia.

The term a cell culture refers to any cell or cells including the recombinant host cells that are in a culture. Culturing is the process in which cells are grown under controlled conditions, typically outside of their natural environment. For example, cells, such as yeast cells, may be grown as a cell suspension in liquid nutrient broth. A cell culture includes, but is not limited to, a bacterial cell culture, fungal cell culture and a yeast cell culture.

In some embodiments, cells are cultured at a temperature of 16° C. to 40° C. For example, cells may be cultured at a temperature of 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C.

In some embodiments, cells are cultured for a period of 12 hours to 72 hours, or more. For example, cells may be cultured for a period of 12, 18, 24, 30, 36, 42, 48, 54, 60, 66, or 72 hours. Typically, cells, such as bacterial cells, are cultured for a period of 12 to 24 hours. In some embodiments, cells are cultured for 12 to 24 hours at a temperature of 37° C. In some embodiments, cells are cultured for 12 to 24 hours at a temperature of 16° C.

In some embodiments, cells are cultured to a density of 1×10⁸ (OD₆₀₀<1) to 2×10¹¹ (OD˜200) viable cells/ml cell culture medium. In some embodiments, cells are cultured to a density of 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, or 2×10¹¹ viable cells/ml. (Conversion factor: OD 1=8×10⁸ cells/ml).

To induce protein expression by the host cell, 0.5 mM isopropyl β-D-1-thiogalactopyranoside (IPTG) was added and the culture was further grown at 16° C. for 22 hr. Cells were harvested by centrifugation (3,000×g; 10 min; 4° C.). The cell pellets were collected and were either used immediately or stored at −80° C.

The terms “nucleic acid” and “nucleotide” are used according to their respective ordinary and customary meanings as understood by a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is used according to its ordinary and customary meaning as understood by a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same acid or polypeptide.

The terms “polypeptide,” “protein,” and “peptide” are used according to their respective ordinary and customary meanings as understood by a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polypeptide product. Thus, exemplary polypeptides include polypeptide products, naturally occurring proteins, homologs, orthologs, paralogs, fragments and other equivalents, variants, and analogs of the foregoing.

The terms “polypeptide fragment” and “fragment,” when used in reference to a reference polypeptide, are used according to their ordinary and customary meanings to a person of ordinary skill in the art, and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full length polypeptide or protein, and has substantially the same biological activity, or carries out substantially the same function as the full length polypeptide or protein (e.g., carrying out the same enzymatic reaction).

The term “functional variant” further includes conservatively substituted variants. The term “conservatively substituted variant” refers to a peptide having an amino acid sequence that differs from a reference peptide by one or more conservative amino acid substitutions, and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., Cell 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical.

“Percent (%) amino acid sequence identity” with respect to the variant polypeptide sequences of the subject technology refers to the percentage of amino acid residues in a candidate sequence that are identical with the amino acid residues of a reference polypeptide, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI-BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, drop off for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Techniques for determining amino acid sequence “similarity” are well known in the art. In general, “similarity” refers to the exact amino acid to amino acid comparison of two or more polypeptides at the appropriate place, where amino acids are identical or possess similar chemical and/or physical properties such as charge or hydrophobicity. A “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

An amino acid position “corresponding to” a reference position refers to a position that aligns with a reference sequence, as identified by aligning the amino acid sequences. Such alignments can be done by hand or by using well-known sequence alignment programs such as ClustalW2, Blast 2, etc.

Unless specified otherwise, the percent identity of two polypeptide or polynucleotide sequences refers to the percentage of identical amino acid residues or nucleotides across the entire length of the shorter of the two sequences.

“Coding sequence” is used according to its ordinary and customary meaning as understood by a person of ordinary skill in the art, and is used without limitation to refer to a DNA sequence that encodes for a specific amino acid sequence.

“Suitable regulatory sequences” is used according to its ordinary and customary meaning as understood by a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is used according to its ordinary and customary meaning as understood by a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different cell types, or at different stages of development, or in response to different environmental conditions. Promoters that cause a gene to be expressed in most cell types at most times are commonly referred to as “constitutive promoters.” It is further recognized that since, in most cases, the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is used according to its ordinary and customary meaning as understood by a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are used according to their ordinary and customary meanings as understood by a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant.” “heterologous,” and “exogenous,” when used herein in connection with polynucleotides, are used according to their ordinary and customary meanings as understood by a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid.” “vector,” and “cassette” are used according to their ordinary and customary meanings as understood by a person of ordinary skill in the art, and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used herein are well known in the art and are described, for example, by Sambrook, J., Fritsch, E. F. and Maniatis, T. Molecular Cloning: A Laboratory Manual, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N. Y., 1984; and by Ausubel, F. M. et al., In Current Protocols in Molecular Biology, published by Greene Publishing and Wiley-Interscience, 1987; the entireties of each of which are hereby incorporated herein by reference to the extent they are consistent herewith.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

The disclosure will be more fully understood upon consideration of the following non-limiting Examples.

### EXAMPLES

### Example 1

### Selecting and Screening Egt1 and Egt 2 Candidate Enzymes

Translated nucleotide databases were searched using SpEgt1 and SpEgt2, respectively, as a protein query (tblastn). Uncharacterized proteins with full length mRNA were selected and aligned using Vector NTI software. The corresponding putative genes were synthesized and optimized to E. coli codon usage without BsaI, BsmBI, BpiI and NotI sites for cloning purpose. The synthesized genes were cloned in the modified pUC57 (pUC57-BsaI-Free) vector (Tier0). Then, Egt1 and Egt2 genes were subcloned into EC088 and EC090 vector using BsaI reaction, providing DVK-Egt1-AE and DVK-Egt2-EF vectors (Tier1), respectively, according to the MoClo protocol (Iverson, et. al. ACS Synth. Biol. 2016, 5, 99-103). Finally, both subcloned Egt1 and Egt2 genes were combined into EC062 vector, generating DVA-Egt1-Egt2-AF vectors (Tier2). The following screening strategy was used. The 25 Egt1 candidates were screened using functional SpEgt2 gene; similarly, 15 Egt2 candidates were screened using functional SpEgt1 gene. The best combinations of Egt1 and Egt2 candidates were transformed into E. coli host such as MG1655 and JM109 for final ET production (Table 1).

For the screening of Egt1 and Egt2 candidates, the LB medium with or without the addition of histidine, cysteine, and methionine substrate was used. For the ergothioneine production, the modified minimum M9 medium was used with glucose as carbon source and yeast extract as nitrogen source, and with or without additional substrate such as histidine, cysteine, and methionine.

### Example 2

### Testing Expression of Candidate Enzymes for Ergothioneine Biosynthesis

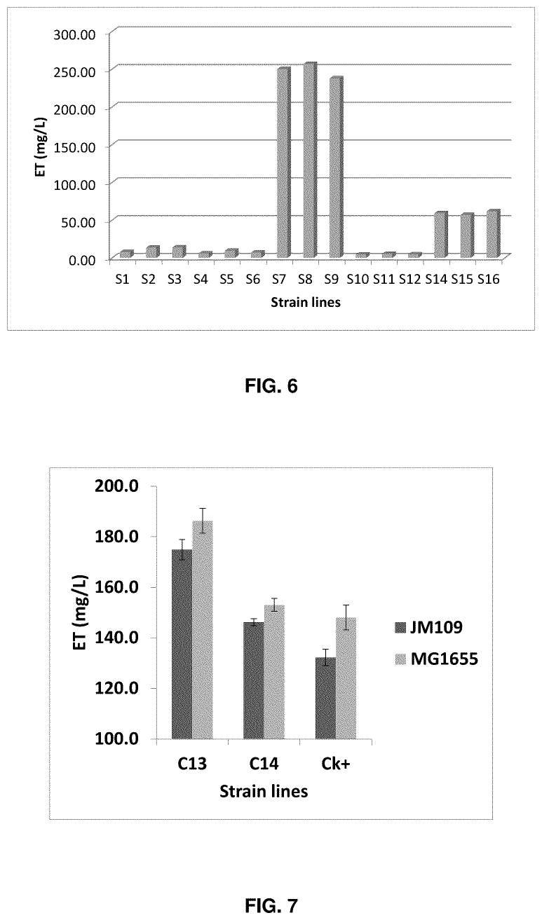

As shown in FIG. 6, among all the strain lines tested, transformants expressing SpEgt1/SpEgt2 enzymes (S7, S8, and S9) produced the highest amount of ergothioneine (up to 257 mg/L), while the strain lines expressing EgtDBCE enzymes (S14, S15, and S16) were able to yield a titer of 62 mg/L. However, other transformants carrying EanB and its homolog EanB3 (S1, S2, S3, S4, S5, S6) could only produce very limited amounts of ergothioneine (˜15 mg/L).

### Example 3

### Screening of Gene Candidates for High Ergothioneine Production

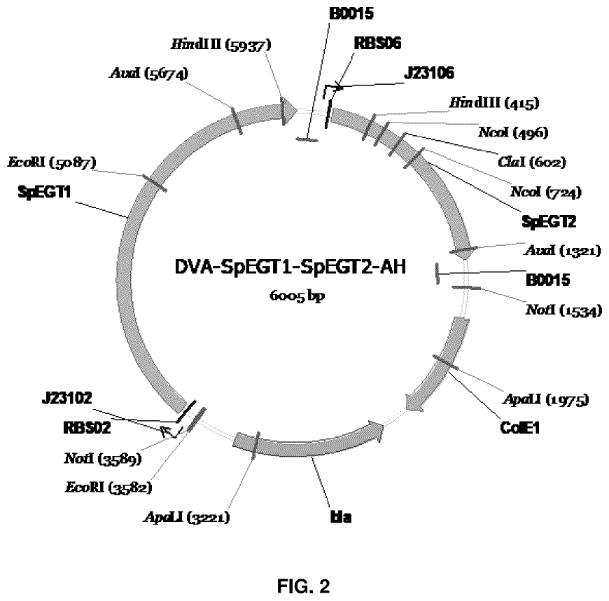

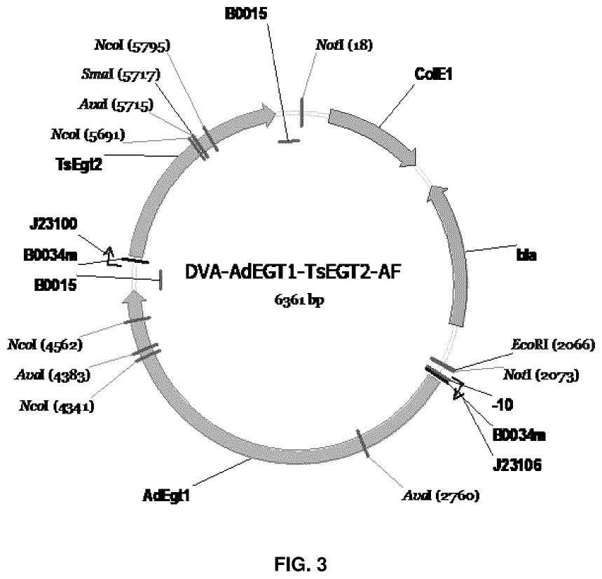

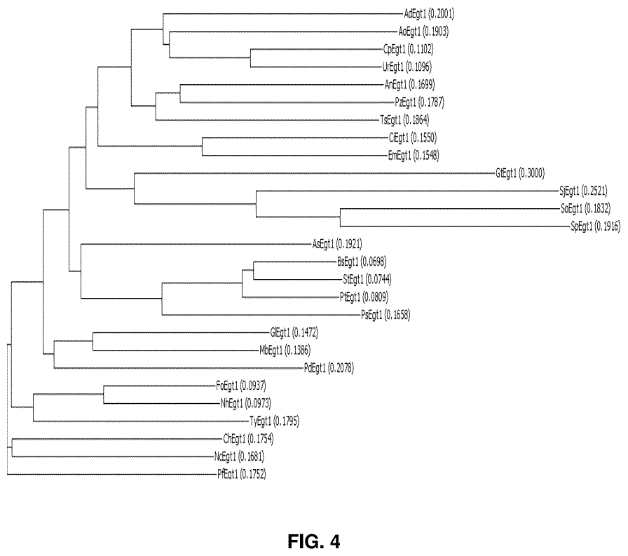

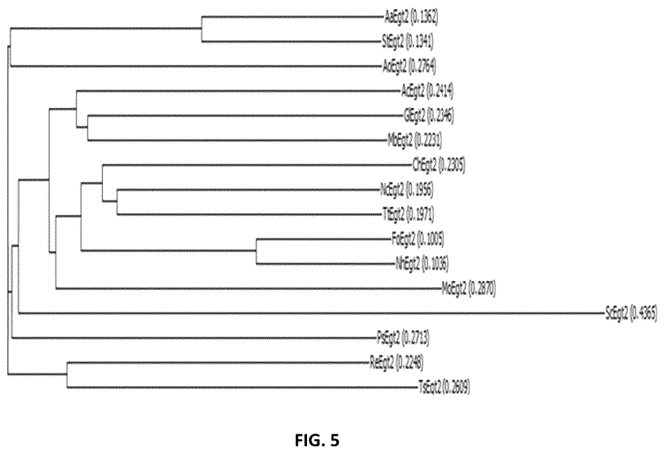

Two sequences encoding for Egt1 and Egt2 from S. pombe, respectively were used as query sequences to blast in databases. Twenty-five (25) sequences for Egt1 candidates and fifteen (15) sequences for Egt2 candidates were chosen based on their similarities. These sequences were optimized to E. coli codon usage without BsaI, BsmBI, BpiI and NotI sites for cloning purpose, and synthesized using GeneUniversal service. The synthesized genes were cloned in the modified pUC57 (pUC57-BsaI-Free) vector (Tier0). Then, Egt1 and Egt2 genes were subcloned into EC088 and EC090 vector using BsaI reaction, resulting DVK-Egt1-AE and DVK-Egt2-EF vectors (Tier1), respectively according to the MoClo protocol. Tier1 parts used were listed in Table 1. Finally, both subcloned Egt1 and Egt2 genes were combined into EC062 vector, generating DVA-Egt1-Egt2-AF vectors (Tier2, see FIG. 2). The screening strategy was used as follows. The 25 Egt1 candidates were screened using functional SpEgt2 gene and 15 Egt2 candidates using functional SpEgt1 gene. The best pairs of Egt1 and Egt2 candidates were combined for final ET production (Table 2 and Table 3).

### Example 4

### Ergothioneine Production in Large Volume Fermentation

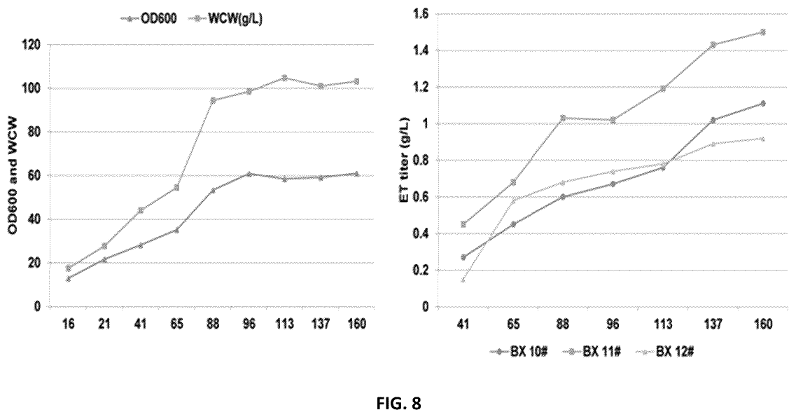

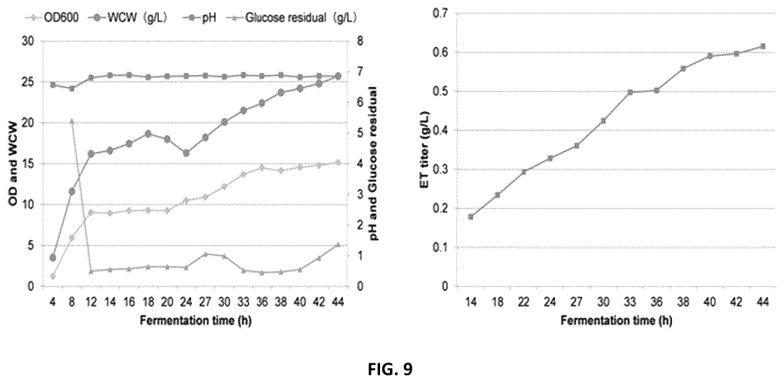

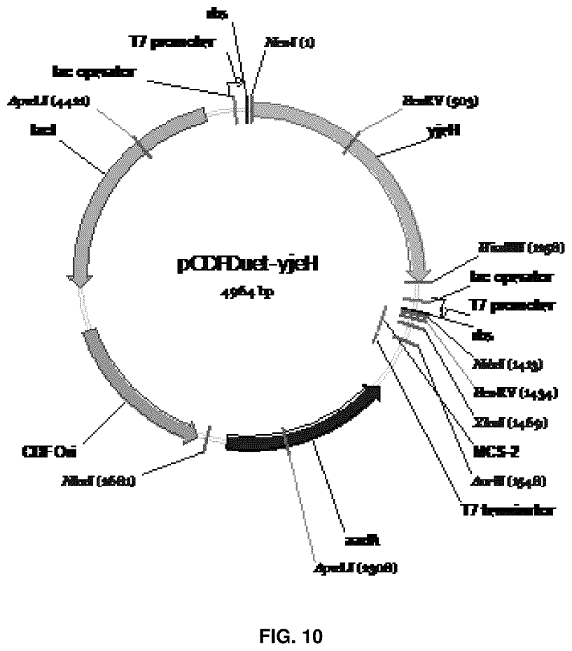

The best pairs of Egt1 and Egt2 candidates were first tested in shaking flasks with triplicates. Cells were cultivated in LB medium with appropriate antibiotics (carb¹⁰⁰) without the addition or feeding of any substrates. Samples were taken from 48 h cell cultures and analyzed by HPLC. The results showed the strain C13 expressing both Egt1 from Ajellomyces dermatitidis (SEQ ID NO: 18) and Egt2 from Talaromyces stipitatus (SEQ ID NO: 90) enzymes produced the highest titer of ergothioneine, compared to the C14 strain expressing Egt1 from Aspergillus niger and Egt2 from Talaromyces stipitatus and ck+ strain expressing Egt1 and Egt2 enzymes from Saccharomyces pompe (FIG. 7). FIG. 8 shows the ergothioneine production with C13 E. coli strain in 3 L fermenter. FIG. 9 shows the ergothioneine production with C13 E. coli strain in 5,000 L fermenter.

### Example 5

### HPLC Analysis

Samples were analyzed using a Dionex UPLC Ultimate 3000 (Sunnyvale, CA). The compounds were separated on a Luna C18(2) column (particle size 5.0 μm, diameter×length=4.6×250 mm; Phenomenex) and detected at 254 nm. The mobile phase consisted of 0.01% triethylamine in water (A) and acetonitrile (B). The isocratic elution (B=0.8% for 10 min) was used for the separation of sample components. The flow rate was 0.8 ml/min and the inject volume was 5 μl.

### Example 6

Increasing Ergothioneine Production by Introducing Gene for Amino Acid Transporter yjeH

As described above, hercynine is a vital intermediate toward ergothioneine biosynthesis. Since the synthesis of hercynine needs one molecular of L-histidine and three molecules of L-methionine, the synthetic steps of L-methionine or S-adenosylmethionine are very likely rate-limiting. Recently, Liu et al. reported that an efflux transporter functions as an exporter of L-methionine and other three branched-chain amino acids, which is important in the extracellular accumulation of amino acids in E. coli (Liu et al 2020, Enhancement of Sulfur Conversion Rate in the Production of L-Cysteine by Engineered Escherichia coli; J. Agric. Food Chem. 68: 250-257; Tanaka et al 2020 Gram-scale fermentative production of ergothioneine driven by overproduction of cysteine in Escherichia coli. Scientific Reports, Vol. 9, Article number: 1895).

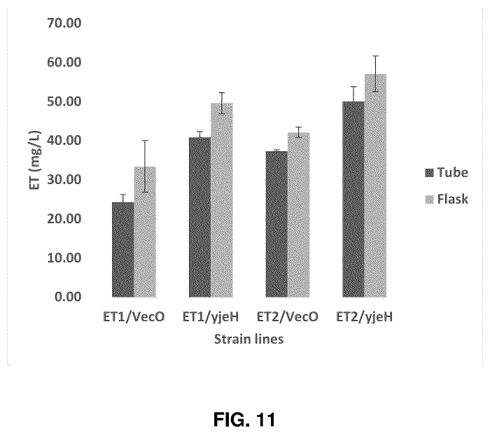

In order to further increase ergothioneine yield, we co-expressed the transporter YjeH with E. coli W strains expressing fungal enzymes Egt1 and Egt2 involved in ergothioneine production. Our data showed that co-expression of Egt1 and Egt2 along with YjeH was able to increase the ergothioneine synthesis when compared to the ergothioneine with only Egt1 and Egt2 enzyme. In fact, ergothioneine titers from co-expression of Egt1-Egt2-YjeH increased by 48.27% (FIG. 11), when compared to the control expressing only Egt1 and Egt2.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### Equivalents and Scope

Those skilled in the art will recognize, or be able to ascertain using no more than routine experimentation, many equivalents to the specific embodiments of the present disclosure described herein. The scope of the present disclosure is not intended to be limited to the above description, but rather is as set forth in the appended claims.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having.” “containing.” “involving.” “holding.” “composed of.” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. All references, patents and patent applications disclosed herein are incorporated by reference with respect to the subject matter for which each is cited, which in some cases may encompass the entirety of the document. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above description, but rather as set forth in the appended claims. Those of ordinary skill in the art will appreciate that various changes and modifications to this description may be made without departing from the spirit or scope of the present disclosure, as defined in the following claims.

TABLE 1
Sequence Information

| Seq. ID | Sequence Description |
|---|---|
| No. | Internal reference number; GenBank accession number; |
| | Source organism; sequence type |
| 1 | SpEgt1; Schizosaccharomyces pombe; |
| | nucleotide sequence |
| 2 | SpEgt1; Schizosaccharomyces pombe; |
| | amino acid sequence |
| 3 | SpEgt2; Schizosaccharomyces pombe; |
| | nucleotide sequence |
| 4 | SpEgt2; Schizosaccharomyces pombe; |
| | amino acid sequence |
| 5 | eanA; Chlorobium limicola; |
| | nucleotide sequence |
| 6 | EanA; Chlorobium limicola; |
| | amino acid sequence |
| 7 | eanB; Chlorobium limicola; |
| | nucleotide sequence |
| 8 | EanB; Chlorobium limicola; |
| | amino acid sequence |
| 9 | J23100; Promoter sequence; |
| | TTGACGGCTAGCTCAGTCCTAGGTACAGTGCTAGC |
| 10 | J23102; Promoter sequence; |
| | TTGACAGCTAGCTCAGTCCTAGGTACTGTGCTAGC |
| 11 | RBS02; ribosome binding site; GTAACATTTAATAGGAGGAATTA |
| 12 | J23106; Promoter sequence; |
| | TTTACGGCTAGCTCAGTCCTAGGTATAGTGCTAGC |
| 13 | RBS06; ribosome binding site; ATTAACGATACTAAGGAGCGAAT |
| 14 | RBS-B0034m; ribosome binding site; AGAGAAAGAGGAGAAATACTA |
| 15 | A1Egt1; 1_XM_002844094.1; Microsporum canis; |
| | nucleotide sequence_ |
| 16 | A1Egt1; 1_XM_002844094.1; Microsporum canis; |
| | amino acid sequence |
| 17 | A2Egt1; 1_XM_002622999.1; Ajellomyces dermatitidis; |
| | nucleotide sequence |
| 18 | A2Egt1; 1_XM_002622999.1; Ajellomyces dermatitidis; |
| | amino acid sequence |
| 19 | A3Egt1; 1_XM_001397080.2; Aspergillus niger; |
| | nucleotide sequence |
| 20 | A3Egt1; 1_XM_001397080.2; Aspergillus niger; |
| | amino acid sequence |
| 21 | A4Egt1; 1_XM_003066635.1; Coccidioides posadasii; |
| | nucleotide sequence |
| 22 | A4Egt1; 1_XM_003066635.1; Coccidioides posadasii; |
| | amino acid sequence |
| 23 | A5Egt1; 1_XM_016386852.1; Cladophialophora immunda; |
| | nucleotide sequence |
| 24 | A5Egt1; 1_XM_016386852.1; Cladophialophora immunda; |
| | amino acid sequence |
| 25 | A6Egt1; 1_XM_008090310.1; Glarea lozoyensis; |
| | nucleotide sequence |
| 26 | A6Egt1; 1_XM_008090310.1; Glarea lozoyensis; |
| | amino acid sequence |
| 27 | A7Egt1; 1_XM_016369232.1; Exophiala mesophila; |
| | nucleotide sequence |
| 28 | A7Egt1; 1_XM_016369232.1; Exophiala mesophila; |
| | amino acid sequence |
| 29 | A8Egt1; 1_XM_018400273.1; Fusarium oxysporum; |
| | nucleotide sequence |
| 30 | A8Egt1; 1_XM_018400273.1; Fusarium oxysporum; |
| | amino acid sequence |
| 31 | B1Egt1; 1_XM_003048838.1; Nectria haematococca; |
| | nucleotide sequence |
| 32 | B1Egt1; 1_XM_003048838.1; Nectria haematococca; |
| | amino acid sequence |
| 33 | B2Egt1; 1_XM_022728240.1; Penicilliopsis zonata; |
| | nucleotide sequence |
| 34 | B2Egt1; 1_XM_022728240.1; Penicilliopsis zonata; |
| | amino acid sequence |
| 35 | B3Egt1; 1_XM_014676787.1; Penicillium digitatum; |
| | nucleotide sequence |
| 36 | B3Egt1; 1_XM_014676787.1; Penicillium digitatum; |
| | amino acid sequence |
| 37 | B4Egt1; 1_XM_001939502.1; Pyrenophora tritici-repentis; |
| | nucleotide sequence |
| 38 | B4Egt1; 1_XM_001939502.1; Pyrenophora tritici-repentis; |
| | amino acid sequence |
| 39 | B5Egt1; 1_XM_002487114.1; Talaromyces stipitatus; |
| | nucleotide sequence |
| 40 | B5Egt1; 1_XM_002487114.1; Talaromyces stipitatus; |
| | amino acid sequence |
| 41 | B6Egt1; 1_XM_014099552.1; Trichoderma virens; |
| | nucleotide sequence |
| 42 | B6Egt1; 1_XM_014099552.1; Trichoderma virens; |
| | amino acid sequence |
| 43 | B7Egt1; 1_XM_002540793.1; Uncinocarpus reesii; |
| | nucleotide sequence |
| 44 | B7Egt1; 1_XM_002540793.1; Uncinocarpus reesii; |
| | amino acid sequence |
| 45 | B8Egt1; 1_XM_013164379.1; Schizosaccharomyces octosporus; |
| | nucleotide sequence |
| 46 | B8Egt1; 1_XM_013164379.1; Schizosaccharomyces octosporus; |
| | amino acid sequence |
| 47 | C1Egt1; 1_XM_002172061.1; Schizosaccharomyces japonicus; |
| | nucleotide sequence |
| 48 | C1Egt1; 1_XM_002172061.1; Schizosaccharomyces japonicus; |
| | amino acid sequence |
| 49 | C2Egt1; 1_XM_007696134.1; Bipolaris sorokiniana; |
| | nucleotide sequence |
| 50 | C2Egt1; 1_XM_007696134.1; Bipolaris sorokiniana; |
| | amino acid sequence |
| 51 | C3Egt1; 1_XM_008027421.1; Exserohilum turcica; |
| | nucleotide sequence |
| 52 | C3Egt1; 1_XM_008027421.1; Exserohilum turcica; |
| | amino acid sequence |
| 53 | C4Egt1; 1_XM_018186317.1; Paraphaeosphaeria sporulosa; |
| | nucleotide sequence |
| 54 | C4Egt1; 1_XM_018186317.1; Paraphaeosphaeria sporulosa; |
| | amino acid sequence |
| 55 | C5Egt1; 1_XM_007289816.1; Marssonina brunnea; |
| | nucleotide sequence |
| 56 | C5Egt1; 1_XM_007289816.1; Marssonina brunnea; |
| | amino acid sequence |
| 57 | C6Egt1; 1_XM_024470276.1; Pseudogymnoascus destructans; |
| | nucleotide sequence |
| 58 | C6Egt1; 1_XM_024470276.1; Pseudogymnoascus destructans; |
| | amino acid sequence |
| 59 | C7Egt1; 1_XM_007834197.1; Pestalotiopsis fici; |
| | nucleotide sequence |
| 60 | C7Egt1; 1_XM_007834197.1; Pestalotiopsis fici; |
| | amino acid sequence |
| 61 | C8Egt1; 1_XM_013493644.1; Aureobasidium subglaciale; |
| | nucleotide sequence |
| 62 | C8Egt1; 1_XM_013493644.1; Aureobasidium subglaciale; |
| | amino acid sequence |
| 63 | D1Egt1; 1_XM_016360447.1; Verruconis gallopava; |
| | nucleotide sequence |
| 64 | D1Egt1; 1_XM_016360447.1; Verruconis gallopava; |
| | amino acid sequence |
| 65 | D2Egt2; 2_XM_001728079.1; Neurospora crassa; |
| | nucleotide sequence |
| 66 | D2Egt2; 2_XM_001728079.1; Neurospora crassa; |
| | amino acid sequence |
| 67 | D3Egt2; 2_XM_003653634.1; Thielavia terrestris; |
| | nucleotide sequence |
| 68 | D3Egt2; 2_XM_003653634.1; Thielavia terrestris; |
| | amino acid sequence |
| 69 | D4Egt2; 2_XM_018300274.1; Colletotrichum higginsianum; |
| | nucleotide sequence |
| 70 | D4Egt2; 2_XM_018300274.1; Colletotrichum higginsianum; |
| | amino acid sequence |
| 71 | D5Egt2; 2_XM_018389754.1; Fusarium oxysporum; |
| | nucleotide sequence |
| 72 | D5Egt2; 2_XM_018389754.1; Fusarium oxysporum; |
| | amino acid sequence |
| 73 | D6Egt2; 2_XM_018216062.1; Phialocephala scopiformis |
| | nucleotide; sequence |
| 74 | D6Egt2; 2_XM_018216062.1; Phialocephala scopiformis; |
| | amino acid sequence |
| 75 | D7Egt2; 2_XM_003045069.1; Nectria haematococca; |
| | nucleotide sequence |
| 76 | D7Egt2; 2_XM_003045069.1; Nectria haematococca; |
| | amino acid sequence |
| 77 | D8Egt2; 2_XM_024886631.1; Hyaloscypha bicolor; |
| | nucleotide sequence |
| 78 | D8Egt2; 2_XM_024886631.1; Hyaloscypha bicolor; |
| | amino acid sequence |
| 79 | E1Egt2; 2_XM_024814247.1; Aspergillus candidus; |
| | nucleotide sequence |
| 80 | E1Egt2; 2_XM_024814247.1; Aspergillus candidus; |
| | amino acid sequence |
| 81 | E2Egt2; 2_XM_003720232.1; Pyricularia oryzae; |
| | nucleotide sequence |
| 82 | E2Egt2; 2_XM_003720232.1; Pyricularia oryzae; |
| | amino acid sequence |
| 83 | E3Egt2; 2_XM_008078420.1; Glarea lozoyensis; |
| | nucleotide sequence |
| 84 | E3Egt2; 2_XM_008078420.1; Glarea lozoyensis; |
| | amino acid sequence |
| 85 | F1Egt2; 2_XM_008028041.1; Exserohilum turcica; |
| | nucleotide sequence |
| 86 | F1Egt2; 2_XM_008028041.1; Exserohilum turcica; |
| | amino acid sequence |
| 87 | F2Egt2; 2_XM_013170142.1; Schizosaccharomyces cryophilus; |
| | nucleotide sequence |
| 88 | F2Egt2; 2_XM_013170142; Schizosaccharomyces cryophilus; |
| | amino acid sequence |
| 89 | F3Egt2; 2_XM_002482656.1; Talaromyces stipitatus; |
| | nucleotide sequence |
| 90 | F3Egt2; 2_XM_002482656.1; Talaromyces stipitatus; |
| | amino acid sequence |
| 91 | F4Egt2; 2_XM_011130091.1; Arthrobotrys oligospora; |
| | nucleotide sequence |
| 92 | F4Egt2; 2_XM_011130091.1; Arthrobotrys oligospora; |
| | amino acid sequence |
| 93 | F5Egt2; 2_XM_013471838.1; Rasamsonia emersonii; |
| | nucleotide sequence |
| 94 | F5Egt2; 2_XM_013471838.1; Rasamsonia emersonii; |
| | amino acid sequence |
| 95 | yjeH; methionine transporter; Escherichia coli; |
| | nucleotide sequence |
| 96 | yjeH; methionine transporter; Escherichia coli; |
| | amino acid sequence |
| 97 | tnaA; Tryptophanase; Escherichia coli; |
| | nucleotide sequence |
| 98 | TnaA; Tryptophanase; Escherichia coli; |
| | amino acid sequence |
| 99 | sdaA; L-serine dehydratase 1; Escherichia coli; |
| | nucleotide sequence |
| 100 | SdaA; L-serine dehydratase 1; Escherichia coli; |
| | nucleotide sequence |
| 101 | serA; D-3-phosphoglycerate dehydrogenase; Escherichia coli; |
| | nucleotide sequence |
| 102 | SerA; D-3-phosphoglycerate dehydrogenase; Escherichia coli; |
| | amino acid sequence |
| 103 | serB; Phosphoserine phosphatase; Escherichia coli; |
| | nucleotide sequence |
| 104 | SerB; Phosphoserine phosphatase; Escherichia coli; |
| | amino acid sequence |
| 105 | serC; Phosphoserine phosphatase; Escherichia coli; |
| | nucleotide sequence |
| 106 | SerC; Phosphoserine phosphatase; Escherichia coli; |
| | amino acid sequence |
| 107 | cysM; Cysteine synthase B; Escherichia coli; |
| | nucleotide sequence |
| 108 | CysM; Cysteine synthase B; Escherichia coli; |
| | amino acid sequence |
| 109 | nrdH; Glutaredoxin-like protein; Escherichia coli; |
| | nucleotide sequence |
| 110 | NrdH; Glutaredoxin-like protein; Escherichia coli; |
| | amino acid sequence |
| 111 | cysE; Serine acetyltransferase; Escherichia coli; |
| | nucleotide sequence |
| 112 | CysE; Serine acetyltransferase; Escherichia coli; |
| | amino acid sequence |
| 113 | ydeE; EamA domain-containing protein; Escherichia coli; |
| | nucleotide sequence |
| 114 | YdeE; EamA domain-containing protein; Escherichia coli; |
| | amino acid sequence |
| 115 | yhaM; UPF0597 protein YhaM; Escherichia coli; |
| | nucleotide sequence |
| 116 | YhaM; UPF0597 protein YhaM; Escherichia coli; |
| | amino acid sequence |
| 117 | cysB; HTH-type transcriptional regulator; Escherichia coli; |
| | nucleotide sequence |
| 118 | CysB; HTH-type transcriptional regulator; Escherichia coli; |
| | amino acid sequence |
| 119 | cysK; Cysteine synthase A; Escherichia coli; |
| | nucleotide sequence |
| 120 | CysK: Cysteine synthase A; Escherichia coli; |
| | amino acid sequence |
| 121 | cysA; Sulfate/thiosulfate import ATP-binding protein; Escherichia coli; |
| | nucleotide sequence |
| 122 | CysA: Sulfate/thiosulfate import ATP-binding protein; Escherichia coli; |
| | amino acid sequence |
| 123 | cysP; Thiosulfate-binding protein; Escherichia coli; |
| | nucleotide sequence |
| 124 | CysP: Thiosulfate-binding protein; Escherichia coli; |
| | amino acid sequence |
| 125 | cysT; Sulfate transport system permease protein; Escherichia coli; |
| | nucleotide sequence |
| 126 | CysT: Sulfate transport system permease protein; Escherichia coli; |
| | amino acid sequence |
| 127 | cysW; Sulfate transport system permease protein; Escherichia coli; |
| | nucleotide sequence |
| 128 | CysW: Sulfate transport system permease protein; Escherichia coli; |
| | amino acid sequence |
| 129 | egtB; Mycobacterium smegmatis; |
| | nucleotide sequence |
| 130 | EgtB; Mycobacterium smegmatis; |
| | amino acid sequence |
| 131 | egtC; Mycobacterium smegmatis; |
| | nucleotide sequence |
| 132 | EgtC; Mycobacterium smegmatis; |
| | amino acid sequence |
| 133 | egtD; Mycobacterium smegmatis; |
| | nucleotide sequence |
| 134 | EgtD; Mycobacterium smegmatis; |
| | amino acid sequence |
| 135 | egtE; Mycobacterium smegmatis; |
| | nucleotide sequence |
| 136 | EgtE; Mycobacterium smegmatis; |
| | amino acid sequence |
| 137 | NcEgt1; Neurospora crassa; |
| | nucleotide sequence |
| 138 | NcEgt1; Neurospora crassa; |
| | amino acid sequence |
| 139 | MzEanA3; Methanosalsum zhilinae; |
| | nucleotide sequence |
| 140 | MzEanA3; Methanosalsum zhilinae; |
| | amino acid sequence |
| 141 | MzEanB3; Methanosalsum zhilinae; |
| | nucleotide sequence |
| 142 | MzEanB3; Methanosalsum zhilinae; |
| | amino acid sequence |
| 143 | metJ; Escherichia coli; |
| | nucleotide sequence |
| 144 | MetJ; Escherichia coli; |
| | amino acid sequence |

TABLE 2
Tier 1 Analysis

| | | | Ergothioneine |
|---|---|---|---|
| No. | Source of Egt1 | Source of Egt2 | titer (mg/L) |
| 1 | Seq. ID No. 15 | Seq. ID No. 3 | 0 |
| 2 | Seq. ID No. 16 | Seq. ID No. 3 | 67.88/70.65 |
| 3 | Seq. ID No. 17 | Seq. ID No. 3 | 51.92/52.05 |
| 4 | Seq. ID No. 18 | Seq. ID No. 3 | 0 |
| 5 | Seq. ID No. 19 | Seq. ID No. 3 | 0 |
| 6 | Seq. ID No. 20 | Seq. ID No. 3 | 0 |
| 7 | Seq. ID No. 21 | Seq. ID No. 3 | 0 |
| 8 | Seq. ID No. 22 | Seq. ID No. 3 | 0 |
| 9 | Seq. ID No. 23 | Seq. ID No. 3 | 0 |
| 10 | Seq. ID No. 24 | Seq. ID No. 3 | 3.1326/3.11 |
| 11 | Seq. ID No. 25 | Seq. ID No. 3 | 80.72/83.84 |
| 12 | Seq. ID No. 26 | Seq. ID No. 3 | 0 |
| 13 | Seq. ID No. 27 | Seq. ID No. 3 | 15.287/14.37 |
| 14 | Seq. ID No. 28 | Seq. ID No. 3 | 0 |
| 15 | Seq. ID No. 29 | Seq. ID No. 3 | 36.284/46.480 |
| 16 | Seq. ID No. 30 | Seq. ID No. 3 | 0 |
| 17 | Seq. ID No. 31 | Seq. ID No. 3 | 0 |
| 18 | Seq. ID No. 32 | Seq. ID No. 3 | 31.249/38.3938 |
| 19 | Seq. ID No. 33 | Seq. ID No. 3 | 0 |
| 20 | Seq. ID No. 34 | Seq. ID No. 3 | 0 |
| 21 | Seq. ID No. 35 | Seq. ID No. 3 | 0 |
| 22 | Seq. ID No. 36 | Seq. ID No. 3 | 17.38/17.13 |
| 23 | Seq. ID No. 37 | Seq. ID No. 3 | 11.837/10.704 |
| 24 | Seq. ID No. 38 | Seq. ID No. 3 | 0.4288/0.8176 |
| 25 | Seq. ID No. 39 | Seq. ID No. 3 | 11.6874/13.09 |
| 26 | Seq. ID No. 1 | Seq. ID No. 40 | 18.16/18.40 |
| 27 | Seq. ID No. 1 | Seq. ID No. 41 | 14.49/11.788 |
| 28 | Seq. ID No. 1 | Seq. ID No. 42 | 3.748/3.573 |
| 29 | Seq. ID No. 1 | Seq. ID No. 43 | 12.6448/11.8198 |
| 30 | Seq. ID No. 1 | Seq. ID No. 44 | 16.9268/16.898 |
| 31 | Seq. ID No. 1 | Seq. ID No. 45 | 0 |
| 32 | Seq. ID No. 1 | Seq. ID No. 46 | 0 |
| 33 | Seq. ID No. 1 | Seq. ID No. 47 | 0 |
| 34 | Seq. ID No. 1 | Seq. ID No. 48 | 0 |
| 35 | Seq. ID No. 1 | Seq. ID No. 49 | 0 |
| 36 | Seq. ID No. 1 | Seq. ID No. 50 | 0 |
| 37 | Seq. ID No. 1 | Seq. ID No. 51 | 0 |
| 38 | Seq. ID No. 1 | Seq. ID No. 52 | 0 |
| 39 | Seq. ID No. 1 | Seq. ID No. 53 | 0 |
| 40 | Seq. ID No. 1 | Seq. ID No. 54 | 0 |

TABLE 3
Tier 2 Escherichia coli strains for Ergothioneine

| | Bacterial | | | Ergothioneine |
|---|---|---|---|---|
| No. | Strain | Source of Egt1 | Source of Egt2 | titer (mg/L) |
| 1 | C1/ | Seq. ID. 18 | Seq. ID. 68 | 109.4 |
| | | Ajellomyces | Thielavia | |
| | | dermatitidis | terrestris | |
| 2 | C5/ | Seq. ID. 18 | Seq. ID. 72 | 107.5 |
| | | Ajellomyces | Fusarium | |
| | | dermatitidis | oxysporum f. | |
| | | | sp. lycopersici | |
| | | | 4287 | |
| 3 | C9/ | Seq. ID. 18 | Seq. ID. 84 | 104.4 |
| | | | Glarea | |
| | | Ajellomyces | lozoyensis | |
| | | dermatitidis | ATCC 20868 | |
| 4 | C13/ | Seq. ID. 18 | Seq. ID. 90 | 125.3 |
| | | Ajellomyces | Talaromyces | |
| | | dermatitidis | stipitatus | |
| | | | ATCC 10500 | |
| 5 | C2/ | Seq. ID. 20 | Seq. ID. 68 | 93.4 |
| | | Aspergillus niger | Thielavia | |
| | | | terrestris | |
| 6 | C6/ | Seq. ID. | Seq. ID. 72 | 86.7 |
| | | 20Aspergillus | Fusarium | |
| | | niger | oxysporum f. | |
| | | | sp. lycopersici | |
| | | | 4287 | |
| 7 | C10/ | Seq. ID. 20 | Seq. ID. 84 | 104.7 |
| | | Aspergillus | Glarea | |
| | | niger | lozoyensis | |
| | | | ATCC 20868 | |
| 8 | C14/ | Seq. ID. 20 | Seq. ID. 90 | 104.5 |
| | | Aspergillus | Talaromyces | |
| | | niger | stipitatus | |
| | | | ATCC 10500 | |
| 9 | C3/ | Seq. ID. 22 | Seq. ID. 68 | 7.5 |
| | | Coccidioides | Thielavia | |
| | | posadasii | terrestris | |
| 10 | C7/ | Seq. ID. 22 | Seq. ID. 72 | 2.6 |
| | | Coccidioides | Fusarium | |
| | | posadasii | oxysporum f. | |
| | | | sp. lycopersici | |
| | | | 4287 | |
| 11 | C11/ | Seq. ID. 22 | Seq. ID. 84 | 9.7 |
| | | Coccidioides | Glarea | |
| | | posadasii | lozoyensis | |
| | | | ATCC 20868 | |
| 12 | C15/ | Seq. ID. 22 | Seq. ID. 90 | 6.9 |
| | | Coccidioides | Talaromyces | |
| | | posadasii | stipitatus | |
| | | | ATCC 10500 | |
| 13 | C4/ | Seq. ID 36 | Seq. ID. 68 | 0.0 |
| | | Penicillium | Thielavia | |
| | | digitatum Pd1 | terrestris | |
| 14 | C8/ | Seq. ID. 36 | Seq. ID 72 | 0.0 |
| | | Penicillium | Fusarium | |
| | | digitatum Pd1 | oxysporum f. | |
| | | | sp. lycopersici | |
| | | | 4287 | |
| 15 | C12/ | Seq. ID. 36 | Seq. ID. 84 | 0.0 |
| | | Penicillium | Glarea | |
| | | digitatum Pd1 | lozoyensis | |
| | | | ATCC 20868 | |
| 16 | C16/ | Seq. ID. 36 | Seq. ID. 90 | 0.0 |
| | | Penicillium | Talaromyces | |
| | | digitatum Pd1 | stipitatus | |
| | | | ATCC 10500 | |

Sequences of Interest
<SEQ ID NO: 1; DNA; SpEgt1;
Schizosaccharomyces pombe>
ATGACAGAAATAGAAAACATTGGCGCATTAGAAGTTCTCTTCTCT
CCTGAATCCATCGAGCAGAGCCTCAAACGGTGTCAACTCCCCTCC
ACTTTATTATACGATGAAAAAGGTTTACGACTGTTTGATGAGATT
ACGAATTTAAAAGAATACTACCTGTATGAAAGTGAGCTTGATATT
CTGAAGAAGTTCAGCGATTCCATTGCCAACCAGTTACTGTCTCCA
GATCTTCCTAACACGGTTATAGAATTAGGGTGTGGAAATATGCGC
AAAACAAAACTTCTTTTAGATGCGTTTGAAAAGAAGGGCTGTGAT
GTGCATTTTTACGCCCTTGACCTTAATGAAGCCGAGTTGCAAAAA
GGACTGCAGGAGCTTCGTCAAACTACCAATTATCAGCATGTTAAG
GTGTCTGGTATTTGCGGTTGCTTTGAAAGATTGCTACAATGTTTG
GACAGGTTTCGTAGTGAGCCCAATAGTCGAATTAGCATGTTGTAC
TTGGGTGCTTCGATTGGTAATTTTGATAGGAAATCCGCAGCATCA
TTTTTACGTTCGTTTGCCAGTCGTTTGAATATTCATGACAACCTT
TTAATCTCCTTCGATCATAGAAACAAGGCTGAGCTAGTCCAACTA
GCTTACGATGATCCTTATCGTATTACTGAAAAGTTTGAAAAGAAT
ATTTTGGCTAGTGTCAATGCGGTTTTTGGTGAAAACCTTTTCGAC
GAAAATGATTGGGAATATAAAAGTGTCTACGATGAAGATCTCGGT
GTTCATAGGGCCTACTTACAAGCCAAAAATGAAGTTACTGTTATT
AAGGGTCCAATGTTTTTTCAATTTAAACCTAGTCATTTAATTTTG
ATCGAAGAAAGTTGGAAGAATAGCGATCAAGAATGTCGTCAAATC
ATTGAGAAAGGTGATTTTAAATTAGTCTCTAAGTATGAAAGTACG
ATTGCAGATTACTCGACCTATGTTATTACCAAACAATTTCCTGCT
ATGCTTCAACTCCCTCTTCAGCCTTGTCCTTCGTTAGCAGAATGG
GATGCTCTACGCAAAGTATGGCTTTTTATTACAAATAAATTGCTT
AACAAAGATAACATGTACACCGCATGGATTCCTTTGAGACATCCT
CCAATTTTTTACATCGGACATGTCCCTGTTTTTAATGATATTTAT
CTCACAAAGATTGTCAAAAACAAAGCAACTGCTAACAAAAAACAT
TTTTGGGAATGGTTTCAACGTGGTATAGATCCGGACATTGAAGAT
CCCTCCAAGTGCCATTGGCATTCTGAAGTTCCTGAAAGCTGGCCT
TCTCCTGACCAACTTCGTGAATATGAGAAAGAGTCTTGGGAATAT
CATATTGTAAAGTTGTGCAAAGCAATGGATGAATTGTCTACTTCT
GAAAAGAGAATTCTCTGGCTTTGTTACGAACATGTAGCCATGCAT
GTGGAGACAACTCTTTACATCTACGTACAGTCATTTCAAAATGCA
AACCAGACTGTATCAATTTGCGGATCACTTCCTGAACCAGCTGAA
AAACTTACGAAAGCTCCGTTATGGGTGAATGTACCTGAAACGGAA
ATTGCAGTTGGTATGCCCTTGACAACACAATACACGAGTGTTGGA
TCAAATTTGCAATCATCCGATCTTAGTGCCCATGAAAATACAGAT
GAACTTTTTTATTTTGCGTGGGATAATGAGAAACCAATGAGGAAG
AAACTGGTTTCTAGCTTTTCTATTGCCAATCGTCCAATTTCTAAC
GGTGAATATTTAGATTTTATCAATAAAAAGTCAAAAACAGAAAGG
GTGTATCCAAAGCAATGGGCGGAGATTGATGGAACGCTTTACATA
CGAACCATGTACGGCTTATTACCCCTTGACGACTACTTGGGTTGG
CCTGTTATGACTTCATACGACGATCTAAACAATTATGCGAGCTCC
CAAGGATGCAGACTACCAACTGAGGATGAACTGAACTGTTTTTAC
GATCGGGTTCTCGAGAGAACTGATGAGCCTTATGTTAGTACCGAA
GGAAAGGCAACTGGTTTTCAACAATTGCACCCTTTAGCCCTAAGT
GATAATTCAAGTAATCAAATATTCACAGGAGCATGGGAATGGACA
AGTACAGTTCTGGAGAAGCACGAGGATTTTGAACCTGAAGAGCTT
TATCCAGATTATACACGAGATTTCTTTGATGGAAAGCATAATGTC
GTTTTGGGTGGTAGCTTTGCTACGGCTACGCGCATTTCAAATAGA
AGAAGCTTCAGGAACTTTTACCAAGCTGGCTATAAATATGCATGG
ATTGGAGCTAGACTAGTCAAAAACTAA
<SEQ ID NO: 2; PRT; SpEgt1;
Schizosaccharomyces pombe>
MTEIENIGALEVLFSPESIEQSLKRCQLPSTLLYDEKGLRLFDEI
TNLKEYYLYESELDILKKFSDSIANQLLSPDLPNTVIELGCGNMR
KTKLLLDAFEKKGCDVHFYALDLNEAELQKGLQELRQTTNYQHVK
VSGICGCFERLLQCLDRFRSEPNSRISMLYLGASIGNEDRKSAAS
FLRSFASRLNIHDNLLISFDHRNKAELVQLAYDDPYRITEKFEKN
ILASVNAVFGENLFDENDWEYKSVYDEDLGVHRAYLQAKNEV
TVIKGPMFFQFKPSHLILIEESWKNSDQECRQIIEKGDFKLVSKY
ESTIADYSTYVITKQFPAMLQLPLQPCPSLAEWDALRKVWLFITN
KLLNKDNMYTAWIPLRHPPIFYIGHVPVENDIYLTKIVKNKATAN
KKHFWEWFQRGIDPDIEDPSKCHWHSEVPESWPSPDQLREYEKES
WEYHIVKLCKAMDELSTSEKRILWLCYEHVAMHVETTLYIYVQSF
QNANQTVSICGSLPEPAEKLTKAPLWVNVPETEIAVGMPLTTQYT
SVGSNLQSSDLSAHENTDELFYFAWDNEKPMRKKLVSSFSIANRP
ISNGEYLDFINKKSKTERVYPKQWAEIDGTLYIRTMYGLLPLDDY
LGWPVMTSYDDLNNYASSQGCRLPTEDELNCFYDRVLERTDEPYV
STEGKATGFQQLHPLALSDNSSNQIFTGAWEWTSTVLEKHEDFEP
EELYPDYTRDFFDGKHNVVLGGSFATATRISNRRSFRNFYQAGYK
YAWIGARLVKN
<SEQ ID NO: 3; DNA; SpEgt2;
Schizosaccharomyces pombe>
ATGGCAGAGAACAACGTTTACGGCCACGAGATGAAGAAGCACTTC
ATGCTCGACCCAGACTACGTTAACGTCAACAACGGCTCCTGCGGT
ACCGAATCCCTGGCTGTTTACAACAAGCACGTTCAGCTCCTGAAG
GAGGCACAGTCCAAGCCAGATTTCATGTGTAACGCTTACATGCCT
ATGTACATGGAGGCAACCCGCAACGAAGTCGCTAAGCTTATCGGC
GCAGACTCTTCCAACATCGTTTTCTGCAACTCCGCTACTGACGGC
ATCTCCACCGTTCTCCTGACCTTCCCATGGGAGCAGAACGATGAG
ATCCTCATGCTGAACGTCGCATACCCAACCTGTACTTACGCTGCA
GACTTCGCTAAGAACCAGCACAACCTTCGTCTCGACGTGATCGAT
GTTGGTGTTGAAATCGACGAGGACCTGTTCCTCAAGGAGGTCGAA
CAGCGCTTCCTGCAGTCTAAGCCTCGTGCATTCATCTGCGATATC
CTTTCCTCCATGCCAGTTATCCTCTTCCCATGGGAGAAGGTTGTC
AAGCTGTGTAAGAAGTACAACATCGTTTCCATCATCGACGGCGCT
CACGCAATCGGCCACATCCCTATGAACCTCGCTAACGTTGACCCA
GATTTCCTGTTCACCAACGCACACAAGTGGCTTAACTCTCCAGCT
GCATGCACCGTCCTCTACGTGTCCGCTAAGAACCACAACCTGATC
GAGGCACTCCCTCTGTCCTACGGTTACGGCCTTCGCGAAAAGGAG
TCCATCGCTGTTGACACCCTCACTAACCGTTTCGTTAACTCTTTC
AAGCAGGACCTGCCAAAGTTCATCGCAGTCGGCGAGGCTATCAAG
TTCCGCAAGTCCATCGGTGGCGAAGAGAAGATCCAGCAGTACTGT
CACGAGATCGCACTCAAGGGCGCTGAAATCATCTCCAAGGAGCTG
GGTACCTCCTTCATCAAGCCACCTTACCCAGTTGCAATGGTTAAC
GTCGAGGTTCCACTTCGTAACATCCCTTCTATCGAAACCCAGAAG
GTTTTCTGGCCAAAGTACAACACCTTCCTCCGCTTCATGGAGTTC
AAGGGCAAGTTCTACACTCGTCTGTCCGGCGCTGTCTACCTCGAG
GAATCCGATTTCTACTACATCGCAAAGGTGATCAAGGACTTCTGC
TCCCTGTAA
<SEQ ID NO: 4; PRT; SpEgt2;
Schizosaccharomyces pombe>
MAENNVYGHEMKKHFMLDPDYVNVNNGSCGTESLAVYNKHVQLLK
EAQSKPDFMCNAYMPMYMEATRNEVAKLIGADSSNIVFCNSATDG
ISTVLLTFPWEQNDEILMLNVAYPTCTYAADFAKNQHNLRLDVID
VGVEIDEDLFLKEVEQRFLQSKPRAFICDILSSMPVILFPWEKVV
KLCKKYNIVSIIDGAHAIGHIPMNLANVDPDFLFTNAHKWLNSPA
ACTVLYVSAKNHNLIEALPLSYGYGLREKESIAVDTLTNRFVNSF
KQDLPKFIAVGEAIKFRKSIGGEEKIQQYCHEIALKGAEIISKEL
GTSFIKPPYPVAMVNVEVPLRNIPSIETQKVFWPKYNTFLRFMEF
KGKFYTRLSGAVYLEESDFYYIAKVIKDFCSL
<SEQ ID NO: 5; DNA; eanA;
Chlorobium limicola>
ATGGCTTATTCCAAGACCAACCTGTCGGAATTGCCACTTGCAGAT
ATAGATAACCACTTGACTGAAATAGGATTTGATACCACAATAAGT
GAGATAATTACCGGTTTGACTGCTAATGCGAAGTACATCCAGTCG
AAGTATTTTTACGATAAAAGAGGCTCAGCACTTTTCGAAAAAATC
ACCAGTCTGTCTGAATACTATCCATCGCGTACAGAAAAGGCTATT
ATTAGCCAACTTCCACCAGCTCTTATAGAAGACCTTGCTGACATA
GATATTATCGAGCTTGGTTGTGGTGACCATTCAAAGATTAGTTTG
TTGATTCGCCGTATACCAGCAGAGTCCGTACCGGGTTTAAGATAT
TTTCCTATCGATATTAGCCAAACGGCACTGAAGCAGTCGATCGAA
GACCTTCGTGATCTTTTCCCTGCGCTGAAAGTTAAAGGGATACTT
GCAGATTATGTCCACCAAATGCATTTATTCCCTGAAGAACGTAAA
CGGCTGTTTTGCTTTTTCGGTTCTACAATCGGGAACCTTAGCCGT
GAGGAGACGCTTGATTTCATGCAGAACATGGGCACTACTATGCAT
CCGGGTGACATGCTTTTGGTTGGCATGGACAGAGTAAAGAATATA
GCATTGCTGGAAAAGGCGTACAATGACGACCAATTTATCACAGCT
ATGTTTAACAAAAATATACTGCGGGTGATTAACGGCTTAATAAAA
TCCGATTTTAATCCCGATGATTTTGAACACCGGGCTTTCTATAAT
GCTGACTTCAACAGAATCGAGATGCACTTGGAAGCAACTGGGAAT
ATTTCTGTCAAGTCCGCGTTCATGCCTGAACTTATTCGGATCAAG
AAAGGCGAAACAATCCATACTGAGAACAGTCACAAATTCGAAAAG
GCCGACATTCTGTTAATGGGTCAGCACGCCGGGTTAGCCATTAAA
AATATTTATTCGGACAAGAACGAATTATTTTCCTTGGCTCATTAC
GAAAAAAAATAA
<SEQ ID NO: 6; PRT; EanA;
Chlorobium limicola>
MAYSKTNLSELPLADIDNHLTEIGFDTTISEIITGLTANAKYIQS
KYFYDKRGSALFEKITSLSEYYPSRTEKAIISQLPPALIEDLADI
DIIELGCGDHSKISLLIRRIPAESVPGLRYFPIDISQTALKQSIE
DLRDLFPALKVKGILADYVHQMHLFPEERKRLFCFFGSTIGNLSR
EETLDFMQNMGTTMHPGDMLLVGMDRVKNIALLEKAYNDDQF
ITAMFNKNILRVINGLIKSDENPDDFEHRAFYNADFNRIEMH
LEATGNISVKSAFMPELIRIKKGETIHTENSHKFEKADILLMGQH
AGLAIKNIYSDKNELFSLAHYEKK
<SEQ ID NO: 7; DNA; eanB;
Chlorobium limicola>
ATGCAGAACAAGAACTTCAGAGCACCCCAATCAGAAGCAATTGGC
ATTCTGTATAAGTTAATCGAAACTGGATCTAAACACAAAAACATG
TATGACCACACGGAAATCACTACCGATTCACTTCTGGCGTTATTA
GGCAGCGAGAAGGTCAAAATAATCGATGTCCGCTCAGCAGATGCG
TATAACGGCTGGCGTATGAGAGGTGAAGTACGCGGTGGGCATATA
AAAGGCGCAAAATCCCTGCCGGCGAAATGGTTAACTGATCCGGAG
TGGCTTAACATAGTGAGATTCAAGCAGATACGGCCGGAGGACGCA
ATTGTTCTGTATGGATACACACCCGAGGAATGCGAACAGACCGCG
ACGCGCTTCAAAGAAAACGGATATAACAACGTGAGTGTTTTTCAT
CGTTTCCATCCGGATTGGACAGGTAACGACGCGTTCCCCATGGAT
CGGCTTGAGCAGTATAATCGTCTGGTCCCTGCGGAATGGGTGAAT
GGCCTGATATCAGGCGAAGAAATTCCCGAATATGATAATGACACA
TTTATCGTCTGCCATGCGCACTACCGTAACCGTGATGCCTACCTT
TCTGGTCACATCCCTGGCGCTACTGATATGGACACTTTGGCACTT
GAGTCCCCAGAAACCTGGAATCGGCGCACACCAGAGGAGTTAAAA
AAGGCGTTAGAAGAACATGGGATAACCGCATCTACCACTGTGGTC
CTGTACGGAAAGTTTATGCACCCTGATAATGCTGACGAATTTCCA
GGATCTGCAGCTGGTCACATTGGAGCTATTCGTCTGGCCTTCATA
ATGATGTATGCGGGCGTCGAGGATGTGCGTGTGTTAAACGGGGGC
TACCAATCCTGGACAGATGCTGGATTTGCGATTTCAAAGGATGAC
GTTCCGAAAACTACAGTACCTGAGTTCGGTGCTCCGATACCCTCC
CGGCCGGAGTTTGCGGTCGATATTGACGAGGCCAAAGAGATGCTG
CAGTCTGAGGATTCCGATTTGGTGTGCGTCCGCTCGTATCCTGAG
TACATTGGAGAGGTTTCGGGATATAACTATATAAAAAAGAAGGGG
CGCATCCCAGGAGCTATCTTTGCCGAATGCGGGTCCGATGCTTAT
CACATGGAGAACTACCGCAACCATGACCATACTACGCGTGAATAC
CACGAGATAGAGGATATATGGGCGAAGAGCGGAATCATACCCAAA
AAACACTTAGCCTTCTACTGCGGGACGGGATGGCGTGGATCTGAA
GCGTGGTTTAATGCTTTGCTGATGGGATGGCCTCGGGTTTCGGTT
TACGACGGCGGGTGGTTTGAGTGGTCAAACGATCCGGAGAATCCT
TACGAGACAGGCGTGCCAAAATAA
<SEQ ID NO: 8; PRT;
EanB;
Chlorobium limicola>
MQNKNFRAPQSEAIGILYKLIETGSKHKNMYDHTEITTDSLLA
LLGSEKVKIIDVRSADAYNGWRMRGEVRGGHIKGAKSLPAKWLTD
PEWLNIVRFKQIRPEDAIVLYGYTPEECEQTATRFKENGYNNVSV
FHRFHPDWTGNDAFPMDRLEQYNRLVPAEWVNGLISGEEIPEYDN
DTFIVCHAHYRNRDAYLSGHIPGATDMDTLALESPETWNRRTPEE
LKKALEEHGITASTTVVLYGKFMHPDNADEFPGSAAGHIGAIRLA
FIMMYAGVEDVRVLNGGYQSWTDAGFAISKDDVPKTTVPEFGAPI
PSRPEFAVDIDEAKEMLQSEDSDLVCVRSYPEYIGEVSGYNYIKK
KGRIPGAIFAECGSDAYHMENYRNHDHTTREYHEIEDIWAKSGII
PKKHLAFYCGTGWRGSEAWFNALLMGWPRVSVYDGGWFEWSNDPE
NPYETGVPK*
<SEQ ID NO: 9; DNA; J23100;
Promoter sequence>
TTGACGGCTAGCTCAGTCCTAGGTACAGTGCTAGC
<SEQ ID NO: 10; DNA; J23102;
Promoter sequence>
TTGACAGCTAGCTCAGTCCTAGGTACTGTGCTAGC
<SEQ ID NO: 11; DNA; RBS02;
ribosome binding site>
GTAACATTTAATAGGAGGAATTA
<SEQ ID NO: 12; DNA; J23106;
Promoter sequence>
TTTACGGCTAGCTCAGTCCTAGGTATAGTGCTAGC
<SEQ ID NO: 13; DNA; RBS06;
ribosome binding site>
ATTAACGATACTAAGGAGCGAAT
<SEQ ID NO: 14; DNA; RBS-B0034m;
ribosome binding site>
AGAGAAAGAGGAGAAATACTA
<SEQ ID NO: 15; DNA; A1Egt1;
1_XM_002844094.1;
Microsporum canis>
ATGAGCCCGATTGCCACCAGCAATGTTGATATTGTGGATATTCGT
CGCAATAGCCTGGAAAGCAGCCTGGTTCAGGATATCTATCATGGT
CTGCAGGCAAAAGAAAAAAGCCTGCCGACCCTGCTGCTGTATGAT
ACCAAAGGTCTGCGTCTGTTTGAAGATATTACCTATCTGGATGAA
TACTATCTGACCAATGCAGAAATTGAAGTGCTGACCGCCAATGCA
GCCAAAATTGCAGCAATTATTCCGGAAAATTGTCAGCTGGTGGAA
CTGGGTAGCGGCAATCTGCGTAAAATTGAAATTCTGCTGAATGAA
CTGGAACGCACCAAAAAATCAGTGGAATATTATGCCCTGGATCTG
AGCCTGGAAGAACTGCATCGCACCTTTGCAGAACTGCCGAGCAAA
AGCTATCGCTATGTGAAATGCGGTGGTCTGTGGGGTACCTATGAT
GATGGCCTGGCCTGGCTGAATAAGCTGGTGAATCGTAATAAGCCG
ACCTGGGTTATGAGTCTGGGCAGCAGCATGGGTAATTTTAATCCG
ACCGAAGCCGCAGGTTTTCTGAGTGGTTTTGCACGTAGTCTGGGC
CCGGTTGATAGCATGGTTATTGCCCTGGACCCTTGTAAAGCAAGT
GAAAAAGTTTTTCGTGCATATAATGACAGTAAGGGCGTTACCAAA
CAGTTTTATCTGAATGGCCTGAGCAATGCAAATACCATTCTGGGT
TTTGAAGCCTTTAAACTGGGTGAATGGGATGCCATTGGTGAATTT
GATCAGACCCAGGGTTGTCATCGTGCCTATTATGTGCCGCTGACC
GATACCGTTATTCGCGATATTCATATTAAGAAAGGCGAAAAAATC
TTCTTCGAACAGGCATTCAAATTTGGCGCAGATGAATGTGAAAAA
CTGTGGCGTGAAGCCGGTCTGCAGCCGACCCGTAAATTTGGTGAC
GAATATAATATCTATATCCTGAGCAGCGCAAGTGCCACCATGAAT
CCGTATCAGCTGCCGACCAAAGGCCCGGAATATGTGAAAGGCGTT
GTGCCGGCCCTGGGCGATTTTGAAGCCGTGTGGAAACTGTGGGAT
ACCGTGACCACCGCCATGGTGCCGCCGAATGATCTGCTGAGCCGT
CCGATTAATCTGCGTAATAGCCTGATTTTCTATCTGGGTCATATT
CCGGCATTCATGGATCGCCATCTGACCTGTGCCACCAGTGGTATT
CCGACCGAACCGGCCAATTTTCATAGTATGTTTGAACGCGGTATT
GATCCGGATGTTGATAATCCGGATCATTGTCATGATCATAGTGCA
ATTCCGGATGAATGGCCGGCAGTTGAAACCCTGGTTGAATATCAG
CAGAAAGTGCGTGTGCGCGCCAAAAGTCTGTATAGCGATGCCAAA
ACCGGCGATCGCCGCATTGCAGAAGCCCTGTGGATTAGTTTTGAA
CATGAAACCATGCATCTGGAAACCTTTCTGTATATGCTGCTGCAG
AGTAGCAGCACCATGCCGCCGCCGCTGGTGCCTGAACCGGATTTT
AAACAGCTGGCAAGCAATAGCGCCGAAAAAGCAGTTCCGAATGAA
TGGTTTGATATTCCGGAACAGACCCTGGAAATTGGTCTGAATGAA
CCGGAAAGCGATGAAATTCCGAGTAATAGCTTTGGTTGGGATAAT
GAAAAACCGCGCCGCGTTGTGCAGGTTCCGGGCTTTGTGGCCAAA
GCACGTCCGATTACCAATGGCGAATATGCAAAATATCTGGAAGAA
CGTGGCATTGGTCAGGTGCCGGCAAGCTGGGTTAAAAAACATACC
GAAAATGAAAGCACCAATGATGGCATTGAAACCCTGAGCAGCGGT
AATAGTAGCCATAATGTTGTTACCGAATATGCCGTGCGTACCGTG
TTTGGCCCGGTTCCGCTGGAATGGGTTCTGGATTGGCCGGTGGCA
GCAAGTTATGAAGAACTGACCCAGTATGCCAAATGGATGAATTGC
CGTATTCCGACCTTTGAAGAAGTGCGCAGCCTGTATAAATATAGC
CTGACCAGCCCGACCGGTACCAATCATAGCACCAATGAACTGCGT
CCGAGCACCACCGAACTGAAAAAACCGGAATATTGTACCAGTCAT
CAGCCGGTTCAGACCCCGATGAAAATTCATGGTCCGGTGTATGTT
GATCTGGATGGTTGTAATGTGGGCTTTACCCATTGGCATCCGACC
CCGGTTACCCAGAAAGGTAATAAGCTGTGTGGTCAGGGTGACTTT
GGCGGTCTGTGGGAATGGACCAGCAGTACCCTGCAGGCACATGAA
GGCTTTAAACCGATGAGCCTGTATCCGGCATATACCGCAGATTTC
TTTGATGGCAAACATAATATTGTGCTGGGCGGTAGCTGGGCCACC
CATCCGCGTCTGGCAGGCCGTAGTACCTTTGTTAATTGGTATCAG
CGTAATTATCCGTATGTGTGGGCCGGCGCCCGTCTGGTGCGTGAT
GATtaa
<SEQ ID NO: 16; PRT; A1Egt1; 1_XM_002844094.1;
Microsporum canis>
MSPIATSNVDIVDIRRNSLESSLVQDIYHGLQAKEKSLPTLLLYD
TKGLRLFEDITYLDEYYLTNAEIEVLTANAAKIAAIIPENCQLVE
LGSGNLRKIEILLNELERTKKSVEYYALDLSLEELHRTFAELPSK
SYRYVKCGGLWGTYDDGLAWLNKLVNRNKPTWVMSLGSSMGNFNP
TEAAGFLSGFARSLGPVDSMVIALDPCKASEKVFRAYNDSKGVTK
QFYLNGLSNANTILGFEAFKLGEWDAIGEFDQTQGCHRAYYVPLT
DTVIRDIHIKKGEKIFFEQAFKFGADECEKLWREAGLQPTRKFGD
EYNIYILSSASATMNPYQLPTKGPEYVKGVVPALGDFEAVWKLWD
TVTTAMVPPNDLLSRPINLRNSLIFYLGHIPAFMDRHLTCATSGI
PTEPANFHSMFERGIDPDVDNPDHCHDHSAIPDEWPAVETLVEYQ
QKVRVRAKSLYSDAKTGDRRIAEALWISFEHETMHLETFLYMLLQ
SSSTMPPPLVPEPDFKQLASNSAEKAVPNEWFDIPEQTLEIGLNE
PESDEIPSNSFGWDNEKPRRVVQVPGFVAKARPITNGEYAKYLEE
RGIGQVPASWVKKHTENESTNDGIETLSSGNSSHNVVTEYAVRTV
FGPVPLEWVLDWPVAASYEELTQYAKWMNCRIPTFEEVRSLYKYS
LTSPTGTNHSTNELRPSTTELKKPEYCTSHQPVQTPMKIHGPVYV
DLDGCNVGFTHWHPTPVTQKGNKLCGQGDFGGLWEWTSSTLQAHE
GFKPMSLYPAYTADFFDGKHNIVLGGSWATHPRLAGRSTFVNWYQ
RNYPYVWAGARLVRDD
<SEQ ID NO: 17; DNA; A2Egt1; 1_XM_002622999.1;
Ajellomyces dermatitidis>
ATGGCCCCTAGTAAACTGAGTAATGTTCCGATTATGGATATTCAT
GTTAACGATCTGAAGGATAGCCTGGTGAATGATATCTATGCCGGT
CTGAAACCGAGTCATGGTGGTGCCAAAAGTCTGCCGACCCTGCTG
CTGTATAGTACCGAAGGTCTGCGTCAGTTTGAAGATATTACCTAT
GTTGATGAATACTACCTGACCAATGCAGAAATTGAAGCACTGACC
ACCCATGCCGCAAAAATTGTGAATCAGCTGCCGGAAAATGCACAG
CTGCTGGAACTGGGTAGCGGCAATCTGCGCAAAATTAAGATTCTG
CTGGATGAATTTGAGCGTAAACAGAAAGCAGTTGAATATTTTGCC
CTGGATCTGAGTCGCGAAGAACTGCATCGTACCTTTGCAGAAATT
CCGCAGGGCGGTTATAAATATGTGCGTTGTCGTGGTCTGCATGGC
ACCTATGATGATGCACTGATTTGGCTGACCCGCCCGGAAAATCGC
CGTAATCCGACCTGTATTCTGAGTATGGGTAGCAGTATTGGCAAT
TTTACCCGCCCGGAGGCCGCACAGTTTCTGAATCGTTTTAGCAAA
ATGCTGGGCCCGAGCGATAGCATGCTGATTGGTATTGATAGTTGT
CAGGACCCTGAACGCGTGTATAAAGCCTATAATGATAGTCAGGGT
GTTACCCGTGATTTTTATATGACCGGCCTGAGCCATGCAAATAGT
ATTCTGGGTTTTGAAGCCTTTAAAAAAGAAGATTGGGGTGTTGCA
GGTCATTATGATGTGGTTAGTGGTGCACATATGGCCTATTATGTG
CCGAATAAGGATGTTACCTTTGATGGCGTGGTTCTGGAAAAAGGT
GAAAAAATTTTCTTTGAGCAGGCATTCAAATACGGCCCGAAAGAT
TGCAAAGATTTGTTTCAGCATGCAGGTCTGACCCCGATTGCACAG
TTTGGTAATAATACCGGCGAATATTATGTTCATCTGCTGAGTAGC
TGCGCACTGGAAATGCCGACCCGCGCCGCACAGTATGCAGAAAAT
GTGATTCCGAATGTGGCCGATTTTGAAAATCTGTGGAAAACCTGG
GATATGGTGACCCTGAGCATGATTCCGCAGGATGATCTGCTGAGT
AAACCGATTCGCCTGCGCAATGCACTGATTTTCTATCTGGGCCAT
ATTCCGGCATTTCTGGATATTCATCTGACCCGTGCAACCGATGGT
ACCCCGACCGAACCGAAACATTTTCAGAGTATGTTTGAACGCGGC
ATTGATCCGGATGTTGATAATCCGGAACTGTGTCATGATCATAGT
GATATTCCGAGTGAATGGCCGAGCCTGGGCGAAATTCTGGCATAT
CGCGATGCCGTTCGTAGTCGCACCCTGGCCCTGCTGGAAAAACGC
ACCAAAGATCGTCGCCTGGCCGAAGCCCTGTGGATTGGTTTTGAA
CATGAAGCCATGCATCTGGAAACCTTTCTGTATATGCTGCTGCAG
AGCGATAAAGCCCTGCCGCCGCCGGGCGTTCTGCGTCCTAATTTT
GAAAGTCTGGCACGCCAGAGTGCACTGGAAAGCCGTGAAAATAGC
TGGTTTACCATTCCGGAACAGCAGATTAGCATTGGCCTGGATGAT
CCGAATGAACATGTTATTCCGGAACATAGCTTTAGTTGGGATAAT
GAAAAACCGAAACGTAATGTTAACGTGAGCGCCTTTGCAGCACAG
GCCCGTCCGATTACCAATGGCGAATATGCAACCTATCTGGAAGAA
CATCATATTACCACCATTCCGGCCACCTGGATTGATCTGACCCCG
AAAACCGGCGATGTGAAACGTAATGGTTATGATAGCTGTAATGGC
TATAGTACCAATGGTGTTCGTCAGAGTAGCACCAGTGCCACCAAA
AAATTTCTGGAAAAATTTGCCGTTCGCACCGTGTTTGGTCCGGTG
CCGCTGAAATGGGCACTGAATTGGCCGCTGATGGCAAGCTATAAT
GAACTGCAGGGTTATGCCGAAAGTGTGAATTGCCGTATTCCGACC
TTTGAAGAAGTGCGCAGTATCTATCAGTATAGCGCATTTCTGAAA
AGCAAACAGCGTAATGGCGCAACCAGTCTGACCAATGGCCATAGC
AATGTTCCGAATGGTACCGTTAAAACCTTTGATCGCAGCGTGGAA
CCGGCCAAAGATACCCATGGCAATCCGCGTAGTCCGGATCATCAG
CCGGTGCAGTGCCCGAGCGCAGATCCGATGCCGGTTTATATTGAT
CTGGATGAAAATTGCAACGTGGGTCTGAAACATTGGCATCCGACC
CCGGTGACCCAGAATGGTGACCGCCTGAGTGGCCAGGGTGAACTG
GGCGGCGTTTGGGAATGGACCAGTACCCCGCTGCATGCCCATGAA
GGCTTTAAAGCCATGGATCTGTATCCGGGCTATAGTGCAGATTTC
TTTGATGGCAAACATAATATTGTGCTGGGTGGTAGTTGGGCCACC
CATCCGCGTATTGCCGGCCGTACCACCTTTATTAATTGGTATCAG
CGCAAATATATCTACGTGTGGGCCGGCGCACGCCTGGTTCGCGAT
ATTTAA
<SEQ ID NO: 18; PRT; A2Egt1; 1_XM_002622999.1;
Ajellomyces dermatitidis>
MAPSKLSNVPIMDIHVNDLKDSLVNDIYAGLKPSHGGAKSLPTLL
LYSTEGLRQFEDITYVDEYYLTNAEIEALTTHAAKIVNQLPENAQ
LLELGSGNLRKIKILLDEFERKQKAVEYFALDLSREELHRTFAEI
PQGGYKYVRCRGLHGTYDDALIWLTRPENRRNPTCILSMGSSIGN
FTRPEAAQFLNRFSKMLGPSDSMLIGIDSCQDPERVYKAYNDSQG
VTRDFYMTGLSHANSILGFEAFKKEDWGVAGHYDVVSGAHMAYYV
PNKDVTFDGVVLEKGEKIFFEQAFKYGPKDCKDLFQHAGLTPIAQ
FGNNTGEYYVHLLSSCALEMPTRAAQYAENVIPNVADFENLWKTW
DMVTLSMIPQDDLLSKPIRLRNALIFYLGHIPAFLDIHLTRATDG
TPTEPKHFQSMFERGIDPDVDNPELCHDHSDIPSEWPSLGEILAY
RDAVRSRTLALLEKRTKDRRLAEALWIGFEHEAMHLETFLYMLLQ
SDKALPPPGVLRPNFESLARQSALESRENSWFTIPEQQISIGLDD
PNEHVIPEHSFSWDNEKPKRNVNVSAFAAQARPITNGEYATYLEE
HHITTIPATWIDLTPKTGDVKRNGYDSCNGYSTNGVRQSSTSATK
KFLEKFAVRTVFGPVPLKWALNWPLMASYNELQGYAESVNCRIPT
FEEVRSIYQYSAFLKSKQRNGATSLINGHSNVPNGTVKTFDRSVE
PAKDTHGNPRSPDHQPVQCPSADPMPVYIDLDENCNVGLKHWHPT
PVTQNGDRLSGQGELGGVWEWTSTPLHAHEGFKAMDLYPGYSADF
FDGKHNIVLGGSWATHPRIAGRTTFINWYQRKYIYVWAGARLVRD
I
<SEQ ID NO: 19; DNA;
A3Egt1; 1_XM_001397080.2;
Aspergillus niger>
ATGAGCCCGCTGTGTCCGGTTGTTAAAGGCGTTGATATTGTGGAT
ATTCGTCAGAATGATGTGGAATTTTCACTGGTGAATGATATTCAG
CGCGGTATTGATCCGCCGGCAGGTACCTGCCGCAGCATGCCTACC
ATGCTGCTGTATGATGCCCAGGGCCTGAAACTGTTTGAAGATATT
ACCTATCTGGAAGAATACTATCTGACCAATGCAGAAATTGATGTG
CTGCGTACCCATGCAAAACGCATTGTTGAACGTATTCCGGATAAT
GCCCAGCTGCTGGAACTGGGTAGCGGCAATCTGCGCAAAATTGAA
ATTCTGCTGCAGGAATTTGAAGCCGCAAGCAAAAAAGTTGATTAT
TATGCACTGGATCTGAGTCTGAGCGAACTGGAACGTACCTTTAGC
GAAGTGAGCCTGGATCAGTATCAGTATGTTAAACTGCATGGTCTG
CATGGTACCTATGATGATGCACTGACCTGGCTGGAAAATCCGGCA
AATCGTAAAGTGCCGACCGTTATTATGAGTATGGGTAGCAGCATT
GGCAATTTTGATCGTCCGGCCGCCGCAAAATTTCTGAGCCAGTTT
GCACGCCTGCTGGGCCCGAGCGATCTGATGGTGCTGGGTCTGGAT
AGCTGCACCGATAGTGATAAAGTTTATAAAGCCTATAACGACAGT
AAGGGCATTACCCGCCAGTTTTATGAAAATGGCCTGCTGCATGCA
AATGCCGTGCTGGGCTATGAAGCCTTTAAACTGGATGAATGGGAT
ATTGTGACCGAATATGATAATGTTGAAGGCCGCCATCAGGCCTTT
TATGCACCGAATCGTGATGTTACCATTAATGGTGTGCTGCTGCAG
AAAGGTGAAAAACTGATTTTTGAAGAAGCATTCAAATACGATCCG
GAACAGTGTGATCAGCTGTGGCATGATGCAGGTCTGATTGAAGAT
GCAGAATTTGGTAATGAAAGTGGTGACTATCTGATTCATGTTCTG
AGCAGCGCCAGTCTGAATTTTAGTACCCGTCCGAGTCAGTATGCC
GCACAGAGCATTCCGAGTTTTGAAGAATTTCAGAGTCTGTGGACC
GCATGGGATATTGTTACCAAAGCCATGGTTCCGCGCGAAGAACTG
CTGAGTAAACCGATTAAGCTGCGTAATGCCCTGATTTTCTATCTG
GGTCATATTCCGACCTTTCTGGATGTTCATCTGACCCGTGCCCTG
GGCGAAAAACCGACCCATCCGAAAAGCTATCGCCTGATTTTTGAG
CGTGGTATTGATCCTGATGTGGATGATCCGGAAAAATGCCATAGT
CATAGCGAAATTCCGGATGAATGGCCGGCACTGGGTGACATTCTG
GATTATCAGGTGCGTGTTCGCAGTCGCGTGCGTAGTATTTTTCAG
AAACATAATGTTGCCGAAAACCGTGTTCTGGGCGAAGCACTGTGG
ATTGGTTTTGAACATGAAGCAATGCATCTGGAAACCTTTCTGTAT
ATGCTGATTCAGAGCGAACGCACCCTGCCGCCGCCGGCAGTTCCT
AGACCGGATTTTCGTAAATTTTTCCATGATGCCCGTCAGGAAAGT
CGCCCGAATGAATGGTTTAGTATTCCGGAAAAAACCCTGAGCGTT
GGCCTGCATGATGATGGTCATAGCGTTCCGCGTGATAGCTTTGGC
TGGGATAATGAAAAACCGCAGCGTAAAATTACCGTGAAAGCATTT
GAAGCACAGGCCCGCCCGATTACCAATGGTGAATATGCAAAATAT
CTGCAGGCAAATCAGCTGCCGCAGAAACCGGAAAGTTGGGTTCTG
ATTAAGCCGGAAACCTATCCGACCTGTAATGGCGTGAGCCAGGAT
GGTAGTTATGCAACCAATGAGTTTATGGCCCATTTTGCCGTTCGT
ACCGTTTTTGGTAGTGTGCCGCTGGAACTGGCACAGGATTGGCCG
GTTATTGCAAGTTATGATGAACTGGCCAAATATGCAAAATGGGTG
GATTGTCGCATTCCGACCTTCGAAGAAGCCAAAAGCATCTATGCA
CATGCAGCACGTCTGAAAGAAACCAGTCATGGTCTGAATGGCCAT
AGCGAAACCAATGGTGTTAATGGCCATGAACATAGCGAAACAAAT
CCGCTGCGTCCGCGTACCCCGGATCATCAGCCGGTTCAGCATCCG
AGTCAGGAAAGTCTGCCGGTGTTTGTGGAACTGGATAATTGCAAT
GTGGGTTTTAAACATTGGCATCCGACCCCGGTTATTCAGAATGGC
GATCGCCTGGCAGGCCATGGTGAACTGGGCGGTGTTTGGGAATGG
ACCAGCACCGAACTGGCCCCGCATGAAGGTTTTGAAGCCATGCAG
ATATATCCGGGCTATACCAGTGATTTCTTTGATGGTAAACATAAT
ATCATCCTGGGCGGTAGTTGGGCAACCCATCCGCGTATTGCCGGC
CGCACCACCTTTGTTAATTGGTATCAGCGTAATTATCCGTATCCG
TGGGCCGGTGCCCGTCTGGTTCGCGATGTGtaa
<SEQ ID NO: 20; PRT;
A3Egt1; 1_XM_001397080.2;
Aspergillus niger>
MSPLCPVVKGVDIVDIRQNDVEFSLVNDIQRGIDPPAGTCRSMPT
MLLYDAQGLKLFEDITYLEEYYLTNAEIDVLRTHAKRIVERIPDN
AQLLELGSGNLRKIEILLQEFEAASKKVDYYALDLSLSELERTFS
EVSLDQYQYVKLHGLHGTYDDALTWLENPANRKVPTVIMSMGSSI
GNFDRPAAAKFLSQFARLLGPSDLMVLGLDSCTDSDKVYKAYNDS
KGITRQFYENGLLHANAVLGYEAFKLDEWDIVTEYDNVEGRHQAF
YAPNRDVTINGVLLQKGEKLIFEEAFKYDPEQCDQLWHDAGLIED
AEFGNESGDYLIHVLSSASLNFSTRPSQYAAQSIPSFEEFQSLWT
AWDIVTKAMVPREELLSKPIKLRNALIFYLGHIPTFLDVHLTRAL
GEKPTHPKSYRLIFERGIDPDVDDPEKCHSHSEIPDEWPALGDIL
DYQVRVRSRVRSIFQKHNVAENRVLGEALWIGFEHEAMH
LETFLYMLIQSERTLPPPAVPRPDFRKFFHDARQESRPNEWFSIP
EKTLSVGLHDDGHSVPRDSFGWDNEKPQRKITVKAFEAQARPITN
GEYAKYLQANQLPQKPESWVLIKPETYPTCNGVSQDGSYATNEFM
AHFAVRTVFGSVPLELAQDWPVIASYDELAKYAKWVDCRIPT
FEEAKSIYAHAARLKETSHGLNGHSETNGVNGHEHSETNPLRPRT
PDHQPVQHPSQESLPVFVELDNCNVGFKHWHPTPVIQNGDRLAGH
GELGGVWEWTSTELAPHEGFEAMQIYPGYTSDFFDGKHNIILGGS
WATHPRIAGRTTFVNWYQRNYPYPWAGARLVRDV
<SEQ ID NO: 21; DNA;
A4Egt1; 1_XM_003066635;
Coccidioides posadasii>
ATGGGTCTGGCAATGGGTGGTGTTAATATTATTGATATTCGCCGT
AATAACCTGAATAATAGCCTGGCCAAAGATGTGACCCGCGGCCTG
GACCCTAAAAATGGCACCCAGCGCAGTCTGCCGACCCTGCTGCTG
TATAATACCGAAGGCCTGCGCCTGTTTGAAGAAATTACCTATCTG
GATGAATACTATCTGACCAATGCCGAAATTGAAGTTCTGACCACC
CATGCCGTTAGTATTGTTGAACGTGTGCCGGAAAATAGTCAGCTG
GTTGAACTGGGCAGTGGTAATCTGCGCAAAGTTGAAATTCTGCTG
AATGAATTTGAGCGTACCAAAAAACCGGTTGAATATCTGGCCCTG
GATGTGAGTCTGGAAGAACTGAATCGCACCTTTGCCGAACTGCCG
AGTAAAAGCTATCAGTATGTTAAATGCAGCGGTCTGCTGGGCACC
TATGATGATGCACTGAGCTGGCTGAAACGTAGTGAAAATCGTCGC
AAACCGACCTGGGTTATGAGCATGGGCAGCAGCATTGGCAATTTT
ACCCGTAGCGAAGCAGCCCAGTTTCTGGGTGGTTTTGCCCGTACC
CTGGGTGCAGATGATGCCCTGCTGGTGGGTCTGGATAGCTGTAAA
GATCCGCAGAAAGTTTTTCGTGCCTATAATGATAGTAAGAATGTT
ACCCGTGAATTTTATCTGAATGGTCTGGCAAATGCAAATAGCATT
CTGGGCTTTGAAGCATTCAAACGTGAAGATTGGGATGTGGCCGGT
ATCTATGATGAAGTTGATGGCTGTCATAAAGCCTATTATACCCCG
ACCCGTGATGTTACCATTGAAGCATGGAGCTTTACCAAAGGTGAA
CGCATTTTCTTTGAACAGGCATTCAAATATGCAGAAAAAGAATAT
CAGGCACTGTGGCAGCAGGCAGGCCTGACCAGTACCGCACGTTTT
ACCAGTAGCACCGGTGACCATAATATTCATCTGCTGAGCAGTAGC
CCGTATATTCTGCCGACCCAGCCGGCAGAATATGCCGCAACCCTG
ACCCCGAGTCTGAAAGAATTTGAAGCACTGTGGAAACTGTGGGAT
ACCGTTACCACCGGCATGCTGCCGCGCAATGAACTGCTGAGCAAA
CCGATTAATCTGCGCAATGCACTGGTGTTTTATCTGGGCCATATT
CCGACCTTTCTGGATATGCATATTACCCGCGCCATTGATGGTCAG
CCGACCGAACCGAAAAGCTATTGGAGCATTTTTGAACGTGGCATT
GATCCGGATGTTGATGATCCGCGTAAATGCCATGATCATAGCGAA
ATTCCGGATGCATGGCCGCCGGTTGAAGAAATTCTGCAGTTTCAG
ACCACCGTGCGCAATCGTGCACGTAGTCTGCTGCAGAAAAGCCAG
CATACCACCAATCGCCGCATTCATGAAGCCCTGTGGATTGGTTTT
GAACATGAAGCAATGCATCTGGAAACCTTTCTGTATATGCTGCTG
CAGAGCGATAAAGTGTGTCCGCCGCCGGAAATTAGCACCCCGGAT
TTTGAATATCTGGCGATGCGCAGCGCCCAGGAAAGTGTTCCGAAT
GAATGGTTTATTGTTCCGGAACAGACCATTTGGATTGGTCTGGAT
GATCCGGACCCTACCCGCATTCCGTTTGGCAGTTTTGGTTGGGAT
AATGAAAAACCGCAGCGCACCGCAAAAGTTAGTAGCTTTGAAGCC
AAAGGCCGTCCGATTACCAATGGTGAATATTGCCGTTATCTGGAA
GCCAATAAGCTGGCCACCGTGCCGGCCAGTTGGACCCGTAGCAGT
AGTGGCTTTAGCGAACCGAATGGTCATGCCGCCAGCCATACCAAT
GGTACCAATGGTCGTGAAACCAGCGAAGCAAGTGCCTTTAGCCAG
CTGCTGAGTAAATATCATGTTCGCACCGTGTTTGGTCCGGTTCCG
CTGCGCTTTGCCCTGGATTGGCCGGTTATTGCAAGCTATAATGAA
CTGGAACGCTATGCAAATTGGGTTAATTGTCGCATTCCGACCTTC
GAAGAAGCACGCAGCATCTATCAGTATAGTGTGTTTCTGAAAGAT
CAGGAAATTGGTGTGCAGAGTAGCCTGATTGATGCAAGCAGCAAT
GATATGGAAGGTCCGCCGCGCGATCTGAATGGTTTTGTTGAACAT
CGTAATGGTCGTCCGCGTGCCCCGGATCATCAGCCGGTTAGCCAG
CCGCCGAGCACCCAGATGCCGGTGAATGTGGATCTGGATGGTTAT
AATGTGGGTTTTAAACATTGGCATCCGACCCCGGTTACCCAGAAT
GGTAATAAGCTGAGTGGTCAGGGTGGTATGGGTGGTGCCTGGGAA
TGGACCAGCAGCACCCTGGAAGCACATGAAGGTTTTAAAGCAATG
GATCTGTATCCGGCCTATACCGCCGATTTCTTTGATGGCAAACAT
AATATTGTGCTGGGCGGCAGCTGGGCAACCCATCCGCGTATTGCC
GGCCGCACCACCTTTGTGAATTGGTATCAGCGCAATTATCCGTAT
GTTTGGGCAGGTGCACGCCTGGTTCGCGATATTtaa
<SEQ ID NO: 22; PRT;
A4Egt1; 1_XM_003066635;
Coccidioides posadasii>
MGLAMGGVNIIDIRRNNLNNSLAKDVTRGLDPKNGTQRSLPTLLL
YNTEGLRLFEEITYLDEYYLTNAEIEVLTTHAVSIVERVPENSQL
VELGSGNLRKVEILLNEFERTKKPVEYLALDVSLEELNRTFAELP
SKSYQYVKCSGLLGTYDDALSWLKRSENRRKPTWVMSMGSSIGNF
TRSEAAQFLGGFARTLGADDALLVGLDSCKDPQKVFRAYNDSKNV
TREFYLNGLANANSILGFEAFKREDWDVAGIYDEVDGCHKAYYTP
TRDVTIEAWSFTKGERIFFEQAFKYAEKEYQALWQQAGLTSTARF
TSSTGDHNIHLLSSSPYILPTQPAEYAATLTPSLKEFEALWKLWD
TVTTGMLPRNELLSKPINLRNALVFYLGHIPTFLDMHITRAIDGQ
PTEPKSYWSIFERGIDPDVDDPRKCHDHSEIPDAWPPVEEILQFQ
TTVRNRARSLLQKSQHTTNRRIHEALWIGFEHEAMHLETFLYMLL
QSDKVCPPPEISTPDFEYLAMRSAQESVPNEWFIVPEQTIWIGLD
DPDPTRIPFGSFGWDNEKPQRTAKVSSFEAKGRPITNGEYCRYLE
ANKLATVPASWTRSSSGFSEPNGHAASHTNGTNGRETSEASAFSQ
LLSKYHVRTVFGPVPLRFALDWPVIASYNELERYANWVNCRIPTF
EEARSIYQYSVFLKDQEIGVQSSLIDASSNDMEGPPRDLNGFVEH
RNGRPRAPDHQPVSQPPSTQMPVNVDLDGYNVGFKHWHPTPVTQN
GNKLSGQGGMGGAWEWTSSTLEAHEGFKAMDLYPAYTADFFDGKH
NIVLGGSWATHPRIAGRTTFVNWYQRNYPYVWAGARLVRDI
<SEQ ID NO: 23; DNA;
A5Egt1; 1_XM_016386852;
Cladophialophora immunda>
ATGGCAACCCATACCCCGGCCGGTGTGCCGATTCTGGATATTCGT
AGCGATCAGAGCGATCAGAGTCTGCTGCATACCCTGAAACAGAGT
CTGAATCCGCCGAAAGGTCAGCCGCGTACCTTTCCGACCCTGCTG
CTGTATGATGAAAAAGGCCTGAAACTGTTTGAAGAAATTACCTAT
GTGGATGAATACTATCTGACCAATGCCGAAATTGATACCCTGACC
CGTCATGCCGGCAAAATTGTTGGCCGCATTCCGGATGGCGCACGT
CTGGTGGAACTGGGTAGTGGCAATCTGCGTAAAGTTAATATTCTG
CTGAAAGCATTTGAGGAAGCCAATAAGAATGTGGAATATTATGCC
CTGGATCTGAGCCTGAGCGAACTGAAACGTACCTTTGCACAGCTG
GATATTAATGCATTTCATCATGTTACCTTCCGTGCCCTGCATGGC
ACCTATGATGATGCCCTGCTGTGGCTGAAAGAAAGCGCCACCGAT
GCCCGCACCACCTGCGTGATGACCATGGGTAGTAGTCTGGGCAAT
TTTACCCGTGAAGAAGCCGCACAGTTTCTGGCAAGCTTTAAAAAA
GTTCTGGCAGCAAGCGATTATGTTATGGTTGGCATTGATGCATGT
CAGCAGCCGGATCGCGTTTTTCGCGCCTATAATGATAGCATGAAT
GTGACCGAACGCTTTTATCGTAATGGCCTGACCCATGCAAATAAT
ATTCTGGGTTATGAAGCATTTCGTCAGGATGAATGGCAGATTGAA
GGCGTGTATGATGAAAATCAGAATAAGCATCAGGCCAGTTATGTG
GCACTGAAAACCATTAATAATAAGGATTTCAGCTTCGAACAGGGC
GAAAAAGTGCATCTGGAAGATGCATTCAAATATAGCGAAGCCCAG
CGTGATGCACTGTGGCATGCCGCAGGTCTGATTCCGCAGACCGCA
TATAATAATAAGACCAATGATTACTACATCCACCTGCTGAGCCCG
AGTACCATTAATTTTCCGACCAAACCGGCCGAATTTGCCGCCAGC
CCGGTTCCGAGTCTGGATGATTGGCGTCAGCTGTGGGCCGCATGG
GATACCGTTACCAAAAGTATGGTTCCGAAAGATGAACTGCTGAAT
AAGCCGATTAAGCTGCGTAATGATCTGATTTTCTATCTGGGCCAT
ATTCCGACCTTTGCCGATATTCATTATATGAAAGCCACCAAAGAA
AAGGCCACCGATCCGGCCTATTATATGAGTATTTTTGAACGCGGC
ATTGATCCGGATGTTGATAATCCGGAACTGTGTCATGATCATAGT
GAAATTCCGGATAGCTGGCCGCCGCTGGAAGATATTCTGAATTAT
AGCCAGCGTGTGCGCGAACGTATTGTTGAAGGCATTCATAGCGGT
CGCGCCTATACCGATCGTCGTCTGAGTCGTGGCCTGTGGCTGGCA
TATGAACATGAAGCCATGCATCTGGAAACCTTTCTGTATATGCTG
CTGCAGAGCGAACGCGTGCTGCCGCCGCCTGGTGAAGGTCTGCCG
GATTTTCGTACCCTGGCCGCCGAAGCACGTGCAAATCGCACCAGC
AATCAGTGGCATCGTATTCCGGCCAGCAAAGTTAAAATTGGTCTG
GATGATCCGGAAAATAATTTTGGTCCGGATCGTTATTTTGGCTGG
GATAATGAACGCCCGAGCCGCACCGTGGCAGTGAATGAATTTGAA
GCCCAGAGCCGCCCGATTAGTAATGGCGAATATGCACGCTTTCTG
GAAGTTACCCATAAAGATAGCCTGCCGGCCAGCTGGACCGCAAGT
AAAGTGGGTGCCGTGCTGAATGGCACCAATGGCACCAACGGCGCC
AATGGCCATGTTCAGGATGAACTGGATATGGCAAGTCCGAGCTTT
GTGGAAGATAAAGCCATTCGCACCGTTTATGGCCTGATTCCGCTG
AAATATGCACTGGATTGGCCGGTTATGGCCAGCTATGATGAACTG
GCCGCATATGCAGAATGGAGCAATGGTCGCATTCCGACCCTGGAA
GAAGCCCGCAGTCTGTATCAGTATGTTGAAAATCAGGATAGTGTT
CTGCAGAAAGTGACCAGCAAACTGATTAGCGCCGTTAATGGTCAT
CTGAGCAATGATGGCGTTCAGGAAACCCCGCCGAGCACCCATCAG
AGCAATGGCGTTGTGAATGGCAGTGCAGATGGTCCGAGTCTGGAC
CCGAATGAACTGTTTGTGGAACTGGGCGATCGTAATGTGGCCTTT
CGTCATTGGCATCCGACCCCGGTTACCGGTAATGCAGATCGTCTG
CGTGGCCAGAGCGATCTGGGTGGTCTGTGGGAATGGACCAGTACC
CCGCTGGCCCCGCATGAAGGCTTTAAAGCCATGGATCTGTATCCG
GGCTATACCGCCGATTTCTTTGATAGTAAACATAATGTGTGCCTG
GGCGGCAGTTGGGCAACCGTTCCGCGTATTGCCCTGAAAAAGACT
TTTGTGAATTGGTATCAGCGCAATTATCCGTATGTTTGGTGCACC
GCACGCCTGGTGCGCGATGTTGTTGTTtaa
<SEQ ID NO: 24; PRT;
A5Egt1; 1_XM_016386852;
Cladophialophora immunda>
MATHTPAGVPILDIRSDQSDQSLLHTLKQSLNPPKGQPRTFPTLL
LYDEKGLKLFEEITYVDEYYLTNAEIDTLTRHAGKIVGRIPDGAR
LVELGSGNLRKVNILLKAFEEANKNVEYYALDLSLSELKRTFAQL
DINAFHHVTFRALHGTYDDALLWLKESATDARTTCVMTMGSSLGN
FTREEAAQFLASFKKVLAASDYVMVGIDACQQPDRVFRAYNDSMN
VTERFYRNGLTHANNILGYEAFRQDEWQIEGVYDENQNKHQASYV
ALKTINNKDFSFEQGEKVHLEDAFKYSEAQRDALWHAAGLIPQTA
YNNKTNDYYIHLLSPSTINFPTKPAEFAASPVPSLDDWRQLWAAW
DTVTKSMVPKDELLNKPIKLRNDLIFYLGHIPTFADIHYMKATKE
KATDPAYYMSIFERGIDPDVDNPELCHDHSEIPDSWPPLEDILNY
SQRVRERIVEGIHSGRAYTDRRLSRGLWLAYEHEAMHLETFLYML
LQSERVLPPPGEGLPDFRTLAAEARANRTSNQWHRIPASKVK
IGLDDPENNFGPDRYFGWDNERPSRTVAVNEFEAQSRPISNGEYA
RFLEVTHKDSLPASWTASKVGAVLNGTNGTNGANGHVQDELDMAS
PSFVEDKAIRTVYGLIPLKYALDWPVMASYDELAAYAEWSNGRIP
TLEEARSLYQYVENQDSVLQKVTSKLISAVNGHLSNDGVQETPPS
THQSNGVVNGSADGPSLDPNELFVELGDRNVAFRHWHPTPVTGNA
DRLRGQSDLGGLWEWTSTPLAPHEGFKAMDLYPGYTADFFDSKHN
VCLGGSWATVPRIALKKTFVNWYQRNYPYVWCTARLVRDVVV
<SEQ ID NO: 25; DNA;
M A6Egt1; 1_XM_008090310;
Glarea lozoyensis>
ATGACCATTCATAGCGCAACCAATGGTACCGCAAAACTGGGTAGC
CATACCAAACCGATGAGCAGCAGCAAAACCAATAATATTCAGCCG
AGTGCAAAAGATGAACTGACCAGCGGCGTTGATATTATTGATATT
CGTCATGATGCAGTTGAAATTAATCTGAAAGAAGAAATCGACAAG
CTGCTGCATCCGGCAGAAGGCCCGAAAAAACTGCCGACCCTGCTG
CTGTATGATGAACAGTATTATCTGACCAATGCAGAAATTGATGTG
CTGAAACGTAGTGCAGGTAGCATTGCAGATAGCATTCCGAGCGGC
AGCATGCTGGTGGAACTGGGCAGTGGCAATCTGCGTAAAGTTAGT
ATTCTGCTGCGTGCACTGGAAGCCGCCGGCAAAGAAATTGATTAT
TATGCCCTGGATCTGAGCCTGAGCGAACTGAAACGCACCCTGGAA
CAGGTTCCGAATTTTAAATATGTTAAGTGCCATGGCCTGCATGGT
ACCTATGATGATGGCCTGGATTGGCTGAAAGCCGCCGAACATGGC
AGTCGCACCAAAGTTGTTATGAGCCTGGGCAGCAGCATTGGTAAT
TTTAAACGCAGCGAAGCAGCAAGCTTTCTGCGCGGTTTTAGTGAT
GCCCTGGGCCCGAGCAATATGATGCTGATTGGTGTGGATGCAACC
AGCGATCCGAGTAAAGTGTATCATGCCTATAATGATCGTAAAGGT
ACCACCCATGAGTTTATTCTGAATGGTCTGACCAATGCCAATGGT
ATTCTGGGTGAAGATGCCTTTGATATTAAGGATTGGAAAGTTATT
GGCGAATATGTGTTTGATCATGAAGGTGGTCGTCATCAGGCATTT
TATGTGCCGAAAACCGATGTGACCTATAAAAATATTCTGCTGAAA
GCAGGCGAACGTATTCAGGTTGAAGAAAGTCTGAAATATAGCCTG
GATGATGCAACCCATCTGTGGGCCGCCAGTGGCCTGCGTGAAGTG
AGTCATTGGACCGCCAGTAGCGATGCCTATAATATTCATCTGCTG
CAGCCGAAAGAACGCATGGAATTTCATACCGCAAGTGCAGTGTAT
GCAGCAACCACCGTGCCGAGTGTTGATGATTGGCTGAATCTGTGG
AAAGTTTGGGATATTGTGACCCGTAAAATGATTCCGGAACAGGAT
CTGCTGGAAAAACCGATTAAGCTGCGTAATGCATGTATTTTCTAT
CTGGGCCATATTCCGACCTTTCTGGATATTCAGCTGTGTAAAGTT
AGTGGTGAACCGCCGTGCGAACCGAGTCATTATCCGAAAATTTTT
GAACGCGGTATTGATCCGGATGTTGATAATCCGGAACATTGTCAT
GCACATAGCGATATTCCGGATGAATGGCCGCCGCTGGAAGAAATT
CTGCAGTATCAGGAACAGGTTCGCGCAAAAGCACTGAAACTGACC
GCAGCAAGCAAAATTCCGCGCGAAATTGGCCGCGCCCTGTGGATT
GGTCTGGAACATGAAATTATGCATCTGGAAACCCTGCTGTATATG
CTGCTGCAGAGCGATCGCACCATTCCGCCGACCAGTCATACCCCG
AATTTTAAGGATGATGCCAAAGCCGCAGAAAGCGCACGCGTTGAA
AATGAATGGTTTGAAGTTCCGCGCCAGCAGATTACCATTGGCCTG
GAAGATCCGGAAGATAATAGCGGCGGTGACCGCCATTTTGGCTGG
GATAATGAAAAACCGCCGCGCCAGGTTCTGGTGCCGAGCTTTCTG
GCCAAAGGTCGTGCCATTACCAATGAAGAATATGCCCGCTATCTG
GAATATACCAATAAGCATGAAATCCCGGCAAGTTGGGCCGATGGT
GGTAGCACCGGCAGTGATACCAATGGCTTTTTCAATGGCGCAAAT
GGCTATAGTAATGGCCATAGTAATGGTGGTGAAAAGAAAAGTAGT
ACCAAAGCATTTCTGGAAGGCAAAAGCGTTCGCACCGTGTATGGT
CTGGTGCCGCTGCAGTATGCACTGGATTGGCCGGTGTTTGCAAGT
TATGATGAACTGGCAGGTTGTGCAGCATTCATGGGCGGCCGTATT
CCGACCGTGGAAGAAGCCCGCAGCATCTATAGTCATGTTGATGGC
CTGAAACTGAAAGAAGCCGAACAGCATCTGGGTAAAACCGTGCCG
GCCGTTAATGGTCATCTGGTGAATGATGGCGTTGAAGAAAGCCCG
CCGAGTCGTGCCGTGGTTAATAGTGGTCGTAGTCGCAATCTGTTT
GCAAATCTGGATGGTGCAAATGTTGGCTTTAAAAATTGGCATCCG
GTTGCCGTGACCGCCAATGGCGATAAACTGGCAGGCCAGGCCGAA
ATGGGTGGCGTTTGGGAATGGACCAGCAGCCCGCTGGTTAAACAT
GAAGGTTTTGAACCGATGCCGCTGTATCCGGCCTATACCAGCGAT
TTCTTTGATGGTAAACATAATATCGCCCTGGGCGGTAGTTGGGCA
ACCCATCCGCGCATTGCCGGTCGCAAAACCTTTGTGAATTGGTAT
CAGCGTAATTATCCGTATGCCTGGGCAGGCGCACGTCTGGTTCGC
GATATTtaa
<SEQ ID NO: 26; PRT;
M A6Egt1; 1_XM_008090310;
Glarea lozoyensis>
MTIHSATNGTAKLGSHTKPMSSSKTNNIQPSAKDELTSGVDIIDI
RHDAVEINLKEEIDKLLHPAEGPKKLPTLLLYDEQYYLTNAE
IDVLKRSAGSIADSIPSGSMLVELGSGNLRKVSILLRALEAAGKE
IDYYALDLSLSELKRTLEQVPNFKYVKCHGLHGTYDDGLDWLKAA
EHGSRTKVVMSLGSSIGNFKRSEAASFLRGFSDALGPSNMMLIGV
DATSDPSKVYHAYNDRKGTTHEFILNGLTNANGILGEDAFDIKDW
KVIGEYVFDHEGGRHQAFYVPKTDVTYKNILLKAGERIQVEESLK
YSLDDATHLWAASGLREVSHWTASSDAYNIHLLQPKERMEFHTAS
AVYAATTVPSVDDWLNLWKVWDIVTRKMIPEQDLLEKPIKLRNAC
IFYLGHIPTFLDIQLCKVSGEPPCEPSHYPKIFERGIDPDVDNPE
HCHAHSDIPDEWPPLEEILQYQEQVRAKALKLTAASKIPREIGRA
LWIGLEHEIMHLETLLYMLLQSDRTIPPTSHTPNFKDDAKAAESA
RVENEWFEVPRQQITIGLEDPEDNSGGDRHFGWDNEKPPRQVLVP
SFLAKGRAITNEEYARYLEYTNKHEIPASWADGGSTGSDTNGFFN
GANGYSNGHSNGGEKKSSTKAFLEGKSVRTVYGLVPLQYALDWPV
FASYDELAGCAAFMGGRIPTVEEARSIYSHVDGLKLKEAEQHLGK
TVPAVNGHLVNDGVEESPPSRAVVNSGRSRNLFANLDGANVGFKN
WHPVAVTANGDKLAGQAEMGGVWEWTSSPLVKHEGFEPMPLYPAY
TSDFFDGKHNIALGGSWATHPRIAGRKTFVNWYQRNYPYAWAGAR
LVRDI
<SEQ ID NO: 27; DNA;
A7Egt1; 1_XM_016369232;
Exophiala mesophila>
ATGACCCGCACCATTAGCCAGGTGCTGCATCCGGCCTTTACCATG
GCCACCAGCCAGCGTCCGGCCGTGCGTTTTCTGGATATTCGTGGT
GACAAAAGCGGCCAGAGTCTGCTGAGCATGCTGAAAGAAAGTCTG
GACCCTCCGAATAAGCAGCCGCGTAGTTTTCCGACCCTGCTGCTG
TATGATGAAAAAGGTCTGAAAATTTTCGAGGAAATTACCTATCTG
GATGAATATTATCTGACCAATGCAGAAATTGAAGCACTGGAAACC
CATGCCCGCGAAATTGCCACCCAGATTCCGCGCAATAGTCGCATT
GTGGAACTGGGTAGTGGTAATCTGCGTAAAATTAATATTCTGCTG
GAAGCCTTTGAAGCAGCAAAAAAGAATGTTGATTACTATGCACTG
GATCTGAGCTTTCCGGAACTGCAGCGTACCTTTGCCGAAATTGAT
ACCAGTCGTTATCAGCATGTGAGCTTTAATGCACTGCATGGCACC
TATGATGATGCCCTGACCTGGCTGAGTAATAGTAGTGGTGACCAG
AGTACCTGTGTTATGACCATGGGTAGCAGCCTGGGTAATTTTAGC
CGTCAGGATGCAGCAGGCTTTCTGACCAAAATTAAGAGCGTTCTG
GGTCCGGCCGATCTGATTCTGGTGGGTCTGGATGCCTGCCAGGAC
CCTCAGCGTGTTTTTAAAGCCTATAATGATAGTCAGCTGGTGACC
GAACGTTTTTATCGCAATGGTCTGGATCATGCCAATAGCCTGCTG
GGTTATGAAGTGTTTCGTCAGGAAGATTGGAGCGTTGAAGGCCGT
TATGATGAACAGCTGGATCGCCATCATGCAACCTATCTGGCCCGC
AAAGATATTATTACCAAAGATTTTAGCTTCAAGCGTGGCGAACGC
CTGCCGTTTGAAGAAAGCTTTAAATATAGCGAAGCACAGAGTGAT
CAGCTGTGGCATGATAGCGGTCTGGTGCAGCAGATGGCCTTTGGC
AATAAGAGCGCCGATTATTTTATTCATCTGCTGAGCCCGGCAGCA
ATTAATTTTGCCACCAAACCGGCCGAATATGCAACCAATCCGATT
CCGAGTAGCGATGATTGGCAGCAGCTGTGGACCGCATGGGATGTT
GTGACCCGTAGCATGATTCCGAAAGATGAACTGCTGAATAAGCCG
ATTAAGCTGCGTAATGATCTGATTTTCTATCTGGGCCATATTCCG
ACCTTTGCAGATATTCATTTTACCAAAGCAACCGATGGCAAACCG
ACCGAACCGGCCAGTTATTGGAGTATTTTTGAACGTGGCATTGAT
CCGGATGTTGATAATCCGGAACTGTGTCATGATCATAGTGAAGTT
CCGGATAGTTGGCCGGCACTGAATGATATTCTGACCTATGCCAAA
CGTGTGCGCAGTCGTATTGCAGATAGCCTGGAAAGCGGCCAGGCC
GTGCAGGATCGTCGCCTGGGTCGTGGTCTGTGGCTGGCCTATGAA
CATGAAGCCATGCATCTGGAAACCTTTCTGTATATGCTGCTGCAG
AGTGATCGCATTCTGCCGCCGCCGGGTACCGAACGCCCGGACTTT
CGCCAGATTGCAGATGAAGCACGTGCAAATCGCGTGGCCAATAAG
TTTCATCGCATTCCGGCAGCAGAAGTGACCCTGGGCCTGGATGAT
CCGGAAAATCATCATGGTCCGGATCGTTATTTTGGCTGGGATAAT
GAACGCCCGAGTCGCACCGTTAGCGTTGCAGCCTTTGAAGCGCAG
AGTCGTCCGATTAGCAATGGTGACTATGCATATTATCTGGAAGTT
ACCGGCAATAGCAGCCTGCCGGCAAGCTGGATTGCACGTCGCAGT
GTTCTGAATGGTGTGAATGGCGCAGTTAGTAATGGCGAAGTTGGC
CGTGAAGTTGTTAGTAGCGAATTTCTGGGCGATAAAGCACTGCGC
ACCGTTTGGGGTCCGCTGCCGCTGAAACATGCCCTGGATTGGCCG
GTTATGGCAAGCTATGATGAACTGGCCGCATATGCAAAATGGGCA
AATGGTCGTATTCCGACCCTGGAAGAAGCACGTAGTATCTATCAT
CATGTTGAAAGCCGCAAAGATACCCTGGAAAAAGTTCCGAGCAAA
CTGATTAGTGCAGTTAATGGCCATCTGAGCAATGAAGGCGTGGAA
GAAACCCCGCCGAGCAATCAGAGTAGCGGTGAAGCCGCCAATGGC
AGCCTGCCGCCGAATCCGAATGAACTGTTTGTGGATCTGAATAAT
TGCCCGGTTGGTTTTAAAACCTGGACCCCGCAGCCGATTACCCAT
AGCAGTAGCCTGCGTGGTCAGGCCGATGTTGGCGGCCTGTGGGAA
TGGACCAGTACCCCGCTGGCCCCGTATGATGGCTTTAAAGCCATG
GATCTGTATCCGGGCTATACCGCAGATTTCTTTGATGGTAAACAT
AATATCTGCCTGGGTGGCAGCTGGGCAACCATTCCGCGTATTGCA
GGTCGTAAAACCTTTGTTAATTGGTATCAGCGTAATTATCCGTAT
GTGTGGTGCACCGCCCGCCTGGTTCGCGATATTGCAGAAtaa
<SEQ ID NO: 28; PRT;
A7Egt1; 1_XM_016369232;
Exophiala mesophila>
MTRTISQVLHPAFTMATSQRPAVRFLDIRGDKSGQSLLSMLKESL
DPPNKQPRSFPTLLLYDEKGLKIFEEITYLDEYYLTNAEIEALET
HAREIATQIPRNSRIVELGSGNLRKINILLEAFEAAKKNVDYYAL
DLSFPELQRTFAEIDTSRYQHVSFNALHGTYDDALTWLSNSSGDQ
STCVMTMGSSLGNFSRQDAAGFLTKIKSVLGPADLILVGLDACQD
PQRVFKAYNDSQLVTERFYRNGLDHANSLLGYEVFRQEDWSVEGR
YDEQLDRHHATYLARKDIITKDFSFKRGERLPFEESFKYSEAQSD
QLWHDSGLVQQMAFGNKSADYFIHLLSPAAINFATKPAEYATNPI
PSSDDWQQLWTAWDVVTRSMIPKDELLNKPIKLRNDLIFYLGHIP
TFADIHFTKATDGKPTEPASYWSIFERGIDPDVDNPELCHDHSEV
PDSWPALNDILTYAKRVRSRIADSLESGQAVQDRRLGRGLWLAYE
HEAMHLETFLYMLLQSDRILPPPGTERPDFRQIADEARANRVANK
FHRIPAAEVTLGLDDPENHHGPDRYFGWDNERPSRTVSVAAFEAQ
SRPISNGDYAYYLEVTGNSSLPASWIARRSVLNGVNGAVSNGEVG
REVVSSEFLGDKALRTVWGPLPLKHALDWPVMASYDELAAYAKWA
NGRIPTLEEARSIYHHVESRKDTLEKVPSKLISAVNGHLSNEGVE
ETPPSNQSSGEAANGSLPPNPNELFVDLNNCPVGFKTWTPQPITH
SSSLRGQADVGGLWEWTSTPLAPYDGFKAMDLYPGYTADFFDGKH
NICLGGSWATIPRIAGRKTFVNWYQRNYPYVWCTARLVRDIAE
<SEQ ID NO: 29; DNA;
A8Egt1; 1_XM_018400273;
Fusarium oxysporum>
ATGCCGAGTATTACCGCCAGCAGTGCAACCCCGCAGCGTGTTAAA
CCGACCACCGCAAAACCGAGCAGTGCCCTGCCGAGTATTATTGAT
ATTCGTGGTGAACATGTTGAAATTAATCTGAAAGATCAGATCGCA
AGCATGTTTAATCCGGATGAAGGTCCGCGCAAACTGCCGACCCTG
CTGCTGTATAATGAAAAAGGTCTGCAGATTTTTGAGGATATTACC
TATCTGGATGAATATTATCTGACCAATTACGAAATCGAAATCCTG
AAAAAATCCAGCGCCGAAATTGCAAGTCAGATTCCGGAAGGTAGT
ATGGTTATTGAACTGGGCAGTGGCAATCTGCGTAAAGTGTGTCTG
CTGCTGCAGGCCTTTGAAGAACTGAAAAAACCGATTCAGTATTTT
GCACTGGATCTGAGTCTGAAAGAACTGGAACGCACCCTGGCCCAG
GTGCCGGAGTTTAAATATGTTAGTTGCCATGGTCTGCATGGCACC
TATGATGATGGCCGTGAATGGCTGAAACATCCGAGTCTGACCAGT
CGCAGTAAATGCATTATTCATCTGGGCAGCAGTATTGGCAATTTT
ACCCGTGATGGTGCAGCCGATTTTCTGGGCGGTTTTGCCGAAGTG
CTGACCCCGAGTGATAGTATGATTGTTGCAGTTGATAGTTGTAGC
AATCCGGCACAGGTGTATCATGCCTATAATGATAGTAAAGGCGTG
ACCCATCAGTTTGTGCTGAATGGTCTGCAGAATGCCAATGAAATT
CTGGGCGAAGAAGCATTCAATACCGATGAATGGCGCGTTATTGGT
GAATATGTTTATGATGTTGAGGGCGGTCGTCATCAGGCCTTTCTG
AGTCCGACCCGCCCGACCGATGCCCTGGGTAGTCGCGTGCTGCCG
CATGAACGCATTGAAATTGAACAGAGCCTGAAATATAGTGAAGCA
GAAAAAGATAAGCTGTGGAAACTGGCCGGTCTGACCGAAATGGGT
CGCTGGAGCCGCGGCGATGAATATGGTCTGCATTTTCTGCAGAAA
AGCAGCATGCCGTTTAGTCGCATTCCGAGCCTGTATGCCGCCGAA
CCGCTGCCGACCGTTCAGGATTGGAAAGCCCTGTGGAAAGCATGG
GATGTTGTGACCAAAGATATGATGCCGGATGAAGAACTGCAGGAA
AAACCGATTAAGCTGCGCAATGCATGTATTTTCTATCTGGGCCAT
ATTCCGACCTTTCTGGATATTCAGCTGACCAAAACCACCGGTAAT
GCCCCGACCGAACCGGCCACCTATTATAGTATTTTTGAACGCGGC
ATTGATCCGGATGTGGATAATCCGGAACATTGTCATACCCATAGC
GAAATTCCGGATGAATGGCCGCTGGTGCAGGAAATTATGATCTAT
CAGGATCGCGTTCGTAGTCGCCTGCAGAATCTGTATAAAAATGGC
CAGGATAAAATCAGTCGCGATATTGGTCGCGCAATTTGGGTGGGC
TTTGAACATGAACTGATGCATATTGAAACCCTGCTGTATATGATG
CTGCAGAGTGATCGCACCCTGCCGCCGCCGCATACCGTGCAACCG
GATTTTGCAAAACTGGCCCAGCAGGCCCATGAAGCCCGCGTGCCT
AATCAGTGGTTTGATGTTCCGGAACAGACCATTACCCTGGGCATG
GATGATCCGGAAGATGGTACCGATAATAGTCGTCATTTTGGCTGG
GATAATGAAAAACCGGAACGTCAGACCAAAGTTCATGCATTTCGC
GCACAGGGTCGCGCAATCACCAATGAAGAATATGCCCAGTATCTG
TATAATAGCAAAATTGAACACATCCCGGCCAGCTGGAGTAGCGTT
ACCAATGCCTATACCAATGGCGCAACCAATGGCAGCCATGCCAAT
GGTAATAGCAATGGCTATAGCAATGGCAATGGTCATCATGCAAGC
CAGGTTCCGGATAGCTTTATTCAGGATAAATTTGTTAAGACCGTG
TATGGTCTGGTGCCGCTGAAATATGCACTGGATTGGCCGGTGTTT
GCCAGCTATGATGAACTGGCAGGCTGTGCAGCATGGATGGGCGGT
CGCATTCCGACCTTCGAAGAAGCCAAAAGTATCTATGCACATGTG
AATAAGCAGAAACGTGCAGAAGCAGAACGCACCTTAAGCAAAACC
GTGCCGGCCGTGAATGGTCATCTGGTGAATGATGGTGTGGAAGAA
ACCCCGCCGAGTAATAGCAGCGCCCTGGTGAAAGATAGCAGTAGT
GAACTGTTTTTCGATCTGACCGGTGCCAATGTTGGTTTTCGCCAT
TGGCATCCGATGCCGGTGACCAGCCGTGGTAATAAGCTGGCCGGT
CAGAGTGAAATGGGCGGCGTGTGGGAATGGACCAGCAGTAGCCTG
GAACGTCATGAAGGCTTTGAACCGATGAGTCTGTATCCGCTGTAT
ACCACCGATTTCTTTGATGGTAAACATAATGTTGTGCTGGGCGGC
AGCTGGGCAACCCATCCGCGTATTGCAGGCCGCGCAAGCTTTGTG
AATTGGTATCAGCGTAATTATCCGTATGCATGGGTTGGTGCACGC
CTGGTGCGCGATGTTtaa
<SEQ ID NO: 30; PRT;
A8Egt1; 1_XM_018400273;
Fusarium oxysporum>
MPSITASSATPQRVKPTTAKPSSALPSIIDIRGEHVEINLKDQIA
SMFNPDEGPRKLPTLLLYNEKGLQIFEDITYLDEYYLTNYEIEIL
KKSSAEIASQIPEGSMVIELGSGNLRKVCLLLQAFEELKKPIQYF
ALDLSLKELERTLAQVPEFKYVSCHGLHGTYDDGREWLKHPSLTS
RSKCIIHLGSSIGNFTRDGAADFLGGFAEVLTPSDSMIVAVDSCS
NPAQVYHAYNDSKGVTHQFVLNGLQNANEILGEEAFNTDEWRVIG
EYVYDVEGGRHQAFLSPTRPTDALGSRVLPHERIEIEQSLKYSEA
EKDKLWKLAGLTEMGRWSRGDEYGLHFLQKSSMPFSRIPSLYAAE
PLPTVQDWKALWKAWDVVTKDMMPDEELQEKPIKLRNACIFYLGH
IPTFLDIQLTKTTGNAPTEPATYYSIFERGIDPDVDNPEHCHTHS
EIPDEWPLVQEIMIYQDRVRSRLQNLYKNGQDKISRDIGRAI
WVGFEHELMHIETLLYMMLQSDRTLPPPHTVQPDFAKLAQQAHEA
RVPNQWFDVPEQTITLGMDDPEDGTDNSRHFGWDNEKPERQTKVH
AFRAQGRAITNEEYAQYLYNSKIEHIPASWSSVTNAYTNGATNGS
HANGNSNGYSNGNGHHASQVPDSFIQDKFVKTVYGLVPLKYALDW
PVFASYDELAGCAAWMGGRIPTFEEAKSIYAHVNKQKRAEAERTL
SKTVPAVNGHLVNDGVEETPPSNSSALVKDSSSELFFDLTGANVG
FRHWHPMPVTSRGNKLAGQSEMGGVWEWTSSSLERHEGFEPMSLY
PLYTTDFFDGKHNVVLGGSWATHPRIAGRASFVNWYQRNYPYAWV
GARLVRDV
<SEQ ID NO: 31; DNA;
B1Egt1; 1_XM_003048838;
Nectria haematococca>
ATGCCGAGCAGCGTGAATGCACCGCCGGCCGTTTTTCAGGGTGCA
CGTCCGGCAGTGAGCAAACCGAGCCCGGCACTGCCGGATATTATT
GATATTCGCGGCGAACATGTTGAAATTAATCTGAAAGATCAGATC
ATCAGCCAGTTTAATCCGGAAGATGGTCCGCGTAAACTGCCGACC
CTGCTGCTGTATAATGAAAAAGGTCTGCAGATTTTTGAGGATATT
ACCTATCTGGATGAATATTATCTGACCAATTACGAAATCGAAGTG
CTGAAACGCAGTAGTACCGAAATTGCACGTCAGATTCCGGAAGGC
AGCATGGTTATTGAACTGGGTAGCGGTAATCTGCGTAAAGTGTGT
CTGCTGCTGCAGGCCTTTGAAGATTTGGCCAAACCGATTCAGTAT
TTTGCCCTGGATCTGAGCCGCAAAGAACTGGAACGTACCCTGGCC
CAGGTGCCGGATTTTAAATATGTGAGTTGCCATGGCCTGCTGGGC
ACCTATGATGATGGTCGTGAATGGCTGAAACATCCGAGTCTGACC
GGTCGCAGTAAATGCATTCTGCATCTGGGCAGTAGCATTGGTAAT
TTTAGCCGTGATGAAGCAGCAGCCTTTCTGGGCGGCTTTGCCGAT
GTTCTGCGCCCGAGTGATAGCATGATTGTTGGCGTGGATGCATGC
AATAATCCGGCAAAAGTTTATAAACCGATTATGAATCAGCCGCGT
CTGAGTCGTACCAATCGCATTCATCGCTTTATTCTGAATGGTCTG
AGTCATGCCAATGAACTGCTGAGCGAAGAAGCCTTTAAAGTTGAA
GAATGGCGTGTTATTGGCGAATATGTTTATGATGATGAAGGTGGC
CGCCATCAGGCCTTTGTTGCCCCGACCCGCCCGACCGATGTGCTG
GGTAGCCGCGTTATGCCGCATGAACGCATTGAAATTGAACAGAGT
CTGAAATATAGCGATGAAGAAACCATGACCCTGTGGGCCCAGAGC
GGTCTGACCGAAATGGGTCGTTGGAGTCGCGGCGATGAATATGGT
CTGCATATGCTGCAGAAAAGCGCAATGCCGTTTAGTCTGATTCCG
AGCCTGTATGCCGCCGAACCGCTGCCGGCAGTTCAGGATTGGGAA
GCACTGTGGAAAGCCTGGGATGTTGTGACCCAGGGTATGCTGCCG
CATGAAGAACTGAATGAAAAACCGATTAAGCTGCGCAATGCCTGT
ATTTTCTATCTGGGTCATATTCCGACCTTTCTGGATATTCAGCTG
ACCAAAACCACCGGTCAGGCCCCGACCGATCCGGCTTATTATTAT
AGCATTTTTGAACGCGGTATCGATCCGGATGTGGATAATCCGGAA
CTGTGCCATACCCATAGCGAAATTCCGGATGAATGGCCGCCGGTT
GGCGAAATTATTGAATATCAGGGTCGTGTGCGCAGCCGCGTGAAA
GCACTGTATCGCGATGGCGCCAGTAAAATTCCGCGCCATATTGCA
CGTGCAATTTGGGTTGGCTTTGAACATGAACTGATGCATATTGAA
ACCCTGCTGTATATGATGCTGCAGAGTGATAAAACCCTGCCGCCG
CCGCATACCGCCCAGCCTAATTTTGAAAAAATGGCAAAACAGGCC
TATGAAGCACGTGTGCCGAATCAGTGGTTTAATGTTCCGGAACAG
ACCATTACCCTGGGTATGGATGATCCGGAAGATTGCACCGATACC
AGTGGCCATTTTGGCTGGGATAATGAAAAACCTGCCCGTCAGGCC
AAAGTTCATACCTTTCAGGCACAGGGCCGCCCGATTACCAATGAA
GAATATGCACAGTATCTGTATAGTACCAAAACCAATAGCGTTCCG
GCCAGCTGGAGCTGGGACCCTAGTAAAACCGTGAATGGTAGCGCA
AATGGTAGCTATACCAATGGCCATAGCAATGTGCCGGATAGTTTT
CTGGAAGGCAAATATGTTCGCACCGTTTATGGCCTGGTGCCGCTG
AAACATGCCCTGGATTGGCCGGTTTTTGCAAGCTATGATGAACTG
AGTGGTTGCGCAAGCTGGATGGGCGGCCGTATTCCGACCTTCGAA
GAAGCCAAAAGCATCTATGCATATGTGAATAAGCAGAAACGCGCC
GATGCAGAACGCATTCTGAGTAAAACCGTTCCGGCAGTGAATGGT
CATCTGGTTAATGATGGTGTGGAAGAAACCCCGCCGAGCCAGAGT
AGTGAAAGTGCCAAAGATAGCCCGAGCAAACTGTTTCTGGATCTG
GCAGATGCCAATGTGGGCTTTCGTCATTGGCATCCGATGCCGGTT
ACCAGTCAGGGTAATCGTCTGGCAGGTCAGAGCGAAATGGGTGGC
GTTTGGGAATGGACCAGTAGCAATCTGAAACCGCATGAAGGCTTT
GAACCGATGAGTCTGTATCCGCTGTATACCACCGATTTCTTTGAT
GGCAAACATAATATTGTGCTGGGCGGCAGTTGGGCCACCCATCCG
CGTATTGCAGGCCGCGCAAGTTTTGTTAATTGGTATCAGCGTAAT
TACCCGTATGCATGGGTGGGTGCCCGCCTGGTTCGTGATATTtaa
<SEQ ID NO: 32; PRT;
B1Egt1; 1_XM_003048838;
Nectria haematococca>
MPSSVNAPPAVFQGARPAVSKPSPALPDIIDIRGEHVEINLKDQI
ISQFNPEDGPRKLPTLLLYNEKGLQIFEDITYLDEYYLTNYEIEV
LKRSSTEIARQIPEGSMVIELGSGNLRKVCLLLQAFEDLAKPIQY
FALDLSRKELERTLAQVPDFKYVSCHGLLGTYDDGREWLKHPSLT
GRSKCILHLGSSIGNFSRDEAAAFLGGFADVLRPSDSMIVGVDAC
NNPAKVYKPIMNQPRLSRTNRIHRFILNGLSHANELLSEEAFKVE
EWRVIGEYVYDDEGGRHQAFVAPTRPTDVLGSRVMPHERIEIEQS
LKYSDEETMTLWAQSGLTEMGRWSRGDEYGLHMLQKSAMPFSLIP
SLYAAEPLPAVQDWEALWKAWDVVTQGMLPHEELNEKPIKLRNAC
IFYLGHIPTFLDIQLTKTTGQAPTDPAYYYSIFERGIDPDVDNPE
LCHTHSEIPDEWPPVGEIIEYQGRVRSRVKALYRDGASKIPRHIA
RAIWVGFEHELMHIETLLYMMLQSDKTLPPPHTAQPNFEKMAKQA
YEARVPNQWFNVPEQTITLGMDDPEDCTDTSGHFGWDNEKPARQA
KVHTFQAQGRPITNEEYAQYLYSTKTNSVPASWSWDPSKTVNGSA
NGSYTNGHSNVPDSFLEGKYVRTVYGLVPLKHALDWPVFASYDEL
SGCASWMGGRIPTFEEAKSIYAYVNKQKRADAERILSKTVPAVNG
HLVNDGVEETPPSQSSESAKDSPSKLFLDLADANVGFRHWHPMPV
TSQGNRLAGQSEMGGVWEWTSSNLKPHEGFEPMSLYPLYTTDFFD
GKHNIVLGGSWATHPRIAGRASFVNWYQRNYPYAWVGARLVRDI
<SEQ ID NO: 33; DNA;
B2Egt1; 1_XM_022728240;
Penicilliopsis zonata>
ATGAGCCCGAGTGTGTGCCCGGCAAATAATGTTGAAATTGTTGAA
ATTCGCCAGGAAAATTTTGAATTTTCACTGGCAGAAGATATCTAT
AATGGTATTAAGCTGAGCGAAAATGGCACCCGTAGTCTGCCGACC
ATGCTGCTGTATGATGCAAAAGGCCTGAATCTGTTTGAAGAAATT
ACCTATCTGGATGAATACTATCTGACCAATACCGAAATTGAAGTT
CTGGAAACCCATGCCCAGCGTATTGTTGAACGCATTCCGGCCAAT
GCACAGCTGGTGGAACTGGGCAGCGGTAATCTGCGTAAAATTGAA
ATTCTGCTGAAAGAATTTGAGCGTACCGAAAAACATGTTCATTAT
TATGCACTGGATCTGAGCCTGAGCGAACTGAAACGTACCTTTAGT
GAAATTCCGGTTGATCAGTTTGAATTTGTGAAACTGCATGGCCTG
CATGGTACCTATGCCGATGCCCTGACCTGGCTGAGTAATCCGAAA
AATCGCACCCGTCCGACCGGCGTGATTAGTATGGGTAGTAGCCTG
GGTAATTTTAGCCGTCCGGATGCCGCCAGCTTTCTGCATGGTTTT
AGTCGCCTGCTGGGTCCGAGCGATTTTATGGTTCTGGGCCTGGAT
GGCTGTAAAAATACCGATAAAGTTTATAAGGCCTACAATGATAGT
CGTGGTGTGACCCGTCAGTTTTATGAAAATGGTCTGGCACATGCC
AATGAAGTTCTGGGTTATGAAGCATTCAAACCGAGCGAATGGGAA
ATTGTTACCCGTTATAATGAAGAAGGTGGTCTGCATCAGACCTTT
GTTCGTCCGAAATGCGATGTGACCATTAATGGCATTAAGATTAGT
AAAGGTGAGAATCTGCTGTATGAAGAAGCATTCAAATATGATCCG
GCCGAACGCGAAAGCCTGTGGCGTGATGCCGGTCTGATTCATAAT
GTTGCCTTTGGCAATACCAGTGATGATTATCATATTCATATGCTG
AGCCCGGCAAATCTGGATCTGCCGACCAATCCGCTGGAATATGCA
GCCAGCCCGATTCCGCGCATGGAAGAATTTCAGAGCCTGTGGACC
GCCTGGGATATTGTGACCAAAGGTATGGTTCCGGGCGCCGAACTG
CTGAGCAAACCGATTAATCTGCGTAATGTTCTGCTGTTTTATCTG
GGTCATATTCCGACCTTTAGTGATATTCATGTTACCCGCGCACTG
CGTGGTAAACTGACCGAACCGCGTCATTATCAGCTGATTTTTGAA
CGTGGTATTGATCCGGATGTTGAAGATCCGGAACAGTGTCATGCC
CATAGTGAAATTCCTGATCAGTGGCCGCCGCTGGTTGAAATTCTG
GAATATCAGTGGAAAGTGCGCAGCCGTATTCAGAGCGTTCTGCAG
AGTGGCGGCCTGAAACATAATCGCACCCTGGGCGAAGCCCTGTGG
ATTGGTTTTGAACATGAAATTATGCATCTGGAAACCTTTCTGTAT
ATGCTGCTGCAGAGTGATAAAACCCTGCCGCCGCCGGGTGTTGAT
ACCCCGGATTTTGAAAAAATTTTTCGTGAAGCACGTAAGCAGGCA
ACCCCGAATCAGTGGTTTGTTGTTCCGGAACAGACCCTGCTGATT
GGTCTGGATGATAAAGATGATGGTGTTATTCCGCCGGTTAGCTTT
GGTTGGGATAATGAAAAACCGCAGCGTACCGCCGCCGTGAGCGCA
TTTGAAGCACAGGGCCGTGCAATTACCAATGGTGAATATGCCGAA
TATCTGGAAGCCAATCATATTGAACAGATTCCGGCCAGCTGGGTT
CTGGCAGGTTTTAATGGCGCCTGCCATGTTAGCAATAAGAGTGGC
AGCAATAGTAGCCTGCGTCCGAATGGTTTTCTGAGTCTGTATACC
GTTCGTACCGTTTTTGGTCCGGTTAGCCTGGAACTGGCCCAGGAT
TGGCCGGTTGTGGCCAGTTATGATGAACTGGCAGGTTATGCAGAA
TGGGTTAAATGCCGCATTCCGACCTATGAAGAAGTGCGTAGTATC
TATCAGTATAGTGAACAGCTGAAACATGCCACCACCCCGCCGAAA
ACCAATGGCCATGGCACCAGTAATGAAAGCAATCGTATTAAGGGT
GAAACCAATGGCACCAAACCGTATAGCAAAGATCATCATCAGCCG
GTTCGCCCGCCGGTTAGTAGCACCAGCCCGGTGTTTCTGGATATT
GAAGGTTGCAATGTGGGTTTTAAACATTGGCATCCGACCCCGGTT
ATTCAGAATGGTAATAAGCTGGCAGGTCAGAGCGAACTGGGCGGT
GTGTGGGAATGGACCAGTACCCCGCTGGTTCCGCATGATGGTTTT
AAACCGATGGATGTTTATCCGGGTTATACCGCCGATTTCTTTGAT
GGCAAACATAATATTGTGCAGGGCGGTAGCTGGGCAACCCATCCG
CGCATTGCAGGTCGTACCAGTTTTGTTAATTGGTATCAGCATAAT
TACCCGTATGCCTGGGCCGGCGCCCGTCTGGTTCGCGACTTAtaa
<SEQ ID NO: 34; PRT;
B2Egt1; 1_XM_022728240;
Penicilliopsis zonata>
MSPSVCPANNVEIVEIRQENFEFSLAEDIYNGIKLSENGTRSLPT
MLLYDAKGLNLFEEITYLDEYYLTNTEIEVLETHAQRIVERIPAN
AQLVELGSGNLRKIEILLKEFERTEKHVHYYALDLSLSELKRTFS
EIPVDQFEFVKLHGLHGTYADALTWLSNPKNRTRPTGVISMGSSL
GNFSRPDAASFLHGFSRLLGPSDFMVLGLDGCKNTDKVYKAYNDS
RGVTRQFYENGLAHANEVLGYEAFKPSEWEIVTRYNEEGGLHQTF
VRPKCDVTINGIKISKGENLLYEEAFKYDPAERESLWRDAGLIHN
VAFGNTSDDYHIHMLSPANLDLPTNPLEYAASPIPRMEEFQSLWT
AWDIVTKGMVPGAELLSKPINLRNVLLFYLGHIPTFSDIHVTRAL
RGKLTEPRHYQLIFERGIDPDVEDPEQCHAHSEIPDQWPPLVEIL
EYQWKVRSRIQSVLQSGGLKHNRTLGEALWIGFEHEIMHLETFLY
MLLQSDKTLPPPGVDTPDFEKIFREARKQATPNQWFVVPEQTLLI
GLDDKDDGVIPPVSFGWDNEKPQRTAAVSAFEAQGRAITNGEYAE
YLEANHIEQIPASWVLAGFNGACHVSNKSGSNSSLRPNGFLSLYT
VRTVFGPVSLELAQDWPVVASYDELAGYAEWVKCRIPTYEEVRSI
YQYSEQLKHATTPPKTNGHGTSNESNRIKGETNGTKPYSKDHHQP
VRPPVSSTSPVFLDIEGCNVGFKHWHPTPVIQNGNKLAGQSELGG
VWEWTSTPLVPHDGFKPMDVYPGYTADFFDGKHNIVQGGSWATHP
RIAGRTSFVNWYQHNYPYAWAGARLVRDL
<SEQ ID NO: 35; DNA;
B3Egt1; 1_XM_014676787;
Penicillium digitatum Pd1>
ATGAGTCCGACCCTGCTGCGCAATGTTGATACCGTGGAAATTGTG
AATATTCATCAGTGTGATATGGAATTTTCCCTGGTGGATGATGTT
TATAAAAATCTGGACCCTCCGGCAGGTAAACAGCGTACCTTTCCG
ACCCTGTTACTGTATGATGCCAAAGGTCTGAAACTGTTTGAAGAA
ATTACCTATCTGGATGAATACTATCTGACCAATACCGAAATTGAA
ATTCTGAAAAAGCACGCAAAAAAGATTGTTGCACATATTCCGGAA
AATGCCCAGCTGGTTGAACTGGGCAGTGGCAATCTGCGTAAAATT
GAAATTTTACTGCGTGAATGCGAACGCAGTGAAAAGAAAGTGGAT
TATTATGCCCTGGATCTGAGCCTGGGTGAACTGCAGCGTACCTTC
AGCGAAATTAGTCCGGAAAGTTTTATTCATGTTGGCTTTCATGGT
CTGCATGGTACCTATGATGATGCCGTGGGTTGGCTGAAAAGTCCG
GAAAATCGTAAACGCCCGACCCTGGTTCTGAGCATGGGCAGCAGT
ATGGGCAATTTTAGTCCGCCGGATGCAGCCGATTTTCTGGGTGGC
TTTAGTAAACTGCTGGGTCCGAGTGATTTTCTGCTGGTGGGTCTG
GATGCATGCAAAAATCCGGAAAAAGTTTTTCGTGCCTATAATGAT
AGCAAAGGTATTACCCGCAAATTTTATGAAAACGGCCTGCTGCAT
GCAAATCGTGTGCTGGGCTTTAAAGCCTTTAAAGCAGATGAATGG
GAAATTCTGACCGATTATGATAATCGTGAAGGTCGTCATCAGGCC
TTTTATGTTAGCAAAGTTGATGTGATTATCAACGGCATTAAGATT
CGTAAAGGTGAAAAACTGATCTTTGAAGAAGCATGGAAATATGGT
CGTAATGAACGCGATCAGCTGTGGCGTAATGCAAATCTGATTAGC
CAGGTGGAATTTGGCAATAGCACCGATGATTATCATCTGCATCTG
CTGAGTCCGGCCGCCCTGGATCTTAGCATGAATCCGAGCAAATAT
GCAGCACAGCCGATTCCGAGTATTGAAAATTTTCAGAGTCTGTGG
ACCGCATGGGATCTGGCAACCCGCACCATGGTGCCGCATGAAGAA
CTGCTGAGCCAGCCGATTAAGCTGCGTAATGCCCTGATTTTCTAT
TTTGGTCATATTCCGACCTTTCTGGATATTCATCTGACCCGTGCC
CTGCAGGAAGAAAGTACCGAACCGAGCAATTATAAAACCATTTTT
GAACGTGGTATCGATCCGGATGTTGAAGATCCGCAGCAGTGTCAT
AGTCATAGCGAAATTCCGGATGAATGGCCGCCGCTGGATGAAATT
CTGGATTATCAGGATCGTGTTCGTAATCGTGCCCTGAGCATTCTG
CAGCAGGGCTATGCAAGCCAGGATCGCGCCCTGGGTGAAGCACTG
TGGATTGGTTATGAACATGAAGCAATGCATCTGGAAACCTTTCTG
TATATGCTGATTCAGAGCGATAAAACCCTGCCGCCGACCGGTGTT
GATCGTCCGGATTTTGAACAGATTAATCGCCAGGCAAAAATTAAT
AAGAAGCCGAATAAGTGGTTCCGCATTCCGCGTCAGACCATTGAA
ATTGGCCTGAATGATAGCAATGAAGAAGTTGTTCCGAATCAGAGT
TTTGGTTGGGATAATGAAAAACCGCAGCGCAAAGTGACCGTGCAT
GCATTTGAAGCCCAGGCCCGTCCGATTACCAATGGTGAATATGCA
AAATATATCCAGGATAAAGGTATCAAAACCTATCCGGCAAGTTGG
GTTTTTAAACCGAGCCAGGATAATCCGGTGAGCAAAGGCATTAGC
AGCAGTGATGCCCAGGCCGGTAGTAGCAGTAGCCCGGCCGGTCTG
AGCCTGAAAGATATTACCGTTCGTACCGTGTTTGGTCCGGTTGCA
CTGGAAGTTGCCCAGGATTGGCCGCTGGCAGCCAGCTATGATGAA
GTGGCCAGTTATGCCAAATGCATGAAATGTCGTATTCCGACCTTC
GAAGAAACCCGTAGTATCTATCATTATAGTGATCAGCTGAAAGGT
GACCGTGTGACCAATGATCATCGTAATGGTGTGAATGGCCTGGCA
AATGATAGCAAGCCGAATAGCACCGACCAGACCGTTTTTCGCGAT
CTGACCGGTTGCAATGTTGGTTTTAATAATTGGCATCCGATTCCG
GTTACCAGCAATGGCGATCAGCTGGCCGGTCAGGGTGAAATGGGC
GGTGTGTGGGAATGGACCAGTACCCCGCTGATGCCGCATGATGAT
TTTAAAGCCATGGATATCTATCCGGGTTATACCAGTGATTTCTTT
GATGGTAAACATAATATCGTGCTGGGTGGCAGTTGGGCCACCCTG
CCGCGTATTGCAGGCCGTACCACCTTTGTGAATTGGTATCAGCAT
AATTATCGTTATGCATGGGCAGGTGCACGCCTGGTTCGCGATATT
taa
<SEQ ID NO: 36; PRT; B3Egt1;
1_XM_014676787;
Penicillium digitatum Pd1>
MSPTLLRNVDTVEIVNIHQCDMEFSLVDDVYKNLDPPAGKQRTFP
TLLLYDAKGLKLFEEITYLDEYYLTNTEIEILKKHAKKIVAHIPE
NAQLVELGSGNLRKIEILLRECERSEKKVDYYALDLSLGELQRTF
SEISPESFIHVGFHGLHGTYDDAVGWLKSPENRKRPTLVLSMGSS
MGNFSPPDAADFLGGFSKLLGPSDFLLVGLDACKNPEKVFRAYND
SKGITRKFYENGLLHANRVLGFKAFKADEWEILTDYDNREGRHQA
FYVSKVDVIINGIKIRKGEKLIFEEAWKYGRNERDQLWRNANLIS
QVEFGNSTDDYHLHLLSPAALDLSMNPSKYAAQPIPSIENFQSLW
TAWDLATRTMVPHEELLSQPIKLRNALIFYFGHIPTFLDIHLTRA
LQEESTEPSNYKTIFERGIDPDVEDPQQCHSHSEIPDEWPPLDEI
LDYQDRVRNRALSILQQGYASQDRALGEALWIGYEHEAMHLE
TFLYMLIQSDKTLPPTGVDRPDFEQINRQAKINKKPNKWFRIPRQ
TIEIGLNDSNEEVVPNQSFGWDNEKPQRKVTVHAFEAQARPITNG
EYAKYIQDKGIKTYPASWVFKPSQDNPVSKGISSSDAQAGSSSSP
AGLSLKDITVRTVFGPVALEVAQDWPLAASYDEVASYAKCMKCRI
PTFEETRSIYHYSDQLKGDRVTNDHRNGVNGLANDSKPNSTDQTV
FRDLTGCNVGFNNWHPIPVTSNGDQLAGQGEMGGVWEWTSTPLMP
HDDFKAMDIYPGYTSDFFDGKHNIVLGGSWATLPRIAGRTTFVNW
YQHNYRYAWAGARLVRDI
<SEQ ID NO: 37; DNA; B4Egt1;
1_XM_001939502;
Pyrenophora tritici-repentis>
ATGGCAACCAAAATTATCGATATCCGCGTGGATACCGCCGAAAGC
GATATTCTGGCCGATATTAAGAAAGGCCTGCGTCCGGAAAATGGC
GGTGAAAAGAAACTGCCGACCCTGCTGCTGTATGATCAGGAAGGT
CTGCGCCTGTTTGAAAAAATTACCTATCAGGAAGAATACTACCTG
ACCAATGCAGAAATTGAAGTGCTGGAAACCTATGCAGATAGTATT
GCAGAACGTATTAGTAGTCCGAGCATTATTGTGGAACTGGGCAGC
GGCAATCTGCGCAAAGTTAATATTCTGCTGCAGGCCCTGGATCGC
CTGGGCAAAGATGTTGAATATTATGCAGTGGATCTGAGTCTGCCG
GAACTGGAACGTACCTTTGGTCAGATTCCGATTGAAGGCTATAAA
CATGTGAAATGTTTCGGTCTGCATGGCACCTATGATGATGCCCTG
GGTTGGCTGAAAAGCCCGGCAATTGAAGCAAAACCGAAAACCATT
CTGTGGCTGGGCAGCAGCCTGGGTAATTTTAAACGCCATGAAGTT
CCGCCGTTTCTGGCCGGCTTTGGCGAAGTGCTGCAGACCGGCGAT
ACCATGCTGATTGGCATTGATAGTTGTAAAGATCCGGAACGCGTT
TTTCATGCATATAATGATCGTAATGGTGTTACCCATCGTTTTATT
CTGAATGGCCTGAAACATGCAAATGCACTGATGGGCGAAAATGCC
TTTAATCTGGATGATTGGGAAGTTATTGGTGAATATGATAAACAG
GCAGGCCGCCATCATGCCTTTGTGGCACCGCGCAAAGATGTTGTG
ATTGATGGCGTGCCGGTGAAAAAAGGTGAACGCATTCGCATTGAA
GAAAGCTATAAATATAGCGGTGAAGAAGCAAAAGAACTGTGGGAA
ATGGCCAAACTGACCGAAAATGTGGTGTGGCCGAATGCAAAAGGT
GACTATGGCCTGCATTTTGTGAGTAAACCGGCCGTGTTTTTCCCG
ACCAAACCGGAAGAATATGCAGCAAAACCGGTGCCGAGCCTGACC
GAATGGCAGGAACTGTGGAAAGCATGGGATGCCGTTAGCAAACAG
ATGATTCCGGAAGAAGAACTGCTGAGTAAACCGATTAAGCTGCGC
AATGAATGCATTTTCTATCTGGGTCATATTCCGACCTTTCTGGAT
ATTCATATTGCACGTGCCACCGGTAAAAAACCGAGCGATCCGGCA
TATTTTTGGAAAATTTTTGAACGTGGTGTGGACCCTGATGTGGAA
GATCCGACCCGCTGCCATGCACATAGCGAAGTGCCGGAAGAATGG
CCGCCGCTGAAAACCATTTTAATGTTTCAGCAGAGTGTTCGCGAT
AATGTTGAAGCACTGTATAGCAGCGGTGAAGCCGAAAGCAATGGT
CGTGTGAGCCGCGCCCTGTGGCTGGCTTTTGAACATGAAGCAATG
CATCTGGAAACCCTGCTGTATATGCTGATTCAGAGTGATAAAGTT
CTGCCGCCGCCGGGTACCACCGTTCCGGATTTTGCAGCATTTGCC
GCCCGTAGTAATAGCCTGGCAGTGGAAAATGAATGGTTTACCATT
CCGGCAAGCGATGTTAGCGTTGGTCTGGAAGATCCGGAAGGTGAC
TATGATAGCCAGCGTTATTTTGGTTGGGATAATGAACGTCCGCAT
CGTAGTACCCATATTAAGAGCTTTCGTGCCAAAGCCCGTCCGATT
ACCAATGGCGAATATGCAACCTATCTGAGCGAAACCGGCAAAACC
GTTATTCCGGCAAGTTGGTGCGAACAGCCGTATTATAATGCAAAA
GCCACCAGTACCGCAAAACGCGATAGTGTTATTAATGGCCATCAG
AATGGTACCAATGGTAGCACCACCGGCATTACCGATGGCAAATTT
GTTCGCACCGTGTTTGGCACCGTTCCGCTGAAACTGGCCCTGAAT
TGGCCGGTTGTGGCAAGTTATGATGAACTGGCAGGCTGTGCCCAG
TGGATGGGTGGTCGTATTCCGACCATGGAAGAAGCCCGCAGCATC
TATAGTTATGTGGAAAGCATGAAAGAAGAATTTGAAAAAAGCCTG
GGTAACACCATTCCGGCCGTGAATGGCCATCTGATTAATGAAGGT
GTGTTTGAAACCCCGCCGAGCAAACCGCTGAGTAATGGTAATAGT
GGTGCAGGTCCGAGCCTGAATCCGCATGATCTGTTTATTGATCTG
GAAGGCACCAATGTGGGTTTTAAACATTGGCATCCGGTTAGCGTG
GCAGAACGCGGTAATAAGCTGTGTGGTCAGAGCGATCTGGGCGGT
GTGTGGGAATGGACCAGTACCGTTCTGGAAAAACATGATGGTTTT
GAACCGATGGAACTGTATCCGGGCTATACCGCAGATTTCTTTGAT
GGCAAACATAATATTACCCTGGGTGGTAGCTGGGCAACCCATCCG
CGTATTGCAGGCCGTAAAACCTTTGTTAATTGGTATCAGCGCAAT
TATCCGTATATGTGGGCCGGCGCACGTATTGTGAGCGATGTTtaa
<SEQ ID NO: 38; PRT;
B4Egt1; 1_XM_001939502;
Pyrenophora tritici-repentis>
MATKIIDIRVDTAESDILADIKKGLRPENGGEKKLPTLLLYDQEG
LRLFEKITYQEEYYLTNAEIEVLETYADSIAERISSPSIIVELGS
GNLRKVNILLQALDRIGKDVEYYAVDLSLPELERTFGQIPIEGYK
HVKCFGLHGTYDDALGWLKSPAIEAKPKTILWLGSSLGNFKRHEV
PPFLAGFGEVLQTGDTMLIGIDSCKDPERVFHAYNDRNGVTH
RFILNGLKHANALMGENAFNLDDWEVIGEYDKQAGRHHAFVAPRK
DVVIDGVPVKKGERIRIEESYKYSGEEAKELWEMAKLTENVVWPN
AKGDYGLHFVSKPAVFFPTKPEEYAAKPVPSLTEWQELWKAWDAV
SKQMIPEEELLSKPIKLRNECIFYLGHIPTFLDIHIARATGKKPS
DPAYFWKIFERGVDPDVEDPTRCHAHSEVPEEWPPLKTILMFQQS
VRDNVEALYSSGEAESNGRVSRALWLAFEHEAMHLETLLYMLIQS
DKVLPPPGTTVPDFAAFAARSNSLAVENEWFTIPASDVSVGLEDP
EGDYDSQRYFGWDNERPHRSTHIKSFRAKARPITNGEYATYLSET
GKTVIPASWCEQPYYNAKATSTAKRDSVINGHQNGTNGSTTGITD
GKFVRTVFGTVPLKLALNWPVVASYDELAGCAQWMGGRIPTMEEA
RSIYSYVESMKEEFEKSLGNTIPAVNGHLINEGVFETPPSKP
LSNGNSGAGPSLNPHDLFIDLEGTNVGFKHWHPVSVAERGNKLCG
QSDLGGVWEWTSTVLEKHDGFEPMELYPGYTADFFDGKHNITLGG
SWATHPRIAGRKTFVNWYQRNYPYMWAGARIVSDV
<SEQ ID NO: 39; DNA;
B5Egt1; 1_XM_002487114;
Talaromyces stipitatus>
ATGAGCCCGGCACTGCTGAGCAATGGCAGTGTTAATATTGTTGAT
ATTCGTGATAAGGACGCCAATTTTAGCGCCGCAGCAGCAATTCAG
GATGGTCTGGACCCTCCGGCCGGCAAAGCCCGTAGCTTTCCGACC
GTGCTGCTGTATGATGCCGTTGGCCTGCGTCTGTTTGAAGAAATT
ACCTATCTGGATGAATACTATCTGACCAATACCGAAATTGAAGTG
CTGGAAAAACATGCACGTACCATTGCAGAACGTCTGCCGGATCAG
AGCCAGCTGGTTGAACTGGGTAGCGGCAATCTGCGCAAAGTTGAA
ATTCTGCTGCGCGAATTTGAAAATCTGCAGAAACGCGTGGATTAT
TATGCACTGGATCTGAGCCTGGAAGAACTGCAGCGTACCTTTGCA
CAGGTTAGTCCGCAGAGTTATCATTATGTTCGTTTTCAGGGCCTG
CATGGCACCTATGATGATGCCCTGGAATGGCTGAAAAATCCGCAG
AATCGTAAACGCCCGACCTGTGTTCTGAGCCTGGGTAGTAGCATT
GGCAATTTTAATCGCAAAGCCGCCGCAGATTTTCTGCGCCAGTAT
AGTCAGCTGCTGGGCCCGACCGATAGCATTATTATTGGTCTGGAT
GGCTGTAAAGATAAAGATCGTGTTTATCGTGCATATAATGATAGT
AAAGGCATTACCCATCAGTTTTATCTGAATGGTCTGAGCCATGCA
AATCAGGTGCTGGGTTATAATAGTTTTCGCCCGGATCAGTGGGAT
ATTGAATGTCTGTATGATGAAGCAGATGGTTGTCATCGCGCCTTT
TATGTGCCGACCCAGGATGTGACCATTAATGGTATTAGTCTGCGT
AAAGGTGAAAAAATTATCTTTGAAGAGGCCTATAAGTACGATGCC
CAGGAACGCGAAGAACTGTGGCGTGATGCCGGTCTGATTAATGTT
GCAGCACTGGGCAATAGCCATGATAATTATCATCTGAATATGCTG
AGCCCGGCAAAAGTGAGTTTTCCGAGTCGCCCGAGCGAATATGCA
CCGAGTGCCGTGCCGGCCTGGGAAGAATGGCGTAGCCTGTGGACC
AGCTGGGATGTGGTTAGCAAAACCATGGTGCCGCGTGATGAACTG
CTGAGCAAACCGATTAAGCTGCGTAATGCACTGATTTTCTATCTG
GGCCATATTCCGACCTTTCTGGATATTCATCTGACCCGCGCCACC
CGCGGCAAACCGACAGATCCGAAATATTATCCGCAGATTTTTGAA
CGCGGTATTGATCCGGATGTGGATAATCCGGAACAGTGCCATGCA
CATAGTGAAATTCCGGATGAATGGCCGGCCCTGGGCGAAATTCTG
CGCTATCAGGAACAGGTGCGTAGCCGTGTGCAGAGCCTGCTGCGT
ACCGAAGATGTTAGCCAGAATCGCCTGCTGGGTGAAGCACTGTGG
ATTGGCTTTGAACATGAAGTTATGCATCTGGAAACCTTTCTGTAT
ATGCTGCTGCAGAGTGATCGTATTCTGCCGCCGCTGGGTGTTGAT
AGCCCGGATTTTAAAGGCATTGCCCGCCAGGCCGAACTGGATGCC
AAACCGAATCAGTGGTTTAGTATTCCGGAACAGACCATTACCATT
GGTGTGGATGATAGTGATCTGAGCAAACTGCCGGCCCAGAGCTTT
ACCTGGGATAATGAAAAACCGAAACGTAGCGTTCGTGTGCATGCA
TTTGAAGCACAGGGCCGCGCCGTTACCAATCGTGAATATGCACAT
TATCTGAAAGAAAATAGCATCCATCGCATTCCGACCAGCTGGGTT
ATTCAGGCAGCAAATAGCTGGAATAATACCTTTAATGGTCTGCAT
ACCAATGGCGTTAGCAATGGTCATGGTAATAGCATGGATAATTAT
GCAGTTCGTACCGTTTTTGGCCCGGTTAGCTTTGCCTGGGCAGCC
GATTGGCCGGTTATGGCAAGCTATGATGAACTGGCAGGCTATGCA
GAATGGAAAAAATGTCGTCTGCCGACCTTTGAAGAAGTGCGCAGT
ATCTATAAATATGCCGCAGCCCTGAAAGGTCAGCCGAATGGTGTG
GATGCCGTGAGTAATGGTCTGGCCATTCTGAAAAAGAAAGATCCG
ACCGATGCCGTTATTAATGGCGCAAATCCGGAAAGCATTTTTGTG
GATCTGGCAGATGCCAATGTTGGTTTTAAAAATTGGCATCCGGTT
CCGGTTACCCCGAATGGTGACAAACTGGCCGGTCAGGGCGAAATG
GGTGGTGTTTGGGAATGGACCAGCACCCCGCTGAGTGCACATGAT
GGCTTTGAAGAAATGAAAATCTATCCGGGCTATACCGCCGATTTC
TTTGATGGCAAACATAATATTGTGCAGGGTGGCAGTTGGGCAACC
CATCCGCGCATTGCAGGCCGCACCAGTTTTGTGAATTGGTATCAG
CATAATTACCCGTATGCATGGGTGGGCGCCCGTCTGGTTCGTGAT
CTGtaa
<SEQ ID NO: 40; PRT;
B5Egt1; 1_XM_002487114;
Talaromyces stipitatus>
MSPALLSNGSVNIVDIRDKDANFSAAAAIQDGLDPPAGKARSFPT
VLLYDAVGLRLFEEITYLDEYYLTNTEIEVLEKHARTIAERLPDQ
SQLVELGSGNLRKVEILLREFENLQKRVDYYALDLSLEELQRTFA
QVSPQSYHYVRFQGLHGTYDDALEWLKNPQNRKRPTCVLSLGSSI
GNFNRKAAADFLRQYSQLLGPTDSIIIGLDGCKDKDRVYRAYNDS
KGITHQFYLNGLSHANQVLGYNSFRPDQWDIECLYDEADGCHRAF
YVPTQDVTINGISLRKGEKIIFEEAYKYDAQEREELWRDAGLINV
AALGNSHDNYHLNMLSPAKVSFPSRPSEYAPSAVPAWEEWRSLWT
SWDVVSKTMVPRDELLSKPIKLRNALIFYLGHIPTFLDIHLTRAT
RGKPTDPKYYPQIFERGIDPDVDNPEQCHAHSEIPDEWPALGEIL
RYQEQVRSRVQSLLRTEDVSQNRLLGEALWIGFEHEVMHLETFLY
MLLQSDRILPPLGVDSPDFKGIARQAELDAKPNQWFSIPEQTITI
GVDDSDLSKLPAQSFTWDNEKPKRSVRVHAFEAQGRAVTNREYAH
YLKENSIHRIPTSWVIQAANSWNNTFNGLHTNGVSNGHGNSMDNY
AVRTVFGPVSFAWAADWPVMASYDELAGYAEWKKCRLPTFEEVRS
IYKYAAALKGQPNGVDAVSNGLAILKKKDPTDAVINGANPESIFV
DLADANVGFKNWHPVPVTPNGDKLAGQGEMGGVWEWTSTPLSAHD
GFEEMKIYPGYTADFFDGKHNIVQGGSWATHPRIAGRTSFVNWYQ
HNYPYAWVGARLVRDL
<SEQ ID NO: 41; DNA;
B6Egt1; 1_XM_014099552;
Trichoderma virens>
ATGCCGGCAGTGCGCGAAAGCGTTCTGCTGGCACAGCAGCATATT
ACCGATGATGCCATGGCACTGAAAACCGTGAATGCCACCGCACGC
AGTCTGGATATTATTGATATTCAGAATAGCCGTATCGATATTAAT
CTGAAAGATGAAATCCTGACCCAGATGAATCCGGAAGAAGGTCCG
CGCACCCTGCCGACCCTGCTGTTATATGATGAACGTGGCCTGCAG
CTGTTTGAAGATATTACCTATCTGGATGAATACTATCTGACCAAT
TATGAAATCGAACTGCTGAAAAAATCCGCAGCCGAAATGGCCAGC
AAAATTCCGGAAGGTGCCATTGTGGTTGAACTGGGCAGTGGTAAT
CTGCGTAAAGTTTGCCTGCTGCTGCAGGCCTTTGAAGATGCAAAA
AAGAAAATTGATTACTACGCACTGGATCTGAGCCAGACCGAACTG
GAACGTACCCTGGCAGAAGCCCCGGCCTTTGAATATGTTAGCTGC
CGTGGTCTGCGCGGTACCTATGATGATGGCTGCGAATGGCTGAAA
CAGGAAGCCATTCTGGCACGTCCGAAATGCATTCTGCATCTGGGC
AGCAGCATTGGTAATTTTAATCGCGATGAAGCAGCAGATTTTCTG
CGCAGTTTTGCCGAAATTCTGCAGCCGACCGATCTGATGATTGTG
GGTGTGGATAGTTGCCAGAATCCGGATAAAGTGTATCATGCATAT
AATGATAGCAAGGGCATTACCCATCAGTTTGTTCTGAATGGCCTG
ACCCATGCCAATGAAATTCTGGGTAATGAAGTTTTTAACGTGGAA
GAATGGAATGTGACCGGTGAATATGTTTATGATGTTGATGGCGGC
CGTCATCAGGCCTTTGTTAGCCCGCTGGAAGTGGCAAGTGTTCTG
GGCCATATTATTAAGCCGCATGAACGCATTAAGATTGAACAGAGC
CTGAAATATAGTGATATTGGTGTTGCCAAACTGTGGAAAACCGCA
GGCCTGGAAGAAGTGACCCGCTGGAGCCATAATGGTGAATATGGC
CTGCATATGCTGAAAAAAGCCAAAATGCCGTTTCCGCGTCTGCCG
GAACTGTATGCCAGCGGTACCCTGCCGACATGGGCAGATTGGGAA
AGCCTGTGGGCAGCATGGGATACCGTGACCCGTAAAATGCTGCCG
GATGAAGAACTGAATGAAAAACCGATTAAGCTGCGCAATGCATGT
ATTTTCTATCTGGGTCATATTCCGGCCTTTCTGGATATTCAGCTG
AAAAAGACTACCAAAGCAGGCGGCACCGAACCGCTGTATTTTCAT
AGCATTTTTGAACGTGGTATCGATCCGGATGTGGATAATCCGGAA
AATTGCCATGATCATAGCGAAATTCCGGATGAATGGCCGCCGCTG
GAAGATATTCTGGCCTATCAGGATCGCGTGCGTGAACGTCTGCAG
AAAATGTATAGTAATCCGGATGAACTGGTTGGTGACGTGCGTCGC
GCAGTGTGGATTGGCTTTGAACATGAAGTTCTGCATCTGGAAACC
CTGCTGTATATGCTGCTGCAGAGTGATAAAACCCTGCCGCCGCCG
CATACCGTTATTCCGGATTTTCCGAAAATGGCCCAGAAAGCCTAT
GCACAGCGCGTTCCGAATCAGTGGTTTGAAATTCCGGAACAGACC
ATTACCATTGGCATGGATGATCCGGAAGATGAACATGATAGCAAA
CGCCATTTTGGTTGGGATAATGAAAAACCTGCACGTCAGGAAAAA
GTGCATGCATTTGAAGCCAAAGCCCGTCCGATTACCAATGAAGAA
TATGCAAAATATCTGTACAGCAGCCATATTGAAGCACTGCCGGCC
AGTTGGAGTATTATTCCGCCGAATTATCATCATAATACCAATGCA
ACCACCCCGGGTAAACCGATTCTGAGTGAACTGCCGGAAAGTTTT
ATTCATGATAAAGCAGTTCGTACCGTTTATGGCCTGGTGCCGCTG
CGCTATGCCCTGGATTGGCCGGTGTTTGCCAGCTATGATGAACTG
GCCGGCTGCGCAGCATGGATGGGCGGTCGTATTCCGACCATGGAA
GAAGCAAAAAGTATCTATGCCTATGTTGAAAAACAGAAAGATATC
GCCAAACAGAGCAAACTGAGCAATAAGGTGCCGGCCGTTAATGGT
CATCTGGTTAATGATGGCGTTCAGGAAACCCCGCCGAGTGCAACC
AGCCCGAGCAGTCTGTTTACCGATCTGAGTACCACCAATACCGGT
TTTCTGCATTGGCATCCGGTGCCGGTTACCCCGAAAGGTGGCAGT
CTGGCCGGCCAGGGCGATTTTGGTGGTGTGTGGGAATGGACCAGC
ACCATTCTGCGCCCGCATGAAGGCTTTCGTCCGATGAGTATCTAT
CCGGGTTATACCGCAGATTTCTTTGATGAAAAACATAATGTGGTG
CTGGGTGGTAGCTGGGCCACCCATCCGCGCGTTGCCGGTCGTAAA
AGCTTTATTAATTGGTATCAGCGCAATTATCTGTATGCATGGGTT
GGTGCCCGTCTGGTTCGCGATCTGtaa
<SEQ ID NO: 42; PRT;
B6Egt1; 1_XM_014099552;
Trichoderma virens>
MPAVRESVLLAQQHITDDAMALKTVNATARSLDIIDIQNSRIDIN
LKDEILTQMNPEEGPRTLPTLLLYDERGLQLFEDITYLDEYYLTN
YEIELLKKSAAEMASKIPEGAIVVELGSGNLRKVCLLLQAFEDAK
KKIDYYALDLSQTELERTLAEAPAFEYVSCRGLRGTYDDGCEWLK
QEAILARPKCILHLGSSIGNFNRDEAADFLRSFAEILQPTDLMIV
GVDSCQNPDKVYHAYNDSKGITHQFVLNGLTHANEILGNEVENVE
EWNVTGEYVYDVDGGRHQAFVSPLEVASVLGHIIKPHERIKIEQS
LKYSDIGVAKLWKTAGLEEVTRWSHNGEYGLHMLKKAKMPFPRLP
ELYASGTLPTWADWESLWAAWDTVTRKMLPDEELNEKPIKLRNAC
IFYLGHIPAFLDIQLKKTTKAGGTEPLYFHSIFERGIDPDVDNPE
NCHDHSEIPDEWPPLEDILAYQDRVRERLQKMYSNPDELVGDVRR
AVWIGFEHEVLHLETLLYMLLQSDKTLPPPHTVIPDFPKMAQKAY
AQRVPNQWFEIPEQTITIGMDDPEDEHDSKRHFGWDNEKPARQEK
VHAFEAKARPITNEEYAKYLYSSHIEALPASWSIIPPNYHHNTNA
TTPGKPILSELPESFIHDKAVRTVYGLVPLRYALDWPVFASYDEL
AGCAAWMGGRIPTMEEAKSIYAYVEKQKDIAKQSKLSNKVPAVNG
HLVNDGVQETPPSATSPSSLFTDLSTTNTGFLHWHPVPVTPKGGS
LAGQGDFGGVWEWTSTILRPHEGFRPMSIYPGYTADFFDEKHNVV
LGGSWATHPRVAGRKSFINWYQRNYLYAWVGARLVRDL
<SEQ ID NO: 43; DNA;
B7Egt1; 1_XM_002540793;
Uncinocarpus reesii>
ATGGTTCTGCCGATGGCAGGCGTGGATATTATTGATATTCGCCGC
AGCAATTTTAATCATAGCCTGGCAAAAGAAGTTCTGGATGGCCTG
CGCGCCAAAGATGGCAGCCAGCGTAGTCTGCCGACCCTGCTGCTG
TATGATACCGAAGGTCTGCGCCTGTTTGAAGAAATTACCTATCTG
GATGAATACTATCTGACCAATGCAGAAATTGAAGTTCTGACCAGT
CATGCCGCAGGTATTGTGGAACGTGTGCCGGAAAATGCACAGCTG
GTTGAACTGGGTAGTGGTAATCTGCGTAAAATTGAAATTCTGCTG
AAAGAATTTGAGCGTGTTCGCAAAAGCGTTGAATATCTGGCCCTG
GATGTTAGTCTGGAAGAACTGCATCGTACCTTTGCCGAAATTCCG
AGTAAAAGTTATAAATATGTGAAGTGCGGTGGCCTGCTGGGTACC
TATGATGATGCCCTGGCATGGCTGAAACGCAGTGAAAATCGCCGT
AAACCGACCTGGGTTATGAGCATGGGTAGTAGCATGGGTAATTTT
ACCCGCACCGAAGCCGCCCAGTTTCTGGGCGGTTTTGCCAAAACC
CTGGGTCCGGATGATGCACTGTTTATTGGTCTGGATAGTTGCAAA
GATCCGCAGAAAGTTTTTCGCGCCTATAATGATAGCAAAAATGTT
ACCCGCGAATTTTATCTGAATGGCCTGGTGAATGCAAATAGTATT
CTGGGTTTTGAAGCCTTTCGTCGTATGGATTGGGATGTTGTTGGC
GAATATGATGAAGAAAATGGTTGCCATAAAGCATATTATAGCCCG
CTGAAAGATGTGACCATTCAGGATCTGAGCATTCAGAAAGGTGAA
AAAATTTTCTTTGAGCAGGCATTCAAATACAGCAAACAGGAATAT
GAAGCCCTGTGGCAGCAGAGTGGCCTGAAACCGATTGCCCGCTTT
AGTAATACCACCGGCGATCATCATATTCATCTGCTGAGTAGCAGC
CCGTATATTGTTCCGACCCAGCCGGCCGAATATGCCCCGAGTGCA
ACCCCGAGTCTGAAAGAATTTGAAGCACTGTGGAAACTGTGGGAT
ACCGTGACCACCGAAATGCTGCCGCGCAATGAACTGCTGAGTAAA
CCGATTAAGCTGCGCAATAGTCTGATTTTCTATCTGGGTCATATT
CCGGCATTTCTGGATATTCAGATTGCAAAAGCCACCACCGGTCAG
CCGACCGAACCGAAAAGCTATCATAGCACCTTTGAACGCGGCATT
GATCCGGATGTTGATGATCCGACCAAATGTCATGATCATAGCGAA
ATTCCGGCAGAATGGCCGCCGGTTGAAGAAATTCTGCGCTATCAG
ACCGCAGTTCGTAATCGCGCCCGTCTGCTGCTGCAGAAAAGCCAG
AGCGTGCTGGATCGTCGCATTCATGAAGCCCTGTGGATTGGCTTT
GAACATGAAGCAATGCATCTGGAAACCTTTCTGTATATGCTGCTG
CAGAGTGATAAAGTTCTGCCGCCGCCGGAAATTATGCAGCCGGAT
TTTGAATATCTGGCAATTCGCAGCGCCCAGGAAAGTGTGCCGAAT
GAATGGTTTACCGTTCCGGAACAGACCATTAGCATTGGTCTGGAC
GATCCGGGTAGTGCCCAGATTCCGACCCAGAGTTTTAGTTGGGAT
AATGAACAGCCGCGTCGTAGTGCCAAAGTGCATAGCTTTGAAGCC
AAAGGCCGCCCGATTACCAATGGTGAATATGCAAAATATCTGGAA
GCAAATGAACCGCGTGCCATTCCGGCCAGCTGGACCAAAAGTCCG
AAAAGTTTTAGCAAAAGTAACGGTCTGGTGAATGGCAATACCAAT
GGTGCAAATGGTCATGGCATTAATGGCGCCAGCACCGCACCGCAG
TTTCTGGAAAAATATTGTGTTCGCACCGTTTTTGGTCCGGTGCCG
CTGCGTTTTGCAGCAGATTGGCCGGTTATTGCAAGTTATAATGAA
CTGGAAGGTTATGCCAATTGGGCAAATTGCCGCATTCCGACCTTT
GAAGAAGCCCGTAGCCTGTATCAGTATAGCGCCTTTCTGAAAAGC
AGTGCAGATAGCAGCGTGTGTGCAGCAGTGAATGGCAACAGCAAT
ACCGTTAAAAAAGCACATGGCAATAGCAATGGTTTTGTGCATCAG
CAGAATGGTAAACCGCGTGCCCCGGATCATCAGCCGGTTAGTCTG
GCAAGCGCAAGCCAGGTTCCGGTTTATATTGATCTGGATGGCTAT
AATGTGGGTTTTAAACATTGGCATCCGAGTCCGGTGACCCAGAAT
GGTAATAAGCTGAGCGGTCAGGGCGATATGGGTGGTGTTTGGGAA
TGGACCAGCAGCGCCCTGCAGCCGCATGAAGGTTTTAAAGCCATG
GATCTGTATCCGGCCTATACCGCAGATTTCTTTGATGGTAAACAT
AATATCGTGCTGGGCGGCAGTTGGGCAACCCATCCGCGCATTGCC
GGTCGCACCACCTTTGTTAATTGGTATCAGCGCAATTATCCGTTT
GCCTGGGCAGGTGCCCGTCTGGTGCGCGATGTGtaa
<SEQ ID NO: 44; PRT; B7Egt1; 1_XM_002540793;
Uncinocarpus reesii>
MVLPMAGVDIIDIRRSNFNHSLAKEVLDGLRAKDGSQRSLPTLLL
YDTEGLRLFEEITYLDEYYLTNAEIEVLTSHAAGIVERVPENAQL
VELGSGNLRKIEILLKEFERVRKSVEYLALDVSLEELHRTFA
EIPSKSYKYVKCGGLLGTYDDALAWLKRSENRRKPTWVMSMGSSM
GNFTRTEAAQFLGGFAKTLGPDDALFIGLDSCKDPQKVFRAYNDS
KNVTREFYLNGLVNANSILGFEAFRRMDWDVVGEYDEENGCHKAY
YSPLKDVTIQDLSIQKGEKIFFEQAFKYSKQEYEALWQQSGLKPI
ARFSNTTGDHHIHLLSSSPYIVPTQPAEYAPSATPSLKEFEALWK
LWDTVTTEMLPRNELLSKPIKLRNSLIFYLGHIPAFLDIQIAKAT
TGQPTEPKSYHSTFERGIDPDVDDPTKCHDHSEIPAEWPPVEEIL
RYQTAVRNRARLLLQKSQSVLDRRIHEALWIGFEHEAMHLETFLY
MLLQSDKVLPPPEIMQPDFEYLAIRSAQESVPNEWFTVPEQTISI
GLDDPGSAQIPTQSFSWDNEQPRRSAKVHSFEAKGRPITNGEYAK
YLEANEPRAIPASWTKSPKSFSKSNGLVNGNTNGANGHGINGAST
APQFLEKYCVRTVFGPVPLRFAADWPVIASYNELEGYANWANCRI
PTFEEARSLYQYSAFLKSSADSSVCAAVNGNSNTVKKAHGNSNGF
VHQQNGKPRAPDHQPVSLASASQVPVYIDLDGYNVGFKHWHPSPV
TQNGNKLSGQGDMGGVWEWTSSALQPHEGFKAMDLYPAYTADFFD
GKHNIVLGGSWATHPRIAGRTTFVNWYQRNYPFAWAGARLVRDV
<SEQ ID NO: 45; DNA;
B8Egt1; 1_XM_013164379;
Schizosaccharomyces octosporus>
ATGATCAGCAATAACATCATCAACATCGGCAGTCTGGAAGTTCTG
TTTAGCCCGGAAATTATTGAACAGTGTCTGAAAGTTTGCCAGCTG
CCGACCAGCCTGCTGTATGATGAAAAAGGTCTGCAGCTGTTTGAT
AAAATTACCGGCACCGAAGAATATTATCTGTTTGATTGCGAACTG
AGCATTCTGCAGCGCGATAGCGATGCAATTGCCCAGGAACTGCTG
AGTCCGGATCTGCCGAATACCGTGGTGGAACTGGGTTGTGGCGCA
ATGCATAAAACCAAACATCTGCTGGATGCATTTGAACGTACCGGC
AAAGATGTGAATTTTTATGCCCTGGATCTGAATGAAGATGAACTG
CGCCGTGGCCTGAGCCAGCTGGAACAGCATGCCAGCTATAAACAT
GTTAAAGTTGCAGGTATTTGCGGCTGCTTTGATATGTTTCTGAAA
AATATTGACAAGTTCCGTGGTAGTAGCAATGGCCAGATTAGCATT
CTGTATCTGGGTAGCAGCATTGGCAATTTTAATCGCGATAGTGCA
ACCAAATTCATTAAGAGTTTTAGCGATCGTCTGGCAATTGGCGAT
AAATTTCTGCTGAGTTTTGATCATCGCAATAGTGCCGAACTGGTG
GAACGCGCCTATGATGATAGCACCCGTGTTACCGAAACCTTTGAA
AAGAATATTCTGACCAGCGCCAATCGCGTGTTTGGCGAAGATTTG
TTTAATGAAAATGACTGGGATTACGTTAGTAAATATGAAGAAGAT
TTGGGTGTGCATCGTGCCTATCTGCGTGCCAAAAAAGATTTGACC
ATTGCCAAAGGCCCGATGGTTTTTAATTTTAAAGCCGGTCATCTG
CTGCTGTGTGAAGAAAGTTGGAAAAGTAATGATAACGAATGTCGC
GAAATTATTCATAATGGCAATTTTGTTGTGGACAATGTTCATACC
AATACCACCCCGAGTTATAGCGTGTATGTTGGTAGTAAAAGTTTT
CCGATTCTGCCGCAGATTCCGAAAGAAGCCAGCATTAGTCTGGAA
GAATGGAGCCAGACCCGTGATATTTGGCTGTTTGTGACCAATAAG
CTGCTGAATGATAGTAATATTTTCAACGTGTGGATTCCTCTGCGC
CATCCGTTTATTTTCTATATGGGCCATATTCCGGTTTTTAATGAT
ATCTATCTGAGTCGTATCTTCGAAAATCCGGCAACCGCCAGCAAA
CGTGAATATTGGGATTGGTTTCAGCGCGGTATTGATCCGGATGTT
GAAAATCCGGAACAGTGCCATTGGCATAGTCAGACCCCGCCGAAA
TGGCCGAGCCCGAATGAACTGCGTAGTTATGAAGTTGCAAGCTGG
CAGAATCATATTCTGAAACTGCTGGATGGCAGCCATGCCCTGAGC
CCGAGTCAGAAACGTATTCTGTGGCTGTGTTATGAACATGTTGCC
ATGCATATTGAAACCACCCTGTATATCTATGTGCAGAGTTTTCAG
CATCCGAAACAGAATAATACCCTGTGTGGTCTGCCGCCGAGTAAA
AATCTGAAACTGAAAAAAGATCCGAGCTGGATTAAGTTTCCGAAT
GCACAGGTTCTGCAGGGTCTGCCGATTCGCAGTGATCAGAAAACC
AAACTGAATAGCGAAGAACCGGATGAACAGGAATTTTTCGGCTGG
GATAATGAAAAACCGCTGCGCATGAAACAGCCGAGTTTTCAGATT
GCAAATCGCCCGATTAGCAATGGTGAATATCTGGATTATCTGGAA
AGTAAACCGGCCGATGATAAACATTATCCGAAAAGCTGGAAAGTG
ATTGATGGTAAACTGTATGTGACCACCATGTATGGCCTGCTGCCG
CTGGAAAGTTATCATAGCTGGCCGGTTATGGCAAGCTTTGAAGAA
CTGAATGATTATGCAGCAAGTAAAGGTTGTCGTCTGCCGACCGAA
GAAGAACTGAACCATTTTTATGATCATGTGCTGCATCGCAAAAGC
GAAACCTATGTGAGTACCAAAGGCATGGCAACCGGTTTTCAGCAG
CTGCATCCGGCCAATCTGAAAGATGATGGCACCCATCAGATTTTT
ACCGGTGCCTGGGAATGGAGTAGCACCGTTCTGGATAAACATGAA
GGCTTTGAACCGGAAGCCCTGTATCCGGATTATACCAAAGATTTC
TTTGATGGTAAACACAATGTGGTGCTGGGCGGCAGTTTTGCAACC
GTTCCGCGCATTGCAAATCGTCGCAGCTTTCGTAATTTTTATCAG
CGTCAGTATCAGTATGCCTGGATTACCGCACGTCTGGCCAAAAGC
ATTtaa
<SEQ ID NO: 46; PRT;
B8Egt1; 1_XM_013164379;
Schizosaccharomyces octosporus>
MISNNIINIGSLEVLFSPEIIEQCLKVCQLPTSLLYDEKGLQLFD
KITGTEEYYLFDCELSILQRDSDAIAQELLSPDLPNTVVELGCGA
MHKTKHLLDAFERTGKDVNFYALDLNEDELRRGLSQLEQHASYKH
VKVAGICGCFDMFLKNIDKFRGSSNGQISILYLGSSIGNFNRDSA
TKFIKSFSDRLAIGDKFLLSFDHRNSAELVERAYDDSTRVTETFE
KNILTSANRVFGEDLENENDWDYVSKYEEDLGVHRAYLRAKKDLT
IAKGPMVFNFKAGHLLLCEESWKSNDNECREIIHNGNFVVDNVHT
NTTPSYSVYVGSKSFPILPQIPKEASISLEEWSQTRDIWLFVTNK
LLNDSNIFNVWIPLRHPFIFYMGHIPVENDIYLSRIFENPATASK
REYWDWFQRGIDPDVENPEQCHWHSQTPPKWPSPNELRSYEVASW
QNHILKLLDGSHALSPSQKRILWLCYEHVAMHIETTLYIYVQSFQ
HPKQNNTLCGLPPSKNLKLKKDPSWIKFPNAQVLQGLPIRSDQKT
KLNSEEPDEQEFFGWDNEKPLRMKQPSFQIANRPISNGEYLDYLE
SKPADDKHYPKSWKVIDGKLYVTTMYGLLPLESYHSWPVMASFEE
LNDYAASKGCRLPTEEELNHFYDHVLHRKSETYVSTKGMATGFQQ
LHPANLKDDGTHQIFTGAWEWSSTVLDKHEGFEPEALYPDYTKDF
FDGKHNVVLGGSFATVPRIANRRSFRNFYQRQYQYAWITARLAKS
I
<SEQ ID NO: 47; DNA;
C1Egt1; 1_XM_002172061;
Schizosaccharomyces japonicus>
ATGATGGCCGAAAGTATTATTGATATCGGCGCCACCGCAGATATT
TTTAGTGCACAGAGCGTGAGCGCAAATCTGAAACAGAGCCGTCTG
AGTAGCAGCCTGCTGTATGATGAAACCGGTCTGCAGCTGTTTGGC
CAGATTACCCAGGAAGATGAATATTATCCGTTTCGTCTGGAAATG
CAGCTGCTGCAGAAACATGCAGATTGGATTGCAGAACATGTGCGT
AGTAAAACCAGTACCACCATTATTCTGGAACTGGGTTGTGGTAGC
ATGCGTAAAACCAAAGTGCTGCTGGATGCCTTTGAAAATACCTGC
AGTCCGGTTCATTATTATGCACTGGATCTGAATCGCAAAGAACTG
CAGAATAGCCTGAATACCCTGGAAGCAAGTACCAGCTATCGCAAT
GTGAAAATTAGTGGTATTTGTGGTTGTTTTAAGCATGCACTGAGT
TATCTGCCGATTCTGCGCAGTAGTCCGAATAGTAAATTTGTGCTG
ACCTATCTGGGTAGCAGCATTGGCAATTTTAGCCGTGAAGAAAGT
GCAACCTTTCTGCAGGCATTTTCTAGTAAACTGAAACCGGATGAT
CAGATTATTGTGAGTTTTGATCATCGTCATGAAAAAGAAACCATT
ATTAGCGCCTATAATGATAAACATCACATTACCGAAAAGTTCGAA
CTGAATATTCTGAATCATGTGAATCATATCTTCGGTGCACGCCTG
TTTCATCTGGATGATTGGCGCTATCAGGGCGAATATGATGAACAT
ACCGGTGTGCATAAAGCATTTCTGATTAGTAAACGCCCGGTGACC
ATTCCGGAACTGCAGCTGAGTTTTCCGCAGAATCATAAACTGCTG
TGCGAAGAAAGCTGGAAAAGCAGCAGTGAAGAAGCCAATAAGATT
CTGCATAATGGCGGTTTCTTTACCGAAGCAGAACTGAAAAGTAAT
CATGGTTTTAGCCTGTTTATCGCAAGCGTGCCGACCTTTGATGTG
AGCCGTAATCCGGAAACCCCGTGCCCGACCCTGGAAGAATGGACC
CAGATTCGCCTGGCATGGCTGTATCTGGTTTTTAAACTGTATCCG
CGCGATCTGTATTTTACCGAACTGATTCCGGTTCGTCATCCGTTT
ATTTTCTATATTGGTCATGTTCCGGCCTTTAATGATATCTATCTG
GCCCGTCTGACCGATGGCAAACCGACCCTGGGCCGCAAAGATTAT
TGGGATTGGTTTCAGCGTGGCATTGATCCGGATCTGGATAATACC
AAAAAATGCCATTGGCATAGCCAGCCGCCGGAAAAATGGCCGAGT
GTTGAAGAAGTGAATGAATATGAACGCAATGTTTGGAGTCGCCTG
GTTAGCATCTATAAACAGGGTGAAATGAGCGCCAATATGCAGCGC
GCAATGTGGATGATCTATGAACATACCGCCATGCATCTGGAAACC
AGCTATTATATTCTGCTGCAGAGCGATTATCATATTATTAGTCCG
AATAACTTCCCGCCGCCGATTGCACCGGCCCTGCAGACAGATCCG
ACCTGGGTTCGCGTGCCGGAAAGCTTTATTACCATGGGTATTCCG
ACCACCGCAGATGGTAAAGAAACCTTTTATTATGGCTGGGATAAT
GAAAAACCGGAACGTCAGGTTAGCGTGCGCTGTTTTGAAATTGCC
AATCGCCCGATTAGTAATGGCGAATATCTGAGCTTTCTGAAAGAA
ACCACCCAGAGCAAAGAAGAATTTGAAGCAGCAATTCCGAAAACC
TGGCTGCTGAAAGATGAAATGCTGTTTGCAAAAAGTATGTATGGT
CCGCTGCCGATTGAACATGTGCTGGGCTGGCCGGTTGCCACCAGC
TATGATGAACTGAAACAGTATGCCAATGCAAAAGGCTGTCGTCTG
CCGACCGATTATGAACTGCGCGCATTTTATGATCATGTGCTGAAA
CCGAATGAAGAAACCTATGTTGATACCGCCGGTTATGCCACCGCC
TTTCAGCAGTGGTATCCGAAAAGTCTGCAGGATGAAGAAAAACCG
CAGATATATACCGGCCTGTGGGAATGGACCAGTACCGTGCTGAAA
GAAGATTTGGATTTTACCCCGGAAGAACTGTATCCGGATTATACC
CGCGATTTCTTTGATGGTAAACATAATGTTGTGATGGGCGGTAGC
TTTACCACCGTTGCACGCATTGCAAATCGTCGTAGTTTTCGTAAT
TTTTATCAGCGTAAATACCCGTATGCCTGGATTGGCGCACGTCTG
GTGAAAGTGACCAATACCCTGAGCtaa
<SEQ ID NO: 48; PRT;
C1Egt1; 1_XM_002172061;
Schizosaccharomyces japonicus>
MMAESIIDIGATADIFSAQSVSANLKQSRLSSSLLYDETGLQLFG
QITQEDEYYPFRLEMQLLQKHADWIAEHVRSKTSTTIILELGCGS
MRKTKVLLDAFENTCSPVHYYALDLNRKELQNSLNTLEASTSYRN
VKISGICGCFKHALSYLPILRSSPNSKFVLTYLGSSIGNESREES
ATFLQAFSSKLKPDDQIIVSFDHRHEKETIISAYNDKHHITEKFE
LNILNHVNHIFGARLFHLDDWRYQGEYDEHTGVHKAFLISKRPVT
IPELQLSFPQNHKLLCEESWKSSSEEANKILHNGGFFTEAELKSN
HGFSLFIASVPTFDVSRNPETPCPTLEEWTQIRLAWLYLVFKLYP
RDLYFTELIPVRHPFIFYIGHVPAFNDIYLARLTDGKPTLGRKDY
WDWFQRGIDPDLDNTKKCHWHSQPPEKWPSVEEVNEYERNVWSRL
VSIYKQGEMSANMQRAMWMIYEHTAMHLETSYYILLQSDYHIISP
NNFPPPIAPALQTDPTWVRVPESFITMGIPTTADGKETFYYGWDN
EKPERQVSVRCFEIANRPISNGEYLSFLKETTQSKEEFEAAIPKT
WLLKDEMLFAKSMYGPLPIEHVLGWPVATSYDELKQYANAKGCRL
PTDYELRAFYDHVLKPNEETYVDTAGYATAFQQWYPKSLQDEEKP
QIYTGLWEWTSTVLKEDLDFTPEELYPDYTRDFFDGKHNVVMGGS
FTTVARIANRRSFRNFYQRKYPYAWIGARLVKVTNTLS
<SEQ ID NO: 49; DNA;
C2Egt1; 1_XM_007696134;
Bipolaris sorokiniana>
ATGGCCGCAAATATTATTGATATCCGCGTTGATAAAGCCGAAAGC
GATATTCTGGCCGATATTAAGAAAGGTCTGCGCCCGGTTGCCGAT
GCAGAAAAAACCCTGCCGACCCTGCTGCTGTATGATCAGGAAGGC
CTGCGCCTGTTTGAACAGATTACCTATCAGGAAGAATATTATCTG
ACCAATGCCGAAATTGAAGTTCTGGAAACCTATGCCGATAAAATT
GCCCAGCGCATTAGTCCGGGCAGCATTGTTGTTGAACTGGGCAGC
GGTAATCTGCGCAAAGTTAATATTCTGCTGCAGGCCGTGGATCGC
CTGGGCAAAGATATTGAATATTATGCCGTGGATCTGAGCCTGCCG
GAACTGGAACGCACCTTTAAACAGATTCCGATTGAAGGTTATAGT
CATGTGAAATGTTTCGGTCTGCATGGTACCTATGATGATGCCCTG
GAATGGCTGAAAAGCCCGGCAGTTGAAGCCAAACCGAAAACCATT
CTGTGGCTGGGTAGCAGCCTGGGCAATTTTAAACGCCATGAAGTG
CCGCCGTTTCTGGCAGGCTTTGGTAAAGTTCTGCAGACCGGTGAC
ACCATGCTGATTGGTATTGATAGCTGCAAAGATCCGAAACGTGTG
TTTCATGCATATAATGATCGTGATGGTGTGACCCATCGCTTTATT
CTGAATGGTCTGAAACATGCAAATGCCCTGATGGGTGAAAATGCA
TTCAATCTGGATGATTGGGAAGTTATTGGCGAATATGATACCAAA
GCAGGTCGCCATCATGCATTTGTTGCCCCGCGCAAAGATGTTGTG
GTGGATGGTGTTCCGATGAAACAGGGTGAACGCATTCGCATTGAA
GAAAGTTATAAATATAGCCGCGAAGAAGCAAAAAAGCTGTGGGAA
CTGGCCAAACTGGCCGAAAATGCAGTGTGGGCCAATAGTAAAGGT
GACTATGGTCTGCATATGGTGAGCAAACCGAGCTTTTTCTTTCCG
ACCACCCCGGAAGAATATGCAGAAAAACCGGTTCCGAGCCTGACC
GAATGGCAGGAACTGTGGAAAGCCTGGGATGCAGTTAGTAAACAG
ATGATTCCGAATAGTGAACTGCTGGCCAAACCGATTAAGCTGCGC
AATGAATGCATTTTCTATCTGGGTCATATTCCGACCTTTCTGGAT
ATTCATATTGCACGTGCCACCGATGGCAAACCGACCGAACCGGCC
TATTTTTGGAAAATTTTTGAACGCGGCGTGGACCCTGATGTTGAT
GATCCGACCCTGTGCCATGCCCATAGCGAAGTGCCGGAAGAATGG
CCGCCGCTGGGTACCATTCTGCAGTATCAGCAGACCATTCGTAAA
AATCTGGAAGCACTGTATGATAGCGGCGAAGCAGAAAAGAATTGT
CGCATTAGTCGTGGTCTGTGGATTGCATTTGAACATGAAGCAATG
CATCTGGAAACCCTGCTGTATATGCTGATTCAGAGCGATAAAGTG
CTGCCGCCGCCGGGCATTAAGCAGCCGGATTTTGCCGCATTTGCA
GCACAGAGTGAAGTTATGGCCGTGGAAAATGAATGGTTTACCATT
CCGGAAAGTGATATTGATATTGGCCTGAATGATCCGGAAAAAGAT
TTTGGTAGTAAACGCTATTTCGGCTGGGATAATGAACGCCCGTGT
CGTAGTGTTCATGTTAAAAGCTTTCGTGCAAAAGCACGTCCGATT
ACCAATGGTGAATATGCAACCTATCTGCTGCAGACCGGCAAAAAA
GAAATTCCGGCCAGTTGGTGTGATAAAGCATATAGTAATGGTCAT
GATACCAATACCACCAAACGTGATAGCGTTGTGAATGGTCAGAGC
AATGGTAATGGTGAAAGTAGCCAGGGCATTATTGAAGGCAAATTT
GTGCGTACCGTTTATGGTACCATTCCGCTGAAACTGGCAATGGGC
TGGCCGGTTGTGGCAAGCTATGATGAACTGGTGGGTTGTGCACAG
TGGATGGGCGGCCGTATTCCGACCATGGAAGAAGCACGCAGTATC
TATGCCTATGTTGATAGTATTAAGCCGGAATTTGAACAGAGCCTG
GGTAATACCATTCCGGCCGTGAATGGTCATCTGCTGAATGAAGGC
GTTTTTGAAACCCCGCCGAGCCATCATCTGAGCAATGGCAATAGT
GGTGCAGTGACCGGCCTGAAACCGCGTGATCTGTTTATTGATCTG
GAAGGCACCAATGTGGGTTTTAAACATTGGCATCCGGTGAGTGTG
GCAGAAAAAGGTGACAAACTGTGTGGTCAGAGTGATCTGGGCGGC
GTTTGGGAATGGACCAGCACCGTGCTGGAAAAACATGATGGCTTT
GAACCGATGGAACTGTATCCGGGCTATACCGCCGATTTCTTTGAT
GGCAAACATAATATTACCCTGGGCGGTAGCTGGGCAACCCATCCG
CGCATTGCAGGCCGTAAAACCTTTGTGAATTGGTATCAGCGTAAT
TATCCGTATGTTTGGGCCGGTGCACGCATTGTTACCGATCTGtaa
<SEQ ID NO: 50; PRT;
C2Egt1; 1_XM_007696134;
Bipolaris sorokiniana>
MAANIIDIRVDKAESDILADIKKGLRPVADAEKTLPTLLLYDQEG
LRLFEQITYQEEYYLTNAEIEVLETYADKIAQRISPGSIVVELGS
GNLRKVNILLQAVDRLGKDIEYYAVDLSLPELERTFKQIPIEGYS
HVKCFGLHGTYDDALEWLKSPAVEAKPKTILWLGSSLGNFKRHEV
PPFLAGFGKVLQTGDTMLIGIDSCKDPKRVFHAYNDRDGVTH
RFILNGLKHANALMGENAFNLDDWEVIGEYDTKAGRHHAFVAPRK
DVVVDGVPMKQGERIRIEESYKYSREEAKKLWELAKLAENAVWAN
SKGDYGLHMVSKPSFFFPTTPEEYAEKPVPSLTEWQELWKAWDAV
SKQMIPNSELLAKPIKLRNECIFYLGHIPTFLDIHIARATDGKPT
EPAYFWKIFERGVDPDVDDPTLCHAHSEVPEEWPPLGTILQYQQT
IRKNLEALYDSGEAEKNCRISRGLWIAFEHEAMHLETLLYMLIQS
DKVLPPPGIKQPDFAAFAAQSEVMAVENEWFTIPESDIDIGLNDP
EKDFGSKRYFGWDNERPCRSVHVKSFRAKARPITNGEYATYLLQT
GKKEIPASWCDKAYSNGHDTNTTKRDSVVNGQSNGNGESSQGIIE
GKFVRTVYGTIPLKLAMGWPVVASYDELVGCAQWMGGRIPTMEEA
RSIYAYVDSIKPEFEQSLGNTIPAVNGHLLNEGVFETPPSHH
LSNGNSGAVTGLKPRDLFIDLEGTNVGFKHWHPVSVAEKGDKLCG
QSDLGGVWEWTSTVLEKHDGFEPMELYPGYTADFFDGKHNITLGG
SWATHPRIAGRKTFVNWYQRNYPYVWAGARIVTDL
<SEQ ID NO: 51; DNA;
C3Egt1; 1_XM_008027421;
Exserohilum turcica>
ATGGCCACCCAGATTATTGATATTCGTGTTGATACCGCCGAAAGT
GATATTCTGGCACATATTAAGAAAGGCCTGCGCCCGGAAGCAGGC
GGCGAAAAAACCCTGCCGACCCTGCTGCTGTATGATCAGGAAGGT
CTGCGTCTGTTTGAACAGATTACCTATGAAGAAGAATACTATCTG
ACCAATGCAGAAATTGAAGTTCTGGAAAAATACGCCCATGAAATT
GCACAGCGTGTTCCGAGCGGCAGCATTGTTGTTGAACTGGGCAGC
GGCAATCTGCGCAAAGTTAATATTCTGCTGCAGGCCATGGATCGC
CTGGCCAAAGATGTTGAATATTATGCAGTTGATCTGAGCCTGCCG
GAACTGCAGCGTACCTTTAGTCAGATTCCGATTGAAGGCTATAGC
CATGTTAAATGCTTTGGCCTGCATGGCACCTATGATGATGCCCTG
GAATGGCTGAAAAGTCCGGCCGTTGAAGCCAAACCGAAAACCATT
CTGTGGCTGGGTAGTAGCCTGGGCAATTTTAAACGCCATGAAGTT
CCGCCGTTTCTGGCAGGCTTTGGTCGCGTTCTGCAGACCGGTGAC
ACCATGCTGATTGGTATTGATAGTTGCAAAGATCCGGAACGTGTG
TTTCATGCATATAATGATCGCAATGGTGTGACCCATAAATTCATT
CTGAATGGCCTGAAACATGCCAATGCCCTGATGGGCGATCGTACC
TTTAATATGGATGATTGGGAAATTATCGGCGAATATGATGTGAAA
GCAGGCCGTCATCATGCCTTTGTGGCACCGCGTAAAGATGTGGTT
GTGGATGGCGTTACCGTGAAACAGGGCGAACGTATTCGCATTGAA
GAAAGCTATAAATATAGCCATAAGGAAGCAAAAAAGCTGTGGGAA
CTGGCACGTCTGAGTGAAAGTGCAGTTTGGGCAAATAGCATGGGC
GATTATGGTCTGCATCTGGTTAGTAAACCGGCATTTTTCTTTCCG
ACCAATCCGGAAGAATATGCCGCAAGTCCGGTTCCGAGCCTGGCA
GAATGGCAGGAACTGTGGAAAAGTTGGGATGCCGTGAGCAAACAG
ATGATTCCGGAAGCCGAACTGCTGAGCAAACCGATTAAGCTGCGC
AATGAATGCATTTTCTATCTGGGTCATATTCCGACCTTTCTGGAT
ATTCATATTGCACGTGCAACCGATGGCCAGCCGACCGAACCGGCA
TATTTTTGGAAAATTTTTGAACGTGGCGTGGACCCTGATGTGGAT
AATCCGACCCAGTGCCATGCCCATAGTGAAGTGCCGGAAGAATGG
CCGGCAGTGAAAACCATTTTAGAATATCAGCAGACCATTCGTAAA
AATACCGAAGCACTGTATCAGAGCGGTGAAGCCGAAAATAATGTG
CGTGTTAGTCGTGGCCTGTGGATTGCCTTTGAACATGAAGCAATG
CATCTGGAAACCCTGCTGTATATGCTGATTCAGAGCGATAAAATT
CTGCCGCCGCCGGGTACCAAAGTTCCGGATTTTGCCGCCTATGCA
GCACATAGTGATGTTCTGGCAGTTGAAAATCAGTGGTTTACCATT
CCGGAAAGCGATATTGATATTGGCCTGGATGATCCGGAAAATGAT
TTTAAAACCAAACGTTATTTCGGCTGGGATAATGAACGTCCGCGT
CGTAGTGCCCATGTGAAAGCCTTTCGTAGTAAAGCCCGCCCGATT
ACCAATGGTGAATATGCCACCTATCTGTTTCAGACCGGTAAAAAA
GAAATTCCGGCAAGCTGGAGCGAAACCAGTTATAGTAATGCCAAT
GGCACCACCATGACCAAACGCGATAGCGTGATTAATGGCCATGCA
AATGGCGATAGTGATCCGAGTTATAGTATGATTGAAGGTAAATTT
GTGCGTACCGTTTATGGTACCGTGCCGCTGCGTTTTGCCATGGGC
TGGCCGGTGGTGGCAAGCTATGAAGAACTGGCCGGCTGCGCACGC
TGGATGGGTGGTCGTATTCCGACCATGGAAGAAGCACGCAGCATC
TATGCCTATGTGGATCGCATGAAACCGGAATTTGAAAAAAGCCTG
GGTAATACCATTCCGGCCGTTAATGGTCATCTGATTAATGAAGGC
GTTTTTGAAACCCCGCCGAGTCCGCATCTGAGTAATGGCAATAGC
GGTGCCGCCACCAGTCTGAATCCGCATGATCTGTTTATTGATCTG
GAAGGCGCCAATATGGGCTTTAAACATTGGCATCCGGTTAGCGTT
GCAGATAAAGGCAATAAGCTGTGTGGTCAGTATGATCTGGGTGGC
GTTTGGGAATGGACCAGTACCGTTCTGGAAAAGCATGAAGGTTTT
GAACCGATGGAACTGTATCCGGGTTATACCGCCGATTTCTTTGAT
GGTAAACATAATGTTACCCTGGGTGGCAGCTGGGCCACCCATCCG
CGTCTGGCAGGCCGTAAAACCTTTATTAATTGGTATCAGCGCAAT
TATCCGTATGTTTGGGCCAGCGCCCGTATTGTTGCAGATTTGtaa
<SEQ ID NO: 52; PRT;
C3Egt1; 1_XM_008027421;
Exserohilum turcica>
MATQIIDIRVDTAESDILAHIKKGLRPEAGGEKTLPTLLLYDQEG
LRLFEQITYEEEYYLTNAEIEVLEKYAHEIAQRVPSGSIVVELGS
GNLRKVNILLQAMDRLAKDVEYYAVDLSLPELQRTFSQIPIEGYS
HVKCFGLHGTYDDALEWLKSPAVEAKPKTILWLGSSLGNFKRHEV
PPFLAGFGRVLQTGDTMLIGIDSCKDPERVFHAYNDRNGVTHKFI
LNGLKHANALMGDRTFNMDDWEIIGEYDVKAGRHHAFVAPRKDVV
VDGVTVKQGERIRIEESYKYSHKEAKKLWELARLSESAVWANSMG
DYGLHLVSKPAFFFPTNPEEYAASPVPSLAEWQELWKSWDAVSKQ
MIPEAELLSKPIKLRNECIFYLGHIPTFLDIHIARATDGQPTEPA
YFWKIFERGVDPDVDNPTQCHAHSEVPEEWPAVKTILEYQQTIRK
NTEALYQSGEAENNVRVSRGLWIAFEHEAMHLETLLYMLIQSDKI
LPPPGTKVPDFAAYAAHSDVLAVENQWFTIPESDIDIGLDDPEND
FKTKRYFGWDNERPRRSAHVKAFRSKARPITNGEYATYLFQTGKK
EIPASWSETSYSNANGTTMTKRDSVINGHANGDSDPSYSMIEGKF
VRTVYGTVPLRFAMGWPVVASYEELAGCARWMGGRIPTMEEARSI
YAYVDRMKPEFEKSLGNTIPAVNGHLINEGVFETPPSPHLSNGNS
GAATSLNPHDLFIDLEGANMGFKHWHPVSVADKGNKLCGQYDLGG
VWEWTSTVLEKHEGFEPMELYPGYTADFFDGKHNVTLGGSWATHP
RLAGRKTFINWYQRNYPYVWASARIVADL
<SEQ ID NO: 53; DNA;
C4Egt1; 1_XM_018186317;
Paraphaeosphaeria sporulosa>
ATGGATGTGACCCGTGCCCTGAGTGGCGCACGTCTGCGCCCTCTG
CGTTTTCTGAAAGAAACCAGCTTTGAAAAAATGAGTGCCAAAACC
AGCACCGAAATTATTGATATTCGCCCGGGCCCGACCGAATTTGAT
ATTCTGCAGGATATTAAGGATGGCCTGCGTCCGGAACATGGCGGC
GAAAAAACCCTGCCGACCATGCTGCTGTATGATGAAGCAGGCCTG
CGTCTGTTTGAAAAAATTACCTATGTTAAGGACTACTACCTGACC
GATAGCGAAATTGAAGTGCTGGATACCTATGCAGATCAGATTGCC
GAACGCATTAAGGCCGGTAGCGTTCTGGTGGAACTGGGTAGTGGT
AATCTGCGTAAAGTGAATATTCTGCTGCAGGCAATTGAACGTCTG
GGTAAAGATGTTGAATATTATGCAGTGGATCTGAGCCTGCCGGAA
CTGGAACGCACCTTTGCCGAAATTCCGACCAATTATCAGCATGTG
AAACTGAAAGGTCTGTATGGTACCTATGATCATGCCCTGGAATGG
CTGAAAAGCCCGAAAGTGAGCGCAAAACCGAAAACCATTCTGTGG
CTGGGCAGTAGCCTGGGTAATTTTACCCGTGCAGATGTGCCGCCG
TTTCTGACCGGTTTTCGTGAAGCACTGCAGCCGGGTGACACCATG
CTGATTGGTATTGATAGCTGCAAAGAACCGGAACGCGTTTTTCGC
GCCTATAATGATACCGATGGTGTTACCCGTGATTTTACCCTGAAT
GGTCTGAAAAATGCAAATCGCATTATGGGCACCGAAGCCTTTAAA
CCGCATGAATGGGAACATTGTGGTGAATTTGTGGAAAAAGATGGT
TATCATCGCGCATTTGTTAGCCCGCTGAAAGATGTTATTATTGAT
GGCGTTCATATCAAAAAGGGTGAACGTATTCGCATTGAAGAAAGC
TGGAAATTTTCTAAAGAGGAAATTGAACACCTGTGGAGCGAAGCC
GGCCTGATTCCGAATACCACCTTTAGTACCGCCCGTGGTGACTAT
GGTCTGCATTTTGTGAGCAAACCGGCATTTTTCTTTCCGACCAAA
CCGGAACGTTATGCAGCAAAACCGGTGCCGAGTATTGCCGAATGG
CGCGAACTGTGGACCGCCTGGGATGATATTGCCCAGAAAATGGTG
CCGACCGAAGCACTGCTGAGCAAACCGATTAAGCTGCGTAATGCA
GTGATTTTCTATCTGGGCCATATTCCGACCTTTCTGGATATTCAT
CTGACCCGCGCAACCGATGAAAAACCGACCGAACCGGCCGCATAT
ACCAAAATTTTTGAACGTGGCATTGATCCGGATGTTGATAATCCG
GAACAGTGTCATGATCATAGTGAAATTCCGGAAACCTGGCCGCCG
CGCGATGAAGTTCTGGCCTTTCAGGATGCAGTTCGTAAACGTACC
AAAGCACTGTATGATAGTAATGCAGCCCATGAAAATCCGCGTGTG
AGCCGTGCCCTGTGGCTGGCTTTTGAACATGAAGCAATGCATCTG
GAAACCCTGCTGTATATGCTGATTCAGAGTGAACGTATTCTGCCG
CCGCCGGGCAGCGTGATGCCGAGCTTTGATGCCCTGGCACGCAAA
AGCAAAAAAGCACGCGTGGAAAATCAGTGGTTTACCATTCCGGAA
GCCGAAATTCGTGATGGTCTGGATGATCCGGAAGATGGCAGCGCC
GCAAAACGCTATTTTGGCTGGGATAATGAAAAACCGACACGCAGC
CTGCGCGTGAAAAGTTTTAAAGCAAAAGCACGCCCGATTACCAAT
GGCGAATATGCCGATTATCTGGTTCAGACCGGCAAATGTGCCGTG
CCGGCAAGTTGGTGCGATGGCCTGGACCCTGCAGCAAAAGTGGTG
GTTAATGGTGTGAATCGCCGTAATGGTACCAATAGCATTAATAGC
AGTATTGATAAGGTTATGCAGGGTAAATATGTGCGCACCGTTTTT
GGTACCGTTCCGCTGCATTATGCCCTGGATTGGCCGGTTGTGGCA
AGCTATGATGAACTGGCAAGTTGTGCCCAGTGGATGGGCGGTCGC
ATTCCGACCCTGGAAGAAGCACGCAGTATCTATAATTATGTGGAA
CATGGCAAAGCCCAGGAATTTGAAAAAACCCATGGCAATAAGATT
CCGGCAGTGAATGGTCATCTGATTAATAATGGTGTTAGTGAAAGC
CCGCCGAGTCAGCATCTGAGCAATGGTAGTAGCGGCACCGGCAGT
GATCCGAAACCGGGCGATCTGTTTATTGATCTGGAAGGCACCAAT
ACCGCCTTTCAGCATTGGCATCCGATTAGCGTGGCAGAAAAAGGT
GACAAACTGTGTGGTCAGGCCGATCTGGGCGGTGCATGGGAATGG
ACCAGTACCGTGCTGGAAAAACATGATGGTTTTAGCGCAATGCCG
CTGTATCCGGGTTATACCGCAGATTTCTTTGATGGCAAACATAAT
ATTATGCTGGGCGGTAGCTGGGCCACCCATAGCCGTATTGCCGGT
CGTAAAACCTTTGTGAATTGGTATCAGCGTAATTATCCGtaa
<SEQ ID NO: 54; PRT;
C4Egt1; 1_XM_018186317;
Paraphaeosphaeria sporulosa>
MDVTRALSGARLRPLRFLKETSFEKMSAKTSTEIIDIRPGPTEFD
ILQDIKDGLRPEHGGEKTLPTMLLYDEAGLRLFEKITYVKDYYLT
DSEIEVLDTYADQIAERIKAGSVLVELGSGNLRKVNILLQAIERL
GKDVEYYAVDLSLPELERTFAEIPTNYQHVKLKGLYGTYDHALEW
LKSPKVSAKPKTILWLGSSLGNFTRADVPPFLTGFREALQPGDTM
LIGIDSCKEPERVFRAYNDTDGVTRDFTLNGLKNANRIMGTEAFK
PHEWEHCGEFVEKDGYHRAFVSPLKDVIIDGVHIKKGERIRIEES
WKFSKEEIEHLWSEAGLIPNTTFSTARGDYGLHFVSKPAFFFPTK
PERYAAKPVPSIAEWRELWTAWDDIAQKMVPTEALLSKPIKLRNA
VIFYLGHIPTFLDIHLTRATDEKPTEPAAYTKIFERGIDPDV
DNPEQCHDHSEIPETWPPRDEVLAFQDAVRKRTKALYDSNAAHEN
PRVSRALWLAFEHEAMHLETLLYMLIQSERILPPPGSVMPSFDAL
ARKSKKARVENQWFTIPEAEIRDGLDDPEDGSAAKRYFGWDNEKP
TRSLRVKSFKAKARPITNGEYADYLVQTGKCAVPASWCDGLDPAA
KVVVNGVNRRNGTNSINSSIDKVMQGKYVRTVFGTVPLHYALDWP
VVASYDELASCAQWMGGRIPTLEEARSIYNYVEHGKAQEFEKTHG
NKIPAVNGHLINNGVSESPPSQHLSNGSSGTGSDPKPGDLFIDLE
GTNTAFQHWHPISVAEKGDKLCGQADLGGAWEWTSTVLEKHDGFS
AMPLYPGYTADFFDGKHNIMLGGSWATHSRIAGRKTFVNWYQRNY
P
<SEQ ID NO: 55; DNA;
C5Egt1; 1_XM_007289816;
Marssonina brunnea>
ATGGCACCGAAAATTGATATTATCGATATTCGTCATAACGCAGTG
GAAATGAGTCTGAAAGATGAAATTGTTAAGAGCCTGAAACCGCAG
GAAGGCCCGAAACGCCTGCCGACCCTGCTGCTGTATGATGAACGC
GGCCTGCAGCTGTTTGAAGAAATTACCTATCTGGAAGAATACTAT
CTGACCAATGCAGAAATTGATGTGCTGCAGCGCAGCGCATGCAAT
ATTGCAGAAGCAATTCCGCCGGGTAGTATGGTGGTGGAACTGGGT
AGTGGTAATCTGCGTAAAGTTAGTATTCTGCTGCAGGCACTGGAT
CAGGCCGGCAAAGATATTGATTATTATGCCCTGGATCTGAGTCTG
AAAGAACTGTATCGTACCCTGGAACAGGTTCCGGCCTTTAAACAT
GTGACCTGCCATGGTCTGCATGGCACCTATGATGATGGTCTGGAT
TGGCTGAAAATTCCGGAAAATATTACCCGCCCGAAATGCGTGATG
AGCCTGGGTAGTAGCATTGGCAATTTTAGTCGTGCCGGTGGCGCA
GAATTTCTGAAAGGTTTTGCCGAAGTGATGCAGGATAGCGATCTG
ATGCTGGTTGGTCTGGATGCCACCGAAGATCCGGCAAAAGTGTAT
CATGCATATAATGATCGTGAAGGTAAAACCCATAAATTCATTCTG
AATGGTCTGACCAATGCCAATGGTATCTATAATGAAGAAATCTTT
GAGCCGAATGATTGGAAAGTGATTGGCGAATATGTGTTTGATGCA
GAAGGCGGCCGTCATCAGGCCTTTTGCAGCCCGGTTCATGATGTG
AGTGTTAAAGGTGTTCAGATTAAGGCAGGTGAACGCGTGCAGATT
GAAGAAAGCCTGAAATATAGTCCGGAAGGTAGCGCCCAGCTGTGG
AAAGCCAGTGGCCTGATTGAAGTTGATCGTATGAGTGCAAGCAGT
GATAGCTATAGCCTGCATCTGCTGAAACGTAATATGGCCTTTAAA
ACCGATCCGAGTCTGTATGCCGCCAGTACCGTGCCGACCCGCAAA
GATTGGAAAGGTCTGTGGACCGTGTGGGATCTGATTACCCAGAAT
ATGATTCCGAAAACCGAACTGAATGAAAAACCGATTAAGCTGCGC
AATGCCTGTATTTTCTATCTGGGTCATATTCCGACCTTTACCGAT
ATTCAGCTGGAAAAAGTGACCAAACAGCCGCGTTGCGAACCGGGC
TATTTTAAAGAAATTTTTGAACGTGGCATCGATCCGGATGTGGAT
AATCCGGAACGCTGTCATGATCATAGCGAAGTTCCGGAAGAATGG
CCGCCGCTGCAGGATATTCTGGGTTATCAGGATCAGGTGCGTGCC
AAAATTGAAAAAATTACCGCAAGCGAAAGTATTCCGCGTGATGTT
GGCCGTGCCCTGTGGATTGGTTTTGAACATGAAATTATGCATCTG
GAAACCCTGCTGTATATGCTGCTGCAGAGTGATAAAACCCTGCCG
CCGACCAAATTCAAACCGAATTTTGAAGAACTGGCAGCCGCCGAT
GAAGCAGCCCGCGTTGGCAATGAATGGTTTGAAATTCCGGAACAG
CGTATTACCATTGGTCTGGATGATCCGGAAGATAATAGTGGCGGT
GACCGCCATTTTGGCTGGGATTGCGAAAAACCGCCGCGCAGCGTT
GTGGTTCCGGCCTTCAAAGCACAGGCCCGCGCCATTACCAATGAA
GATTATGCCCGTTATCTGGAACAGACCCATGCCAGTAAAATTCCG
GCAAGCTGGACCGAAAGCGTGACCAATGGTCATACCAATGGCGTG
AGCAATGTGTATAGCAATGGTAATAGCAATGGTCATGCAATTACC
AGCACCCCGCTGACCAAAGAATATCTGGATGGTAAATTTGTGCGC
ACCGTGTATGGCCTGGTTCCGCTGATGTTTGCACTGCATTGGCCG
GTTTTTGCAAGCTATGATGAACTGGCAGGCTGTGCAAAATGGATG
GGTGGTCGCATTCCGACCCTGGAAGAAGCACGTAGCATCTATAGT
CATGTGGATGGCCTGCGTCTGAAAGAAGCCGAACAGCATCTGGTT
AAAACCGTTCCGGCCGTTAATGGTCATCTGGTTAATGAAGGCGTT
GAAGAAAGCCCGCCGAGTCGCGGCGCATGGCCTGGTGAAGGCAGC
GAACTGTTTACCGATCTGGCCAATGCCAATGTTGGCTTTAAACAT
TGGCATCCGATTGGCGTGACCAGCAATGGCGATAAACTGGCAGGT
CAGGCAGAAATGGGTGGCGTGTGGGAATGGACCAGTAGCGAACTG
CTGCGCCATGATGGCTTTGAACCGATGAAACTGTATCCGGCCTAT
ACCGCAGATTTCTTTGATGGCAAACATAATATTGTGCTGGGTGGC
AGCTGGGCAACCCATCCGCGCATTGCAGGCCGCAAAACCTTTATT
AATTGGTATCAGCGTAACTATCCGTATGCCTGGGCCGGCGCACGC
CTGGTTCGCGACGTTtaa
<SEQ ID NO: 56; PRT;
C5Egt1; 1_XM_007289816;
Marssonina brunnea>
MAPKIDIIDIRHNAVEMSLKDEIVKSLKPQEGPKRLPTLLLY
DERGLQLFEEITYLEEYYLTNAEIDVLQRSACNIAEAIPPGSMVV
ELGSGNLRKVSILLQALDQAGKDIDYYALDLSLKELYRTLEQVPA
FKHVTCHGLHGTYDDGLDWLKIPENITRPKCVMSLGSSIGNESRA
GGAEFLKGFAEVMQDSDLMLVGLDATEDPAKVYHAYNDREGKTHK
FILNGLTNANGIYNEEIFEPNDWKVIGEYVFDAEGGRHQAFCSPV
HDVSVKGVQIKAGERVQIEESLKYSPEGSAQLWKASGLIEVDRMS
ASSDSYSLHLLKRNMAFKTDPSLYAASTVPTRKDWKGLWTVWDLI
TQNMIPKTELNEKPIKLRNACIFYLGHIPTFTDIQLEKVTKQPRC
EPGYFKEIFERGIDPDVDNPERCHDHSEVPEEWPPLQDILGYQDQ
VRAKIEKITASESIPRDVGRALWIGFEHEIMHLETLLYMLLQSDK
TLPPTKFKPNFEELAAADEAARVGNEWFEIPEQRITIGLDDPEDN
SGGDRHFGWDCEKPPRSVVVPAFKAQARAITNEDYARYLEQTHAS
KIPASWTESVINGHTNGVSNVYSNGNSNGHAITSTPLTKEYLDGK
FVRTVYGLVPLMFALHWPVFASYDELAGCAKWMGGRIPTLEEARS
IYSHVDGLRLKEAEQHLVKTVPAVNGHLVNEGVEESPPSRGAWPG
EGSELFTDLANANVGFKHWHPIGVTSNGDKLAGQAEMGGVWEWTS
SELLRHDGFEPMKLYPAYTADFFDGKHNIVLGGSWATHPRIAGRK
TFINWYQRNYPYAWAGARLVRDV
<SEQ ID NO: 57; DNA;
C6Egt1; 1_XM_024470276;
Pseudogymnoascus destructans>
ATGACCAATAGTACCACCCCGCCGCCGGATGTTGTTGATCTGGAT
AAATTTCATACCCATGATGATCCGCGTCATACCCGCCTGACCACC
CCGAGCAAAGCCACCCTGCCGCCGGCTACCCCGCCTAGCCCTGCA
CAAAGCACCCTGGATTTTATTGATATTATTGATATCCGCCGTGAT
GCCCTGGGCAGTAGTCTGGATCTGGGTCGCGATATTATGGCCCAG
CTGGCACCGGCCCGCGGTCCTAAAAAGATGCCGACCCTGCTGCTG
TATGATGAAAAAGGCCTGCAGACCTTTGAAGAAATTACCTATCTG
GAAGAATACTATCTGACCAATGCAGAAATTGAAGTGCTGGAACGC
AATGCAGAAGAAATGGCCCGCAATATTCAGGCAGAAAGCATGGTT
ATTGAACTGGGTAGCGGTAATCTGCGTAAAGTGAGCATTCTGCTG
AATGCCCTGGAAAAAGCCGAAAAAAGTATTCATTATTACGCACTG
GATCTGAGCAAACGCGAACTGGAACGTACCCTGAGCAGTGTTCCG
CGTTTTGAACATGTGGTTTGCCATGGTCTGCTGGGCACCTATGAT
GATGGCCTGGAATGGATTCGTAGTGGTTGCAATGCAAGCTGGCCG
AAATGCATTATGAGTCTGGGTAGTAGTATTGGTAATTTTAATCGC
GGCGATGCCGCAGAATTTCTGAAAGGTTTTGCAGATATGCTGCGT
CCGAGTGATAGCATGATTATTGGCCTGGATGCCTGCAATGATCCG
GCCAAAGTTTATCATGCCTATAATGATAGCCTGGGCATTACCCAT
AAATTCATTCTGAATGGTCTGGATAATGCAAATAGCATTCTGGGC
GAAAATGTGTTTGATACCAATGATTGGGAAGTGATTGGTGAATAT
GTTTGTGATAAAGATGGTGGTCGCCATCGCGCCTTTTATGCCCCG
AAACGTGATATTACCATTCGTGGTGTTTTTATTGAACAGGGTGAA
CGCGTTCAGGTTGAACAGAGCCTGAAATATAGCCAGGCCGAAAGC
GAAGGCATGTGGGCAGCAGCCGGCCTGAAAGAAGTGGGCAAATGG
GGTGCAACCAAAGAACAGTATAATATTCATATGCTGACCAAACGT
GCAAAACCGTTTCAGCTGCATCCGAGTCAGTATGCCCTGACCCCG
ACCCCGACCCTGGAAGATTGGCGTGGCCTGTGGAGCACCTGGAAT
ACCGTGGCCCGTGGTATGATTCCGAATAATGAACTGCTGGCAAAA
CCGATTAAGCTGCGTAATGCCTGCATTTTCTATCTGGGCCATATT
CCGACCTTTCTGGATCTGCAGCTGAGCAAAGCCACAGGTGTGCCG
CTGTGTGAACCGAGTCATTATCCGCAGATTTTTGAACGTGGTATT
GATCCGGATGTGGATAATCCGGATAATTGCCATGCACATAGTGAA
ATTCCGGATCAGTGGCCGCCGGTGGAAGAAATTCTGGAATATCAG
GCACAGGTGCGCCGTAAAGTGGAAGGTCTGTATGCCAGCGGTGTG
CCGGAAGCAAGTCGTAAAGTGGGTCGTAGTCTGTGGATTGGCCTG
GAACATGAAATTATGCATCTGGAAACCCTGCTGTATATGCTGCTG
CAGAGCGATAATTGTATGCCGCCGCCGCGCACCGTGAAACCGGAT
TTTGAAGAACAGGCCCGTCGTGATGCAGAACGTGAAGTGGAAAAT
CAGTGGTTTACCATTCCGGAACAGGATATTGCCCTGGGCCTGGAT
GATCCGGAAGATAATAGCGGTGACGGTCATTTTGGCTGGGATAAT
GAAAAACCGGTTCGTAAAGCCCATGTTCGTAGCTTTCAGGCAAAA
GGTCGTCCGATTACCAATGAAGAATATGCAATCTATCTGGATGCA
ACCGATAATGAAAATCTGCCGGCAAGTTGGACCCGTCAGCATGCC
AATGGCGATCTGAGCGCACATACCCCGAATGGCAATACCAATGGT
TATACCAATGGCAATGGTCATACCAATGGTAATGGTCTGACCAAT
GGCAACGGCCATACCGATGGTAGCGGCCATACCAATGGCAATGGC
TATCTGAGTAATGGCTATACCAATGGTCTGACAAAACTGCATCCG
TCATATATTAGTAATATCCTGGTGCGTACCGTGTATGGTCCGGTT
AGCCTGGCCCATGCACTGCATTGGCCGGTTAGTGCCTGTTATGAT
GAACTGCGTCGCTGCGCAAAATGGATGGGTGGCCGCATTCCGACC
GTTGAAGAAGCCCGTAGCATCTATAGCTATGTTGATGAACGCCGT
CTGAAAGAAGTTCGCAATGCCCGCCGTGTTCCGGCCGTGAATGCA
CATCTGGTTAATAATGGCGTGGAAGAAAGTCCGCCGCTGCGTGAT
CCGGCCGGTAGTCCTGCCAATCCGCATAGTGCCCTGTTTACCGAT
CTGGAAGGTGCCAATGTGGGCTTTAAACATTGGCATCCGGTGGCC
GTTACCGCCGATGGTGACAAACTGGCCGGTCAGGGCGAAATGGGC
GGTGTTTGGGAATGGACCAGCAGTGTGCTGGAACGTCATGAAGGC
TTTCGCGAAATGGAACTGTATCCGGCCTATAGTGAAGATTTCTTT
GATGGCAAACATAATGTGGTGCTGGGTGGCAGCTGGGCAACCCAT
CCGCGCATTGCCGGCCGCAAAAGTTTTATTAATTGGTATCAGCGT
AACTACCCGTATGTGTGGGCAGGCGCCCGCCTGGTGCGCGACATT
taa
<SEQ ID NO: 58; PRT;
C6Egt1; 1_XM_024470276;
Pseudogymnoascus destructans>
MTNSTTPPPDVVDLDKFHTHDDPRHTRLTTPSKATLPPATPPSPA
QSTLDFIDIIDIRRDALGSSLDLGRDIMAQLAPARGPKKMPTLLL
YDEKGLQTFEEITYLEEYYLTNAEIEVLERNAEEMARNIQAESMV
IELGSGNLRKVSILLNALEKAEKSIHYYALDLSKRELERTLSSVP
RFEHVVCHGLLGTYDDGLEWIRSGCNASWPKCIMSLGSSIGNFNR
GDAAEFLKGFADMLRPSDSMIIGLDACNDPAKVYHAYNDSLGITH
KFILNGLDNANSILGENVEDTNDWEVIGEYVCDKDGGRHRAFYAP
KRDITIRGVFIEQGERVQVEQSLKYSQAESEGMWAAAGLKEV
GKWGATKEQYNIHMLTKRAKPFQLHPSQYALTPTPTLEDWRGLWS
TWNTVARGMIPNNELLAKPIKLRNACIFYLGHIPTFLDLQLSKAT
GVPLCEPSHYPQIFERGIDPDVDNPDNCHAHSEIPDQWPPVEEIL
EYQAQVRRKVEGLYASGVPEASRKVGRSLWIGLEHEIMHLETLLY
MLLQSDNCMPPPRTVKPDFEEQARRDAEREVENQWFTIPEQDIAL
GLDDPEDNSGDGHFGWDNEKPVRKAHVRSFQAKGRPITNEEYAIY
LDATDNENLPASWTRQHANGDLSAHTPNGNTNGYTNGNGHTNGNG
LTNGNGHTDGSGHTNGNGYLSNGYTNGLTKLHPSYISNILVRTVY
GPVSLAHALHWPVSACYDELRRCAKWMGGRIPTVEEARSIYSYVD
ERRLKEVRNARRVPAVNAHLVNNGVEESPPLRDPAGSPANPHSAL
FTDLEGANVGFKHWHPVAVTADGDKLAGQGEMGGVWEWTSSVLER
HEGFREMELYPAYSEDFFDGKHNVVLGGSWATHPRIAGRKSFINW
YQRNYPYVWAGARLVRDI
<SEQ ID NO: 59; DNA;
C7Egt1; 1_XM_007834197;
Pestalotiopsis fici>
ATGCCGAGCGCCACCGAAATGTTTTATGAAAGTCAGGCAATTCCG
ACCGCCTTTGAACTGAGTAAAGGTCTGAGCAAAAATCCGAAAAGC
AGTCGCCGTCTGGATATTATTGATATTCGCCAGGCCGCAGTGGAA
CTGAATCTGAAAGAAGAAATTCATCAGCTGCTGCGTCCGCAGGAA
GGTCCGCGCAAACTGCCGACCCTGCTGCTGTATGATGAACGTGGT
CTGCAGCTGTTTGAACAGATTACCTATCTGGAAGAATATTATCTG
ACCAATAGCGAAATTCAGGTGCTGCGTAGTAGTGCACAGGCAATT
GCCAAAGCAATTCCGAGTGGTAGCATGGTGGTTGAACTGGGTAGC
GGTAATCTGCGTAAAGTTCAGATTCTGCTGCAGGCCCTGGAAGAT
GCCGGTAAAGATATTGATTATTATGCCCTGGATCTGGATAAACGT
GAACTGGAACGTACCCTGGCACAGGTGCCGGCCTTTCGTTTTGTT
ACCTGCCATGGCCTGCATGGTACCTATGATGATGGCCGCGTGTGG
CTGAAAAATAGTAGCGTTAGCGCACGCCCGAAATGTGTGATGAGC
CTGGGTAGCAGTATTGGTAATTTTCATCGCAGTGATGCAGCCGCA
TTTCTGCGCAGTTTTAGTGATGTGCTGCAGCCGAGCGATACCTTT
CTGCTGGGTCTGGATAGTTGTACCAATCCGAGCAAAGTGTATCAT
GCATATAATGATCGTCATGGCGTTACCCATCAGTTTATTCTGAAT
GGTCTGCGTCATGCCAATGAAGTGCTGGAAGATGAAGTTTTTAAT
CTGGATGAATGGCAGGTTATTGGTGAATATGTGTATGATGTGGAA
GGCGGCCGTCATCAGGCATTTTATAGCCCGAGCCGCGATGTGACC
ATTCTGGGCGAAAATATTAAGGCCCAGGAACGTATTCAGGTTGAA
CAGAGCCTGAAATATAGTGAAGATGGTATGAAAAAGCTGTGGAGC
GAAGCCGGTGTTGTTGAAACCGATCGCTGGATGACCGATAATAAT
GAATATGGTCTGCATCTGCTGACCAAACCGACCACCATGCCGTTT
AGCCTGGACCCTCGCCAGTATGCAGGTAGTGTGCTGCCGACCTTA
GATGATTGGAAAGCCCTGTGGAGCACCTGGGATACCGTTACCCAG
CGTATGCTGCCGGATGAAGAACTGCTGGAAAAACCGATTAAGCTG
CGTAATGCATGTATTTTCTATCTGGGTCATATTCCGACCTTTCTG
GATATTCAGCTGACCAAAACCACCAAACAGCCGCCGACCGAACCG
GTTAGCTATAGCGCCATTTTTGAACGCGGTATTGATCCGGATGTG
GATAATCCGGAACATTGCCATAGTCATAGCGAAATTCCGGATGAA
TGGCCGAAACAGGCAGATATTCTGCGCTATCAGAATAATGTGCGC
GTGCGTCTGACCGGTCTGTATAGCCATGGCCCGGAAAGCATTCCG
CGTGATGTTGCCCGTGCAATTTGGGTGGGTTATGAACATGAACTG
ATGCATATGGAAACCCTGCTGTATATGATGCTGCAGAGTGATAAA
ACCCTGCCGCCGCCGCATATTCCGCGTCCGGATTTTAAAGGTCTG
GCACGTAAAGCATATGCCGAACGTACCGCAAATGAATGGTTTACC
ATTCCGGAACAGCATATTCTGGTTGGTCTGAATGATCCGGAAGAT
GATGAACGTTTTAAAGGTCATTTTGGTTGGGATAATGAAAAACCG
GCCCGCAAAATTAATGTGAAAAGTTTTCAGGCAAAGGGTCGCCCG
ATTACCAATGAAGAATATGCATATTATATGTACGAGACAAAGGTG
ACCAAAATTCCGGCCAGTTGGGCAGAAGCCCCGCAGCATCATGTG
AATGGTAGCAATGGCACCAGTCATGAACATACCAATGGTCAGGCC
AATGGTCATGCAAATGGCCATGTGAATGGCCATGTTAATGGTAGC
CATGATACCGGTAGCACCCTGCTGCCGAGCAGCTTTCTGGATGAT
AAAACCGTGCGCACCATCTATGGCCTGGTTCCGCTGGAATATGCC
CTGGACTGGCCGGTTTTTGCAAGTTATGATGAACTGGCCGGTTGT
GCAGCATGGATGGGCGGTCGCATTCCGACCTTCGAAGAAGCACGT
AGTGTTTATGCATATGTGGATTATCTGAAAAAGAAGGAAGCCGAA
CGTAAACTGGGTAAAACCGTTCCGGCCGTGAATGGCCACCTGGTG
AATGATGGCGTGGAAGAAACCCCGCCGAGTCGTGGTTTTGGTATG
CAGGTGAATGGCGAAGCAAGTGAAAATGATGATAGCTTTGTGGAT
CTGGAAGGTAGCAATGTTGGTTTTAATCATTGGCATCCGATGCCG
ATTACCAGCCGCGGCAATCGTCTGGCCGGCCAGAGTGAAATGGGT
GGTGTGTGGGAATGGACCAGTAGTCATCTGACCCGTCATGAAGGC
TTTGAACCGATGGCACTGTATCCGGCATATACCAGTGATTTCTTT
GATGGCAAACATAATGTTGTTCTGGGCGGTAGCTGGGCAACCCAT
CCGCGTATTGCCGGTCGCAAAAGCCTGtaa
<SEQ ID NO: 60; PRT;
C7Egt1; 1_XM_007834197;
Pestalotiopsis fici>
MPSATEMFYESQAIPTAFELSKGLSKNPKSSRRLDIIDIRQAAVE
LNLKEEIHQLLRPQEGPRKLPTLLLYDERGLQLFEQITYLEEYYL
TNSEIQVLRSSAQAIAKAIPSGSMVVELGSGNLRKVQILLQALED
AGKDIDYYALDLDKRELERTLAQVPAFRFVTCHGLHGTYDDGRVW
LKNSSVSARPKCVMSLGSSIGNFHRSDAAAFLRSFSDVLQPSDTF
LLGLDSCTNPSKVYHAYNDRHGVTHQFILNGLRHANEVLEDEVEN
LDEWQVIGEYVYDVEGGRHQAFYSPSRDVTILGENIKAQERIQVE
QSLKYSEDGMKKLWSEAGVVETDRWMTDNNEYGLHLLTKPTTMPF
SLDPRQYAGSVLPTLDDWKALWSTWDTVTQRMLPDEELLEKPIKL
RNACIFYLGHIPTFLDIQLTKTTKQPPTEPVSYSAIFERGIDPDV
DNPEHCHSHSEIPDEWPKQADILRYQNNVRVRLTGLYSHGPESIP
RDVARAIWVGYEHELMHMETLLYMMLQSDKTLPPPHIPRPDFKGL
ARKAYAERTANEWFTIPEQHILVGLNDPEDDERFKGHFGWDNEKP
ARKINVKSFQAKGRPITNEEYAYYMYETKVTKIPASWAEAPQHHV
NGSNGTSHEHTNGQANGHANGHVNGHVNGSHDTGSTLLPSSFLDD
KTVRTIYGLVPLEYALDWPVFASYDELAGCAAWMGGRIPTFEEAR
SVYAYVDYLKKKEAERKLGKTVPAVNGHLVNDGVEETPPSRGFGM
QVNGEASENDDSFVDLEGSNVGFNHWHPMPITSRGNRLAGQSEMG
GVWEWTSSHLTRHEGFEPMALYPAYTSDFFDGKHNVVLGGSWATH
PRIAGRKSL
<SEQ ID NO: 61; DNA;
C8Egt1; 1_XM_013493644;
Aureobasidium subglaciale>
ATGGATACCAGTACCGCACCGAAAATTATTGATATTCGTCAGGAT
GGCGGTGGTCTGACCCCGCTGGTGCCGGAAATTCGTGAAGGTCTG
AATGCCCGTGAAGGTCAGGAAAAGAAACTGCCGACCCTGCTGCTG
TATAGTGAAGATGGTCTGAAACTGTTTGAAAAAATTACCTATCTG
GAGGAATACTATCCGACCGGTCAGGAAATTCAGGTTCTGGAAGCA
TATGCAGATCGCATTGCCGATCGTATTGCCCTGGAAAGTAATAGC
ATGCTGGTGGAACTGGGTAGTGGCAATCTGCGCAAAGTGCGCATT
CTGCTGGATGCCCTGGATCGCAAAGGTAAAGATGTTAGTTATTAT
GCCCTGGATGTTAGCGAAGTTGAACTGGAACGCACCCTGGCCGAA
GTTCCGCAGGGCACCTTTAAACATGTTCAGTGCCATGGTCTGCTG
GGTACCTATGATGAAGGTCTGGATTGGCTGAAAAAACCGGAAAAT
GCACATCGCAGTAAAACCGTTCTGAGCCTGGGTAGCAGTATTGGT
AATTTTAGTCGTGATGAAGCCGCAAAATTTCTGAGCCAGTTTAGC
GAAACCCTGGACCCTAATGATACCCTGCTGTTAGGTATTGATGCA
TGTACCGATGCAGATAAAGTTTATCATGCATATAACGATCGCGAA
GGTCTGACCCATGAGTTTATTCTGTGTGGCCTGAAACAGGCCAAT
CGCCTGCTGGGTTATGATGCCTTTGATACCAAAATGTGGGAAGTT
ATTGGCCGTTATAATAAGGAAACCGATCGTCATGAAGCCTTTGTT
AGCCCGAAAAAAGATGTTACCATTGAAGGTGCACTGATTCGTGCC
GGTGAACAGGTTCGCATTGAAGAAAGTTATAAATATAACAGCGTG
CAGAGTGAACGCCTGTGGAGTGATGCCGGCCTGACCGAAGGTGCC
AAATGGACCAATACCGATGGCGATTATGCACTGCATCTGCTGAAT
AAGCCGAAAGTTCAGTATCCGCTGGTGGCCGAAAAATATGCCGCC
CAGCCGGTGCCGAGCCTGGAAGAATGGGATCAGCTGTGGGCAGCA
TGGGATGCAGTGACCCTGGAAATGATTCCGGAAGAAGATTTGCTG
GAAAAACCGATTAAGCTGCGCAATGCCTGTATTTTCTATCTGGGC
CATATTCCGACCTTTATGGATATTCATCTGACCCGCGCAACCCAT
GGCAAACCGACCGAACCGAGCAGCTATACCAGTATTTTTGAACGT
GGTATTGATCCGGATGTTGATGATCCGGAACAGTGTCATGCCCAT
AGCGAAATTCCGGATAGCTGGCCGCCGGCCACCGAAATTCTGGAT
TTTCAGAGCAAAGTTCGCATTCGTGTGAAAAAACTGTATGCAACC
GGCCAGGCCGTTAAAGATCGTGCCGTTAGTCGTGCAATGTGGCTG
AGTTATGAACATGAAATTATGCATCTGGAAACCCTGCTGTATATG
CTGATTCAGAGTGAAAAAACCATGGCCCCGCCGACCACCGTTATG
CCGGATTTTGAAGCACTGGCAGTTCAGGCACGCCAGCGCGCAGTG
GAAAATCAGTGGTTTGATATTCCGGCACAGAAAGTTGAAATTGGC
ATTGAAGATCCGGATGATAATAGTGGTCCGGAACATTTCTTTGGC
TGGGATAATGAAAAACCGAAACGCACCGTTCATGTTCCGGCCTTT
AAAGCAAAAGGTCGTCCGATTACCAATGGTGAATTTGCCAAATAT
CTGGAAGAAAATCATCTGGATACCCTGCCGGCCAGCTGGCATACC
CTGAGCCATACCCGTGGCACCGAAACCAATGGTCATACCAGCGGT
ATTAGTAGCAGCTTTCTGCAGGGTAAAGCCGTTCGTACCGTTTAT
GGTCCGATTAGTCTGAAACTGGCACTGGATTGGCCGGTTTGCGCC
AGTTATGATGAACTGCGCGGCTGTGCCCGTTGGATGGGCGGTCGT
ATTCCGAGCATGGAAGAAGCCCGCAGCATCTATCGCCATGTTGAT
GAAGCCAAAACCCTGCAGGCCCATGAAAGCCTGGGTGCCAATATT
CCGGCAGTTAATGCCCATCTGGTGAATGATGGCGTGGAAGAAAGT
CCGCCGAGCAAAGCCCTGAGCAGCAGCAGCACCGGTCCGAATCCG
AATGATCTGTTTGTTGATCTGCTGGGTGCAAATGTTGGTTTTCGT
CATTGGCATCCGATGCCGGTGAGCCAGCATGGTAATAAGCTGGCC
GGTCAGAGTGAAATGGGCGGTGTTTGGGAATGGACCAGTAGCGTG
CTGGATAAACAGGAAGGCTTTGAAGCCATGCCGCTGTATCCGGGT
TATACCGCAGATTTCTTTGATGGCAAACATAATATTGTGCTGGGT
GGCAGTTGGGCAACCCATCCGCGTATTGCCGGCCGTAAAAGTTTT
GTTAATTGGTATCAGCGCAATTATCCGTTTGTTTGGGCCGGCGCC
CGCATTGTGAAAGATGCCtaa
<SEQ ID NO: 62; PRT;
C8Egt1; 1_XM_013493644;
Aureobasidium subglaciale>
MDTSTAPKIIDIRQDGGGLTPLVPEIREGLNAREGQEKKLPTLLL
YSEDGLKLFEKITYLEEYYPTGQEIQVLEAYADRIADRIALESNS
MLVELGSGNLRKVRILLDALDRKGKDVSYYALDVSEVELERTLAE
VPQGTFKHVQCHGLLGTYDEGLDWLKKPENAHRSKTVLSLGSSIG
NFSRDEAAKFLSQFSETLDPNDTLLLGIDACTDADKVYHAYNDRE
GLTHEFILCGLKQANRLLGYDAFDTKMWEVIGRYNKETDRHEAFV
SPKKDVTIEGALIRAGEQVRIEESYKYNSVQSERLWSDAGLTEGA
KWTNTDGDYALHLLNKPKVQYPLVAEKYAAQPVPSLEEWDQLWAA
WDAVTLEMIPEEDLLEKPIKLRNACIFYLGHIPTFMDIHLTRATH
GKPTEPSSYTSIFERGIDPDVDDPEQCHAHSEIPDSWPPATEILD
FQSKVRIRVKKLYATGQAVKDRAVSRAMWLSYEHEIMHLETLLYM
LIQSEKTMAPPTTVMPDFEALAVQARQRAVENQWFDIPAQKVEIG
IEDPDDNSGPEHFFGWDNEKPKRTVHVPAFKAKGRPITNGEFAKY
LEENHLDTLPASWHTLSHTRGTETNGHTSGISSSFLQGKAVRTVY
GPISLKLALDWPVCASYDELRGCARWMGGRIPSMEEARSIYRHVD
EAKTLQAHESLGANIPAVNAHLVNDGVEESPPSKALSSSSTGPNP
NDLFVDLLGANVGFRHWHPMPVSQHGNKLAGQSEMGGVWEWTSSV
LDKQEGFEAMPLYPGYTADFFDGKHNIVLGGSWATHPRIAGRKSF
VNWYQRNYPFVWAGARIVKDA
<SEQ ID NO: 63; DNA;
DIEgt1; 1_XM_016360447;
Verruconis gallopava>
ATGATCGGTGACAGTGCAGGTCTGGGCTTTGCAGATTTTAGTAGT
AGTAATGTTCACACCAAAGCCGCCAAACCGCATAGTCCGCTGAGT
AGCATTATTGAAATTCGTCAGGATCGTGAAGAACTGGATCTGCTG
ATTGATATTAAGAGTGGTCTGCGTGCAAGCGGCCCGGGCCGTAAA
ACCCTGCCGACCCTGCTGCTGTATGATGAACCGGGTCTGAAACTG
TTTGAAAAAATTACCTTTCTGGACGAATACTATCTGACCAATGCC
GAAATTGAAGTGCTGCATAAATGGGCAGGCAATATTGCAGATCGT
ATTAGTCCGAATAGCATTGTGCTGGAACTGGGTAGCGGCAATCTG
CGTAAAATTAAGATTCTGCTGGATGCATTTGAAGCAGCAAAAAAG
CCGGTGGAATATTATGCACTGGATGTGAGCCGTGTGGAACTGGAA
CGCACCCTGGCAGCCATTCCGGTTGGCGCATTCAAACATGTGAAA
TGTTTTGGTCTGCATGGCACCTATGATGATGGCCTGCAGTGGCTG
AAAAGCGAACAGATTGCAAAACGTAGTAAAGCAATTCTGAGTATG
GGTAGCAGCATTGGTAATTTTGCCCGCCATGAAGCAGTGCGCTTT
CTGCGTAGTTTTAGCGATGTGCTGCAGACCAGTGATGTTCTGCTG
ATTGGCATTGATGCATGTAAAGATCCGGATAAAGTGTTTCGCGCA
TATAATGATAGCCAGGGTGTTACCCATGAATTTGTGCTGAATGGC
CTGCAGCATGCAAATCAGCTGCTGGGCCATGATGCCTTTGATGTG
GAAAAATGGCGTGTGATTGGTGAATATGATGAAGTTAATGGTAAA
CATCACGCATTTGTTAGTCCGGTTCAGGATATGAATATTGATGGT
ATTCTGATTAAGGCAGGTGAACGCATTCGTATTGAAGAAAGTTTT
AAATACGACCTGATCGATCGTAGCCGTCTGTGGGAAGGTGCAGGT
CTGATTGAAGGTGCAAGTTGGACCAATGCCGAGGCCAATTATGGT
CTGCATATGGCATATAAACCGAAAGTTCAGTTTAGTAGCAAACCG
GAAGAATATGCCGCCAGCGCAGTGCCGACCCTGGCCGAGTGGAAA
GATTTGTGGACCATTTGGGATCTGGTTACCCAGCAGATGATTCCG
AAAGAAGAACTGCTGGCAAAACCGATTAAGCTGCGTAATGCATGC
ATTTTCTATCTGGGCCATATTCCGACCTTTCTGGCCATTCATCTG
GAAAAAGCCAGCTGTGAAGGCGTGGCCGGCCTGCGTGATTATCAG
CGTATTTTTGAACGCGGCATTGATCCGGATGTTGATAATCCGGAA
CTGTGTCATGATCATAGTGAAATTCCGGATGAATGGCCGCCGGAA
GTTGAAATTCTGGCCTTTCAGAGTAAAGTGCGTGATCAGGTTAAA
CAGATATATGATAGTCGTATGCATGAAAGCAGTCATGCCATTCGT
AAAGCACTGTGGATTAGTTTTGAACATGAAGTTATGCATATCGAA
ACCCTGCTGTATATGCTGATTCAGAGTGAAAAAACCCTGCCTCCG
CCGGGCGTTATTCATCCGGATTTTGAAGCACTGGCCTGTGAAGCC
AAAGCAAAAACCGTTCCGAATAAGTGGTTTACCGTGCCGAGTCGT
ACCGTTACCCTGGGCCTGAATGATGATGATAAAGATACCACCACC
AATCGTTATTTTGGTTGGGATAATGAAAAGCCGCAGCGTCGCGTT
AAAGTTAAAAGCTTTAGCGCCCAGGCACGCCCGATTACCAATGGC
GAATATGTGGAATATCTGAAAGCAATTGGCAGTGAAAAACTGCCG
GCAAGCTGGAGTAGTACCAAAAGTACCCCGAATGGTCATTTTAAT
GGCCATATGAATGGCGATAGCAATGGTGTGGCAAATGCCGCAGTG
AATGGTGAAAATTTTGTTGATGGTAAAGAGGTGAAAACCGTTTTT
GGTAGCATTCCGCTGAAACTGGCACTGGATTGGCCGGTTATGGCA
AGCTATGATGAACTGCTGGGTTTTGCCCATTGGGCAGGTGGCCGC
ATTCCGACCATGGAAGAAGTGAAAAGCATCTATGAATATGCCGAA
GAACTGAAAGTTAAAGATTTTGTGAACGCACTGGGCGAAACCATT
CCGGCAGTTAATGGCCATCTGATTAATGATGGCGTGGAAGAAACC
CCGCCGCATCGCCATAGTGCAAATGGCGAACCGAGTGCCAGCGTT
GGTCCGAGTCCGCATCGCCTGTTTGTTGATCTGGAAGATGCAAAT
GTTGGCTTTAAACATTGGCATCCGCTGCCGGTTACCGCACATGGT
GACAATCTGGCAGGCCAGGGTGAACTGGGCGGCGTTTGGGAATGG
ACCAGTACCGTTCTGGAAAAACATGAAGGCTTTGAACCGATGCAG
CTGTATCCGGGCTATACCGCCGATTTCTTTGATAAAAAACATAAT
ATCGTGCTGGGTGGCAGCTGGGCAACCCATCCGCGTATTGCCGGC
CGTCGTAGCTTTGTTAATTGGTATCAGCGTAATTATCCGTTTGTG
TGGGCCGGTGCACGCCTGGTTCGCGATCTGtaa
<SEQ ID NO: 64; PRT;
D1Egt1; 1_XM_016360447;
Verruconis gallopava>
MIGDSAGLGFADFSSSNVHTKAAKPHSPLSSIIEIRQDREELDLL
IDIKSGLRASGPGRKTLPTLLLYDEPGLKLFEKITFLDEYYLTNA
EIEVLHKWAGNIADRISPNSIVLELGSGNLRKIKILLDAFEAAKK
PVEYYALDVSRVELERTLAAIPVGAFKHVKCFGLHGTYDDGLQWL
KSEQIAKRSKAILSMGSSIGNFARHEAVRFLRSFSDVLQTSDVLL
IGIDACKDPDKVFRAYNDSQGVTHEFVLNGLQHANQLLGHDAFDV
EKWRVIGEYDEVNGKHHAFVSPVQDMNIDGILIKAGERIRIEESF
KYDLIDRSRLWEGAGLIEGASWTNAEANYGLHMAYKPKVQFSSKP
EEYAASAVPTLAEWKDLWTIWDLVTQQMIPKEELLAKPIKLRNAC
IFYLGHIPTFLAIHLEKASCEGVAGLRDYQRIFERGIDPDVDNPE
LCHDHSEIPDEWPPEVEILAFQSKVRDQVKQIYDSRMHESSHAIR
KALWISFEHEVMHIETLLYMLIQSEKTLPPPGVIHPDFEALACEA
KAKTVPNKWFTVPSRTVTLGLNDDDKDTTTNRYFGWDNEKPQRRV
KVKSFSAQARPITNGEYVEYLKAIGSEKLPASWSSTKSTPNGHEN
GHMNGDSNGVANAAVNGENFVDGKEVKTVFGSIPLKLALDWPVMA
SYDELLGFAHWAGGRIPTMEEVKSIYEYAEELKVKDFVNALGETI
PAVNGHLINDGVEETPPHRHSANGEPSASVGPSPHRLFVDLEDAN
VGFKHWHPLPVTAHGDNLAGQGELGGVWEWTSTVLEKHEGFEPMQ
LYPGYTADFFDKKHNIVLGGSWATHPRIAGRRSFVNWYQRNYPFV
WAGARLVRDL
<SEQ ID NO: 65; DNA;
D2Egt2; 2_XM_001728079;
Neurospora crassa>
ATGGTGGCCACCACCGTTGAACTGCCGCTGCAGCAGAAAGCCGAT
GCCGCACAGACCGTTACCGGTCCGCTGCCGTTTGGTAATAGTCTG
CTGAAAGAATTTGTGCTGGACCCTGCATATCGTAATCTGAATCAT
GGTAGTTTTGGCACCATTCCGAGTGCCATTCAGCAGAAACTGCGT
AGTTATCAGACCGCCGCCGAAGCCCGCCCGTGTCCTTTTCTGCGT
TATCAGACCCCGGTTCTGCTGGATGAAAGTCGTGCAGCAGTGGCA
AATCTGCTGAAAGTTCCGGTTGAAACCGTTGTGTTTGTGGCAAAT
GCCACCATGGGCGTGAATACCGTTCTGCGTAATATTGTTTGGAGT
GCAGATGGCAAAGATGAAATTCTGTATTTTGATACCATCTACGGC
GCATGTGGCAAAACCATTGATTATGTTATTGAAGATAAGCGTGGT
ATTGTGAGTAGTCGCTGCATTCCGCTGATCTATCCGGCAGAAGAT
GATGATGTTGTTGCAGCATTTCGCGATGCCATTAAGAAAAGCCGC
GAAGAAGGTAAACGCCCGCGTCTGGCAGTTATTGATGTTGTTAGT
AGTATGCCGGGCGTGCGTTTTCCGTTTGAAGATATTGTTAAAATC
TGCAAAGAGGAAGAAATCATTAGCTGTGTGGATGGTGCACAGGGC
ATTGGCATGGTGGATCTGAAAATTACCGAAACCGATCCGGATTTT
CTGATTAGTAATTGCCATAAATGGCTGTTTACCCCGCGCGGTTGC
GCAGTGTTTTATGTTCCGGTTCGCAATCAGCATCTGATTCGTAGT
ACCCTGCCGACCAGTCATGGTTTTGTGCCGCAGGTGGGCAATCGC
TTTAATCCGCTGGTTCCGGCCGGCAATAAGAGCGCATTTGTGAGC
AATTTTGAATTTGTGGGTACCGTGGATAATAGTCCGTTTTTCTGT
GTGAAAGATGCCATTAAGTGGCGCGAAGAAGTGCTGGGTGGTGAA
GAACGCATTATGGAATATATGACCAAACTGGCCCGTGAAGGTGGC
CAGAAAGTGGCCGAAATTCTGGGCACCCGCGTTCTGGAAAATAGC
ACCGGTACCCTGATTCGCTGTGCCATGGTGAATATTGCACTGCCG
TTTGTTGTTGGCGAAGATCCGAAAGCCCCGGTTAAACTGACCGAA
AAAGAAGAAAAAGATGTTGAAGGCCTGTATGAAATTCCGCATGAA
GAAGCAAATATGGCATTCAAATGGATGTATAATGTGCTGCAGGAT
GAGTTTAATACCTTTGTGCCGATGACCTTTCATCGTCGTCGCTTT
TGGGCACGCCTGAGTGCCCAGGTTTATCTGGAAATGAGCGATTTT
GAATGGGCCGGCAAAACCCTGAAAGAACTGTGCGAACGCGTTGCC
AAAGGTGAATATAAAGAAAGTGCAtaa
<SEQ ID NO: 66; PRT;
D2Egt2; 2_XM_001728079;
Neurospora crassa>
MVATTVELPLQQKADAAQTVTGPLPFGNSLLKEFVLDPAYRNLNH
GSFGTIPSAIQQKLRSYQTAAEARPCPFLRYQTPVLLDESRAAVA
NLLKVPVETVVFVANATMGVNTVLRNIVWSADGKDEILYFDTIYG
ACGKTIDYVIEDKRGIVSSRCIPLIYPAEDDDVVAAFRDAIKKSR
EEGKRPRLAVIDVVSSMPGVRFPFEDIVKICKEEEIISCVDGAQG
IGMVDLKITETDPDFLISNCHKWLFTPRGCAVFYVPVRNQHLIRS
TLPTSHGFVPQVGNRFNPLVPAGNKSAFVSNFEFVGTVDNSPFFC
VKDAIKWREEVLGGEERIMEYMTKLAREGGQKVAEILGTRVLENS
TGTLIRCAMVNIALPFVVGEDPKAPVKLTEKEEKDVEGLYEIPHE
EANMAFKWMYNVLQDEFNTFVPMTFHRRRFWARLSAQVYLEMSDF
EWAGKTLKELCERVAKGEYKESA
<SEQ ID NO: 67; DNA;
D3Egt2; 2_XM_003653634;
Thielavia terrestris NRRL 8126>
ATGGGCCTGGCACCGCTGGAACTGCCGGTGCGTCAGAAAGCAGAT
GTGGATGCCACCCAGAATGGTCCGGTGAAATTTGGTCATGAACTG
CGCGAACAGCATTTTCTGTTTGATCCGAGCTATCGTAATCTGAAT
CATGGCAGCTTTGGCACCATTCCGCGCGCAATTCAGGCAAAACTG
CGTAGCTATCAGGATCAGGCCGAAGCAGCCCCGGATGTGTTTATT
CGTTATGATTATCCGAAACTGCTGGATCAGAGCCGTGCCGCAATT
GCAAAACTGCTGCGCGTGCCGACCGATACCGTTGTTTTTGTGCCG
AATGCAACCACCGGCGTTAATACCGTGCTGCGTAATCTGGATTGG
AATGCCGATGGCAAAGATGAAATTCTGTATTTTGATACCATCTAC
GGCGGCTGTGCCCGCACCATTGATTATGTTGTTGAAGATCGTCAG
GGTCGCGTTAGTCATCGTTGCATTCCGCTGAGTTATCCGTGCGAA
GATGATGCAGTTGTGGCAGCCTTTGAAAGCGCAGTTGAAGCCAGC
CGTCGCGATGGTAAACGTCCGCGTCTGTGCCTGTTTGATGTTGTG
AGCAGTCTGCCGGGCGTTCGTTTTCCGTTTGAAGCAATTGCCGCC
GCATGTCGTGCCGCCGGCCTGTTAAGCCTGGTTGATGGCGCACAG
GGCGTTGGTATGGTGGATCTGGATCTGGCAGCCGTTGATCCGGAT
TTCTTTGTTAGCAATTGCCATAAATGGCTGCATGTTCCGCGTGGC
TGTGCAGTTTTCTATGTGCCGGAACGCAATCAGCCGCTGATGCGT
AGTCCGCTGGTGACCAGTCATCGCTTTGTTCCGCGTGCAGGTGCA
ACCCAGCCGCTGTTTAATCCGCTGCCGCCGACCGATAAAACCGAA
TTTGTTAGTAATTTCGAGTTCGTGGGCACCGTGGATAATGCACCG
TATCTGTGTGTTCGCGATAGTCTGCGCTGGCGCGAAGAAGTGCTG
GGCGGTGAAGCCCGTATTCTGGCCGCACTGACCGCCCAGGCCCGC
GAGGGTGGTAGACGTGCAGCAGCAATTCTGGGTACCGAAGTGCTG
GATAATGCAAGCCAGAGCCTGACCCGTTGCAGCATGGTTAATGTG
GCCCTGCCGCTGGCCGTTCAGCCGGATGGTGAAGGCGAAGCACCG
CCGGCCGCAGGTGGTTTTCCGGCTCTGCCGAAAGAAGATGTTAGT
GCAGTGACCAATTGGATGCTGGAAACCCTGATGGATGAGTTTAAA
ACCTTTATTGCACTGTTTGTGTACAAAGATCGTTGGTGGGCCCGC
CTGAGTGCACAGGTGTATCTGGAACTGGATGATTTTGAATGGGCC
GGCCAGACCCTGAAAACCGTGTGCGAACGTGCAGGCCGCGGTGAA
TATAAACAGGATCGTCCGtaa
<SEQ ID NO: 68; PRT;
D3Egt2; 2_XM_003653634;
Thielavia terrestris NRRL 8126>
MGLAPLELPVRQKADVDATQNGPVKFGHELREQHFLFDPSYRNLN
HGSFGTIPRAIQAKLRSYQDQAEAAPDVFIRYDYPKLLDQSRAAI
AKLLRVPTDTVVFVPNATTGVNTVLRNLDWNADGKDEILYFDTIY
GGCARTIDYVVEDRQGRVSHRCIPLSYPCEDDAVVAAFESAVEAS
RRDGKRPRLCLFDVVSSLPGVRFPFEAIAAACRAAGLLSLVDGAQ
GVGMVDLDLAAVDPDFFVSNCHKWLHVPRGCAVFYVPERNQPLMR
SPLVTSHRFVPRAGATQPLENPLPPTDKTEFVSNFEFVGTVDNAP
YLCVRDSLRWREEVLGGEARILAALTAQAREGGRRAAAILGTEVL
DNASQSLTRCSMVNVALPLAVQPDGEGEAPPAAGGFPALPKEDVS
AVTNWMLETLMDEFKTFIALFVYKDRWWARLSAQVYLELDDFEWA
GQTLKTVCERAGRGEYKQDRP
<SEQ ID NO: 69; DNA;
D4Egt2; 2_XM_018300274;
Colletotrichum higginsianum>
ATGGGCGAACTGGTGAAAGAAACCAGCCAGCTGCAGCTGAGCAGC
ATTCCGTTTGGTAAACCGATGCTGAAAGAATTTCTGATTGATCCG
GCATATCATAATATGAATCATGGCAGTTTTGGCACCATTCCGCGC
CATATTCAGACCATTCTGCGCAGTTATCAGGATAAAGCAGAAGCC
CGCCCGGACCCTTTTATTCGTTGGGAATATCATACCTATCTGAAA
GAAAGCCGTCAGGCCGTTGCAGATTTGATTAATGCACCGGTGGAT
TGTACCGTTTTTGTTCCGAATGCCACCGTGGGCATTAATACCGTT
CTGCGTAATCTGATTTGGGCCCCGGATGGTCTGGATGAAATTCTG
TATTTTAGCACCGTGTATGGTGGCTGTGCAAAAACCATTGATTAT
ATTGTGGATACCCGTCTGGGCCTGGTGAGCAGCCGCAGTATTCCG
CTGACCTATCCGCTGGAAGATGATGAAGTGGTGGCACTGTTTCGC
GATGCAGTGGCACAGAGTCATGCCGAAGGTAAACGCCCGAAAATT
TGTCTGTTTGATGTGGTTAGTTGTCTGCCGGGTATTCGCTTTCCG
TTTGAAGCAATTACCGCCGCATGTCGTGAACTGGGCATTCTGAGT
CTGGTGGATGGCGCACAGGGCGTGGGTATGGTGCCGCTGGATATT
GCAGCACTGGACCCTGATTTCTTTATTAGTAATTGTCATAAGTGG
ACCTTCACCCCGCGTGCAAGTGCCGTGTTTTATGTGAGCGAACGC
AATCATCATCTGGTTCCGAGCACCATTCCGACCAGTCATGGCTAT
GTTCCGCGTACCGGTGTTCAGCGTCATAATCCGCTGCCGCCGAGT
GGTGAACCGCCGTTTGTGACCCGTTTTGGCTTTGTGGCAACCTTT
GATAATAGTCCGAATCTGTGCGTGAAACATAGTATTGAATGGCGC
AAAAGTATTGGCGGCGAAGATAAAATTATGGAATATCTGTGGGCA
CTGGCCAAAAATGGCGGTAAAAAAGCAGCAGCAATTCTGGGTACC
TTTATTCTGGATAATAAGAGTGAAACCCTGACCCGCTGCGCAATG
GTTAATGTTGCACTGCCGATTGTTATGGGCGCCGATGCCGAAACC
CTGAGTGTTGGCCCGGATGGCACCATTACCGTTCCGGAAAAAGAA
GCCAGTGTTATTGTTAATTGGATGCTGAGTGCCCTGGTTAATGAA
TATCTGACCTTTGTGGCCCTGTTTTGGCATCAGGGCCGCTGGTAT
AGTCGTATTAGTGCCCAGATATATCTGGATGAAACCGATTTTGAA
TGGGTTGGCAATACCATTAAGGAACTGTGTCAGCGCGTTGCCAAA
CAGGAATATAAAGTGAAAGCAtaa
<SEQ ID NO: 70; PRT;
D4Egt2; 2_XM_018300274;
Colletotrichum higginsianum>
MGELVKETSQLQLSSIPFGKPMLKEFLIDPAYHNMNHGSFGTIPR
HIQTILRSYQDKAEARPDPFIRWEYHTYLKESRQAVADLINAPVD
CTVFVPNATVGINTVLRNLIWAPDGLDEILYFSTVYGGCAKTIDY
IVDTRLGLVSSRSIPLTYPLEDDEVVALFRDAVAQSHAEGKRPKI
CLFDVVSCLPGIRFPFEAITAACRELGILSLVDGAQGVGMVPLDI
AALDPDFFISNCHKWTFTPRASAVFYVSERNHHLVPSTIPTSHGY
VPRTGVQRHNPLPPSGEPPFVTRFGFVATFDNSPNLCVKHSIEWR
KSIGGEDKIMEYLWALAKNGGKKAAAILGTFILDNKSETLTRCAM
VNVALPIVMGADAETLSVGPDGTITVPEKEASVIVNWMLSALVNE
YLTFVALFWHQGRWYSRISAQIYLDETDFEWVGNTIKELCQRVAK
QEYKVKA
<SEQ ID NO: 71; DNA;
D5Egt2; 2_XM_018389754;
Fusarium oxysporum f. sp.
lycopersici
4287>
ATGGGCAGCATTGGTCAGGGTAGTAGTCAGCTGCCGGTGCGTGGC
AAAACCAATACCAGTGTGTTTGGCAGCGCCATTAAGAAAGAGTTT
ATGTTTGATCCGGAATGGCGCAATCTGAATCATGGCAGCTTTGGC
ACCTATCCGCAGGCAGTTCGTACCAAATTTCGCGAATATCAGGAT
GCCAGTGAAGCCCGCCCGGACCCTTTTATTCGCTATGAATATCCG
AAAATTCTGGATGAAAACCGTGCAGCAGTGGCCAAACTGCTGAAT
GCCCCGGTTGATAGCGTGGTTTTTGTTAGTAATGCAACCACCGGC
GTTAATACCGTGTATCGCAATATGAAATGGAATGAAGATGGCAAA
GATGTTATTATTAGCTTTAGCACCATCTATGAAGCCTGTGGTAAA
GTGGCAGATTATTATGTTGATTATTACAACGAGAAGGTGACCCAT
CGTGAAATTGAACTGCCGTATCCGCTGGATGATGATGAAATTATT
AAGAAATTCGAGGACGCCGTTAAAAAGATTGAAAGTGAAGGCAAA
CGCGTGCGTATTTGCACCTTTGATGTTGTGAGTAGCCGTCCGGGC
GTTGTTTTTCCGTGGGAAGAAATGGTTAAAACCTGTCGCCGTCTG
AATGTTCTGAGCATGGTGGATGGTGCCCAGGGTGTGGGTATGGTG
AAACTGGATCTGAGTGCCGCCGATCCGGATTTCTTTGTGAGCAAT
TGCCATAAATGGCTGCATGTGCCGCGTGGTTGCGCCGTTTTCTAT
GTTCCGCAGCGTAATCAGGCACTGATTCAGACCACCCTGGCAACC
AGCCATGGCTATGTTCCGAAACTGGCAAATCGTATTACCCCGCTG
CCGCCGAGTAGTAAAAGTCCGTTTGTTATTAATTTCGAGTTCGTG
GGTACCCTGGATAATAGTCCGTATCTGTGCGTGAAAGATGCAATT
AAGTGGCGTGAAGAAGCACTGGGTGGCGAAGATGCCATTCTGGAA
TATATTTGGGATCTGAATAAGAAAGGCAGTGAACTGGTTGCAGAA
AAACTGGGTACCACCTATATGGAAAATAGCACCGGCACCATGCGT
AATTGCGGTATGGCAAATATTGCCCTGCCGGTGTGGACCGTTGAA
GGCAAAGAAGGCGAAGTGGTTATTAGCGCAGAAGAAACCCAGACC
GCCTTTCAGTGGATTCTGAATACCCTGATTGGCGATTATAAAACC
TTTGTGGCACTGTTTCTGCATGGCGGTCGCTTTTGGATTCGTACC
AGCGCACAGGTGTATCTGGAAATTGAAGATTATGAATGGCTGGGC
GGTGTTCTGAAAGAAGTTTGTGAACGTGTTGGTAAAAAAGAATAT
CTGAAAtaa
<SEQ ID NO: 72; PRT;
D5Egt2; 2_XM_018389754;
Fusarium oxysporum f. sp.
lycopersici
4287>
MGSIGQGSSQLPVRGKTNTSVFGSAIKKEFMFDPEWRNLNHGSFG
TYPQAVRTKFREYQDASEARPDPFIRYEYPKILDENRAAVAKLLN
APVDSVVFVSNATTGVNTVYRNMKWNEDGKDVIISFSTIYEACGK
VADYYVDYYNEKVTHREIELPYPLDDDEIIKKFEDAVKKIESEGK
RVRICTFDVVSSRPGVVFPWEEMVKTCRRLNVLSMVDGAQGVGMV
KLDLSAADPDFFVSNCHKWLHVPRGCAVFYVPQRNQALIQTTLAT
SHGYVPKLANRITPLPPSSKSPFVINFEFVGTLDNSPYLCVKDAI
KWREEALGGEDAILEYIWDLNKKGSELVAEKLGTTYMENSTGTMR
NCGMANIALPVWTVEGKEGEVVISAEETQTAFQWILNTLIGDYKT
FVALFLHGGRFWIRTSAQVYLEIEDYEWLGGVLKEVCERVGKKEY
LK
<SEQ ID NO: 73; DNA;
D6Egt2; 2_XM_018216062;
Phialocephala scopiformis>
ATGACCATTAAGCCGCCGTTTGGTCATCCGATTCGTAATACCCAT
TTTAGCTTTAGCCCGACCTATGTTCCGCTGAATCATGGTAGTTTT
GGCACCTTTCCGCTGAGTGTTACCCAGCATCAGAATCAGCTGCAG
ACCCAGGCCCTGGAACGCCCGGATACCTTTATTGTGTTTGATCTG
CCGGTGCTGATTGATGAAAGCCGCGCAGCAATTGCACCGCTGCTG
GGTGTTGATGTGGATGAAGTTGTGTTTGTGCCGAATGCCACCACC
GGCGTTAATGTTGTTCTGCGTAATCTGCGTTGGGAAGAAGGTGAC
GTGGTTGTTTGTTTTAGTACCATCTATGGCGCATGCGAAAAAAGC
CTGGTTAGTGTTGGTGAAGTTCTGCCGGTTCAGATGGAAGTGGTG
GAACTGCAGTATCCGGTTGAAGATGAAGAAATTCTGGGTCGTCTG
GAAGAACGTGTTGGTAAAGTGCGTCAGGAAGGCAAACGCATTCGT
CTGGCAATGTTTGATACCGTGCTGACCTTTCCGGGCGCACGTATG
CCGTGGGAACGCCTGGTGGCCAAATGCAAAGAACTGGAAGTGCTG
AGCCTGATTGATGGTGCACATGGTATTGGCCATATTGATCTGCGT
GAACTGGGCAAAGTGGCCCCGGATTTCTTTGTGAGTAATTGCCAT
AAATGGCTGTATACCCCGCGTGGTTGCGCCGTTTTTCATGTGCCG
TTTAAAAATCAGCATCTGATTCGTACCAGCCTGCCGACCAGTCAT
GGTTATCAGCATCCGAATAAGCCGCCGGAAAAAATTGATGGCAAA
ACCCCGTTTGTGCATCTGTTTGAATTTGTTGCAACCATTGATTAT
AGCCCGTATGCATGCGTGCCGGCAGCCCTGAGCTTTCGTCAGAAA
ATTTGTGGTGGCGAAGAAGAAATTCGCAAATATTGTTTTAACCTG
GCACGTACCGGCGGCGCCGCAGTGGCAAAAATTCTGGGCACCCAT
GTGATGGATACCAAAAGTGGTACCATGAGCCAGTGTTGTTTTGCA
AATGTTGCACTGCCGCTGGCATTTGGCGAAGGCAAAAAATTTGGC
ACCGATGAAGCCCCGCGCATTCAGAAATGGCTGAATGGCACCGCA
GTGCGTGAATTTGATACCTATCTGCAGATTGCCCTGCATGGTGGT
ATTATGTGGGTTCGCCTGAGTGCCCAGATATATCTGGAAGGTAAA
GATTTTGAATGGGTGGGTTATCGTCTGAAAGAACTGTGCGTTCGT
ATTGAAGGTGGCGAAGTGGATCGCtaa
<SEQ ID NO: 74; PRT;
D6Egt2; 2_XM_018216062;
Phialocephala scopiformis>
MTIKPPFGHPIRNTHFSFSPTYVPLNHGSFGTFPLSVTQHQNQLQ
TQALERPDTFIVEDLPVLIDESRAAIAPLLGVDVDEVVFVPNATT
GVNVVLRNLRWEEGDVVVCFSTIYGACEKSLVSVGEVLPVQMEVV
ELQYPVEDEEILGRLEERVGKVRQEGKRIRLAMEDTVLTFPGARM
PWERLVAKCKELEVLSLIDGAHGIGHIDLRELGKVAPDFFVSNCH
KWLYTPRGCAVFHVPFKNQHLIRTSLPTSHGYQHPNKPPEKIDGK
TPFVHLFEFVATIDYSPYACVPAALSFRQKICGGEEEIRKYCFNL
ARTGGAAVAKILGTHVMDTKSGTMSQCCFANVALPLAFGEGKKFG
TDEAPRIQKWLNGTAVREFDTYLQIALHGGIMWVRLSAQIYLEGK
DFEWVGYRLKELCVRIEGGEVDR
<SEQ ID NO: 75; DNA;
D7Egt2; 2_XM_003045069;
Nectria haematococca>
ATGGGTAGCGTGACCCAGGAACTGCCGCTGCGCGGTAAACCGAGT
GCAAGTGTTTTTGGCGCCGCAATGAAAGATGAATTTCTGTTTGAT
CCGGAATGGCGCAATCTGAATCATGGTAGCTTTGGCACCTATCCG
AAAGCAATTAAGGCCAAATTTCGCGATGAAGCACGTCCGGATGTT
TTTATTCGTTATGAATATCCGAAGCTGCTGGATGAAAGTCGTGTT
GCCGTTGCCAAAATTCTGAATGCACCGGAAGATGGCGTGGTGTTT
GTGAGCAATGCCACCGTGGGCGTTAATACCGTTTTTCGTAATATG
GCATGGAATAAGGATGGCAAAGATGTGATTATTAGCTTTAGTACC
ATCTATGAAGCCTGTGGTAAAGTTGCCGATTATCTGGCCGATTAT
TATGAAGGCAATGTTACCCATCGTGAAATTGAAATTACCTATCCG
ATTGATGATGATGTGATTCTGAAACGCTTTGAAGATACCGTGAAA
AAGATTGAAGAAGAAGGTAAACGCGCACGTATTTGTACCTTTGAT
GTTGTTAGTAGTCGCCCGGGCGTGGTGTTCCCGTGGGAAGAAATG
ATTAAGACCTGTCGCCGCCTGAATGTGCTGAGCATGGTTGATGGC
GCCCAGGGTATTGGCATGGTGAAACTGGATCTGAGTGCAGCCGAT
CCGGATTTCTTTGTTAGCAATTGCCATAAATGGCTGCATGTGCCG
CGTGGTTGCGCCGTTTTCTATGTTCCGCAGCGTAATCAGGCACTG
CTGCCGACCACCCTGGCAACCAGTCATGGCTATGTTCCGAAACTG
GCAAATCGCATTAGTCCGCTGCCGCCGAGTAGCAAACCGCGCTTT
GTGACCAATTTTGAATTTGTTGGCACCCTGGATAATAGTCCGTAT
CTGTGCGTTAAAGATGCAATTAAGTGGCGCCAGGATGTTCTGGGT
GGTGAAGATGCAGTTCTGAAATATCTGTGGGATCTGAATAAGAAA
GGTACCGATATTGTGGCAAAAGCACTGAATACCCCGGTTATGGAA
AATAGTACCGGTACCCTGCGTAATTGTGGCATGGGCAATGTTGCC
CTGCCGCTGTGGGCAGGTGAAGGTGAAGGCACCGTGGTGCCGGCA
GATGAAACCCAGAAAGCATTTCAGTGGATGCTGACCACCCTGATT
GATGATTATAAAACCTTTCTGAGTCTGTTTATCCATGGCGGCCGT
TTTTGGGCACGCATTAGCGCCCAGGTTTATCTGGGCATTGAAGAT
TATGAATGGGCCGGTAAAGTTCTGAAAGAACTGTGTGAACGTGTG
GCAAAAAAGGAATATCTGtaa
<SEQ ID NO: 76; PRT;
D7Egt2; 2_XM_003045069;
Nectria haematococca>
MGSVTQELPLRGKPSASVFGAAMKDEFLFDPEWRNLNHGSFGTYP
KAIKAKFRDEARPDVFIRYEYPKLLDESRVAVAKILNAPEDGVVF
VSNATVGVNTVFRNMAWNKDGKDVIISFSTIYEACGKVADYLADY
YEGNVTHREIEITYPIDDDVILKRFEDTVKKIEEEGKRARICTFD
VVSSRPGVVFPWEEMIKTCRRLNVLSMVDGAQGIGMVKLDLSAAD
PDFFVSNCHKWLHVPRGCAVFYVPQRNQALLPTTLATSHGYVPKL
ANRISPLPPSSKPRFVTNFEFVGTLDNSPYLCVKDAIKWRQDVLG
GEDAVLKYLWDLNKKGTDIVAKALNTPVMENSTGTLRNCGMGNVA
LPLWAGEGEGTVVPADETQKAFQWMLTTLIDDYKTFLSLFIHGGR
FWARISAQVYLGIEDYEWAGKVLKELCERVAKKEYL
<SEQ ID NO: 77; DNA;
D8Egt2; 2_XM_024886631;
Hyaloscypha bicolor>
ATGGGCGAAGTGCTGAATATTAAGCTGGAAGAAGTTAGTCTGAAT
AATGAACGTACCCCGTTTGGTAAAGAAATGCTGAAACATTTTCTG
TTTGACCCGGATTATAAAAATCTGAATCAGGGCAGTTTTGGTAGC
TTTCCGCGTGTGGTGCGCGAAAAACAGCAGCAGTATCAGCGCGCA
TGCGAACTGCGTCCGGACCCTTTTATTCGTTATGAACATCCGAAA
CTGCTGGATGAAAGCCGTGCAGCACTGGCCAAAGTGCTGAATGCC
CCGCTGAGCACCGTTGTTTGTGTTCCGAATGCAACCACCGGCGTG
AATACCATTATTCGTAATATTGTGTGGAACGCAGATCGCAAAGAT
GAAGTGCTGTATTTTAGCACCGCATATTGCAGCTGCAGCAATACC
ATTGATTATAATAGTGAAGTGCACCCGAATCTGGTGGGCAGTCGT
GAAATTAGTCTGACCTATCCGATTGAAGATGAAGATTTGCTGAAA
ATGTTCAAAGATGCAATTAAGGCCAGCCGTGCAAGCGGCAAACGT
CCGCGTCTGGCCATGTTTGATACCGTGAGTAGCCTGCCGGGCGTT
CGTATGCCGTTTGAAAGTCTGACCGCCATTTGCAAACAGGAAGGC
ATTTTTAGCCTGATTGATGGTGCACATGGTATTGGTCTGCTGCCG
CTGGATCTGAGCGCCCTGGACCCTGATTTCTTTACCAGCAATACC
CATAAATGGTTTTTCGTGCCGCGCGGCTGTGCAGTGCTGTATGTT
CCGGAACGCAATCAGCATCTGATTCGTAGCAGCCTGCCGACCAGC
GATGGCTTTGTGAGTAAAACCGGCATGGCCAGTCGCAATCCGCTG
CCGCCGAGTAGTAAAAGTGAATTTGTTAATACCTTCGAGTTCGTG
GGTACCCTGGATAATGCCAATTATCTGGTTATTCCGGAAGCAATT
GAATTTCGTGAAAAAGTTTGTGGCGGCGAAAAAGCAATTATGGAA
TATTGTGTTCACCTGGCCAAAGATGGCGGCAAAGCCGCCGCCAAA
ATTCTGGGTACCAGCATTATGGATAATAGCACCGAAACCCTGACC
AAAGGTTGTGGTATGGTGAATATTCTGCTGCCGTTAGAAATTAGC
CCGACCAAAGTGCATGGCAAAAATTGTATTGATCCGCGTAATCGT
ACCGTGGCAACCGAATGGATGCAGGAAACCCTGATTGCAGATTTT
AAAACCTTTATTCCGATCTATCTGTTCCAGGAAAAATGGTGGGCC
CGTCTGAGTGCACAGATATATCTGGAACTGGTTGATTTTGAATGG
GCAGGTAAAGCACTGAAAGCAATTTGCGAACGTGCCGGCAAAGGC
GAATTTCTGAAAGCCGAAAAGAAAGTGAAAAAGATGGGTGAAGTT
GTTGGTGGCGGTGGTGAACATGGCCCGCGTACCCGTCCGGAACTG
GAATGCAAACTGtaa
<SEQ ID NO: 78; PRT;
D8Egt2; 2_XM_024886631;
Hyaloscypha bicolor>
MGEVLNIKLEEVSLNNERTPFGKEMLKHFLFDPDYKNLNQGSFGS
FPRVVREKQQQYQRACELRPDPFIRYEHPKLLDESRAALAKVLNA
PLSTVVCVPNATTGVNTIIRNIVWNADRKDEVLYFSTAYCSCSNT
IDYNSEVHPNLVGSREISLTYPIEDEDLLKMFKDAIKASRASGKR
PRLAMFDTVSSLPGVRMPFESLTAICKQEGIFSLIDGAHGIGLLP
LDLSALDPDFFTSNTHKWFFVPRGCAVLYVPERNQHLIRSSLPTS
DGFVSKTGMASRNPLPPSSKSEFVNTFEFVGTLDNANYLVIPEAI
EFREKVCGGEKAIMEYCVHLAKDGGKAAAKILGTSIMDNSTETLT
KGCGMVNILLPLEISPTKVHGKNCIDPRNRTVATEWMQETLIADF
KTFIPIYLFQEKWWARLSAQIYLELVDFEWAGKALKAICERAGKG
EFLKAEKKVKKMGEVVGGGGEHGPRTRPELECKL
<SEQ ID NO: 79; DNA;
E1Egt2; 2_XM_024814247; Aspergillus candidus>
ATGGGCGAAGCCAATGTTGTTCTGGGCAGTGGTCCGACCCCGTTT
GGTAAAGAAATGAAAAAACATTTCAGCTTCGCACCGGGCTATCAT
AATCTGAATCATGGCAGTTATGGCACCTGCCCGACCGCAATTCAG
CGTGAAGCCAATCGCCTGCGCGATGAATGCGAAGCCCGTCCGTGC
CCGTTTATTAAGTATCGTTTTCCGGAACTGCTGGATGAAAGCCGC
GCAGCAGTGGCCCAGTTTCTGGGTGTTCCGCGTAGTACCGTTGTG
TTTGTTACCAATGCAACCACCGGCGTTAATACCGTTTTTCGCAAT
ATGATTTGGAATACCGATGGCAAAGATGAAATTATTGAATTTGAC
GTTGTGTACGGTGCCTGTGGTAAAACCGCCGATTATATTTGCGAA
ACCAGTCGTGATCTGGTGCGTACCCGCCAGATTCAGCTGACCTAT
CCGGTTGAAGATGATGATTTTGTTGCAGCCTTTCGTGAAGCCATT
GATGCAAGTCGTCGCGATGGCCGTCGTCCGCGTATTGCAATTTTT
GATACCATTAGCAGCAATCCGGGTATTCGCCTGCCGTTTGAAGCC
CTGACCGCCGTTTGTCGTAGCGAAGGTGTTCTGAGCCTGATTGAT
GCCGCACATGGTATTGGCCAGATTGATCTGAATCTGCCGAGCCTG
GACCCTGATTTTCTGGTGAGTAATTGCCATAAATGGCTGTTTACC
CCGCGTGGTTGTGCCGTTTTCTATGTGCCGGAACGCAATCAGGCA
ATGATGCGCAGCACCATTCCGACCAGCCATGGTTTTCGTCCGCGT
CTGGCACAGAATGAAGAAAAGAAAGTGAGCATTGCACCGCATACC
CATAGCGAATTTGAACTGAATTTTGAACATACCGGTACCTATGAT
AATATTGCATTTCTGACCGTGCCGGCCGCCATTAAGTGGCGTCAG
AATGTGTGTGGCGGCGAAGAAAAAATTCGCGGTTATTGTACCAAT
CTGGCCCGCGAAGGCGGCAAAATTGTTGCCGCAGCACTGGGCACC
AGTGTTCTGGATAATCCGACCCATACCTATACCGATTGCTTTATG
GTGAATATTCTGCTGCCGGTTCCGCCGAAAGAAAATGAATGTATG
AATTGGCGTGGTCGTCCGGTTAATATTAGTGAATGGATGCAGCGC
ACCATGATTGAAGAATGGCAGACCTATATGCCGGTGTTTTGGTTT
AAAGGCGCCTGGTGGTTTCGCATTAGCGCACAGGTTTATCTGGAA
CTGAGCGATTTTGAATGGGCCGGTAGCGCCATGAAAGAAGTGTGT
CAGAAAGTGAATAAGCTGCTGGGTtaa
<SEQ ID NO: 80; PRT;
E1Egt2; 2_XM_024814247; Aspergillus candidus>
MGEANVVLGSGPTPFGKEMKKHFSFAPGYHNLNHGSYGTCPTAIQ
REANRLRDECEARPCPFIKYRFPELLDESRAAVAQFLGVPRSTVV
FVTNATTGVNTVFRNMIWNTDGKDEIIEFDVVYGACGKTADYICE
TSRDLVRTRQIQLTYPVEDDDFVAAFREAIDASRRDGRRPRIAIF
DTISSNPGIRLPFEALTAVCRSEGVLSLIDAAHGIGQIDLNLPSL
DPDFLVSNCHKWLFTPRGCAVFYVPERNQAMMRSTIPTSHGFRPR
LAQNEEKKVSIAPHTHSEFELNFEHTGTYDNIAFLTVPAAIKWRQ
NVCGGEEKIRGYCTNLAREGGKIVAAALGTSVLDNPTHTYTDCFM
VNILLPVPPKENECMNWRGRPVNISEWMQRTMIEEWQTYMPVFWF
KGAWWFRISAQVYLELSDFEWAGSAMKEVCQKVNKLLG
<SEQ ID NO: 81; DNA;
E2Egt2; 2_XM_003720232; Pyricularia oryzae>
ATGGCAACCCCGCTGCGTCATTATGGCACCGAAAAACCGTATAGC
CATCCGGAACCGGTTACCAAACGCCAGTTTGGTAAAAATGTGCTG
CAGGATTTTCTGATTGATCCGAAATTTCGCAATATGAATCATGGT
AGTTTCGGCGTTATTCCGCGTCCGGTTCATGCCGCACGCCGTTAT
TATCAGGATAAAAGCGAAGAACGTCCGGATGTGTGGATTCGCTAT
AATTGGAGCCAGCTGCTGGAAGGCAGCCGCGCCGCTGTGGCACCT
CTGTTAGGTGTGGATAAAGATACCATTGCATTTGTGCCGAATGCC
ACCGTTGGTGTTAATACCGTTCTGCGCAATCTGGTGTGGAATGAT
GATAAAAAAGATGAAATCCTGTACTTCAACACCATCTATGCAGCA
TGCGGCAAAACCGTGCAGTATATGATTGAAATTAGTCGCGGCCAT
GTTAGCGGTCGTAGCGTGCCGCTGGAATATCCGCTGACCGATGAT
GAACTGGTGGCCCTGTTTAAAAAAGGTATTCAGGATTGTCGTGCA
GCAGGTAAACGTCCGCGCGCCGCCGTGATTGATACCGTTAGCAGT
ATTCCGGCAGTTCGCCTGCCGTTTGAAGCCCTGGTGCAGGTTTGT
CATGATGAAGGTATTCTGAGTATTGTTGATGGTGCCCAGGGTGTG
GGTATGATTGATCTGAAACATCTGGGCACCCAGGTTAAACCGGAT
TTCTTTATTACCAATTGTCATAAATGGCTGTACACCCCGCGCGGT
TGCGCCGTTCTGCATGTGCCGAAACATAATCAGGCCCTGATGCGT
AGTACCCTGCCGACCAGCTGGGGTTGGGTTCCGAGTGGCGAAGGT
GACCCGGATTTTATTGATAATTTTGCCTTTGCAAGCACCCTGGAT
AATAGTAATTATATGGCCGTTCAGCATGCAGTTCAGTGGATTCAG
GAAGCACTGGGCGGTGAAGATGCCGTTATTGAATATATGATGAGT
CTGAATAAGAAGGGCGGCAATATGGTGGCAGAAATGCTGGGCACC
AAAGTTCTGGATAATGCAGAAGGCACCCTGACCAATTGCGCCATG
AGTAATGTTCTGCTGCCGCTGGGTATTAAGGGCCGCGAAAGTAGT
GCAAAAGTTCTGGTGGATGAAGAAGATGCCGCCCGTCTGGGCGAT
TGGTGCCAGAAAACCCTGGCCAGTGATTATAATACCTGGCTGCCG
GTTACCCTGATTAAGGGTCAGTGGTGGACCCGCATTAGTGCCCAG
GCCTATCTGGATGAAAGTGATTATGAAGCAGTTGGCAAAATTTTT
CTGGAACTGGTTGAACGTATTGGCAAAGGCGATCATAAAAAAtaa
<SEQ ID NO: 82; PRT;
E2Egt2; 2_XM_003720232; Pyricularia oryzae>
MATPLRHYGTEKPYSHPEPVTKRQFGKNVLQDFLIDPKFRNMNHG
SFGVIPRPVHAARRYYQDKSEERPDVWIRYNWSQLLEGSRAAVAP
LLGVDKDTIAFVPNATVGVNTVLRNLVWNDDKKDEILYENTIYAA
CGKTVQYMIEISRGHVSGRSVPLEYPLTDDELVALFKKGIQDCRA
AGKRPRAAVIDTVSSIPAVRLPFEALVQVCHDEGILSIVDGAQGV
GMIDLKHLGTQVKPDFFITNCHKWLYTPRGCAVLHVPKHNQALMR
STLPTSWGWVPSGEGDPDFIDNFAFASTLDNSNYMAVQHAVQWIQ
EALGGEDAVIEYMMSLNKKGGNMVAEMLGTKVLDNAEGTLTN
CAMSNVLLPLGIKGRESSAKVLVDEEDAARLGDWCQKTLASDYNT
WLPVTLIKGQWWTRISAQAYLDESDYEAVGKIFLELVERIGKGDH
KK
<SEQ ID NO: 83; DNA;
E3Egt2; 2_XM_008078420; Glarea lozoyensis>
ATGCCGGCTCCGCTGAATATGCCGATTCATCTGAAAGCCGGCGAA
GTTAGTAGCGATGTTAGTAATAAGTATAAGCGTGTTCCGTTTGGT
AAAGAAATGCTGAAACAGTTTAGTTTTGACCCGGAATATCGTAAT
GTGAATCATGGTAGCTATGGTAGCTTTCCGAAACCGATTAGCGAA
CTGCGTCGCCATTATCTGGATGAATGTGAAAAAAGCCCGGACCCT
TTTATTCGCTATGATTTTGGTAGTATTCTGGATGAAAACCGTGCC
GCAGTGGCCAAACTGGTGGATGCCCCGCTGCATACCGTTGTTTTT
GTGCCGAATGCAACCACCGGCATTAATGTTGTTCTGCGCAATCTG
CAGTGGAATGAAAATGGCAAAGATGAAATTCTGTACTTTAACACC
ATCTATGGTGCATGTGGTAAAACCGTTAGTTATACCAGTGAATAT
AGTCGTGGTCTGGTGCAGGGCCGTGAAATTACCCTGGATTATCCG
ATTAGCGATGAAGCACTGATTGAACAGTTTAGCAGTACCATTCAG
GCCAGCATTGATGCAGGCAAAAATCCGCGCATTGCAATTTTTGAT
ACCATTAGTAGTCTGCCGGGCGTTCGCATGCCGTTTGAAGCCCTG
ACCGCAGTGTGCGCCAGTAGCGGTGTGCTGAGCCTGATTGATGGC
GCCCATGGCATTGGTCATATTCCGCTGAGTCTGAGCACCCTGAAT
CCGGATTTCTTTGTGAGCAATCTGCATAAATGGCTGTTTGTGCCG
CGTGGTTGCGCACTGTTTTATGTTCCGCTGCGCAATCAGCATCTG
ATTCGTACCAGCCTGCCGACCAGTCATTATTTTGAACCGAAACAG
CTGAGCCTGGGCGCCCCGAATCCGTTTGCCCCGACCACCAAAAGT
GGCTTTGTGATGCAGTTTGAAAGTAATGGCACCATTGATAATAGT
CCGTATCTGACCGTGGCCGAAGCAATTCGTTGGCGCCGCGAAGCA
TGTGGCGGCGAAGAAGCAATTCATGATTATTGTCTGGATCTGAGC
CGTAAAGGCGCAACCCTGATTGCAAGTATTCTGAATACCCATATT
CTGGATAATCCGCAGCATACCCTGACCAATTGTCATCTGAGCAAT
ATTCTGCTGCCGGTTAGCACCAAACCGTATCAGGATTTTCATGTT
ATTCCGGAAGAACATGCACATCTGGTGGGTGAATGGATTCATACC
ACCATGATTAAGGATCATAAAACCTTTGTGGCCATTTTCTATTTT
CAGGAAAAATGGTGGGGCCGCCTGAGTGCACAGATATATCTGGAA
ATTGAAGATTATGAGTGGGCAGGTAATGTTCTGAAAGGCCTGTGC
GAACGCGTGGGCCGCCTGGAATTTCTGGGCGAAGAAGTTCCGCGC
GGCGATGCAGCACCGtaa
<SEQ ID NO: 84; PRT;
E3Egt2; 2_XM_008078420;
Glarea lozoyensis>
MPAPLNMPIHLKAGEVSSDVSNKYKRVPFGKEMLKQFSFDPEYRN
VNHGSYGSFPKPISELRRHYLDECEKSPDPFIRYDFGSILDENRA
AVAKLVDAPLHTVVFVPNATTGINVVLRNLOWNENGKDEILYFNT
IYGACGKTVSYTSEYSRGLVQGREITLDYPISDEALIEQFSSTIQ
ASIDAGKNPRIAIFDTISSLPGVRMPFEALTAVCASSGVLSLIDG
AHGIGHIPLSLSTLNPDFFVSNLHKWLFVPRGCALFYVPLRNQHL
IRTSLPTSHYFEPKQLSLGAPNPFAPTTKSGFVMQFESNGTIDNS
PYLTVAEAIRWRREACGGEEAIHDYCLDLSRKGATLIASILNTHI
LDNPQHTLTNCHLSNILLPVSTKPYQDFHVIPEEHAHLVGEWIHT
TMIKDHKTFVAIFYFQEKWWGRLSAQIYLEIEDYEWAGNVLKGLC
ERVGRLEFLGEEVPRGDAAP
<SEQ ID NO: 85; DNA;
F1Egt2; 2_XM_008028041;
Exserohilum turcica>
ATGACCAGTAATAGCAAATACGGTGTTCCGGATATTAAGACCAAA
GATGGTATTGAATTTGGTAAAGAACTGCAGGAAAAAGAATTTCTG
TTTGATAAAGGTTACATCGGTCTGAATCATGGTAGTTTTGGTACC
TATCCGCGCCCGGTGCGTGATCGTCTGCGTGCATTTCAGGATGCC
AGCGAAGCCCAGCCGGATAAATTCATTCTGTATGATTATCCGCGT
TATCTGGATGAAGCCCGTGAAGCCATGGCAAAACTGCTGAATACC
CCGAGTAGCACCCTGGTTTTTGTTCCGAATGCAACCACCGGTGTG
AATATTGTTCTGCGTAATCTGGTTTTTACCCCGGAAGATCATATT
CTGATTTTTAGTAATATCTACGGCGCCTGCGAACGTACCGTTAGT
TATATTACCGAAACCACCCCGGCACAGAGTGTTAAAGTTGAATAT
GCCCTGCCGTTTGAAGATGATTGGCTGGTTGAACAGTTTGAAAGC
AAAGTTCGTGATGTTGAAGCAAAAGGCGGTAAAGTGAAAATTGCA
ATTTTTGATACCGTGGTGAGCATGCCGGGCATTCGTCTGCCGTTT
GAGCGCCTGACCGCAAAAAGCAAAGAACTGGGCATTCTGAGTTGC
ATTGATGGTGCCCATGGCGTTGGCCATGTGGAAATTGATCTGGGT
ACCCTGGACCCTGATTTCTTTGTGAGTAATTGTCATAAATGGCTG
CATGTGCCGCGTGGTACCGCCATTTTTCATGTTGCCCATCGTGCC
CAGCATCTGATTCGTAGCACCCTGCCGACCAGTCATGGTTTTACC
CCGAAAAATGGTAAATTTGTGAGCCCGTTTAGCAAACCGGTGTAT
CATAATCGTAGTCAGCAGACCGGTGCCGAACAGAATACCAGCGAA
CAGCAGACCGCCGGTACCGCCGCAAGTAGCGAAAAACCGGAATTT
GTGGCAAATTTTGAATTTGTTGGCACCATTGATAGTAGCCCGTAT
CTGTGCGTTCCGACCGCACTGAAATGGCGTGAAAGCCTGGGCGGC
GAAGCAGTGATTCGCAGTTATTGTACCACCCTGGCCCAGGCAGCC
GGCCAGCATGTGGCTAGTGTTCTGGGTACCCATGTGCTGGAAAAT
CGCACCCGCACCCTGGGCCAGTGCTGTCTGAGTAATGTTCTGCTG
CCGATTAGCCTGGAAAAAGTTCATGCCACCGCACGCCTGGCCGGT
ATTGATCCGGATGATGCCGGTCTGAAAGTTCGCGATTGGATGAAA
AAACTGAGCAGCGAACAGTATAATACCTTTATTATGGTTTACTGG
TACGCCGGCAAATGGTGGACCCGCCTGAGTGGTCAGGTTTATCTG
GATATGCGTGATTTTGAATGGGCCGCACATACCCTGAAAGAAATG
TGCGCCCGTGTGGAAAGCGGTGAATGGGCAGGTGTGAAAGGTCGC
CTGtaa
<SEQ ID NO: 86; PRT;
F1Egt2; 2_XM_008028041;
Exserohilum turcica>
MTSNSKYGVPDIKTKDGIEFGKELQEKEFLFDKGYIGLNHGSFGT
YPRPVRDRLRAFQDASEAQPDKFILYDYPRYLDEAREAMAKLLNT
PSSTLVFVPNATTGVNIVLRNLVFTPEDHILIFSNIYGACERTVS
YITETTPAQSVKVEYALPFEDDWLVEQFESKVRDVEAKGGKVKIA
IFDTVVSMPGIRLPFERLTAKSKELGILSCIDGAHGVGHVEIDLG
TLDPDFFVSNCHKWLHVPRGTAIFHVAHRAQHLIRSTLPTSHGFT
PKNGKFVSPFSKPVYHNRSQQTGAEQNTSEQQTAGTAASSEKPEF
VANFEFVGTIDSSPYLCVPTALKWRESLGGEAVIRSYCTTLAQAA
GQHVASVLGTHVLENRTRTLGQCCLSNVLLPISLEKVHATARLAG
IDPDDAGLKVRDWMKKLSSEQYNTFIMVYWYAGKWWTRLSGQVYL
DMRDFEWAAHTLKEMCARVESGEWAGVKGRL
<SEQ ID NO: 87; DNA;
F2Egt2; 2_XM_013170142;
Schizosaccharomyces cryophilus>
ATGAGCGATTGTATGCCGTTTGGCCATGCACTGAAACCGTATTAT
ATGCTGGATAAAAATTACGTGAGCGTGAATAATGGTAGTTATGGC
GTGGTGTGTGCCAGTGCATTTCAGCGTCATCTGCAGCTGCTGGAA
GAAAGCGAAAAAACCCAGGATCTGCAGATGAAATATCGTCTGCCG
AAACTGGCAAATAATACCCTGCTGCAGATTGCCGAACTGCTGGAT
ACCACCAGTAGCAATCTGGCATTTTGCTTTAGTGCAACCCAGGCC
ATTAGTAGTATTCTGCTGACCTTTCCGTGGAGTGCAAATGATAAA
ATTCTGAGTCTGAATGTTGCCTATCCGACCTGCCAGTTTGCCCTG
GATTTTGTTCGCAATCGTTATGATGTGCAGGTTGATACCCTGGAA
GTGGAGTTTATCTATGATCCGAGCGAATTTCTGAGCCGCGTTGAA
AGCTATCTGGTTAAAAATAAGCCGCGCGTTTTTATTTTTGATTTT
ATTACCAGCATGCCGGTTACCCAGCTGCCGTGTAAAGAACTGATT
CAGCTGTGCAAAAAATATGGTGTGATTAGTGTGGTTGATGCAGCA
CATGGCATTGGTTTTTGCCCGCTGAGCCTGAGTAGTCTGGACCCT
GATTTTCTGTATACCAATGCACATAAATGGCTGAATGCCCCGAGC
GGTACCACCATTCTGTATGTTAGCAAAAAATATCACAACTTCATC
GAAGCACTGCCGATTAGCTATGGCTATCATATTCGTAAACAGAAT
AGTCCGCCGGCCGATAGTCTGGGTATTCGTTTTCTGAATGCAAGC
TTTATGGATCTGCCGAAATTCATTGCCATTGATGCCGCCATTGCA
TTTCGTAAAAGTATTGGCGGCGAACATAAAATTCAGAGTTATAAT
CATGACATCGCCGTGCGTGGCAGTAAAATTATTGCCGAAAGCCTG
GGTACCAGCTATTTTGCACTGGCCAGCCCGATTGCAATGGTTAAT
GTTGAAGTTCCGCTGCGCTGCATTCCGAGTGCCGATTTTCTGGAA
GAATTTTGGCAGAGCAAAAATACCTTTCTGCGCTTTGTGGAATAT
CAGGGTCGTTATTATACCCGCGTGGGTGGTGCCCCGTTTCTGGAA
GAGAGTGATTTTGTGTATGTGGCCGATGTGCTGAAAGAACTGTGC
CAGAAAtaa
<SEQ ID NO: 88; PRT;
F2Egt2; 2_XM_013170142;
Schizosaccharomyces cryophilus>
MSDCMPFGHALKPYYMLDKNYVSVNNGSYGVVCASAFQRHLQLLE
ESEKTQDLQMKYRLPKLANNTLLQIAELLDTTSSNLAFCFSATQA
ISSILLTFPWSANDKILSLNVAYPTCQFALDFVRNRYDVQVDTLE
VEFIYDPSEFLSRVESYLVKNKPRVFIFDFITSMPVTQLPCKELI
QLCKKYGVISVVDAAHGIGFCPLSLSSLDPDFLYTNAHKWLNAPS
GTTILYVSKKYHNFIEALPISYGYHIRKQNSPPADSLGIRFLNAS
FMDLPKFIAIDAAIAFRKSIGGEHKIQSYNHDIAVRGSKIIAESL
GTSYFALASPIAMVNVEVPLRCIPSADFLEEFWQSKNTFLRFVEY
QGRYYTRVGGAPFLEESDFVYVADVLKELCQK
<SEQ ID NO: 89; DNA;
F3Egt2; 2_XM_002482656;
Talaromyces stipitatus>
ATGAGCACCAGTAATCCGACCTTTGGTGCACCGCTGCTGCCGTAT
TTTCCGTTTCAGAGCGATTATCTGAATATTAATCACGGTAGCTTT
GGTGGTTATCCGATTAAGGTGCGTGATGCCCTGCGTGAATATCAG
CGCCAGACCGATGCCAAACCGGATGATTTTATTCGTTATCGCCTG
CCGGGTCTGATTGATAAAAGCCGCGCCGCAGTTGCAGAACTGATT
AATGCCGATGTGGGCAATGTTGTGCTGATTCCGAATGCCACCACC
GGTGTTAATACCGTGCTGCGTAATCTGGTGTATAATCCGGGTGAC
AAAATTGTGTATCTGGGCACCACCTATGGCGCATGTGAAAAAGCC
GTGATGCATATTGTGGATACCTGTATTCCGGCCGGTGCCGTTGAA
GCAATTAAGGTTGAAGTTGAATATCCGGTTACCAGCAAAGAAATT
CTGCGCCGCTTTGAAGATGCCATTAGTCAGAAAGGTGTGCGTATT
GCCCTGTTTGATACCGTTAGTAGTCTGCCGGCCCTGCGTCTGCCG
TATGAAAATATGATTAGCCTGTGTAAAAAGTACCATGTGCTGAGC
CTGATTGATGGTGCCCATGCAGTTGGCGCCATTGAACTGGATATG
CAGCGCCTGGACCCTGATTTCTTTATTAGCAATCTGCATAAATGG
CTGTATACCCCGCGTAGCTGCGCAGTTTTTCATGTGGCAGCCCGT
AGCCAGCATCTGATTAAGACCAGCCTGCCGACCAGCCATGGCTAT
CGTCCGGAAGAACGCCCGGGTCGTCTGCGCGTGAGCAATCCGCTG
CCGACCAGTAGTAAAACCGGTTTTGTGGAACTGTTTGGTTATGTG
GGCACCATGGATTATACCCCGTATCTGTGCATTCCGGAAGCCATT
AAGTTTCGTAAAGAAGTGTGTGGCGGCGAACAGAAACTGCTGCAG
TATATTACCACCCTGGCCAAACAGGGCGGCAATCTGGTTGCAAAT
ATTCTGGGCACCGAACTGCTGGGTGACGAAGATCAGCGCCGCAGC
CCGATGGTTATGGTGCGCCTGCCGCTGAAATTCACTGCCGATGAA
CTGCAGCAGGGTAAACAGCATCTGCTGCTGGAAGAAATTGAACGT
ACCATTAGCGAAAAATATCGCACCTTTGTTCCGCTGATCTATCAT
GGCGGTCATGCCTATGGTCGTCTGAGTGGCCAGGTGTATCTGACC
CTGGAAGATTTTGAAAAAGCCGGCCAGATTCTGGCCAAAGCCTGT
AAAGAATTTGAACAGAAAAGCAAACTGtaa
<SEQ ID NO: 90; PRT;
F3Egt2; 2_XM_002482656;
Talaromyces stipitatus>
MSTSNPTFGAPLLPYFPFQSDYLNINHGSFGGYPIKVRDALREYQ
RQTDAKPDDFIRYRLPGLIDKSRAAVAELINADVGNVVLIPNATT
GVNTVLRNLVYNPGDKIVYLGTTYGACEKAVMHIVDTCIPAGAVE
AIKVEVEYPVTSKEILRRFEDAISQKGVRIALFDTVSSLPALRLP
YENMISLCKKYHVLSLIDGAHAVGAIELDMQRLDPDFFISNLHKW
LYTPRSCAVFHVAARSQHLIKTSLPTSHGYRPEERPGRLRVSNPL
PTSSKTGFVELFGYVGTMDYTPYLCIPEAIKFRKEVCGGEQKLLQ
YITTLAKQGGNLVANILGTELLGDEDQRRSPMVMVRLPLKFTADE
LQQGKQHLLLEEIERTISEKYRTFVPLIYHGGHAYGRLSGQVYLT
LEDFEKAGQILAKACKEFEQKSKL
<SEQ ID NO: 91; DNA;
F4Egt2; 2_XM_011130091;
Arthrobotrys oligospora>
ATGGCCGCTAGTAATCCGCCGAAAACCCCGACCTTTGGCCATAGC
CTGCGTCGCCAGTTTCTGTTTCCGGAAAATTATACCAATCTGAAT
CATGGCAGCTTTGGTGCAATTCCGGCCCCGGTTCTGACCCATCGT
CAGAAACTGCATATTCTGAGCGAACAGCATCCGGATAATTTTATG
CGCTATCATAGTATTAGCCTGCTGGATGAAAGCCGCGCCGCCGTT
GCCAAAGTGCTGAATGCACCGAGCGAAGAAGTTGTGTTTGTTACC
AATGCAACCACCGGTGTGAATATTGTTCTGCGCAATCTGGTTTAT
GAAGAAGGTGACGTGATTCTGCATTTTGGTACCATCTATGGTGCA
TGCGGCCGTACCGTTCAGTATATTGCCGATACCACCCCGGCAACC
TGTATTAGCATTCCGCTGGCATATCCGGTTAGCGATGCAAGTATT
CTGAGTAGCTTTAATACCACCGTTCAGGAAATTAAGGCCGCAGGT
AAAAAACCGAAACTGGTTATTTTTGATACCGTGAGCAGCATGCCG
GGCATGCGCTTTCCGTGGGAAAAAATGATTGTGGCCGCAAAAGAA
GCCGGCGTTCTGAGCCTGATTGATGGCGCCCATGGTGTTGGTAAT
ATTAAGATTGATCTGGGTGCCAATCAGCCGGATTTCTTTGTGAGC
AATTGCCATAAATGGCTGTATACCCCGCGCCCGGCAGCAGTTCTG
TTTGTTCCGATTCGTAATCAGCCGCTGATTACCACCAGCGTGCCG
ACCAGTCATTATTATATTCCGAAAAGTGCAGCCCAGTATTGGAGC
CCGCTGAGTCCGGGTACCAAAAGCAATTTTATTCTGCAGTTTGAG
TTTAATGGCACCATTGATGCAACCCCGTATCTGTGCGTGCCGGCA
GCCCTGAAATTTCGCCAGGAAATTGGCGGTGAAGATGCCATTATT
AATTATTGTAACACCCTGGCATTCGAAGGTGGCGAAGCAGTTGCA
AAAATTCTGGGTACCGAAATTATGGCCCCGGACCCTGCAGCCGTT
GATGGTGGTCGCTGCCCGATGGTGAATATTCGTCTGCCGCTGCTG
AGTGTGCCGAAAACCGAAGTTGAACCGGTGTATAATACCTTTACC
AAAGAAGTTGGTATCCGCGAAAATACCTTTGTGCAGGTTTATGTT
CATAATGCCCGTTGGTGGGTGCGTATTAGTGCCCAGGTGTATCTG
GAAATGAAAGATTTTGTTTGGATCGCCGGCGTGCTGAAAAAAGAA
TGCGAAAAAATTAACGAGCGTATTAAGAGTCTGGCCACCATTGCA
GCAGCAACCGGCGAAAAAGCAGATGTTGCAAATGGTGCAGATGTT
CATGTTGAAGAAGTTCGCAGCGCCAAAAAAGTGGTGAGCGGCATG
GGTGACCTGAAAGTTAGCGAAGCCGAAGGCGAAACCGTGACCGTT
AAAGGCtaa
<SEQ ID NO: 92; PRT;
F4Egt2; 2_XM_011130091;
Arthrobotrys oligospora>
MAASNPPKTPTFGHSLRRQFLFPENYTNLNHGSFGAIPAPVLTHR
QKLHILSEQHPDNFMRYHSISLLDESRAAVAKVLNAPSEEVVFVT
NATTGVNIVLRNLVYEEGDVILHFGTIYGACGRTVQYIADTTPAT
CISIPLAYPVSDASILSSENTTVQEIKAAGKKPKLVIFDTVSSMP
GMRFPWEKMIVAAKEAGVLSLIDGAHGVGNIKIDLGANQPDFFVS
NCHKWLYTPRPAAVLFVPIRNQPLITTSVPTSHYYIPKSAAQYWS
PLSPGTKSNFILQFEFNGTIDATPYLCVPAALKFRQEIGGEDAII
NYCNTLAFEGGEAVAKILGTEIMAPDPAAVDGGRCPMVNIRLPLL
SVPKTEVEPVYNTFTKEVGIRENTFVQVYVHNARWWVRISAQVYL
EMKDFVWIAGVLKKECEKINERIKSLATIAAATGEKADVANGADV
HVEEVRSAKKVVSGMGDLKVSEAEGETVTVKG
<SEQ ID NO: 93; DNA;
F5Egt2; 2_XM_013471838;
Rasamsonia emersonii>
ATGAGTACCCCGTTTGGCCGTCCGATGCGCGAACATTTTCTGTTT
GAAGAAGGTAATATCAACATCAATCACGGCAGTTTTGGCACCTAT
CCGAAACCGGTTCTGGATGCACTGCGTAGTTATCAGCTGCAGGGC
GAAGCCAATCCGGATCGCTTTCTGCGCTATGAAGTGAGCGAACTG
ATTGATCGTAGTCGCGAACAGCTGGCAAAACTGCTGCATGTTGTT
GATGTTGATGAACTGGTGCTGGTTCAGAATGCAACCACCGGCGTT
AATACCGTGCTGCGTAATCTGACCTATGCCCCGGGCGATAAAATT
CTGTATCTGAGCACCGCATATGGTGCATGTGAAAAAACCGTTGAT
TATCTGACCGAAACCACCCCGGCCGAAGCAGTGCGTGTTGAAGTT
GCATATCCGATTAGCGATGATGAACTGGTTGCACGTGTGGAAAAA
GTGCTGAAAGAAAATGCACCGGTTAAAGTTGCAATGTTTGATACC
GTGAGTAGTCTGCCGGGCGTGCGTATTCCGTTTGAACGTCTGGTT
GCCGTTTGCCGTGCCGCAGGTGTTCTGAGCCTGATTGATGGTGCC
CATGGTGTTGGCTGCATTCCGCTGGATCTGGGCAAACTGGATGCC
GATTTCTTTGTGAGTAATTGTCATAAATGGCTGTATGTGCCGCGT
GGTTGCGCAGTGCTGCATGTGCCGAAACGTAATCAGGATCTGATT
CGTAGCAGTATGCCGACCAGTCATGGTTATCAGCCGCGTGAACGT
CCGGGCAAAAAGAAAATTAGTAATCCGCTGCCGCCGAGCACCAAA
AGCGGTTTTGTTCGCATGTTTGAGTTTATTGGTAGCATGGATTAT
GCCCCGTATCTGTGCGTGCCGGCCGCCTTAAAATTTCGTCAGGAA
GTTTGCGGTGGTGAAGAAGCAATTATGAGCTATTGTACCCAGGTT
GCACGCGATGGTAGCCGTCGTGTGGCCGAAATTCTGGGTACCGAA
GTTATGCGTCATGATCAGCCGTGCCCGGTTGTTAATGTGCGCCTG
CCGATTGATCCGCCGGCCGGTGACGTTACCGCCGCTGCAGCACAG
GCACGTATTGGTGCCGTGAATGCCTTTGTTGAAAAAATGATGCTG
AGCGAATATAAAACCTTTGTTCCGGCCTTTTTCCATAATGGCCGT
TTTTGGGTTCGTCTGAGCGGCCAGATATATCTGACCGTGGATGAT
TTTGAAGAAGTTGGCCGTCAGCTGCGCGATATTTGTAGCCGTGTG
GGTCGTACCAGTCATCTGACCGAACTGGAAGATAAAtaa
<SEQ ID NO: 94; PRT;
F5Egt2; 2_XM_013471838;
Rasamsonia emersonii>
MSTPFGRPMREHFLFEEGNININHGSFGTYPKPVLDALRSYQLQG
EANPDRFLRYEVSELIDRSREQLAKLLHVVDVDELVLVQNATTGV
NTVLRNLTYAPGDKILYLSTAYGACEKTVDYLTETTPAEAVRVEV
AYPISDDELVARVEKVLKENAPVKVAMFDTVSSLPGVRIPFERLV
AVCRAAGVLSLIDGAHGVGCIPLDLGKLDADFFVSNCHKWLYVPR
GCAVLHVPKRNQDLIRSSMPTSHGYQPRERPGKKKISNPLPPSTK
SGFVRMFEFIGSMDYAPYLCVPAALKFRQEVCGGEEAIMSYCTQV
ARDGSRRVAEILGTEVMRHDQPCPVVNVRLPIDPPAGDVTAAAAQ
ARIGAVNAFVEKMMLSEYKTFVPAFFHNGRFWVRLSGQIYLTVDD
FEEVGRQLRDICSRVGRTSHLTELEDK
<SEQ ID NO: 95; DNA;
yjeH; methionine transporter;
Escherichia coli>
ATGAGTGGACTCAAACAAGAACTGGGGCTGGCCCAGGGCATTGGC
CTGCTATCGACGTCATTATTAGGCACTGGCGTGTTTGCCGTTCCT
GCGTTAGCTGCGCTGGTAGCGGGCAATAACAGCCTGTGGGCGTGG
CCCGTTTTGATTATCTTAGTGTTCCCGATTGCGATTGTGTTTGCG
ATTCTGGGTCGCCACTATCCCAGCGCAGGCGGCGTCGCGCACTTC
GTCGGTATGGCGTTTGGTTCGCGGCTTGAGCGAGTCACCGGCTGG
CTGTTTTTATCGGTCATTCCCGTGGGTTTGCCTGCCGCACTACAA
ATTGCCGCCGGGTTCGGCCAGGCGATGTTTGGCTGGCATAGCTGG
CAACTGTTGTTGGCAGAACTCGGTACGCTGGCGCTGGTGTGGTAT
ATCGGTACTCGCGGTGCCAGTTCCAGTGCTAATCTACAAACCGTT
ATTGCCGGACTTATCGTCGCGCTGATTGTCGCTATCTGGTGGGCG
GGCGATATCAAACCTGCGAATATCCCCTTTCCGGCACCTGGTAAT
ATCGAACTTACCGGGTTATTTGCTGCGTTATCAGTGATGTTCTGG
TGTTTTGTCGGTCTGGAGGCATTTGCCCATCTCGCCTCGGAATTT
AAAAATCCAGAGCGTGATTTTCCTCGTGCTTTGATGATTGGTCTG
CTGCTGGCAGGATTAGTCTACTGGGGCTGTACGGTAGTCGTCTTA
CACTTCGACGCCTATGGTGAAAAAATGGCGGCGGCAGCATCGCTT
CCAAAAATTGTAGTGCAGTTGTTCGGTGTAGGAGCGTTATGGATT
GCCTGCGTGATTGGCTATCTGGCCTGCTTTGCCAGTCTCAACATT
TATATACAGAGCTTCGCCCGCCTGGTCTGGTCGCAGGCGCAACAT
AATCCTGACCACTACCTGGCACGCCTCTCTTCTCGCCATATCCCG
AATAATGCCCTCAATGCGGTGCTCGGCTGCTGTGTGGTGAGCACT
TTGGTGATTCATGCTTTAGAGATCAATCTGGACGCTCTTATTATT
TATGCCAATGGCATCTTTATTATGATTTATCTGTTATGCATGCTG
GCAGGCTGTAAATTATTGCAAGGACGTTATCGACTACTGGCGGTG
GTTGGCGGGCTGTTATGCGTTCTGTTACTGGCAATGGTCGGCTGG
AAAAGTCTCTATGCGCTGATCATGCTGGCGGGGTTATGGCTGTTG
CTGCCAAAACGAAAAACGCCGGAAAATGGCATAACCACATAA
<SEQ ID NO: 96; PRT;
yjeH; methionine transporter;
Escherichia coli>
MSGLKQELGLAQGIGLLSTSLLGTGVFAVPALAALVAGNNSLWAW
PVLIILVFPIAIVFAILGRHYPSAGGVAHFVGMAFGSRLERVTGW
LFLSVIPVGLPAALQIAAGFGQAMFGWHSWQLLLAELGTLALVWY
IGTRGASSSANLQTVIAGLIVALIVAIWWAGDIKPANIPFPAPGN
IELTGLFAALSVMFWCFVGLEAFAHLASEFKNPERDFPRALMIGL
LLAGLVYWGCTVVVLHFDAYGEKMAAAASLPKIVVQLFGVGALWI
ACVIGYLACFASLNIYIQSFARLVWSQAQHNPDHYLARLSSRHIP
NNALNAVLGCCVVSTLVIHALEINLDALIIYANGIFIMIYLLCML
AGCKLLQGRYRLLAVVGGLLCVLLLAMVGWKSLYALIMLAGLWLL
LPKRKTPENGITT
<SEQ ID NO: 97; DNA;
tnaA; Tryptophanase;
Escherichia coli>
ATGGAAAACTTTAAACATCTCCCTGAACCGTTCCGCATTCGTGTT
ATTGAGCCAGTAAAACGTACCACTCGCGCTTATCGTGAAGAGGCA
ATTATTAAATCCGGTATGAACCCGTTCCTGCTGGATAGCGAAGAT
GTTTTTATCGATTTACTGACCGACAGCGGCACCGGGGCGGTGACG
CAGAGCATGCAGGCTGCGATGATGCGCGGCGACGAAGCCTACAGC
GGCAGTCGTAGCTACTATGCGTTAGCCGAGTCAGTGAAAAATATC
TTTGGTTATCAATACACCATTCCGACTCACCAGGGCCGTGGCGCA
GAGCAAATCTATATTCCGGTACTGATTAAAAAACGCGAGCAGGAA
AAAGGCCTGGATCGCAGCAAAATGGTGGCGTTCTCTAACTATTTC
TTTGATACCACGCAGGGCCATAGCCAGATCAACGGCTGTACCGTG
CGTAACGTCTATATCAAAGAAGCCTTCGATACGGGCGTGCGTTAC
GACTTTAAAGGCAACTTTGACCTTGAGGGATTAGAACGCGGTATT
GAAGAAGTTGGTCCGAATAACGTGCCGTATATCGTTGCAACCATC
ACCAGTAACTCTGCAGGTGGTCAGCCGGTTTACTGGCAAACTTAA
AAGCGATGTACAGCATCGCGAAGAAATACGATATTCCGGTGGTAA
TGGACTCCGCGCGCTTTGCTGAAAACGCCTATTTCATCAAGCAGC
GTGAAGCAGAATACAAAGACTGGACCATCGAGCAGATCACCCGCG
AAACCTACAAATATGCCGATATGCTGGCGATGTCCGCCAAGAAAG
ATGCGATGGTGCCGATGGGCGGCCTGCTGTGCATGAAAGACGACA
GCTTCTTTGATGTGTACACCGAGTGCAGAACCCTTTGCGTGGTGC
AGGAAGGCTTCCCGACATATGGCGGCCTGGAAGGCGGCGCGATGG
AGCGTCTGGCGGTAGGTCTGTATGACGGCATGAATCTCGACTGGC
TGGCTTATCGTATCGCGCAGGTACAGTATCTGGTCGATGGTCTGG
AAGAGATTGGCGTTGTCTGCCAGCAGGCGGGCGGTCACGCGGCAT
TCGTTGATGCCGGTAAACTGTTGCCGCATATCCCGGCAGACCAGT
TCCCGGCACAGGCGCTGGCCTGCGAGCTGTATAAAGTCGCCGGTA
TCCGTCGGTAGAAATTGGCTCTTTCCTGTTAGGCCGCGATCCGAA
AACCGGTAAACAACTGCCATGCCCGGCTGAACTGCTGCGTTTAAC
CATTCCGCGCGCAACATATACTCAAACACATATGGACTTCATTAT
TGAAGCCTTTAAACATGTGAAAGAGAACGCGGCGAATATTAAAGG
ATTAACCTTTACGTACGAACCGAAAGTATTGCGTCACTTCACCGC
AAAACTTAAAGAAGTTTAA
<SEQ ID NO: 98; PRT;
TnaA; Tryptophanase;
Escherichia coli>
MENFKHLPEPFRIRVIEPVKRTTRAYREEAIIKSGMNPFLLDSED
VFIDLLTDSGTGAVTQSMQAAMMRGDEAYSGSRSYYALAESVKNI
FGYQYTIPTHQGRGAEQIYIPVLIKKREQEKGLDRSKMVAFSNYF
FDTTQGHSQINGCTVRNVYIKEAFDTGVRYDFKGNFDLEGLERGI
EEVGPNNVPYIVATITSNSAGGQPVSLANLKAMYSIAKKYDIPVV
MDSARFAENAYFIKOREAEYKDWTIEQITRETYKYADMLAMSAKK
DAMVPMGGLLCMKDDSFFDVYTECRTLCVVQEGFPTYGGLEGGAM
ERLAVGLYDGMNLDWLAYRIAQVQYLVDGLEEIGVVCQQAGGHAA
FVDAGKLLPHIPADQFPAQALACELYKVAGIRAVEIGSFLLGRDP
KTGKQLPCPAELLRLTIPRATYTQTHMDFIIEAFKHVKENAANIK
GLTFTYEPKVLRHFTAKLKEV
<SEQ ID NO: 99; DNA;
sdaA; L-serine dehydratase;
Escherichia coli>
GTGATTAGTCTATTCGACATGTTTAAGGTGGGGATTGGTCCCTCA
TCTTCCCATACCGTAGGGCCTATGAAGGCAGGTAAACAGTTCGTC
GATGATCTGGTCGAAAAAGGCTTACTGGATAGCGTTACTCGCGTT
GCCGTGGACGTTTATGGTTCACTGTCGCTGACGGGTAAAGGCCAC
CACACCGATATCGCCATTATTATGGGTCTTGCAGGTAACGAACCT
GCCACCGTGGATATCGACAGTATTCCCGGTTTTATTCGCGACGTA
GAAGAGCGCGAACGTCTGCTGCTGGCACAGGGACGGCATGAAGTG
GATTTCCCGCGCGACAACGGGATGCGTTTTCATAACGGCAACCTG
CCGCTGCATGAAAACGGTATGCAAATCCACGCCTATAACGGCGAT
GAAGTCGTCTACAGCAAAACTTATTATTCCATCGGCGGCGGTTTT
ATCGTCGATGAAGAACACTTTGGTCAGGATGCTGCCAACGAAGTA
AGCGTGCCGTATCCGTTCAAATCTGCCACCGAACTGCTCGCGTAC
TGTAATGAAACCGGCTATTCGCTGTCTGGTCTCGCTATGCAGAAC
GAACTGGCGCTGCACAGCAAGAAAGAGATCGACGAGTATTTCGCG
CATGTCTGGCAAACCATGCAGGCATGTATCGATCGCGGGATGAAC
ACCGAAGGTGTACTGCCAGGCCCGCTGCGCGTGCCACGTCGTGCG
TCTGCCCTGCGCCGGATGCTGGTTTCCAGCGATAAACTGTCTAAC
GATCCGATGAATGTCATTGACTGGGTAAACATGTTTGCGCTGGCA
GTTAACGAAGAAAACGCCGCCGGTGGTCGTGTGGTAACTGCGCCA
ACCAACGGTGCCTGCGGTATCGTTCCGGCAGTGCTGGCTTACTAT
GACCACTTTATTGAATCGGTCAGCCCGGACATCTATACCCGTTAC
TTTATGGCAGCGGGCGCGATTGGTGCATTGTATAAAATGAACGCC
TCTATTTCCGGTGCGGAAGTTGGTTGCCAGGGCGAAGTGGGTGTT
GCCTGTTCAATGGCTGCTGCGGGTCTTGCAGAACTGCTGGGCGGT
AGCCCGGAACAGGTTTGCGTGGCGGCGGAAATTGGCATGGAACAC
AACCTTGGTTTAACCTGCGACCCGGTTGCAGGGCAGGTTCAGGTG
CCGTGCATTGAGCGTAATGCCATTGCCTCTGTGAAGGCGATTAAC
GCCGCGCGGATGGCTCTGCGCCGCACCAGTGCACCGCGCGTCTCG
CTGGATAAGGTCATCGAAACGATGTACGAAACCGGTAAGGACATG
AACGCCAAATACCGCGAAACCTCACGCGGTGGTCTGGCAATCAAA
GTCCAGTGTGACTAA
<SEQ ID NO: 100; PRT;
SdaA; L-serine dehydratase;
Escherichia coli>
MISLFDMFKVGIGPSSSHTVGPMKAGKQFVDDLVEKGLLDSVTRV
AVDVYGSLSLTGKGHHTDIAIIMGLAGNEPATVDIDSIPGFIRDV
EERERLLLAQGRHEVDFPRDNGMRFHNGNLPLHENGMQIHAYNGD
EVVYSKTYYSIGGGFIVDEEHFGQDAANEVSVPYPFKSATELLAY
CNETGYSLSGLAMQNELALHSKKEIDEYFAHVWQTMQACIDRGMN
TEGVLPGPLRVPRRASALRRMLVSSDKLSNDPMNVIDWVNMFALA
VNEENAAGGRVVTAPTNGACGIVPAVLAYYDHFIESVSPDIYTRY
FMAAGAIGALYKMNASISGAEVGCQGEVGVACSMAAAGLAELLGG
SPEQVCVAAEIGMEHNLGLTCDPVAGQVQVPCIERNAIASVKAIN
AARMALRRTSAPRVSLDKVIETMYETGKDMNAKYRETSRGGLAIK
VQCD
<SEQ ID NO: 101; DNA;
serA; D-3-phosphoglycerate dehydrogenase;
Escherichia coli>
ATGGCAAAGGTATCGCTGGAGAAAGACAAGATTAAGTTTCTGCTG
GTAGAAGGCGTGCACCAAAAGGCGCTGGAAAGCCTTCGTGCAGCT
GGTTACACCAACATCGAATTTCACAAAGGCGCGCTGGATGATGAA
CAATTAAAAGAATCCATCCGCGATGCCCACTTCATCGGCCTGCGA
TCCCGTACCCATCTGACTGAAGACGTGATCAACGCCGCAGAAAAA
CTGGTCGCTATTGGCTGTTTCTGTATCGGAACAAACCAGGTTGAT
CTGGATGCGGCGGCAAAGCGCGGGATCCCGGTATTTAACGCACCG
TTCTCAAATACGCGCTCTGTTGCGGAGCTGGTGATTGGCGAACTG
CTGCTGCTATTGCGCGGCGTGCCGGAAGCCAATGCTAAAGCGCAC
CGTGGCGTGTGGAACAAACTGGCGGCGGGTTCTTTTGAAGCGCGC
GGCAAAAAGCTGGGTATCATCGGCTACGGTCATATTGGTACGCAA
TTGGGCATTCTGGCTGAATCGCTGGGAATGTATGTTTACTTTTAT
GATATTGAAAATAAACTGCCGCTGGGCAACGCCACTCAGGTACAG
CATCTTTCTGACCTGCTGAATATGAGCGATGTGGTGAGTCTGCAT
GTACCAGAGAATCCGTCCACCAAAAATATGATGGGCGCGAAAGAA
ATTTCACTAATGAAGCCCGGCTCGCTGCTGATTAATGCTTCGCGC
GGTACTGTGGTGGATATTCCGGCGCTGTGTGATGCGCTGGCGAGC
AAACATCTGGCGGGGGCGGCAATCGACGTATTCCCGACGGAACCG
GCGACCAATAGCGATCCATTTACCTCTCCGCTGTGTGAATTCGAC
AACGTCCTTCTGACGCCACACATTGGCGGTTCGACTCAGGAAGCG
CAGGAGAATATCGGCCTGGAAGTTGCGGGTAAATTGATCAAGTAT
TCTGACAATGGCTCAACGCTCTCTGCGGTGAACTTCCCGGAAGTC
TCGCTGCCACTGCACGGTGGGCGTCGTCTGATGCACATCCACGAA
AACCGTCCGGGCGTGCTAACTGCGCTGAACAAAATCTTCGCCGAG
CAGGGCGTCAACATCGCCGCGCAATATCTGCAAACTTCCGCCCAG
ATGGGTTATGTGGTTATTGATATTGAAGCCGACGAAGACGTTGCC
GAAAAAGCGCTGCAGGCAATGAAAGCTATTCCGGGTACCATTCGC
GCCCGTCTGCTGTACTAA
<SEQ ID NO: 102; PRT;
SerA; D-3-phosphoglycerate dehydrogenase;
Escherichia coli>
MAKVSLEKDKIKFLLVEGVHQKALESLRAAGYTNIEFHKGALDDE
QLKESIRDAHFIGLRSRTHLTEDVINAAEKLVAIGCFCIGTNQVD
LDAAAKRGIPVFNAPFSNTRSVAELVIGELLLLLRGVPEANAKAH
RGVWNKLAAGSFEARGKKLGIIGYGHIGTQLGILAESLGMYVYFY
DIENKLPLGNATQVQHLSDLLNMSDVVSLHVPENPSTKNMMGAKE
ISLMKPGSLLINASRGTVVDIPALCDALASKHLAGAAIDVFPTEP
ATNSDPFTSPLCEFDNVLLTPHIGGSTQEAQENIGLEVAGKLIKY
SDNGSTLSAVNFPEVSLPLHGGRRLMHIHENRPGVLTALNKIFAE
QGVNIAAQYLQTSAQMGYVVIDIEADEDVAEKALQAMKAIPGTIR
ARLLY
<SEQ ID NO: 103; DNA;
serB; Phosphoserine phosphatase;
Escherichia coli>
ATGCCTAACATTACCTGGTGCGACCTGCCTGAAGATGTCTCTTTA
TGGCCGGGTCTGCCTCTTTCATTAAGTGGTGATGAAGTGATGCCA
CTGGATTACCACGCAGGTCGTAGCGGCTGGCTGCTGTATGGTCGT
GGGCTGGATAAACAACGTCTGACCCAATACCAGAGCAAACTGGGT
GCGGCGATGGTGATTGTTGCCGCCTGGTGCGTGGAAGATTATCAG
GTGATTCGTCTGGCAGGTTCACTCACCGCACGGGCTACACGCCTG
GCCCACGAAGCGCAGCTGGATGTCGCCCCGCTGGGGAAAATCCCG
CACCTGCGCACGCCGGGTTTGCTGGTGATGGATATGGACTCCACC
GCCATCCAGATTGAATGTATTGATGAAATTGCCAAACTGGCCGGA
ACGGGCGAGATGGTGGCGGAAGTAACCGAACGGGCGATGCGCGGC
GAACTCGATTTTACCGCCAGCCTGCGCAGCCGTGTGGCGACGCTG
AAAGGCGCTGACGCCAATATTCTGCAACAGGTGCGTGAAAATCTG
CCGCTGATGCCAGGCTTAACGCAACTGGTGCTCAAGCTGGAAACG
CTGGGCTGGAAAGTGGCGATTGCCTCCGGCGGCTTTACTTTCTTT
GCTGAATACCTGCGCGACAAGCTGCGCCTGACCGCCGTGGTAGCC
AATGAACTGGAGATCATGGACGGTAAATTTACCGGCAATGTGATC
GGCGACATCGTAGACGCGCAGTACAAAGCGAAAACTCTGACTCGC
CTCGCGCAGGAGTATGAAATCCCGCTGGCGCAGACCGTGGCGATT
GGCGATGGAGCCAATGACCTGCCGATGATCAAAGCGGCAGGGCTG
GGGATTGCCTACCATGCCAAGCCAAAAGTGAATGAAAAGGCGGAA
GTCACCATCCGTCACGCTGACCTGATGGGGGTATTCTGCATCCTC
TCAGGCAGCCTGAATCAGAAGTAA
<SEQ ID NO: 104; PRT;
SerB; Phosphoserine phosphatase;
Escherichia coli>
MPNITWCDLPEDVSLWPGLPLSLSGDEVMPLDYHAGRSGWLLYGR
GLDKQRLTQYQSKLGAAMVIVAAWCVEDYQVIRLAGSLTARATRL
AHEAQLDVAPLGKIPHLRTPGLLVMDMDSTAIQIECIDEIAKLAG
TGEMVAEVTERAMRGELDFTASLRSRVATLKGADANILQQVRENL
PLMPGLTQLVLKLETLGWKVAIASGGFTFFAEYLRDKLRLTAVVA
NELEIMDGKFTGNVIGDIVDAQYKAKTLTRLAQEYEIPLAQTVAI
GDGANDLPMIKAAGLGIAYHAKPKVNEKAEVTIRHADLMGVFCIL
SGSLNQK
<SEQ ID NO: 105; DNA;
serC; Phosphoserine phosphatase;
Escherichia coli>
ATGGCTCAAATCTTCAATTTTAGTTCTGGTCCGGCAATGCTACCG
GCAGAGGTGCTTAAACAGGCTCAACAGGAACTGCGCGACTGGAAC
GGTCTTGGTACGTCGGTGATGGAAGTGAGTCACCGTGGCAAAGAG
TTCATTCAGGTTGCAGAGGAAGCCGAGAAGGATTTTCGCGATCTT
CTTAATGTCCCCTCCAACTACAAGGTATTATTCTGCCATGGCGGT
GGTCGCGGTCAGTTTGCTGCGGTACCGCTGAATATTCTCGGTGAT
AAAACCACCGCAGATTATGTTGATGCCGGTTACTGGGCGGCAAGT
GCCATTAAAGAAGCGAAAAAATACTGCACGCCTAATGTCTTTGAC
GCCAAAGTGACTGTTGATGGTCTGCGCGCGGTTAAGCCAATGCGT
GAATGGCAACTCTCTGATAATGCTGCTTATATGCATTATTGCCCG
AATGAAACCATCGATGGTATCGCCATCGACGAAACGCCAGACTTC
GGCGCAGATGTGGTGGTCGCCGCTGACTTCTCTTCAACCATTCTT
TCCCGTCCGATTGACGTCAGCCGTTATGGTGTAATTTACGCTGGC
GCGCAGAAAAATATCGGCCCGGCTGGCCTGACAATCGTCATCGTT
CGTGAAGATTTGCTGGGCAAAGCGAATATCGCGTGTCCGTCGATT
CTGGATTATTCCATCCTCAACGATACGGCTCCATGTTTAACACGC
CGCCGACATTTGCCTGGTATCTATCTGGTCTGGTCTTTAAATGGC
TGAAAGCGAACGGCGGTGTAGCTGAAATGGATAAAATCAATCAGC
AAAAAGCAGAACTGCTATATGGGGTGATTGATAACAGCGATTTCT
ACCGCAATGACGTGGCGAAAGCTAACCGTTCGCGGATGAACGTGC
CGTTCCAGTTGGCGGACAGTGCGCTTGACAAATTGTTCCTTGAAG
AGTCTTTTGCTGCTGGCCTTCATGCACTGAAAGGTCACCGTGTGG
TCGGCGGAATGCGCGCTTCTATTTATAACGCCATGCCGCTGGAAG
GCGTTAAAGCGCTGACAGACTTCATGGTTGAGTTCGAACGCCGTC
ACGGTTAA
<SEQ ID NO: 106; PRT;
SerC; Phosphoserine phosphatase;
Escherichia coli>
MAQIFNFSSGPAMLPAEVLKQAQQELRDWNGLGTSVMEVSHRGKE
FIQVAEEAEKDFRDLLNVPSNYKVLFCHGGGRGQFAAVPLNILGD
KTTADYVDAGYWAASAIKEAKKYCTPNVFDAKVTVDGLRAVKPMR
EWQLSDNAAYMHYCPNETIDGIAIDETPDFGADVVVAADESSTIL
SRPIDVSRYGVIYAGAQKNIGPAGLTIVIVREDLLGKANIACPSI
LDYSILNDNGSMFNTPPTFAWYLSGLVFKWLKANGGVAEMDKINQ
QKAELLYGVIDNSDFYRNDVAKANRSRMNVPFQLADSALDKLFLE
ESFAAGLHALKGHRVVGGMRASIYNAMPLEGVKALTDFMVEFERR
HG
<SEQ ID NO: 107; DNA;
cysM; Cysteine synthase B;
Escherichia coli>
GTGAGTACATTAGAACAAACAATAGGCAATACGCCTCTGGTGAAG
TTGCAGCGAATGGGGCCGGATAACGGCAGTGAAGTGTGGTTAAAA
CTGGAAGGCAATAACCCGGCAGGTTCGGTGAAAGATCGTGCGGCA
CTTTCGATGATCGTCGAGGCGGAAAAGCGCGGGGAAATTAAACCG
GGTGATGTCTTAATCGAAGCCACCAGTGGTAACACCGGCATTGCG
CTGGCAATGATTGCCGCGCTGAAAGGCTATCGCATGAAATTGCTG
ATGCCCGACAACATGAGCCAGGAACGCCGTGCGGCGATGCGTGCT
TATGGTGCGGAACTGATTCTTGTCACCAAAGAGCAGGGCATGGAA
GGTGCGCGCGATCTGGCGCTGGAGATGGCGAATCGTGGCGAAGGA
AAGCTGCTCGATCAGTTCAATAATCCCGATAACCCTTATGCGCAT
TACACCACCACTGGGCCGGAAATCTGGCAGCAAACCGGCGGGCGC
ATCACTCATTTTGTCTCCAGCATGGGGACGACCGGCACTATCACC
GGCGTCTCACGCTTTATGCGCGAACAATCCAAACCGGTGACCATT
GTCGGCCTGCAACCGGAAGAGGGCAGCAGCATTCCCGGCATTCGC
CGCTGGCCTACGGAATATCTGCCGGGGATTTTCAACGCTTCTCTG
GTGGATGAGGTGCTGGATATTCATCAGCGCGATGCGGAAAACACC
ATGCGCGAACTGGCGGTGCGGGAAGGAATATTCTGTGGCGTCAGC
TCCGGCGGCGCGGTTGCCGGAGCACTGCGGGTGGCAAAAGCTAAC
CCTGACGCGGTGGTGGTGGCGATCATCTGCGATCGTGGCGATCGC
TACCTTTCTACCGGGGTGTTTGGGGAAGAGCATTTTAGCCAGGGG
GCGGGGATTTAA
<SEQ ID NO: 108; PRT;
CysM; Cysteine synthase B;
Escherichia coli>
MSTLEQTIGNTPLVKLQRMGPDNGSEVWLKLEGNNPAGSVKDRAA
LSMIVEAEKRGEIKPGDVLIEATSGNTGIALAMIAALKGYRMKLL
MPDNMSQERRAAMRAYGAELILVTKEQGMEGARDLALEMANRGEG
KLLDQFNNPDNPYAHYTTTGPEIWQQTGGRITHFVSSMGTTGTIT
GVSRFMREQSKPVTIVGLQPEEGSSIPGIRRWPTEYLPGIFNASL
VDEVLDIHQRDAENTMRELAVREGIFCGVSSGGAVAGALRVAKAN
PDAVVVAIICDRGDRYLSTGVFGEEHFSQGAGI
<SEQ ID NO: 109; DNA;
nrdH; Glutaredoxin-like protein;
Escherichia coli>
ATGCGCATTACTATTTACACTCGTAACGATTGCGTTCAGTGCCAC
GCCACCAAACGGGCGATGGAAAACCGGGGCTTTGATTTTGAAATG
ATTAATGTCGATCGCGTTCCTGAAGCGGCAGAAGCGTTGCGTGCT
CAGGGCTTTCGTCAGTTGCCGGTAGTGATTGCTGGCGATCTTAGC
TGGTCTGGTTTCCGTCCGGACATGATTAACCGTCTGCATCCAGCG
CCACACGCGGCCAGTGCATGA
<SEQ ID NO: 110; PRT;
NrdH; Glutaredoxin-like protein;
Escherichia coli>
MSQLVYFSSSSENTQRFIERLGLPAVRIPLNERERIQVDEPYILI
VPSYGGGGTAGAVPRQVIRFLNDEHNRALLRGVIASGNRNFGEAY
GRAGDVIARKCGVPWLYRFELMGTQSDIENVRKGVTEFWQRQPQN
A
<SEQ ID NO: 111; DNA;
cysE; Serine acetyltransferase;
Escherichia coli>
ATGTCGTGTGAAGAACTGGAAATTGTCTGGAACAATATTAAAGCC
GAAGCCAGAACGCTGGCGGACTGTGAGCCAATGCTGGCCAGTTTT
TACCACGCGACGCTACTCAAGCACGAAAACCTTGGCAGTGCACTG
AGCTACATGCTGGCGAACAAGCTGTCATCGCCAATTATGCCTGCT
ATTGCTATCCGTGAAGTGGTGGAAGAAGCCTACGCCGCTGACCCG
GAAATGATCGCCTCTGCGGCCTGTGATATTCAGGCGGTGCGTACC
CGCGACCCGGCAGTCGATAAATACTCAACCCCGTTGTTATACCTG
AAGGGTTTTCATGCCTTGCAGGCCTATCGCATCGGTCACTGGTTG
TGGAATCAGGGGCGTCGCGCACTGGCAATCTTTCTGCAAAACCAG
GTTTCTGTGACGTTCCAGGTCGATATTCACCCGGCAGCAAAAATT
GGTCGCGGTATCATGCTTGACCACGCGACAGGCATCGTCGTTGGT
GAAACGGCGGTGATTGAAAACGACGTATCGATTCTGCAATCTGTG
ACGCTTGGCGGTACGGGTAAATCTGGTGGTGACCGTCACCCGAAA
ATTCGTGAAGGTGTGATGATTGGCGCGGGCGCGAAAATCCTCGGC
AATATTGAAGTTGGGCGCGGCGCGAAGATTGGCGCAGGTTCCGTG
GTGCTGCAACCGGTGCCGCCGCATACCACCGCCGCTGGCGTTCCG
GCTCGTATTGTCGGTAAACCAGACAGCGATAAGCCATCAATGGAT
ATGGACCAGCATTTCAACGGTATTAACCATACATTTGAGTATGGG
GATGGGATCTAA
<SEQ ID NO: 112; PRT;
CysE; Serine acetyltransferase;
Escherichia coli>
MSCEELEIVWNNIKAEARTLADCEPMLASFYHATLLKHENLGSAL
SYMLANKLSSPIMPAIAIREVVEEAYAADPEMIASAACDIQAVRT
RDPAVDKYSTPLLYLKGFHALQAYRIGHWLWNQGRRALAIFLQNQ
VSVTFQVDIHPAAKIGRGIMLDHATGIVVGETAVIENDVSILQSV
TLGGTGKSGGDRHPKIREGVMIGAGAKILGNIEVGRGAKIGAGSV
VLQPVPPHTTAAGVPARIVGKPDSDKPSMDMDQHENGINHTFEYG
DGI
<SEQ ID NO: 113; DNA;
ydeE; EamA domain-containing protein
Escherichia coli>
ATGTCGCGAAAAGATGGGGTGTTGGCGCTACTGGTAGTGGTCGTA
TGGGGGCTAAATTTTGTGGTCATCAAAGTGGGGCTTCATAACATG
CCACCGCTGATGCTGGCCGGTTTGCGCTTTATGCTGGTCGCTTTT
CCGGCTATCTTTTTTGTCGCACGACCGAAAGTACCACTGAATTTG
CTGCTGGGGTATGGATTAACCATCAGTTTTGCGCAGTTTGCTTTT
CTTTTTTGTGCCATTAACTTCGGTATGCCTGCTGGACTGGCTTCG
CTGGTGTTACAGGCACAGGCGTTTTTTACTATCATGCTTGGCGCG
TTTACTTTCGGGGAGCGACTGCATGGCAAACAATTGGCGGGGATC
GCCTTAGCGATTTTTGGCGTACTGGTGTTAATCGAAGATAGTCTG
AACGGTCAGCATGTGGCGATGCTCGGCTTTATGTTGACCCTGGCG
GCAGCATTTAGTTGGGCGTGTGGCAACATCTTCAATAAAAAGATC
ATGTCGCACTCAACGCGTCCGGCGGTGATGTCGCTGGTAATCTGG
AGCGCTTTAATCCCAATCATTCCCTTCTTTGTTGCCTCGCTGATT
CTCGATGGTTCCGCAACCATGATTCACAGTCTGGTTACTATCGAT
ATGACCACCATCTTGTCTCTGATGTATCTGGCGTTTGTGGCGACA
ATTGTTGGTTATGGGATCTGGGGGACGTTACTGGGACGCTATGAA
ACCTGGCGGGTTGCACCGTTATCGTTACTGGTGCCCGTAGTAGGA
CTGGCAAGTGCGGCACTATTGTTGGATGAACGCTTAACGGGTCTG
CAATTTTTAGGTGCGGTGCTCATTATGACCGGGCTGTATATCAAT
GTATTTGGCTTGCGGTGGCGTAAAGCGGTAAAGGTGGGAAGTTAA
<SEQ ID NO: 114; PRT;
YdeE; EamA domain-containing protein;
Escherichia coli>
MSRKDGVLALLVVVVWGLNFVVIKVGLHNMPPLMLAGLRFMLVAF
PAIFFVARPKVPLNLLLGYGLTISFAQFAFLFCAINFGMPAGLAS
LVLQAQAFFTIMLGAFTFGERLHGKQLAGIALAIFGVLVLIEDSL
NGQHVAMLGFMLTLAAAFSWACGNIFNKKIMSHSTRPAVMSLVIW
SALIPIIPFFVASLILDGSATMIHSLVTIDMTTILSLMYLAFVAT
IVGYGIWGTLLGRYETWRVAPLSLLVPVVGLASAALLLDERLTGL
QFLGAVLIMTGLYINVFGLRWRKAVKVGS
<SEQ ID NO: 115; DNA;
yhaM; UPF0597 protein YhaM;
Escherichia coli>
ATGTTTGATTCGACTTTAAATCCGTTATGGCAGCGTTACATCCTC
GCCGTTCAGGAGGAAGTAAAACCGGCGCTGGGATGTACTGAACCG
ATTTCACTGGCGCTGGCGGCGGCGGTTGCTGCGGCAGAACTGGAA
GGTCCGGTTGAACGTGTAGAAGCCTGGGTTTCGCCAAATCTGATG
AAGAACGGTCTGGGCGTCACCGTTCCCGGCACGGGAATGGTGGGG
CTGCCGATTGCGGCGGCGCTGGGGGCGTTAGGTGGAAATGCCAAC
GCCGGGCTGGAAGTGCTGAAAGACGCAACAGCGCAGGCAATTGCC
GATGCCAAAGCACTGCTGGCGGCGGGGAAAGTCTCCGTTAAGATC
CAGGAACCTTGCGATGAAATCCTCTTCTCACGCGCCAAAGTCTGG
AACGGTGAGAAGTGGGCGTGTGTCACCATCGTCGGCGGGCATACC
AACATTGTGCATATCGAGACGCACGATGGTGTGGTGTTTACCCAG
CAGGCGTGTGTGGCAGAGGGCGAGCAAGAGTCTCCGCTGACGGTG
CTTTCCAGAACGACGCTGGCTGAGATCCTGAAGTTCGTCAATGAA
GTCCCGTTTGCGGCGATCCGCTTTATTCTCGATTCCGCGAAGCTA
AATTGTGCGTTATCGCAGGAAGGTTTGAGCGGTAAGTGGGGGCTG
CATATTGGCGCGACGCTGGAAAAACAGTGCGAGCGCGGTTTGCTG
GCGAAAGATCTCTCTTCATCCATTGTGATTCGTACCAGCGCGGCA
TCCGATGCGCGTATGGGCGGCGCTACGCTTCCGGCTATGAGTAAC
TCCGGCTCGGGTAACCAGGGGATTACCGCAACAATGCCTGTGGTG
GTGGTAGCAGAACACTTCGGAGCGGATGATGAACGGCTGGCGCGT
GCGCTGATGCTTTCGCATTTGAGCGCAATTTACATCCATAACCAG
TTACCGCGTTTGTCTGCGCTGTGTGCCGCAACGACCGCAGCAATG
GGGGCCGCCGCCGGGATGGCATGGCTGGTGGATGGGCGTTATGAA
ACCATCTCGATGGCGATCAGCAGTATGATCGGCGATGTCAGCGGC
ATGATTTGCGATGGTGCGTCGAACAGCTGCGCGATGAAGGTTTCG
ACCAGTGCTTCGGCTGCGTGGAAAGCGGTGTTAATGGCGCTGGAT
GATACCGCCGTGACCGGCAATGAAGGGATTGTGGCGCATGATGTT
GAGCAGTCGATTGCCAACCTGTGTGCGTTAGCAAGCCATTCGATG
CAGCAAACGGATCGGCAGATTATCGAGATTATGGCGAGCAAGGCC
AGATAA
<SEQ ID NO: 116; PRT;
YhaM; UPF0597 protein YhaM;
Escherichia coli>
MFDSTLNPLWQRYILAVQEEVKPALGCTEPISLALAAAVAAAELE
GPVERVEAWVSPNLMKNGLGVTVPGTGMVGLPIAAALGALGGNAN
AGLEVLKDATAQAIADAKALLAAGKVSVKIQEPCDEILFSRAKVW
NGEKWACVTIVGGHTNIVHIETHDGVVFTQQACVAEGEQESPLTV
LSRTTLAEILKFVNEVPFAAIRFILDSAKLNCALSQEGLSGKWGL
HIGATLEKQCERGLLAKDLSSSIVIRTSAASDARMGGATLPAMSN
SGSGNQGITATMPVVVVAEHFGADDERLARALMLSHLSAIYIHNQ
LPRLSALCAATTAAMGAAAGMAWLVDGRYETISMAISSMIGDVSG
MICDGASNSCAMKVSTSASAAWKAVLMALDDTAVTGNEGIVAHDV
EQSIANLCALASHSMQQTDRQIIEIMASKAR
<SEQ ID NO: 117; DNA;
cysB; HTH-type transcriptional regulator;
Escherichia coli>
ATGAAATTACAACAACTTCGCTATATTGTTGAGGTGGTCAATCAT
AACCTGAATGTCTCATCAACAGCGGAAGGACTTTACACATCACAA
CCCGGGATCAGTAAACAAGTCAGAATGCTGGAAGACGAGCTAGGC
ATTCAAATTTTTTCCCGAAGCGGCAAGCACCTGACGCAGGTAACG
CCAGCAGGGCAAGAAATAATTCGTATCGCTCGCGAAGTCCTGTCG
AAAGTCGATGCCATAAAATCGGTTGCCGGAGAGCACACCTGGCCG
GATAAAGGTTCACTGTATATCGCCACCACGCATACCCAGGCACGC
TACGCATTACCAAACGTCATCAAAGGCTTTATTGAGCGTTATCCT
CGCGTTTCTTTGCATATGCACCAGGGCTCGCCGACACAAATTGCT
GATGCCGTCTCTAAAGGCAATGCTGATTTCGCTATCGCCACAGAA
GCGCTGCATCTGTATGAAGATTTAGTGATGTTACCGTGCTACCAC
TGGAATCGGGCTATTGTAGTCACTCCGGATCACCCGCTGGCAGGC
AAAAAAGCCATTACCATTGAAGAACTGGCGCAATATCCGTTGGTG
ACATATACCTTCGGCTTTACCGGACGTTCAGAACTGGATACTGCC
TTTAATCGCGCAGGGTTAACGCCGCGTATCGTTTTCACGGCAACG
GATGCTGACGTCATTAAAACTTACGTCCGGTTAGGGCTGGGGGTA
GGGGTCATTGCCAGCATGGCGGTGGATCCGGTCGCCGATCCCGAC
CTTGTGCGTGTTGATGCTCACGATATCTTCAGCCACAGTACAACC
AAAATTGGTTTTCGCCGTAGTACTTTCTTGCGCAGTTATATGTAT
GATTTCATTCAGCGTTTTGCACCGCATTTAACGCGTGATGTCGTT
GATGCGGCTGTCGCATTGCGCTCTAATGAAGAAATTGAGGTCATG
TTTAAAGATATAAAACTGCCGGAAAAATAA
<SEQ ID NO: 118; PRT;
CysB; HTH-type transcriptional regulator;
Escherichia coli>
MKLQQLRYIVEVVNHNLNVSSTAEGLYTSQPGISKQVRMLEDELG
IQIFSRSGKHLTQVTPAGQEIIRIAREVLSKVDAIKSVAGEHTWP
DKGSLYIATTHTQARYALPNVIKGFIERYPRVSLHMHQGSPTQIA
DAVSKGNADFAIATEALHLYEDLVMLPCYHWNRAIVVTPDHPLAG
KKAITIEELAQYPLVTYTFGFTGRSELDTAFNRAGLTPRIVFTAT
DADVIKTYVRLGLGVGVIASMAVDPVADPDLVRVDAHDIFSHSTT
KIGFRRSTFLRSYMYDFIQRFAPHLTRDVVDAAVALRSNEEIEVM
FKDIKLPEK
<SEQ ID NO: 119; DNA;
cysK; Cysteine synthase A;
Escherichia coli>
ATGAGTAAGATTTTTGAAGATAACTCGCTGACTATCGGTCACACG
CCGCTGGTTCGCCTGAATCGCATCGGTAACGGACGCATTCTGGCG
AAGGTGGAATCTCGTAACCCCAGCTTCAGCGTTAAGTGCCGTATC
GGTGCCAACATGATTTGGGATGCCGAAAAGCGCGGCGTGCTGAAA
CCAGGCGTTGAACTGGTTGAACCGACCAGCGGTAATACCGGGATT
GCACTGGCCTATGTAGCTGCCGCTCGCGGTTACAAACTCACCCTG
ACCATGCCAGAAACCATGAGTATTGAACGCCGCAAGCTGCTGAAA
GCGTTAGGTGCAAACCTGGTGCTGACGGAAGGTGCTAAAGGCATG
AAAGGCGCAATCCAAAAAGCAGAAGAAATTGTCGCCAGCAATCCA
GAGAAATACCTGCTGCTGCAACAATTCAGCAATCCGGCAAACCCT
GAAATTCACGAAAAGACCACCGGTCCGGAGATATGGGAAGATACC
GACGGTCAGGTTGATGTATTTATTGCTGGCGTTGGGACTGGCGGT
ACGCTGACTGGCGTCAGCCGCTACATTAAAGGCACCAAAGGCAAG
ACCGATCTTATCTCTGTCGCCGTTGAGCCAACCGATTCTCCAGTT
ATCGCCCAGGCGCTGGCAGGTGAAGAGATTAAACCTGGCCCGCAT
AAAATTCAGGGTATTGGCGCTGGTTTTATCCCGGCTAACCTCGAT
CTCAAGCTGGTCGATAAAGTCATTGGCATCACCAATGAAGAAGCG
ATTTCTACCGCGCGTCGTCTGATGGAAGAAGAAGGTATTCTTGCA
GGTATCTCTTCTGGAGCAGCTGTTGCCGCGGCGTTGAAACTACAA
GAAGATGAAAGCTTTACCAACAAGAATATTGTGGTTATTCTACCA
TCATCGGGTGAGCGTTATTTAAGCACCGCATTGTTTGCCGATCTC
TTCACTGAGAAAGAATTGCAACAGTAA
<SEQ ID NO: 120; PRT;
CysK: Cysteine synthase A;
Escherichia coli>
MSKIFEDNSLTIGHTPLVRLNRIGNGRILAKVESRNPSFSVKCRI
GANMIWDAEKRGVLKPGVELVEPTSGNTGIALAYVAAARGYKLTL
TMPETMSIERRKLLKALGANLVLTEGAKGMKGAIQKAEEIVASNP
EKYLLLQQFSNPANPEIHEKTTGPEIWEDTDGQVDVFIAGVGTGG
TLTGVSRYIKGTKGKTDLISVAVEPTDSPVIAQALAGEEIKPGPH
KIQGIGAGFIPANLDLKLVDKVIGITNEEAISTARRLMEEEGILA
GISSGAAVAAALKLQEDESFTNKNIVVILPSSGERYLSTALFADL
FTEKELQQ
<SEQ ID NO: 121; DNA; cysA; Sulfate/
thiosulfate import ATP-binding protein;
Escherichia coli>
ATGAGCATTGAGATTGCCAATATTAAGAAGTCGTTTGGTCGCACC
CAGGTGCTGAACGATATCTCACTGGATATTCCTTCAGGTCAGATG
GTCGCGTTGCTGGGGCCGTCCGGTTCCGGGAAAACCACGCTGCTG
CGCATTATCGCCGGGCTGGAGCATCAAACCAGCGGGCATATTCGC
TTCCACGGCACCGACGTGAGCCGCCTGCACGCACGTGATCGTAAA
GTCGGTTTCGTGTTCCAGCATTACGCGCTGTTCCGCCATATGACG
GTGTTCGACAATATCGCTTTTGGCCTGACGGTGCTGCCGCGTCGC
GAGCGCCCGAATGCCGCAGCCATCAAAGCGAAAGTGACAAAATTG
CTGGAAATGGTCCAGCTTGCCCATCTGGCGGATCGTTATCCGGCG
CAGCTTTCCGGCGGCCAGAAACAGCGCGTGGCGCTGGCGCGCGCG
CTGGCTGTGGAACCGCAAATTCTGCTGCTTGATGAACCGTTTGGC
GCGCTGGATGCGCAGGTGCGTAAAGAGCTGCGTCGCTGGCTGCGT
CAACTCCATGAAGAACTAAAATTCACCAGCGTTTTTGTGACCCAC
GATCAGGAAGAAGCGACCGAAGTAGCTGATCGTGTAGTTGTGATG
AGCCAGGGCAATATTGAACAGGCTGACGCGCCGGATCAGGTATGG
CGCGAACCGGCGACCCGTTTTGTGCTCGAATTTATGGGCGAAGTG
AACCGCCTGCAGGGAACCATTCGCGGCGGGCAGTTCCATGTTGGC
GCGCATCGCTGGCCGCTGGGCTACACACCTGCGTATCAGGGGCCG
GTGGATCTCTTCCTGCGCCCTTGGGAAGTGGATATCAGCCGCCGT
ACCAGCCTCGATTCGCCGCTGCCGGTACAGGTACTGGAAGCCAGC
CCGAAAGGTCACTACACCCAATTAGTGGTGCAGCCGCTGGGGTGG
TACAACGAACCGCTGACGGTCGTGATGCATGGCGACGATGCCCCG
CAGCGTGGCGAGCGTTTATTCGTTGGTCTGCAACATGCGCGGCTG
TATAACGGCGACGAGCGTATCGAAACCCGCGATGAGGAACTTGCT
CTCGCACAAAGCGCCTGA
<SEQ ID NO: 122; PRT; CysA; Sulfate/
thiosulfate import ATP-binding protein;
Escherichia coli>
MSIEIANIKKSFGRTQVLNDISLDIPSGQMVALLGPSGSGKTTLL
RIIAGLEHQTSGHIRFHGTDVSRLHARDRKVGFVFQHYALFR
HMTVFDNIAFGLTVLPRRERPNAAAIKAKVTKLLEMVQLAHLADR
YPAQLSGGQKQRVALARALAVEPQILLLDEPFGALDAQVRKELRR
WLRQLHEELKFTSVFVTHDQEEATEVADRVVVMSQGNIEQADAPD
QVWREPATRFVLEFMGEVNRLQGTIRGGQFHVGAHRWPLGYTPAY
QGPVDLFLRPWEVDISRRTSLDSPLPVQVLEASPKGHYTQLVVQP
LGWYNEPLTVVMHGDDAPQRGERLFVGLQHARLYNGDERIETRDE
ELALAQSA
<SEQ ID NO: 123; DNA;
cysP; thio-sulfate binding protein;
Escherichia coli>
ATGGCCGTTAACTTACTGAAAAAGAACTCACTCGCGCTGGTCGCT
TCTCTGCTGCTGGCGGGCCATGTACAGGCAACGGAACTGCTGAAC
AGTTCTTATGACGTCTCCCGCGAGCTGTTTGCCGCCCTGAATCCG
CCGTTTGAGCAACAATGGGCAAAAGATAACGGCGGCGACAAACTG
ACGATAAAACAATCTCATGCCGGGTCATCAAAACAGGCGCTGGCG
ATTTTACAGGGCTTAAAAGCCGACGTTGTCACTTATAACCAGGTG
ACCGACGTACAAATCCTGCACGATAAAGGCAAGCTGATCCCGGCC
GACTGGCAGTCGCGCCTGCCGAATAATAGCTCGCCGTTCTACTCC
ACCATGGGCTTCCTGGTGCGTAAGGGTAACCCGAAGAATATCCAC
GATTGGAACGACCTGGTGCGCTCCGACGTGAAGCTGATTTTCCCG
AACCCGAAAACGTCGGGTAACGCGCGTTATACCTATCTGGCGGCA
TGGGGCGCAGCGGATAAAGCTGACGGTGGTGACAAAGGCAAAACC
GAACAGTTTATGACCCAGTTCCTGAAAAACGTTGAAGTGTTCGAT
ACTGGCGGTCGTGGCGCGACCACCACTTTTGCCGAGCGCGGCCTG
GGCGATGTGCTGATTAGCTTCGAATCGGAAGTGAACAACATCCGT
AAACAGTATGAAGCGCAGGGCTTTGAAGTGGTGATTCCGAAAACC
AACATTCTGGCGGAATTCCCGGTGGCGTGGGTTGATAAAAACGTG
CAGGCCAACGGTACGGAAAAAGCCGCCAAAGCCTATCTGAACTGG
CTCTATAGCCCGCAGGCGCAAACCATCATCACCGACTATTACTAC
CGCGTGAATAACCCGGAGGTGATGGACAAACTGAAAGACAAATTC
CCGCAGACCGAGCTGTTCCGCGTGGAAGACAAATTTGGCTCCTGG
CCGGAAGTGATGAAAACCCACTTCACCAGCGGCGGCGAGTTAGAC
AAGCTGTTAGCGGCGGGGCGTAACTGA
<SEQ ID NO: 124; PRT;
CysP; thio-sulfate binding protein A;
Escherichia coli>
MAVNLLKKNSLALVASLLLAGHVQATELLNSSYDVSRELFAALNP
PFEQQWAKDNGGDKLTIKQSHAGSSKQALAILQGLKADVVTYNQV
TDVQILHDKGKLIPADWQSRLPNNSSPFYSTMGFLVRKGNPKNIH
DWNDLVRSDVKLIFPNPKTSGNARYTYLAAWGAADKADGGDKGKT
EQFMTQFLKNVEVFDTGGRGATTTFAERGLGDVLISFESEVNNIR
KQYEAQGFEVVIPKTNILAEFPVAWVDKNVQANGTEKAAKAYLNW
LYSPQAQTIITDYYYRVNNPEVMDKLKDKFPQTELFRVEDKFGSW
PEVMKTHFTSGGELDKLLAAGRN
<SEQ ID NO: 125; DNA;
cysT; Sulfate transport system permease protein;
Escherichia coli>
ATGACGGAATCGTTGGTCGGCGAACGCCGCGCGCCGCAGTTCCGC
GCGCGCCTTTCCGGCCCCGCGGGCCCCCCTTCCGTTCGGGTCGGT
ATGGCAGTGGTGTGGCTTTCGGTGATCGTGCTGTTGCCGCTGGCC
GCCATCGTCTGGCAGGCCGCGGGCGGTGGTTGGCGGGCCTTCTGG
CTGGCGGTCTCGTCGCATGCCGCGATGGAGTCGTTCCGGGTAACG
CTGACGATTTCGACCGCAGTCACGGTCATCAACCTGGTGTTCGGC
TTGCTGATCGCCTGGGTGCTGGTGCGTGACGACTTCGCTGGCAAG
CGGATCGTCGATGCGATTATCGATCTGCCGTTTGCGTTGCCCACC
ATCGTCGCCAGCCTGGTGATGTTGGCACTGTACGGGAACAACAGC
CCGGTGGGGCTTCATTTTCAACACACCGCGACCGGTGTTGGGGTG
GCGTTGGCGTTCGTCACATTGCCGTTCGTGGTGCGCGCCGTGCAG
CCGGTGCTGCTGGAAATCGATCGCGAGACCGAGGAGGCGGCGGCG
TCGCTGGGCGCTAATGGTGCCAAAATCTTCACTTCGGTGGTGTTG
CCGTCGCTGACGCCGGCATTGTTATCCGGTGCGGGCCTGGCGTTT
TCGCGCGCTATCGGCGAGTTCGGTTCGGTGGTTCTGATCGGCGGG
GCCGTGCCGGGCAAGACCGAGGTGTCCTCGCAATGGATTCGCACC
CTGATCGAGAACGACGACCGCACCGGAGCGGCCGCGATATCGGTT
GTATTGCTCTCGATTTCGTTCATTGTGCTGCTCATCCTACGTGTC
GTCGGCGCGCGTGCGGCCAAACGTGAGGAGATGGCCGCATGA
<SEQ ID NO: 126; PRT;
cysT; Sulfate transport system permease protein;
Escherichia coli>
MTESLVGERRAPQFRARLSGPAGPPSVRVGMAVVWLSVIVLLPLA
AIVWQAAGGGWRAFWLAVSSHAAMESFRVTLTISTAVTVINLVFG
LLIAWVLVRDDFAGKRIVDAIIDLPFALPTIVASLVMLALYGNNS
PVGLHFQHTATGVGVALAFVTLPFVVRAVQPVLLEIDRETEEAAA
SLGANGAKIFTSVVLPSLTPALLSGAGLAFSRAIGEFGSVVLIGG
AVPGKTEVSSQWIRTLIENDDRTGAAAISVVLLSISFIVLLILRV
VGARAAKREEMAA
<SEQ ID NO: 127; DNA;
cysW; Sulfate transport system permease protein;
Escherichia coli>
ATGGCGGAAGTTACCCAATTGAAGCGTTATGACGCGCGCCCGATT
AACTGGGGCAAATGGTTTCTGATTGGCATCGGGATGCTGGTTTCG
GCGTTCATCCTGCTGGTGCCGATGATTTACATCTTCGTGCAGGCA
TTCAGCAAGGGGCTGATGCCGGTTTTACAGAATCTGGCCGATCCG
GACATGCTGCACGCCATCTGGCTGACGGTGATGATCGCGCTGATT
GCCGTACCGGTAAACCTGGTGTTCGGCATTCTGCTGGCCTGGCTG
GTGACGCGCTTTAACTTCCCTGGACGCCAGTTACTGCTGACGCTA
CTGGACATTCCGTTTGCCGTATCGCCGGTGGTTGCCGGTCTGGTG
TATTTGCTGTTCTACGGCTCTAACGGCCCGCTCGGCGGTTGGCTC
GACGAGCATAACCTGCAAATTATGTTCTCCTGGCCGGGAATGGTG
CTGGTCACCATCTTCGTGACGTGTCCGTTTGTGGTGCGCGAACTG
GTGCCGGTGATGTTAAGCCAGGGCAGCCAGGAAGACGAAGCGGCG
ATTTTGCTTGGCGCGTCCGGCTGGCAGATGTTCCGTCGCGTCACA
TTACCGAACATCCGCTGGGCGCTGCTTTATGGCGTGGTGTTGACC
AACGCCCGCGCAATTGGCGAGTTTGGCGCGGTGTCGGTGGTTTCC
GGCTCGATTCGCGGCGAAACCCTGTCGCTGCCGTTACAGATTGAA
TTGCTGGAGCAGGACTACAACACCGTCGGCTCCTTTACCGCTGCG
GCGCTGTTAACGCTGATGGCGATTATCACCCTGTTTTTAAAAAGT
ATGTTGCAGTGGCGCCTGGAGAATCAGGAAAAACGCGCACAGCAG
GAGGAACATCATGAGCATTGA
<SEQ ID NO: 128; PRT;
CysW; Sulfate transport system permease protein;
Escherichia coli>
MAEVTQLKRYDARPINWGKWFLIGIGMLVSAFILLVPMIYIFVQA
FSKGLMPVLQNLADPDMLHAIWLTVMIALIAVPVNLVFGILLAWL
VTRFNFPGRQLLLTLLDIPFAVSPVVAGLVYLLFYGSNGPLGGWL
DEHNLQIMFSWPGMVLVTIFVTCPFVVRELVPVMLSQGSQEDEAA
ILLGASGWQMFRRVTLPNIRWALLYGVVLTNARAIGEFGAVSVVS
GSIRGETLSLPLQIELLEQDYNTVGSFTAAALLTLMAIITLFLKS
MLQWRLENQEKRAQQEEHHEH
<SEQ ID NO: 129; DNA; egtB;
Mycobacterium smegmatis>
ATGATCGCAC GCGAGACACT GGCCGACGAG CTGGCCCTGG
CCCGCGAACG CACGTTGCGG CTCGTGGAGT TCGACGACGC
GGAACTGCAT CGCCAGTACA ACCCGCTGAT GAGCCCGCTC
GTGTGGGACC TCGCGCACAT CGGGCAGCAG GAAGAACTGT
GGCTGCTGCG CGACGGCAAC CCCGACCGCC CCGGCATGCT
CGCACCCGAG GTGGACCGGC TTTACGACGC GTTCGAGCAC
TCACGCGCCA GCCGGGTCAA CCTCCCGTTG CTGCCGCCTT
CGGATGCGCG CGCCTACTGC GCGACGGTGC GGGCCAAGGC
GCTCGACACC CTCGACACGC TGCCCGAGGA CGATCCGGGC
TTCCGGTTCG CGCTGGTGAT CAGCCACGAG AACCAGCACG
ACGAGACCAT GCTGCAGGCA CTCAACCTGC GCGAGGGCCC
ACCCCTGCTC GACACCGGAA TTCCCCTGCC CGCGGGCAGG
CCAGGCGTGG CAGGCACGTC GGTGCTGGTG CCGGGCGGCC
CGTTCGTGCT CGGGGTCGAC GCGCTGACCG AACCGCACTC
ACTGGACAAC GAACGGCCCG CCCACGTCGT GGACATCCCG
TCGTTCCGGA TCGGCCGCGT GCCGGTCACC AACGCCGAAT
GGCGCGAGTT CATCGACGAC GGTGGCTACG ACCAACCGCG
CTGGTGGTCG CCACGCGGCT GGGCGCACCG CCAGGAGGCG
GGCCTGGTGG CCCCGCAGTT CTGGAACCCC GACGGCACCC
GCACCCGGTT CGGGCACATC GAGGAGATCC CGGGTGACGA
ACCCGTGCAG CACGTGACGT TCTTCGAAGC CGAGGCCTAC
GCGGCGTGGG CCGGTGCTCG GTTGCCCACC GAGATCGAAT
GGGAGAAGGC CTGCGCGTGG GATCCGGTCG CCGGTGCTCG
GCGCCGGTTC CCCTGGGGCT CAGCACAACC CAGCGCGGCG
CTGGCCAACC TCGGCGGTGA CGCACGCCGC CCGGCGCCGG
TCGGGGCCTA CCCGGCGGGG GCGTCGGCCT ATGGCGCCGA
GCAGATGCTG GGCGACGTGT GGGAGTGGAC CTCCTCGCCG
CTGCGGCCGT GGCCCGGTTT CACGCCGATG ATCTACGAGC
GCTACAGCAC GCCGTTCTTC GAGGGCACCA CATCCGGTGA
CTACCGCGTG CTGCGCGGCG GGTCATGGGC CGTTGCACCG
GGAATCCTGC GGCCCAGCTT CCGCAACTGG GACCACCCGA
TCCGGCGGCA GATATTCTCG GGTGTCCGCC TGGCCTGGGA
CGTCTGA
<SEQ ID NO: 130; PRT; EgtB;
Mycobacterium smegmatis>
Met Ile Ala Arg Glu Thr Leu Ala Asp Glu Leu
Ala Leu Ala Arg Glu Arg Thr Leu Arg Leu Val
Glu Phe Asp Asp Ala Glu Leu His Arg Gln Tyr
Asn Pro Leu Met Ser Pro Leu Val Trp Asp Leu
Ala His Ile Gly Gln Gln Glu Glu Leu Trp Leu
Leu Arg Asp Gly Asn Pro Asp Arg Pro Gly Met
Leu Ala Pro Glu Val Asp Arg Leu Tyr Asp Ala
Phe Glu His Ser Arg Ala Ser Arg Val Asn Leu
Pro Leu Leu Pro Pro Ser Asp Ala Arg Ala Tyr
Cys Ala Thr Val Arg Ala Lys Ala Leu Asp Thr
Leu Asp Thr Leu Pro Glu Asp Asp Pro Gly Phe
Arg Phe Ala Leu Val Ile Ser His Glu Asn Gln
His Asp Glu Thr Met Leu Gln Ala Leu Asn Leu
Arg Glu Gly Pro Pro Leu Leu Asp Thr Gly Ile
Pro Leu Pro Ala Gly Arg Pro Gly Val Ala Gly
Thr Ser Val Leu Val Pro Gly Gly Pro Phe Val
Leu Gly Val Asp Ala Leu Thr Glu Pro His Ser
Leu Asp Asn Glu Arg Pro Ala His Val Val Asp
Ile Pro Ser Phe Arg Ile Gly Arg Val Pro Val
Thr Asn Ala Glu Trp Arg Glu Phe Ile Asp Asp
Gly Gly Tyr Asp Gln Pro Arg Trp Trp Ser Pro
Arg Gly Trp Ala His Arg Gln Glu Ala Gly Leu
Val Ala Pro Gln Phe Trp Asn Pro Asp Gly Thr
Arg Thr Arg Phe Gly His Ile Glu Glu Ile Pro
Gly Asp Glu Pro Val Gln His Val Thr Phe Phe
Glu Ala Glu Ala Tyr Ala Ala Trp Ala Gly Ala
Arg Leu Pro Thr Glu Ile Glu Trp Glu Lys Ala
Cys Ala Trp Asp Pro Val Ala Gly Ala Arg Arg
Arg Phe Pro Trp Gly Ser Ala Gln Pro Ser Ala
Ala Leu Ala Asn Leu Gly Gly Asp Ala Arg Arg
Pro Ala Pro Val Gly Ala Tyr Pro Ala Gly Ala
Ser Ala Tyr Gly Ala Glu Gln Met Leu Gly Asp
Val Trp Glu Trp Thr Ser Ser Pro Leu Arg Pro
Trp Pro Gly Phe Thr Pro Met Ile Tyr Glu Arg
Tyr Ser Thr Pro Phe Phe Glu Gly Thr Thr Ser
Gly Asp Tyr Arg Val Leu Arg Gly Gly Ser Trp
Ala Val Ala Pro Gly Ile Leu Arg Pro Ser Phe
Arg Asn Trp Asp His Pro Ile Arg Arg Gln Ile
Phe Ser Gly Val Arg Leu Ala Trp Asp Val
<SEQ ID NO: 131; DNA; egtC;
Mycobacterium smegmatis>
ATGTGCCGGC ATGTGGCGTG GCTGGGCGCG CCGCGGTCGT
TGGCCGACCT GGTGCTCGAC CCGCCGCAGG GACTGCTGGT
GCAGTCCTAC GCACCGCGAC GACAGAAGCA CGGTCTGATG
AACGCCGACG GTTGGGGCGC AGGGTTTTTC GACGACGAGG
GAGTGGCCCG CCGCTGGCGC AGCGACAAAC CGCTGTGGGG
TGATGCGTCG TTCGCGTCGG TGGCACCCGC ACTACGCAGT
CGTTGCGTGC TGGCCGCGGT GCGCTCGGCC ACCATCGGCA
TGCCCATCGA ACCGTCGGCG TCGGCGCCGT TCAGCGACGG
GCAGTGGCTG CTGTCGCACA ACGGCCTGGT CGACCGCGGG
GTGCTCCCGT TGACCGGTGC CGCCGAGTCC ACGGTGGACA
GCGCGATCGT CGCGGCGCTC ATCTTCTCCC GTGGCCTCGA
CGCGCTCGGC GCCACCATCG CCGAGGTCGG CGAACTCGAC
CCGAACGCGC GGTTGAACAT CCTGGCCGCC AACGGTTCCC
GGCTGCTCGC CACCACCTGG GGGGACACGC TGTCGGTCCT
GCACCGCCCC GACGGCGTCG TCCTCGCGAG CGAACCCTAC
GACGACGATC CCGGCTGGTC CACGTCGTCG GGACATCCCG
TCGTCGACGT CCGCGACGCC GACCGGCACC TGACACCCCT
GTGA
<SEQ ID NO: 132; PRT; egtC;
Mycobacterium smegmatis>
Met Cys Arg His Val Ala Trp Leu Gly Ala Pro
Arg Ser Leu Ala Asp Leu Val Leu Asp Pro Pro
Gln Gly Leu Leu Val Gln Ser Tyr Ala Pro Arg
Arg Gln Lys His Gly Leu Met Asn Ala Asp Gly
Trp Gly Ala Gly Phe Phe Asp Asp Glu Gly Val
Ala Arg Arg Trp Arg Ser Asp Lys Pro Leu Trp
Gly Asp Ala Ser Phe Ala Ser Val Ala Pro Ala
Leu Arg Ser Arg Cys Val Leu Ala Ala Val Arg
Ser Ala Thr Ile Gly Met Pro Ile Glu Pro Ser
Ala Ser Ala Pro Phe Ser Asp Gly Gln Trp Leu
Leu Ser His Asn Gly Leu Val Asp Arg Gly Val
Leu Pro Leu Thr Gly Ala Ala Glu Ser Thr Val
Asp Ser Ala Ile Val Ala Ala Leu Ile Phe Ser
Arg Gly Leu Asp Ala Leu Gly Ala Thr Ile Ala
Glu Val Gly Glu Leu Asp Pro Asn Ala Arg Leu
Asn Ile Leu Ala Ala Asn Gly Ser Arg Leu Leu
Ala Thr Thr Trp Gly Asp Thr Leu Ser Val Leu
His Arg Pro Asp Gly Val Val Leu Ala Ser Glu
Pro Tyr Asp Asp Asp Pro Gly Trp Ser Asp Ile
Pro Asp Arg His Leu Val Asp Val Arg Asp Ala
His Val Val Val Thr Pro Leu
<SEQ ID NO: 133; DNA; egtD;
Mycobacterium smegmatis>
ATGACGCTCT CACTGGCCAA CTACCTGGCA GCCGACTCGG
CCGCCGAAGC ACTGCGCCGT GACGTCCGCG CGGGCCTCAC
CGCGGCACCG AAGAGTCTGC CGCCCAAGTG GTTCTACGAC
GCCGTCGGCA GTGATCTGTT CGACCAGATC ACCCGGCTCC
CCGAGTATTA CCCCACCCGC ACCGAGGCGC AGATCCTGCG
GACCCGGTCG GCGGAGATCA TCGCGGCCGC GGGTGCCGAC
ACCCTGGTGG AACTGGGCAG TGGTACGTCG GAGAAAACCC
GCATGCTGCT CGACGCCATG CGCGACGCCG AGTTGCTGCG
CCGCTTCATC CCGTTCGACG TCGACGCGGG CGTGCTGCGC
TCGGCCGGGG CGGCAATCGG CGCGGAGTAC CCCGGTATCG
AGATCGACGC GGTATGTGGC GATTTCGAGG AACATCTGGG
CAAGATCCCG CATGTCGGAC GGCGGCTCGT GGTGTTCCTG
GGGTCGACCA TCGGCAACCT GACACCCGCG CCCCGCGCGG
AGTTCCTCAG TACTCTCGCG GACACGCTGC AGCCGGGCGA
CAGCCTGCTG CTGGGCACCG ATCTGGTGAA GGACACCGGC
CGGTTGGTGC GCGCGTACGA CGACGCGGCC GGCGTCACCG
CGGCGTTCAA CCGCAACGTG CTGGCCGTGG TGAACCGCGA
ACTGTCCGCC GATTTCGACC TCGACGCGTT CGAGCATGTC
GCGAAGTGGA ACTCCGACGA GGAACGCATC GAGATGTGGT
TGCGTGCCCG CACCGCACAG CATGTCCGCG TCGCGGCACT
GGACCTGGAG GTCGACTTCG CCGCGGGTGA GGAGATGCTC
ACCGAGGTGT CCTGCAAGTT CCGTCCCGAG AACGTCGTCG
CCGAGCTGGC GGAAGCCGGT CTGCGGCAGA CGCATTGGTG
GACCGATCCG GCCGGGGATT TCGGGTTGTC GCTGGCGGTG
CGGTGA
<SEQ ID NO: 134; PRT; EgtD;
Mycobacterium smegmatis>
Met Thr Leu Ser Leu Ala Asn Tyr Leu Ala Ala
Asp Ser Ala Ala Glu Ala Leu Arg Arg Asp Val
Arg Ala Gly Leu Thr Ala Ala Pro Lys Ser Leu
Pro Pro Lys Trp Phe Tyr Asp Ala Val Gly Ser
Asp Leu Phe Asp Gln Ile Thr Arg Leu Pro Glu
Tyr Tyr Pro Thr Arg Thr Glu Ala Gln Ile Leu
Arg Thr Arg Ser Ala Glu Ile Ile Ala Ala Ala
Gly Ala Asp Thr Leu Val Glu Leu Gly Ser Gly
Thr Ser Glu Lys Thr Arg Met Leu Leu Asp Ala
Met Arg Asp Ala Glu Leu Leu Arg Arg Phe Ile
Pro Phe Asp Val Asp Ala Gly Val Leu Arg Ser
Ala Gly Ala Ala Ile Gly Ala Glu Tyr Pro Gly
Ile Glu Ile Asp Ala Val Cys Gly Asp Phe Glu
Glu His Leu Gly Lys Ile Pro His Val Gly Arg
Arg Leu Val Val Phe Leu Gly Ser Thr Ile Gly
Asn Leu Thr Pro Ala Pro Arg Ala Glu Phe Leu
Ser Thr Leu Ala Asp Thr Leu Gln Pro Gly Asp
Ser Leu Leu Leu Gly Thr Asp Leu Val Lys Asp
Thr Gly Arg Leu Val Arg Ala Tyr Asp Asp Ala
Ala Gly Val Thr Ala Ala Phe Asn Arg Asn Val
Leu Ala Val Val Asn Arg Glu Leu Ser Ala Asp
Phe Asp Leu Asp Ala Phe Glu His Val Ala Lys
Trp Asn Ser Asp Glu Glu Arg Ile Glu Met Trp
Leu Arg Ala Arg Thr Ala Gln His Val Arg Val
Ala Ala Leu Asp Leu Glu Val Asp Phe Ala Ala
Gly Glu Glu Met Leu Thr Glu Val Ser Cys Lys
Phe Arg Pro Glu Asn Val Val Ala Glu Leu Ala
Glu Ala Gly Leu Arg Gln Thr His Trp Trp Thr
Asp Pro Ala Gly Asp Phe Gly Leu Ser Leu Ala
Val Arg
<SEQ ID NO: 135; DNA; egtE;
Mycobacterium smegmatis>
ATGCTCGCGC AGCAGTGGCG TGACGCCCGT CCCAAGGTTG
CCGGGTTGCA CCTGGACAGC GGGGCATGTT CGCGGCAGAG
CTTCGCGGTG ATCGACGCGA CCACCGCACA CGCACGCCAC
GAGGCCGAGG TGGGTGGTTA TGTGGCGGCC GAGGCTGCGA
CGCCGGCGCT CGACGCCGGG CGGGCCGCGG TCGCGTCGCT
CATCGGTTTT GCGGCGTCGG ACGTGGTGTA CACCAGCGGA
TCCAACCACG CCATCGACCT GTTGCTGTCG AGCTGGCCGG
GGAAGCGCAC GCTGGCCTGC CTGCCCGGCG AGTACGGGCC
GAATCTGTCT GCCATGGCGG CCAACGGTTT CCAGGTGCGT
GCGCTACCGG TCGACGACGA CGGGCGGGTG CTGGTCGACG
AGGCGTCGCA CGAACTGTCG GCCCATCCCG TCGCGCTCGT
ACACCTCACC GCATTGGCAA GCCATCGCGG GATCGCGCAA
CCCGCGGCAG AACTCGTCGA GGCCTGCCAC AATGCGGGGA
TCCCCGTGGT GATCGACGCC GCGCAGGCGC TGGGGCATCT
GGACTGCAAT GTCGGGGCCG ACGCGGTGTA CTCATCGTCG
CGCAAGTGGC TCGCCGGCCC GCGTGGTGTC GGGGTGCTCG
CGGTGCGGCC CGAACTCGCC GAGCGTCTGC AACCGCGGAT
CCCCCCGTCC GACTGGCCAA TTCCGATGAG CGTCTTGGAG
AAGCTCGAAC TAGGTGAGCA CAACGCGGCG GCGCGTGTGG
GATTCTCCGT CGCGGTTGGT GAGCATCTCG CAGCAGGGCC
CACGGCGGTG CGCGAACGAC TCGCCGAGGT GGGGCGTCTC
TCTCGGCAGG TGCTGGCAGA GGTCGACGGG TGGCGCGTCG
TCGAACCCGT CGACCAACCC ACCGCGATCA CCACCCTTGA
GTCCACCGAT GGTGCCGATC CCGCGTCGGT GCGCTCGTGG
CTGATCGCGG AGCGTGGCAT CGTGACCACC GCGTGTGAAC
TCGCGCGGGC ACCGTTCGAG ATGCGCACGC CGGTGCTGCG
AATCTCGCCG CACGTCGACG TGACGGTCGA CGAACTGGAG
CAGTTCGCCG CAGCGTTGCG TGAGGCGCCC TGA
<SEQ ID NO: 136; PRT; EgtE;
Mycobacterium smegmatis>
Met Leu Ala Gln Gln Trp Arg Asp Ala Arg Pro
Lys Val Ala Gly Leu His Leu Asp Ser Gly Ala
Cys Ser Arg Gln Ser Phe Ala Val Ile Asp Ala
Thr Thr Ala His Ala Arg His Glu Ala Glu Val
Gly Gly Tyr Val Ala Ala Glu Ala Ala Thr Pro
Ala Leu Asp Ala Gly Arg Ala Ala Val Ala Ser
Leu Ile Gly Phe Ala Ala Ser Asp Val Val Tyr
Thr Ser Gly Ser Asn His Ala Ile Asp Leu Leu
Leu Ser Ser Trp Pro Gly Lys Arg Thr Leu Ala
Cys Leu Pro Gly Glu Tyr Gly Pro Asn Leu Ser
Ala Met Ala Ala Asn Gly Phe Gln Val Arg Ala
Leu Pro Val Asp Asp Asp Gly Arg Val Leu Val
Asp Glu Ala Ser His Glu Leu Ser Ala His Pro
Val Ala Leu Val His Leu Thr Ala Leu Ala Ser
His Arg Gly Ile Ala Gln Pro Ala Ala Glu Leu
Val Glu Ala Cys His Asn Ala Gly Ile Pro Val
Val Ile Asp Ala Ala Gln Ala Leu Gly His Leu
Asp Cys Asn Val Gly Ala Asp Ala Val Tyr Ser
Ser Ser Arg Lys Trp Leu Ala Gly Pro Arg Gly
Val Gly Val Leu Ala Val Arg Pro Glu Leu Ala
Glu Arg Leu Gln Pro Arg Ile Pro Pro Ser Asp
Trp Pro Ile Pro Met Ser Val Leu Glu Lys Leu
Glu Leu Gly Glu His Asn Ala Ala Ala Arg Val
Gly Phe Ser Val Ala Val Gly Glu His Leu Ala
Ala Gly Pro Thr Ala Val Arg Glu Arg Leu Ala
Glu Val Gly Arg Leu Ser Arg Gln Val Leu Ala
Glu Val Asp Gly Trp Arg Val Val Glu Pro Val
Asp Gln Pro Thr Ala Ile Thr Thr Leu Glu Ser
Thr Asp Gly Ala Asp Pro Ala Ser Val Arg Ser
Trp Leu Ile Ala Glu Arg Gly Ile Val Thr Thr
Ala Cys Glu Leu Ala Arg Ala Pro Phe Glu Met
Arg Thr Pro Val Leu Arg Ile Ser Pro His Val
Asp Val Thr Val Asp Glu Leu Glu GIn Phe Ala
Ala Ala Leu Arg Glu Ala Pro
<SEQ ID NO: 137; DNA; NcEgt1;
Neurospora crassa>
ATGCCGAGTGCCGAATCCATGACCCCAAGCAGTGCCCTCGGACAG
CTCAAAGCAACTGGACAACATGTGCTATCCAAGCTTCAGCAGCAG
ACATCAAACGCCGATATCATCGACATCCGCCGCGTTGCTGTAGAG
ATCAACCTCAAGACCGAGATAACCTCCATGTTCCGACCTAAAGAT
GGCCCTAGACAGCTACCCACCTTGCTTCTCTACAACGAGAGAGGC
CTGCAGCTGTTCGAGCGTATCACATACCTTGAAGAGTACTATCTT
ACCAATGACGAGATCAAAATCCTCACCAAACATGCGACCGAAATG
GCTAGCTTCATCCCGTCAGGTGCCATGATCATTGAGCTCGGAAGC
GGAAATCTGCGCAAAGTAAACCTTCTATTGGAAGCCCTAGACAAC
GCCGGCAAGGCAATTGACTATTATGCCCTTGACCTGTCTCGGGAG
GAGCTGGAGCGCACTCTCGCTCAGGTACCATCCTACAAGCACGTC
AAGTGCCACGGTCTTCTGGGTACATATGACGATGGACGTGACTGG
CTCAAGGCCCCAGAGAACATCAATAAACAGAAATGCATCTTGCAC
CTCGGGTCAAGCATTGGCAACTTTAACCGCAGTGACGCCGCTACC
TTTCTCAAGGGCTTTACGGACGTCCTTGGACCCAATGACAAGATG
CTCATTGGGGTTGACGCTTGCAATGACCCGGCGAGGGTATACCAC
GCTTACAACGACAAGGTTGGTATTACTCACGAGTTCATCTTGAAT
GGTCTTCGCAACGCCAATGAAATTATCGGAGAGACGGCCTTCATC
GAGGGCGATTGGAGAGTCATTGGCGAATATGTGTATGACGAAGAG
GGCGGCAGACACCAGGCCTTTTACGCCCCCACTCGCGACACCATG
GTTATGGGGGAGTTGATTAGGTCACACGACAGGATCCAGATCGAA
CAGAGCCTAAAGTACTCGAAAGAGGAGTCAGAGAGGCTCTGGAGC
ACGGCGGGATTGGAACAAGTCTCGGAATGGACGTACGGCAACGAA
TATGGACTCCATCTGCTTGCCAAGTCAAGGATGTCTTTCAGTCTC
ATCCCTTCGGTGTACGCTCGCAGCGCACTCCCAACTCTGGACGAC
TGGGAGGCCCTTTGGGCGACATGGGATGTCGTCACACGTCAGATG
CTTCCCCAGGAAGAGCTTCTGGAGAAGCCCATCAAGCTCCGAAAC
GCCTGCATCTTTTACCTCGGTCACATCCCGACCTTCCTCGACATC
CAGCTCACAAAGACCACCAAGCAGGCTCCGTCAGAGCCCGCTCAC
TTTTGCAAGATCTTCGAGCGAGGCATTGATCCTGATGTCGACAAC
CCGGAGCTGTGTCATGCGCACTCGGAGATTCCTGATGAATGGCCG
CCGGTGGAAGAAATCCTGACCTACCAGGAGACGGTACGGTCCCGG
TTACGCGGCCTCTATGCGCATGGCATCGCGAATATTCCGCGGAAT
GTGGGTCGGGCCATTTGGGTTGGGTTTGAGCACGAGCTTATGCAT
ATCGAGACGCTGTTGTACATGATGCTACAGAGCGACAAGACGCTG
ATCCCAACCCATATTCCACGGCCCGACTTTGACAAGCTCGCGAGG
AAGGCAGAGTCCGAGAGGGTTCCCAATCAGTGGTTTAAGATTCCG
GCACAGGAGATCACCATCGGTTTGGATGATCCTGAGGATGGATCT
GATATCAACAAGCATTATGGCTGGGACAACGAGAAGCCTCCAAGG
CGCGTTCAAGTTGCTGCCTTTCAGGCTCAAGGGAGGCCGATCACC
AACGAAGAGTACGCGCAATATCTGCTTGAAAAGAACATCGACAAG
CTCCCTGCCTCTTGGGCCCGCCTGGACAACGAGAACATTAGCAAT
GGAACAACAAACAGCGTGAGCGGTCACCACAGCAACAGAACCTCC
AAGCAGCAGCTCCCTTCATCTTTCCTCGAGAAGACAGCAGTCCGC
ACAGTCTACGGTCTCGTGCCTCTCAAGCACGCTCTCGACTGGCCC
GTGTTTGCCTCTTACGACGAACTTGCCGGTTGCGCAGCTTACATG
GGCGGCCGTATTCCCACCTTCGAAGAGACCCGGAGCATTTACGCT
TACGCCGATGCTCTCAAGAAGAAGAAGGAAGCTGAGAGACAATTG
GGAAGGACGGTTCCGGCTGTTAATGCCCACCTAACCAACAACGGC
GTGGAAATCACTCCCCCATCCTCTCCCTCTTCCGAGACCCCCGCC
GAGTCTTCCTCCCCCTCCGACAGCAACACCACCCTCATCACCACC
GAAGACCTCTTCTCTGACCTAGACGGTGCCAATGTCGGTTTTCAC
AACTGGCACCCTATGCCCATCACCTCCAAAGGCAACACCCTTGTC
GGGCAAGGCGAGCTCGGCGGCGTGTGGGAATGGACTTCATCGGTC
CTCCGCAAGTGGGAGGGGTTCGAGCCGATGGAGCTGTACCCCGGC
TATACGGCGGATTTTTTCGATGAGAAGCACAACATTGTGCTGGGA
GGGAGCTGGGCTACGCATCCGAGGATTGCGGGGAGGAAGAGCTTT
GTGAATTGGTACCAGAGGAATTATCCTTATGCTTGGGTGGGGGCG
AGAGTTGTTAGGGATTTGTGA
<SEQ ID NO: 138; PRT; NcEgt1;
Neurospora crassa>
MPSAESMTPSSALGQLKATGQHVLSKLQQQTSNADIIDIRRVAVE
INLKTEITSMFRPKDGPRQLPTLLLYNERGLQLFERITYLEEYYL
TNDEIKILTKHATEMASFIPSGAMIIELGSGNLRKVNLLLEALDN
AGKAIDYYALDLSREELERTLAQVPSYKHVKCHGLLGTYDDGRDW
LKAPENINKQKCILHLGSSIGNFNRSDAATFLKGFTDVLGPNDKM
LIGVDACNDPARVYHAYNDKVGITHEFILNGLRNANEIIGETAFI
EGDWRVIGEYVYDEEGGRHQAFYAPTRDTMVMGELIRSHDRIQIE
QSLKYSKEESERLWSTAGLEQVSEWTYGNEYGLHLLAKSRMSFSL
IPSVYARSALPTLDDWEALWATWDVVTRQMLPQEELLEKPIKLRN
ACIFYLGHIPTFLDIQLTKTTKQAPSEPAHFCKIFERGIDPDVDN
PELCHAHSEIPDEWPPVEEILTYQETVRSRLRGLYAHGIANIPRN
VGRAIWVGFEHELMHIETLLYMMLQSDKTLIPTHIPRPDFDKLAR
KAESERVPNQWFKIPAQEITIGLDDPEDGSDINKHYGWDNEKPPR
RVQVAAFQAQGRPITNEEYAQYLLEKNIDKLPASWARLDNENISN
GTTNSVSGHHSNRTSKQQLPSSFLEKTAVRTVYGLVPLKHALDWP
VFASYDELAGCAAYMGGRIPTFEETRSIYAYADALKKKKEAERQL
GRTVPAVNAHLTNNGVEITPPSSPSSETPAESSSPSDSNTTLITT
EDLESDLDGANVGFHNWHPMPITSKGNTLVGQGELGGVWEWTSSV
LRKWEGFEPMELYPGYTADFFDEKHNIVLGGSWATHPRIAGRKSF
VNWYQRNYPYAWVGARVVRDL
<SEQ ID NO: 139; DNA; MzEan3;
Methanosalsum zhilinae>
ATGATCATACAGAATTTTATGCCTGAGATTGGAGAACGTTCAGTT
CAAGAAAGACTTTTAACTTGTTTAAGGTCTGAGCCAAAGACATTA
CCATCTGTGTTCTTCTATGACCAGAAAGGTTCGGAACTGTTCGAA
CAAATAACAAAACTTGAAGAATATTATTTACCTGACATTGAGATC
CCACTTTTAAGATCGACTGCTAAAAAAGTTAATTCTGAACTGAAG
AACTGTAACCTTGTAGAACTCGGGAGTGGTGACTGTTCTAAGATT
TCAGTGTTCCTTGATGCAGTTCCAAAAGACATTCGTGAAACCATC
ATTTATTATCCAATAGATGTTTCCAAGGATGCTATGGAAAAATCT
GGTCATATTCTACAGAACAGATTCCCTGAGATAGGTATTCATGGA
ATTAATGCCGATTTCCTTGAAAGTATGGATTTAATACCTGGAGAT
AGGAACAGGTTCTTCTGTTTTTTCGGGAGTACAATAGGTAATCTT
ACTCGTGCTAAGACTACTGAATTCATGAAACGGCTTGGACAAGTC
ATGAATGAAAATGATAGGCTTCTTCTCGGAGTTGATATGGTGAAA
GATATCAATGTACTTGAGAGAGCATATAATGATAGTCTGGGTATT
ACTGCGGAGTTTAATAAGAATATTTTAAAGGTCGCAAACAATCAT
ATAGGAACTGACTTTGATCCAGATGATTTTGAACATGTTGCTTTT
TTCAACAAAGAATTTTCACGAATCGAGATGCACTTGAAAGCAAAA
AGGGATCTAGTAGTTAAAAGTGATTTGTTTAAGGAACGTATTATC
TTCAAGAAAGGGGATACCATTCATACAGAAAATTCACATAAGTAT
ACTGTACAGCACATCTATGATATGGCAGATACTGCTGGTCTTTTT
GTATCTGATATTTATTCTGATGATAAAAAATGGTTCTCACTTGTT
GAAATGGTGAAAGAATG
<SEQ ID NO: 140; PRT; MzEanA3;
Methanosalsum zhilinae>
MIIQNFMPEIGERSVQERLLTCLRSEPKTLPSVFFYDQKGSELFE
QITKLEEYYLPDIEIPLLRSTAKKVNSELKNCNLVELGSGDCSKI
SVFLDAVPKDIRETIIYYPIDVSKDAMEKSGHILQNRFPEIGIHG
INADFLESMDLIPGDRNRFFCFFGSTIGNLTRAKTTEFMKRLGQV
MNENDRLLLGVDMVKDINVLERAYNDSLGITAEFNKNILKVANNH
IGTDFDPDDFEHVAFFNKEFSRIEMHLKAKRDLVVKSDLFKERII
FKKGDTIHTENSHKYTVQHIYDMADTAGLFVSDIYSDDKKWFSLV
EMVKE
<SEQ ID NO: 141; DNA; MzEanB3;
Methanosalsum zhilinae>
ATGAAATCCATTTCAACTGATGAATTATTAGAAAACTTGCATAGA
TACAAAGTCATTGATATTAGATCTGTAGATGCTTATAATGGATGG
AAGGAGAATGGGGAAAACAGAGGTGGGCATATAAGAAGTGCAAAA
TCACTACCTTACAAGTGGGTAAACTACATTGACTGGATCGAGATC
GTTAGGAGCAAGGATATTCTCCCACAAGACAAACTTGTAATTTAC
GGTTATGACTCAGAGAAAGCAGAAGAGGTTGCCAGAATGTTTGAA
AAGGCTGGTTATACTGACCTGAACATATACCCTTCTTTTTTCGAG
TGGGTAGAAAGGAATCTGCCAATGGACCGACTTGAGAGGTACCGA
CACTTAGTATCTCCTGATTGGCTGAACCAATTGATAACTACCGAC
AATGCACCTGAATATGATAATGATAAGTATGTCATATGCCATTGC
CATTACAGAAATCCAGTGGATTATGAAAAAGGTCATATTCCAGGC
TCGATCCCACTTGATACCAATTCACTCGAATCCGAGGATACATGG
AACCGTCGTTCACCAGAAGAACTAAAAGATGCACTTGAAAATGCA
GGTATTTCCAGTGAAACAACAGTTATTGTATATGGAAGGTTCTCC
TACCCAAAGAACGATGACCCATTTCCAGGCAGTAGCGCGGGTCAC
CTTGGTGCAATGCGATGTGCATTCATAATGCTTTATGCTGGAGTC
AAGGATGTAAGGATCCTTAATGGTGGACTCCAGTCCTGGCTTGAT
GCAGGTTATAATGTCACAACAGAACCTGCTAAAATAAGTAAAGTA
TCTTTTGGTGCCAATATTCCTTTAAACCCTAAAATTGCTGTTGAT
CTTGAGGAAGCAAAGGAGATACTTTCAGACCCTGGCAAAAAACTG
GTAAGTGTCAGGAGTTGGAGAGAATATATTGGTGAAGTAAGTGGT
TATAACTATATTGAGAAAAAAGGTCGTATCCCGGGATCTGTGTTC
GGGGATTGCGGAACTGATGCTTATCACATGGAGAACTACAGGAAC
CTGGACCACACTATGCGAGAATACCATGAAATTGAAGATAAATGG
AAAGAATTAGGTATAACTCCCGAAAAACGCAATGCCTTCTATTGT
GGTACTGGATGGAGAGGAAGTGAAGCATTCCTTAACGCTTGGCTC
ATGGGCTGGGACAATGCAGCGGTCTTTGACGGTGGATGGTTTGAG
TGGAGTAATAATGATCTTCCTTTTGAAACAGGTGTGCCAGAAAAA
TGA
<SEQ ID NO: 142; PRT; MzEanB3;
Methanosalsum zhilinae>
MKSISTDELLENLHRYKVIDIRSVDAYNGWKENGENRGGHIRSAK
SLPYKWVNYIDWIEIVRSKDILPQDKLVIYGYDSEKAEEVARMFE
KAGYTDLNIYPSFFEWVERNLPMDRLERYRHLVSPDWLNQLITTD
NAPEYDNDKYVICHCHYRNPVDYEKGHIPGSIPLDTNSLESEDTW
NRRSPEELKDALENAGISSETTVIVYGRFSYPKNDDPFPGSSAGH
LGAMRCAFIMLYAGVKDVRILNGGLQSWLDAGYNVTTEPAKISKV
SFGANIPLNPKIAVDLEEAKEILSDPGKKLVSVRSWREYIGEVSG
YNYIEKKGRIPGSVFGDCGTDAYHMENYRNLDHTMREYHEIEDKW
KELGITPEKRNAFYCGTGWRGSEAFLNAWLMGWDNAAVFDGGWFE
WSNNDLPFETGVPEK
<SEQ ID NO: 143; DNA; metJ;
Escherichia coli>
ATGGCTGAATGGAGCGGCGAATATATCAGCCCATACGCTGAGCAC
GGCAAGAAGAGTGAACAAGTCAAAAAGATTACGGTTTCCATTCCT
CTTAAGGTGTTAAAAATCCTCACCGATGAACGCACGCGTCGTCAG
GTGAACAACCTGCGTCACGCTACCAACAGCGAGCTGCTGTGCGAA
GCGTTTCTGCATGCCTTTACCGGGCAACCTTTGCCGGATGATGCC
GATCTGCGTAAAGAGCGCAGCGACGAAATCCCGGAAGCGGCAAAA
GAGATCATGCGTGAGATGGGGATTAACCCGGAGACGTGGGAATAC
TAA
<SEQ ID NO: 144; DNA; MetJ;
Escherichia coli>
MAEWSGEYISPYAEHGKKSEQVKKITVSIPLKVLKILTDERTRRQ
VNNLRHATNSELLCEAFLHAFTGQPLPDDADLRKERSDEIPEAAK
EIMREMGINPETWEY

1. An engineered microbial host cell capable of producing ergothioneine, wherein the host cell comprises a) a first exogenous nucleic acid sequence coding for an Egt1 enzyme capable of converting L-histidine and/or L-cysteine to hercynylcysteine sulfoxide; b) a second exogenous nucleic acid sequence coding for an Egt2 enzyme capable of converting hercynylcystenie sulfoxide to 2-sulfenohercynine; and c) a third exogenous nucleic acid sequence coding for a methionine transporter having at least 70% amino acid sequence identity to SEQ ID NO: 96.

2. The engineered microbial host cell of claim 1, wherein the methionine transporter is a YjeH protein comprising the amino acid sequence set forth in SEQ ID NO: 96.

3. The engineered microbial host cell for claim 1, wherein:
(i) the third exogenous nucleic acid sequence has at least 70% sequence identity to SEQ ID NO: 95; and/or
(ii) the third exogenous nucleic acid sequence comprises the nucleic acid sequence of SEQ ID NO: 95.

4. (canceled)

5. The engineered microbial host cell for claim 1, wherein the first exogenous nucleic acid sequence encodes a heterologous enzyme Egt1 comprising:
(i) an amino acid sequence having at least 70% sequence identity to SEQ ID NO: 18; or
(ii) the amino acid sequence of SEQ ID NO: 18.

6. (canceled)

7. The engineered microbial host cell of claim 1, wherein the second exogenous nucleic acid sequence encodes a heterologous enzyme Egt2 comprising:
(i) an amino acid sequence having at least 70% identity to SEQ ID NO: 90; or
(ii) the amino acid sequence of SEQ ID NO: 90.

8. (canceled)

9. The engineered microbial host cell of claim 1, wherein the first exogenous nucleic acid sequence encodes a heterologous enzyme Egt1 comprising the amino acid sequence of SEQ ID NO: 18 and the second exogenous nucleic acid sequence encodes a heterologous enzyme Egt2 comprising the amino acid sequence of SEQ ID NO: 90.

10. The engineered microbial host cell of claim 9, wherein the first exogenous nucleic acid sequence comprises the nucleic acid sequence of SEQ ID NO: 17 and the second exogenous nucleic acid sequence comprises the nucleic acid sequence of SEQ ID NO: 89.

11. The engineered microbial host cell for claim 1, wherein the first exogenous nucleic acid sequence encodes a heterologous enzyme Egt1 comprising:
(i) an amino acid sequence having at least 70% sequence identity to SEQ ID NO: 20; or
(ii) the amino acid sequence of SEQ ID NO: 20.

12. (canceled)

13. The engineered microbial host cell of claim 1, wherein the second exogenous nucleic acid sequence encodes a heterologous enzyme Egt2 comprising:
(i) an amino acid sequence having at least 70% identity to SEQ ID NO: 90; or
(ii) the amino acid sequence of SEQ ID NO: 90.

14. (canceled)

15. The engineered microbial host cell of claim 1, wherein:
(i) the first exogenous nucleic acid sequence encodes a heterologous enzyme Egt1 comprising the amino acid sequence of SEQ ID NO: 20 and the second exogenous nucleic acid sequence encodes a heterologous enzyme Egt2 comprising the amino acid sequence of SEQ ID NO: 90; and/or
(ii) the first exogenous nucleic acid sequence comprises the nucleic acid sequence of SEQ ID NO: 19 and the second exogenous nucleic acid sequence comprises the nucleic acid sequence of SEQ ID NO: 89.

16. (canceled)

17. The engineered microbial host cell for claim 1, wherein the first exogenous nucleic acid sequence encodes a heterologous enzyme Egt1 comprising:
(i) an amino acid sequence having at least 70% sequence identity to SEQ ID NO: 138; or
(ii) the amino acid sequence of SEQ ID NO: 138.

18. (canceled)

19. The engineered microbial host cell of claim 1, wherein the second exogenous nucleic acid sequence encodes a heterologous enzyme Egt2 comprising:
(i) an amino acid sequence having at least 70% identity to SEQ ID NO: 4; or
(ii) the amino acid sequence of SEQ ID NO: 4.

20. (canceled)

21. The engineered microbial host cell of claim 1, wherein:
(i) the first exogenous nucleic acid sequence encodes a heterologous enzyme Egt1 comprising the amino acid sequence of SEQ ID NO: 138 and the second exogenous nucleic acid sequence encodes a heterologous enzyme Egt2 comprising the amino acid sequence of SEQ ID NO: 4; and/or
(ii) the first exogenous nucleic acid sequence comprises the nucleic acid sequence of SEQ ID NO: 137 and the second exogenous nucleic acid sequence comprises the nucleic acid sequence of SEQ ID NO: 3.

22. (canceled)

23. The engineered microbial host cell of claim 1, wherein:
(i) the first exogenous nucleic acid sequence and the second exogenous nucleic acid sequence are on a self-replicating plasmid; or
(ii) the first exogenous nucleic acid sequence and the second exogenous nucleic acid sequence are integrated into the host chromosomal DNA.

24. (canceled)

25. The engineered microbial host cell of claim 1, wherein:
(i) the first exogenous nucleic acid sequence and the second exogenous nucleic acid sequence are under a constitutive promoter; or
(ii) the first exogenous nucleic acid sequence and the second exogenous nucleic acid sequence are under an inducible promoter.

26. (canceled)

27. The engineered host cell of claim 1, wherein the host cell is;
(i) a bacterial cell selected from a group consisting of Escherichia, Salmonella, Bacillus, Acinetobacter, Streptomyces, Corynebacterium, Methylosinus, Methylomona, Rhodococcus, Pseudomonas, Rhodobacter, Synechocystis, Arthrobotlys, Brevibacteria, Microbacterium, Arthrobacter, Citrobacter, Klebsiella, Pantoea, and Clostridium;
(ii) a fungal cell selected from the group consisting of Saccharomyces; Zygosaccharomyces, Kluyveromyces, Candida, Hansenula, Debaryomyces, Mucor, Pichia, Torulopsis, and Aspergillus;
(iii) an Escherichia coli cell;
(iv) a Saccharomyces cerevisiae cell; and/or
(v) a Pichia pastoris cell.

28.-31. (canceled)

32. The engineered microbial host cell of claim 1, further comprising:
(i) a mutation in tnaA gene, wherein the mutation is deletion, frameshift or point mutation and wherein such mutation leads to decrease or elimination of tryptophanase activity, and wherein the tnaA gene comprises a nucleic acid sequence having at least 70% sequence identity to SEQ ID NO: 97;
(ii) a mutation in sdaA gene, wherein the mutation is deletion, frameshift or point mutation and wherein the sdaA gene comprises a nucleic acid sequence having at least 70% sequence identity to SEQ ID NO: 99;
(iii) a mutation in yhaM gene, wherein the mutation is deletion, frameshift or point mutation and wherein the yhaM gene comprises a nucleic acid sequence having at least 70% sequence identity to SEQ ID NO: 115;
(iv) a mutation in one or more of genes associated with serine biosynthesis selected from the group consisting of serA gene with a nucleic acid sequence having at least 70% sequence identity to SEQ ID NO: 101; serB gene with a nucleic acid sequence having at least 70% sequence identity to SEQ ID NO: 102; and serC gene with a nucleic acid sequence having at least 70% sequence identity to SEQ ID NO: 105; wherein the mutation involves the use of a constitutively active promoter to upregulate the gene expression;
(v) a mutation in cysM gene coding for cysteine synthase A with an amino acid sequence having at least 70% sequence identity to SEQ ID NO: 108, wherein the mutation involves the use of a constitutively active promoter to upregulate the gene expression;
(vi) a mutation in nrdH gene having an amino acid sequence having at least 70% sequence identity to SEQ ID NO: 110, wherein the mutation involves the use of a constitutively active promoter to upregulate the gene expression; and/or
(vii) an exogenous cysE gene, wherein the cysE gene comprises an amino acid sequence having at least 70% sequence identity to SEQ ID NO: 112.

33.-38. (canceled)

39. The engineered host cell of claim 2, further comprising:
(i) an exogenous ydeE gene, wherein the ydeE gene comprises an amino acid sequence having at least 70% sequence identity to SEQ ID NO: 114;
(ii) comprising an exogenous cysB gene on a plasmid vector under an inducible promoter, wherein the cysB gene comprise an amino acid sequence having at least 70% identity SEQ ID NO: 118; and/or
(iii) an exogenous gene encoding for a protein selected from a group consisting of CysA, CysP, CysT and CysW and wherein the transporter proteins CysA, CysP, CysT and CysW comprise amino acid sequence having at least 70% identity to SEQ ID NOS: 122, 124, 126 and 128 respectively.

40.-41. (canceled)

42. The engineered microbial host cell of claim 1, further comprising a mutation in metJ gene wherein the mutation is deletion, frameshift or point mutation and wherein:
(i) the metJ gene comprises a nucleic acid sequence having at least 70% sequence identity to SEQ ID NO: 143; or
(ii) the metJ gene comprises a nucleic acid sequence as in SEQ ID NO: 143.

43. (canceled)

44. A method for producing ergothioneine comprising:
(a) culturing an engineered microbial host cell capable of producing ergothioneine, wherein the host cell comprises a) a first exogenous nucleic acid sequence coding for an Egt1 enzyme capable of converting L-histidine and/or L-cysteine to hercynylcysteine sulfoxide; b) a second exogenous nucleic acid sequence coding for an Egt2 enzyme capable of converting hercynylcystenie sulfoxide to 2-sulfenohercynine; and c) a third exogenous nucleic acid sequence coding for a methionine transporter having at least 70% amino acid sequence identity to SEQ ID NO: 96;
(b) expressing the Egt1 enzyme, the Egt2 enzyme, and the methionine transporter;
(c) feeding the engineered microbial host cell at least one substrate selected from the group consisting of histidine, methionine, cysteine and combinations thereof; and
(d) collecting ergothioneine.

45.-65. (canceled)
